SEQUENCE LISTING (338 sequences)

SEQ ID NO: 1 - protein, 6 aa, Artificial Sequence
  source: /note=“Description of Artificial Sequence Synthetic 6xHis tag”
HHHHHH

SEQ ID NO: 2 - protein, 9 aa, Homo sapiens
FLDEFMEGV

SEQ ID NO: 3 - protein, 10 aa, Homo sapiens
LTDDRLFTCY

SEQ ID NO: 4 - protein, 10 aa, Homo sapiens
LLLDDLLVSI

SEQ ID NO: 5 - protein, 9 aa, Homo sapiens
KTLTSVFQK

SEQ ID NO: 6 - protein, 9 aa, Homo sapiens
EAFIQPITR

SEQ ID NO: 7 - protein, 9 aa, Homo sapiens
RPHVPESAF

SEQ ID NO: 8 - protein, 10 aa, Homo sapiens
SLADEAEVYL

SEQ ID NO: 9 - protein, 9 aa, Homo sapiens
KIFSEVTLK

SEQ ID NO: 10 - protein, 9 aa, Homo sapiens
ETVSEQSNV

SEQ ID NO: 11 - protein, 10 aa, Homo sapiens
GIVEGLITTV

SEQ ID NO: 12 - protein, 10 aa, Homo sapiens
SLFEGIDIYT

SEQ ID NO: 13 - protein, 10 aa, Homo sapiens
FIASNGVKLV

SEQ ID NO: 14 - protein, 9 aa, Homo sapiens
CILGKLFTK

SEQ ID NO: 15 - protein, 9 aa, Homo sapiens
QTACEVLDY

SEQ ID NO: 16 - protein, 9 aa, Homo sapiens
ILNAMIAKI

SEQ ID NO: 17 - protein, 9 aa, Homo sapiens
YTDFHCQYV

SEQ ID NO: 18 - protein, 11 aa, Homo sapiens
SELFRSGLDSY

SEQ ID NO: 19 - protein, 9 aa, Homo sapiens
AEPIDIQTW

SEQ ID NO: 20 - protein, 10 aa, Homo sapiens
TLDWLLQTPK

SEQ ID NO: 21 - protein, 10 aa, Homo sapiens
GLFGDIYLAI

SEQ ID NO: 22 - protein, 9 aa, Homo sapiens
ILDKVLVHL

SEQ ID NO: 23 - protein, 9 aa, Homo sapiens
SYLDSGIHF

SEQ ID NO: 24 - protein, 10 aa, Homo sapiens
KILDAVVAQK

SEQ ID NO: 25 - protein, 9 aa, Homo sapiens
KELEGILLL

SEQ ID NO: 26 - protein, 9 aa, Homo sapiens
KINKNPKYK

SEQ ID NO: 27 - protein, 11 aa, Homo sapiens
FLEGNEVGKTY

SEQ ID NO: 28 - protein, 9 aa, Homo sapiens
AQQITKTEV

SEQ ID NO: 29 - protein, 10 aa, Homo sapiens
ACDPHSGHFV

SEQ ID NO: 30 - protein, 9 aa, Homo sapiens
FLDEFMEAV

SEQ ID NO: 31 - protein, 10 aa, Homo sapiens
LTDDRLFTCH

SEQ ID NO: 32 - protein, 10 aa, Homo sapiens
LLLDDSLVSI

SEQ ID NO: 33 - protein, 9 aa, Homo sapiens
ETLTSVFQK

SEQ ID NO: 34 - protein, 9 aa, Homo sapiens
EASIQPITR

SEQ ID NO: 35 - protein, 9 aa, Homo sapiens
GPHVPESAF

SEQ ID NO: 36 - protein, 10 aa, Homo sapiens
SLADEAEVHL

SEQ ID NO: 37 - protein, 9 aa, Homo sapiens
KIFSEVTPK

SEQ ID NO: 38 - protein, 9 aa, Homo sapiens
ETVSEESNV

SEQ ID NO: 39 - protein, 10 aa, Homo sapiens
GIVEGLMTTV

SEQ ID NO: 40 - protein, 10 aa, Homo sapiens
SLFEGIDFYT

SEQ ID NO: 41 - protein, 10 aa, Homo sapiens
FIASKGVKLV

SEQ ID NO: 42 - protein, 9 aa, Homo sapiens
CILGELFTK

SEQ ID NO: 43 - protein, 9 aa, Homo sapiens
QTTCEVLDY

SEQ ID NO: 44 - protein, 9 aa, Homo sapiens
ILNAMITKI

SEQ ID NO: 45 - protein, 9 aa, Homo sapiens
YTDFPCQYV

SEQ ID NO: 46 - protein, 11 aa, Homo sapiens
SELFRSRLDSY

SEQ ID NO: 47 - protein, 9 aa, Homo sapiens
AEPINIQTW

SEQ ID NO: 48 - protein, 10 aa, Homo sapiens
TLGWLLQTPK

SEQ ID NO: 49 - protein, 10 aa, Homo sapiens
GSFGDIYLAI

SEQ ID NO: 50 - protein, 9 aa, Homo sapiens
ILDKVLVHP

SEQ ID NO: 51 - protein, 9 aa, Homo sapiens
SYLDSGIHS

SEQ ID NO: 52 - protein, 10 aa, Homo sapiens
KILDAVVAQE

SEQ ID NO: 53 - protein, 9 aa, Homo sapiens
KELEGILLP

SEQ ID NO: 54 - protein, 9 aa, Homo sapiens
EINKNPKYK

SEQ ID NO: 55 - protein, 11 aa, Homo sapiens
FLGGNEVGKTY

SEQ ID NO: 56 - protein, 9 aa, Homo sapiens
AQQITQTEV

SEQ ID NO: 57 - protein, 10 aa, Homo sapiens
ARDPHSGHFV

SEQ ID NO: 58 - protein, 11 aa, Homo sapiens
RPHAIRRPLAL

SEQ ID NO: 59 - protein, 9 aa, Homo sapiens
VLHDDLLEA

SEQ ID NO: 60 - protein, 9 aa, Homo sapiens
DYLQYVLQI

SEQ ID NO: 61 - protein, 10 aa, Homo sapiens
KEFEDDIINW

SEQ ID NO: 62 - protein, 10 aa, Homo sapiens
EEKRGSLHVW

SEQ ID NO: 63 - protein, 11 aa, Homo sapiens
RPRAIRRPLAL

SEQ ID NO: 64 - protein, 9 aa, Homo sapiens
VLRDDLLEA

SEQ ID NO: 65 - protein, 9 aa, Homo sapiens
DYLQCVLQI

SEQ ID NO: 66 - protein, 10 aa, Homo sapiens
KEFEDGIINW

SEQ ID NO: 67 - protein, 10 aa, Homo sapiens
EEKRGSLYVW

SEQ ID NO: 68 - protein, 10 aa, Homo sapiens
ELWCRQPPYR

SEQ ID NO: 69 - protein, 10 aa, Homo sapiens
ELWCRQPPYR

SEQ ID NO: 70 - protein, 10 aa, Homo sapiens
QSYCEPSSYR

SEQ ID NO: 71 - protein, 10 aa, Homo sapiens
TVPSSSFSHR

SEQ ID NO: 72 - protein, 9 aa, Homo sapiens
EVQASKHTK

SEQ ID NO: 73 - protein, 10 aa, Homo sapiens
WVCYQYSGYR

SEQ ID NO: 74 - protein, 9 aa, Homo sapiens
SYCEPSSYR

SEQ ID NO: 75 - protein, 10 aa, Homo sapiens
ATIESVQGAK

SEQ ID NO: 76 - protein, 9 aa, Homo sapiens
TPTVPSSSF

SEQ ID NO: 77 - protein, 9 aa, Homo sapiens
WIMVLVLPK

SEQ ID NO: 78 - protein, 9 aa, Homo sapiens
ELWCRQPPY

SEQ ID NO: 79 - protein, 10 aa, Homo sapiens
DWIMVLVLPK

SEQ ID NO: 80 - protein, 10 aa, Homo sapiens
MPIEPGDIGC

SEQ ID NO: 81 - protein, 10 aa, Homo sapiens
LISACKDGKR

SEQ ID NO: 82 - protein, 9 aa, Homo sapiens
YQYSGYRGY

SEQ ID NO: 83 - protein, 9 aa, Homo sapiens
LLNEVQASK

SEQ ID NO: 84 - protein, 10 aa, Homo sapiens
DWIMVLVLPK

SEQ ID NO: 85 - protein, 9 aa, Homo sapiens
ISACKDGKR

SEQ ID NO: 86 - protein, 9 aa, Homo sapiens
KTVVNKLFK

SEQ ID NO: 87 - protein, 9 aa, Homo sapiens
NSAENGDAK

SEQ ID NO: 88 - protein, 9 aa, Homo sapiens
LWCRQPPYR

SEQ ID NO: 89 - protein, 10 aa, Homo sapiens
VQKIFHINPR

SEQ ID NO: 90 - protein, 10 aa, Homo sapiens
QSYCEPSSYR

SEQ ID NO: 91 - protein, 9 aa, Homo sapiens
SSSFSHREK

SEQ ID NO: 92 - protein, 9 aa, Homo sapiens
SYCEPSSYR

SEQ ID NO: 93 - protein, 10 aa, Homo sapiens
TVPSSSFSHR

SEQ ID NO: 94 - protein, 9 aa, Homo sapiens
TIESVQGAK

SEQ ID NO: 95 - protein, 9 aa, Homo sapiens
MIWNVQKIF

SEQ ID NO: 96 - protein, 9 aa, Homo sapiens
QSYCEPSSY

SEQ ID NO: 97 - protein, 10 aa, Homo sapiens
SSIRSFVLQY

SEQ ID NO: 98 - protein, 10 aa, Homo sapiens
FLQEETLTQM

SEQ ID NO: 99 - protein, 9 aa, Homo sapiens
VVMSWAPPV

SEQ ID NO: 100 - protein, 9 aa, Homo sapiens
CSDSKLIGY

SEQ ID NO: 101 - protein, 9 aa, Homo sapiens
EMLIKPKEL

SEQ ID NO: 102 - protein, 9 aa, Homo sapiens
ILLMTVTSI

SEQ ID NO: 103 - protein, 9 aa, Homo sapiens
SLMEHWALG

SEQ ID NO: 104 - protein, 9 aa, Homo sapiens
LLRVHTEHV

SEQ ID NO: 105 - protein, 10 aa, Homo sapiens
SLMEHWALGA

SEQ ID NO: 106 - protein, 9 aa, Homo sapiens
KMTFLFPNL

SEQ ID NO: 107 - protein, 9 aa, Homo sapiens
GLVDEQQEV

SEQ ID NO: 108 - protein, 10 aa, Homo sapiens
ALPDPILQSI

SEQ ID NO: 109 - protein, 9 aa, Homo sapiens
GVWALPDPI

SEQ ID NO: 110 - protein, 10 aa, Homo sapiens
AVVMSWAPPV

SEQ ID NO: 111 - protein, 9 aa, Homo sapiens
TSIDRFLAV

SEQ ID NO: 112 - protein, 9 aa, Homo sapiens
GPSWGLSLM

SEQ ID NO: 113 - protein, 9 aa, Homo sapiens
LLRVHTEHV

SEQ ID NO: 114 - protein, 10 aa, Homo sapiens
WVNCSSMTFL

SEQ ID NO: 115 - protein, 10 aa, Homo sapiens
VMSWAPPVGL

SEQ ID NO: 116 - protein, 9 aa, Homo sapiens
ILYKDDMGV

SEQ ID NO: 117 - protein, 9 aa, Homo sapiens
NIQARAVVM

SEQ ID NO: 118 - protein, 10 aa, Homo sapiens
HVRCKSGNKF

SEQ ID NO: 119 - protein, 10 aa, Homo sapiens
LPDPILQSIL

SEQ ID NO: 120 - protein, 10 aa, Homo sapiens
SILLMTVTSI

SEQ ID NO: 121 - protein, 9 aa, Homo sapiens
LMEHWALGA

SEQ ID NO: 122 - protein, 9 aa, Homo sapiens
LPDPILQSI

SEQ ID NO: 123 - protein, 10 aa, Homo sapiens
VLLRVHTEHV

SEQ ID NO: 124 - protein, 10 aa, Homo sapiens
FPNLKDRDFL

SEQ ID NO: 125 - protein, 9 aa, Homo sapiens
MLIKPKELV

SEQ ID NO: 126 - protein, 10 aa, Homo sapiens
ILCSLMEHWA

SEQ ID NO: 127 - protein, 9 aa, Homo sapiens
FPNLKDRDF

SEQ ID NO: 128 - protein, 10 aa, Homo sapiens
SILYKDDMGV

SEQ ID NO: 129 - protein, 9 aa, Homo sapiens
NTFRHRVVV

SEQ ID NO: 130 - protein, 10 aa, Homo sapiens
EVRTISALAI

SEQ ID NO: 131 - protein, 10 aa, Homo sapiens
VPTKLSPISI

SEQ ID NO: 132 - protein, 10 aa, Homo sapiens
LLLQDSECKA

SEQ ID NO: 133 - protein, 9 aa, Homo sapiens
SQPGPSWGL

SEQ ID NO: 134 - protein, 10 aa, Homo sapiens
KPAVSSDSDI

SEQ ID NO: 135 - protein, 9 aa, Homo sapiens
ITHTGEKPY

SEQ ID NO: 136 - protein, 10 aa, Homo sapiens
ITHTGEKPYK

SEQ ID NO: 137 - protein, 9 aa, Homo sapiens
KFSNSNIYK

SEQ ID NO: 138 - protein, 10 aa, Homo sapiens
LTRGTFANIK

SEQ ID NO: 139 - protein, 10 aa, Homo sapiens
LTDFGLSKIM

SEQ ID NO: 140 - protein, 9 aa, Homo sapiens
LTDFGLSKI

SEQ ID NO: 141 - protein, 9 aa, Homo sapiens
KLTDFGLSK

SEQ ID NO: 142 - protein, 9 aa, Homo sapiens
SLSLGAHQK

SEQ ID NO: 143 - protein, 10 aa, Homo sapiens
FLKHKQSCAV

SEQ ID NO: 144 - protein, 9 aa, Homo sapiens
GVMTSCFLK

SEQ ID NO: 145 - protein, 9 aa, Homo sapiens
FLKHKQSCA

SEQ ID NO: 146 - protein, 9 aa, Homo sapiens
GLLCAFTLK

SEQ ID NO: 147 - protein, 9 aa, Homo sapiens
HLCGLLCAF

SEQ ID NO: 148 - protein, 9 aa, Homo sapiens
LSIFKISSL

SEQ ID NO: 149 - protein, 10 aa, Homo sapiens
VMTSCFLKHK

SEQ ID NO: 150 - protein, 9 aa, Homo sapiens
MTSCFLKHK

SEQ ID NO: 151 - protein, 10 aa, Homo sapiens
KISSLEGRSK

SEQ ID NO: 152 - protein, 9 aa, Homo sapiens
LSIFKISSL

SEQ ID NO: 153 - protein, 9 aa, Homo sapiens
RPTFYRQGL

SEQ ID NO: 154 - protein, 9 aa, Homo sapiens
EVAGFVFDK

SEQ ID NO: 155 - protein, 10 aa, Homo sapiens
EVAGFVFDKK

SEQ ID NO: 156 - protein, 10 aa, Homo sapiens
FTAGGEPCLY

SEQ ID NO: 157 - protein, 9 aa, Homo sapiens
FSIVGGYGR

SEQ ID NO: 158 - protein, 9 aa, Homo sapiens
YMKKAEAPL

SEQ ID NO: 159 - protein, 9 aa, Homo sapiens
APITTTTTV

SEQ ID NO: 160 - protein, 10 aa, Homo sapiens
HSIPLRQSVK

SEQ ID NO: 161 - protein, 10 aa, Homo sapiens
ISYLGRDRLR

SEQ ID NO: 162 - protein, 9 aa, Homo sapiens
APGFNTTPA

SEQ ID NO: 163 - protein, 10 aa, Homo sapiens
KAFFCDDHTR

SEQ ID NO: 164 - protein, 10 aa, Homo sapiens
RPHGDLPIYV

SEQ ID NO: 165 - protein, 10 aa, Homo sapiens
RLRQEVYLSL

SEQ ID NO: 166 - protein, 10 aa, Homo sapiens
YMKKAEAPLL

SEQ ID NO: 167 - protein, 10 aa, Homo sapiens
RLRQEVYLSL

SEQ ID NO: 168 - protein, 9 aa, Homo sapiens
CLTKRSIAF

SEQ ID NO: 169 - protein, 10 aa, Homo sapiens
FTAGGEPCLY

SEQ ID NO: 170 - protein, 9 aa, Homo sapiens
EYVLNTTAR

SEQ ID NO: 171 - protein, 10 aa, Homo sapiens
DPKLGTAQPL

SEQ ID NO: 172 - protein, 9 aa, Homo sapiens
QTHEPEFDY

SEQ ID NO: 173 - protein, 10 aa, Homo sapiens
APITTTTTVT

SEQ ID NO: 174 - protein, 9 aa, Homo sapiens
EAPLLEEQR

SEQ ID NO: 175 - protein, 10 aa, Homo sapiens
CLTKRSIAFL

SEQ ID NO: 176 - protein, 10 aa, Homo sapiens
APGFNTTPAT

SEQ ID NO: 177 - protein, 9 aa, Homo sapiens
TTAPITTTT

SEQ ID NO: 178 - protein, 9 aa, Homo sapiens
YMKKAEAPL

SEQ ID NO: 179 - protein, 9 aa, Homo sapiens
IPGFKFDNL

SEQ ID NO: 180 - protein, 10 aa, Homo sapiens
ELWCRQPPYR

SEQ ID NO: 181 - protein, 10 aa, Homo sapiens
ELWCRQPPYR

SEQ ID NO: 182 - protein, 10 aa, Homo sapiens
QSYCEPSSYR

SEQ ID NO: 183 - protein, 10 aa, Homo sapiens
TVPSSSFSHR

SEQ ID NO: 184 - protein, 9 aa, Homo sapiens
EVQASKHTK

SEQ ID NO: 185 - protein, 10 aa, Homo sapiens
WVCYQYSGYR

SEQ ID NO: 186 - protein, 9 aa, Homo sapiens
SYCEPSSYR

SEQ ID NO: 187 - protein, 10 aa, Homo sapiens
ATIESVQGAK

SEQ ID NO: 188 - protein, 9 aa, Homo sapiens
TPTVPSSSF

SEQ ID NO: 189 - protein, 9 aa, Homo sapiens
WIMVLVLPK

SEQ ID NO: 190 - protein, 9 aa, Homo sapiens
ELWCRQPPY

SEQ ID NO: 191 - protein, 10 aa, Homo sapiens
DWIMVLVLPK

SEQ ID NO: 192 - protein, 10 aa, Homo sapiens
MPIEPGDIGC

SEQ ID NO: 193 - protein, 10 aa, Homo sapiens
LISACKDGKR

SEQ ID NO: 194 - protein, 9 aa, Homo sapiens
YQYSGYRGY

SEQ ID NO: 195 - protein, 9 aa, Homo sapiens
LLNEVQASK

SEQ ID NO: 196 - protein, 10 aa, Homo sapiens
DWIMVLVLPK

SEQ ID NO: 197 - protein, 9 aa, Homo sapiens
ISACKDGKR

SEQ ID NO: 198 - protein, 9 aa, Homo sapiens
KTVVNKLFK

SEQ ID NO: 199 - protein, 9 aa, Homo sapiens
NSAENGDAK

SEQ ID NO: 200 - protein, 9 aa, Homo sapiens
LWCRQPPYR

SEQ ID NO: 201 - protein, 10 aa, Homo sapiens
VQKIFHINPR

SEQ ID NO: 202 - protein, 10 aa, Homo sapiens
QSYCEPSSYR

SEQ ID NO: 203 - protein, 9 aa, Homo sapiens
SSSFSHREK

SEQ ID NO: 204 - protein, 9 aa, Homo sapiens
SYCEPSSYR

SEQ ID NO: 205 - protein, 10 aa, Homo sapiens
TVPSSSFSHR

SEQ ID NO: 206 - protein, 9 aa, Homo sapiens
TIESVQGAK

SEQ ID NO: 207 - protein, 9 aa, Homo sapiens
MIWNVQKIF

SEQ ID NO: 208 - protein, 9 aa, Homo sapiens
QSYCEPSSY

SEQ ID NO: 209 - protein, 10 aa, Homo sapiens
SSIRSFVLQY

SEQ ID NO: 210 - protein, 10 aa, Homo sapiens
ELWRRQPPYR

SEQ ID NO: 211 - protein, 10 aa, Homo sapiens
ELWRRQPPYR

SEQ ID NO: 212 - protein, 10 aa, Homo sapiens
QSYCEPPSYR

SEQ ID NO: 213 - protein, 10 aa, Homo sapiens
TVPSGSFSHR

SEQ ID NO: 214 - protein, 9 aa, Homo sapiens
EVQASEHTK

SEQ ID NO: 215 - protein, 10 aa, Homo sapiens
WVCYQYPGYR

SEQ ID NO: 216 - protein, 9 aa, Homo sapiens
SYCEPPSYR

SEQ ID NO: 217 - protein, 10 aa, Homo sapiens
AAIESVQGAK

SEQ ID NO: 218 - protein, 9 aa, Homo sapiens
TPTVPSGSF

SEQ ID NO: 219 - protein, 9 aa, Homo sapiens
WIMALVLPK

SEQ ID NO: 220 - protein, 9 aa, Homo sapiens
ELWRRQPPY

SEQ ID NO: 221 - protein, 10 aa, Homo sapiens
DWIMALVLPK

SEQ ID NO: 222 - protein, 10 aa, Homo sapiens
MPIEPGDIGY

SEQ ID NO: 223 - protein, 10 aa, Homo sapiens
LISACKDGKP

SEQ ID NO: 224 - protein, 9 aa, Homo sapiens
YQYPGYRGY

SEQ ID NO: 225 - protein, 9 aa, Homo sapiens
LLNEVQASE

SEQ ID NO: 226 - protein, 10 aa, Homo sapiens
DWIMALVLPK

SEQ ID NO: 227 - protein, 9 aa, Homo sapiens
ISACKDGKP

SEQ ID NO: 228 - protein, 9 aa, Homo sapiens
KTVVNKLFE

SEQ ID NO: 229 - protein, 9 aa, Homo sapiens
NPAENGDAK

SEQ ID NO: 230 - protein, 9 aa, Homo sapiens
LWRRQPPYR

SEQ ID NO: 231 - protein, 10 aa, Homo sapiens
AQKIFHINPR

SEQ ID NO: 232 - protein, 10 aa, Homo sapiens
QSYCEPPSYR

SEQ ID NO: 233 - protein, 9 aa, Homo sapiens
SGSFSHREK

SEQ ID NO: 234 - protein, 9 aa, Homo sapiens
SYCEPPSYR

SEQ ID NO: 235 - protein, 10 aa, Homo sapiens
TVPSGSFSHR

SEQ ID NO: 236 - protein, 9 aa, Homo sapiens
AIESVQGAK

SEQ ID NO: 237 - protein, 9 aa, Homo sapiens
MIWNAQKIF

SEQ ID NO: 238 - protein, 9 aa, Homo sapiens
QSYCEPPSY

SEQ ID NO: 239 - protein, 10 aa, Homo sapiens
SSIRGFVLQY

SEQ ID NO: 240 - protein, 10 aa, Homo sapiens
FLQEETLTQM

SEQ ID NO: 241 - protein, 9 aa, Homo sapiens
VVMSWAPPV

SEQ ID NO: 242 - protein, 9 aa, Homo sapiens
CSDSKLIGY

SEQ ID NO: 243 - protein, 9 aa, Homo sapiens
EMLIKPKEL

SEQ ID NO: 244 - protein, 9 aa, Homo sapiens
ILLMTVTSI

SEQ ID NO: 245 - protein, 9 aa, Homo sapiens
SLMEHWALG

SEQ ID NO: 246 - protein, 9 aa, Homo sapiens
LLRVHTEHV

SEQ ID NO: 247 - protein, 10 aa, Homo sapiens
SLMEHWALGA

SEQ ID NO: 248 - protein, 9 aa, Homo sapiens
KMTFLFPNL

SEQ ID NO: 249 - protein, 9 aa, Homo sapiens
GLVDEQQEV

SEQ ID NO: 250 - protein, 10 aa, Homo sapiens
ALPDPILQSI

SEQ ID NO: 251 - protein, 9 aa, Homo sapiens
GVWALPDPI

SEQ ID NO: 252 - protein, 10 aa, Homo sapiens
AVVMSWAPPV

SEQ ID NO: 253 - protein, 9 aa, Homo sapiens
TSIDRFLAV

SEQ ID NO: 254 - protein, 9 aa, Homo sapiens
GPSWGLSLM

SEQ ID NO: 255 - protein, 9 aa, Homo sapiens
LLRVHTEHV

SEQ ID NO: 256 - protein, 10 aa, Homo sapiens
WVNCSSMTFL

SEQ ID NO: 257 - protein, 10 aa, Homo sapiens
VMSWAPPVGL

SEQ ID NO: 258 - protein, 10 aa, Homo sapiens
FLQEERLTQM

SEQ ID NO: 259 - protein, 9 aa, Homo sapiens
VVLSWAPPV

SEQ ID NO: 260 - protein, 9 aa, Homo sapiens
CWDSKLIGY

SEQ ID NO: 261 - protein, 9 aa, Homo sapiens
EMLSKPKEL

SEQ ID NO: 262 - protein, 9 aa, Homo sapiens
ILLMTVISI

SEQ ID NO: 263 - protein, 9 aa, Homo sapiens
SLMEPWALG

SEQ ID NO: 264 - protein, 9 aa, Homo sapiens
LLRVHTEQV

SEQ ID NO: 265 - protein, 10 aa, Homo sapiens
SLMEPWALGA

SEQ ID NO: 266 - protein, 9 aa, Homo sapiens
KMTFLFANL

SEQ ID NO: 267 - protein, 9 aa, Homo sapiens
GLVDEQQKV

SEQ ID NO: 268 - protein, 10 aa, Homo sapiens
ALPGPILQSI

SEQ ID NO: 269 - protein, 9 aa, Homo sapiens
GVWALPGPI

SEQ ID NO: 270 - protein, 10 aa, Homo sapiens
AVVLSWAPPV

SEQ ID NO: 271 - protein, 9 aa, Homo sapiens
ISIDRFLAV

SEQ ID NO: 272 - protein, 9 aa, Homo sapiens
GPSRGLSLM

SEQ ID NO: 273 - protein, 9 aa, Homo sapiens
LLRVHTEQV

SEQ ID NO: 274 - protein, 10 aa, Homo sapiens
WVNRSSMTFL

SEQ ID NO: 275 - protein, 10 aa, Homo sapiens
VLSWAPPVGL

SEQ ID NO: 276 - DNA, 24 bp, Artificial Sequence
  source: /note=“Description of Artificial Sequence Synthetic primer”
gcacaacagttccctgacttgcac

SEQ ID NO: 277 - DNA, 24 bp, Artificial Sequence
  source: /note=“Description of Artificial Sequence Synthetic primer”
tcatcaaccatgcaagcctgacct

SEQ ID NO: 278 - DNA, 24 bp, Artificial Sequence
  source: /note=“Description of Artificial Sequence Synthetic primer”
gtctctagagagaagaaggagcgc

SEQ ID NO: 279 - DNA, 24 bp, Artificial Sequence
  source: /note=“Description of Artificial Sequence Synthetic primer”
acatatgagagtggatttgtcatt

SEQ ID NO: 280 - DNA, 25 bp, Artificial Sequence
  source: /note=“Description of Artificial Sequence Synthetic primer”
atacttcagtgagacacagagaaac

SEQ ID NO: 281 - DNA, 24 bp, Artificial Sequence
  source: /note=“Description of Artificial Sequence Synthetic primer”
ttccctaactatagctctgagctg

SEQ ID NO: 282 - DNA, 20 bp, Artificial Sequence
  source: /note=“Description of Artificial Sequence Synthetic primer”
aggcctgagggatccgtctc

SEQ ID NO: 283 - DNA, 22 bp, Artificial Sequence
  source: /note=“Description of Artificial Sequence Synthetic primer”
cctgaatgccccaacagctctc

SEQ ID NO: 284 - DNA, 24 bp, Artificial Sequence
  source: /note=“Description of Artificial Sequence Synthetic primer”
atttactttaacaacaacgttccg

SEQ ID NO: 285 - DNA, 24 bp, Artificial Sequence
  source: /note=“Description of Artificial Sequence Synthetic primer”
cctaaatctccagacaaagctcac

SEQ ID NO: 286 - DNA, 24 bp, Artificial Sequence
  source: /note=“Description of Artificial Sequence Synthetic primer”
ccacggagtcaggggacacagcac

SEQ ID NO: 287 - DNA, 24 bp, Artificial Sequence
  source: /note=“Description of Artificial Sequence Synthetic primer”
tccaacctgcaaagcttgaggact

SEQ ID NO: 288 - DNA, 18 bp, Artificial Sequence
  source: /note=“Description of Artificial Sequence Synthetic primer”
catgggctgaggctgatc

SEQ ID NO: 289 - DNA, 18 bp, Artificial Sequence
  source: /note=“Description of Artificial Sequence Synthetic primer”
caaggagaagtccccaat

SEQ ID NO: 290 - DNA, 18 bp, Artificial Sequence
  source: /note=“Description of Artificial Sequence Synthetic primer”
ggtgagggtacaactgcc

SEQ ID NO: 291 - DNA, 24 bp, Artificial Sequence
  source: /note=“Description of Artificial Sequence Synthetic primer”
gtctctcgaaaagagaagaggaat

SEQ ID NO: 292 - DNA, 24 bp, Artificial Sequence
  source: /note=“Description of Artificial Sequence Synthetic primer”
agtgtctctcgacaggcacaggct

SEQ ID NO: 293 - DNA, 24 bp, Artificial Sequence
  source: /note=“Description of Artificial Sequence Synthetic primer”
aaagagtctaaacaggatgagtcc

SEQ ID NO: 294 - DNA, 20 bp, Artificial Sequence
  source: /note=“Description of Artificial Sequence Synthetic primer”
ggagatatagctgaagggta

SEQ ID NO: 295 - DNA, 24 bp, Artificial Sequence
  source: /note=“Description of Artificial Sequence Synthetic primer”
gatgagtcaggaatgccaaaggaa

SEQ ID NO: 296 - DNA, 24 bp, Artificial Sequence
  source: /note=“Description of Artificial Sequence Synthetic primer”
tcctctcactgtgacatcggccca

SEQ ID NO: 297 - DNA, 24 bp, Artificial Sequence
  source: /note=“Description of Artificial Sequence Synthetic primer”
agctctgaggtgccccagaatctc

SEQ ID NO: 298 - DNA, 24 bp, Artificial Sequence
  source: /note=“Description of Artificial Sequence Synthetic primer”
aagtgatcttgcgctgtgtcccca

SEQ ID NO: 299 - DNA, 22 bp, Artificial Sequence
  source: /note=“Description of Artificial Sequence Synthetic primer”
aggacccccagttcctcatttc

SEQ ID NO: 300 - DNA, 24 bp, Artificial Sequence
  source: /note=“Description of Artificial Sequence Synthetic primer”
cccagtttggaaagccagtgaccc

SEQ ID NO: 301 - DNA, 24 bp, Artificial Sequence
  source: /note=“Description of Artificial Sequence Synthetic primer”
tcaacagtctccagaataaggacg

SEQ ID NO: 302 - DNA, 23 bp, Artificial Sequence
  source: /note=“Description of Artificial Sequence Synthetic primer”
gacagcggaagtggttgcggggt

SEQ ID NO: 303 - DNA, 24 bp, Artificial Sequence
  source: /note=“Description of Artificial Sequence Synthetic primer”
cgggctgctccttgaggggctgcg

SEQ ID NO: 304 - DNA, 43 bp, Artificial Sequence
  source: /note=“Description of Artificial Sequence Synthetic primer”
gacgtcggatcccaccatgggtcccggaattaagaaaacagag

SEQ ID NO: 305 - DNA, 61 bp, Artificial Sequence
  source: /note=“Description of Artificial Sequence Synthetic primer”
cccggggcggccgcctaatggtgatggtgatggtgacattctaattcttctccactgtaaa

SEQ ID NO: 306 - protein, 9 aa, Homo sapiens
LTYSGRKTA

SEQ ID NO: 307 - protein, 9 aa, Homo sapiens
LTYSHRKYA

SEQ ID NO: 308 - protein, 10 aa, Homo sapiens
LTYSEGRKTA

SEQ ID NO: 309 - protein, 9 aa, Homo sapiens
LTYSAPNLV

SEQ ID NO: 310 - protein, 15 aa, Homo sapiens
LTYSGLFARYMSWEL

SEQ ID NO: 311 - protein, 11 aa, Homo sapiens
ESVANGHPVLT

SEQ ID NO: 312 - protein, 14 aa, Homo sapiens
ESVANGFTLISNQR

SEQ ID NO: 313 - protein, 5 aa, Homo sapiens
NGHSE

SEQ ID NO: 314 - protein, 15 aa, Homo sapiens
NGHSESLKHIVANSE

SEQ ID NO: 315 - DNA, 21 bp, Homo sapiens
cttgatcggatcgtagctacg

SEQ ID NO: 316 - DNA, 21 bp, Homo sapiens
cttgaacggatcgtagctacg

SEQ ID NO: 317 - protein, 7 aa, Homo sapiens
LDRIVAT

SEQ ID NO: 318 - protein, 7 aa, Homo sapiens
LERIVAT

SEQ ID NO: 319 - protein, 14 aa, Homo sapiens
LYSGLFARYMSWEL

SEQ ID NO: 320 - protein, 13 aa, Homo sapiens
ESVANGFTLSNQR

SEQ ID NO: 321 - protein, 10 aa, Homo sapiens
MPIEPGDIGY

SEQ ID NO: 322 - protein, 10 aa, Homo sapiens
MPIEPGDIGC

SEQ ID NO: 323 - protein, 9 aa, Homo sapiens
TPTVPSGSF

SEQ ID NO: 324 - protein, 9 aa, Homo sapiens
TPTVPSSSF

SEQ ID NO: 325 - protein, 9 aa, Homo sapiens
VVLSWAPPV

SEQ ID NO: 326 - protein, 78 aa, Homo sapiens
GPGIKKTERRARSSPKSNDSDLQEYELEVKRVQDILSGIEKPQVSNIQARAVVLSWAPPVGLSCGPHSGLSFPYSYEV

SEQ ID NO: 327 - protein, 78 aa, Mus musculus
GPGIKKTERRARSSPKSSDSDLQEYELEVKRVQDILSGIEKPQVSNIQARAVVLSWAPPVGLSCGPHGGLSFPYSYEV

SEQ ID NO: 328 - protein, 80 aa, Gallus gallus
APGVKKPERRARSSPKSTEQEPHEYDSETKRVQDILSGMEKPQVTNIQARTVLLSWSPPAGLLNTDRHNNGLPYACTYEV

SEQ ID NO: 329 - protein, 76 aa, Danio rerio
KKPTRGARSSPRSSEPELQDHDSEAKRVQDVLSGMEKPQVLNIQSRTARLTWAPPAGLQNRERHSNGHPFTCSYEV

SEQ ID NO: 330 - protein, 78 aa, Homo sapiens
IPGLTDQKTVPTPTVPSGSFSHREKPSIFYQQEWPDSYATEKALKVSTGPGPADQKTEIPAVQSSSYPQREKPSVLYP

SEQ ID NO: 331 - protein, 77 aa, Mus musculus
SEWLARPSEVSEALIQATSETSSDLANSCFSISQHPLTEGLQGKAESGVLTRCGDAKYSSLYENLGAQSERIAVLQR

SEQ ID NO: 332 - protein, 76 aa, Gallus gallus
EIKAELLLSAKKSGQAKGTRSYSSLAASVYSCNQEADEEHSKASSDKRFHSDSQTQAFRTKELLEPSLQHVVPLYR

SEQ ID NO: 333 - protein, 78 aa, Homo sapiens
LPFPKDASLNKCSFLHPEPVVGSKMHKMPDLFIIGSGEAMLQLIPPFQCRRHCQSVAMPIEPGDIGYVDTTHWKVYVI

SEQ ID NO: 334 - protein, 78 aa, Mus musculus
LPFPKDSSLNKCFLIQPEPVVGSKMHKVHDLFTLGSGEAMLQLIPPFQCRTHCQSVAMPIESGDIGYADAAHWKVYIV

SEQ ID NO: 335 - protein, 77 aa, Gallus gallus
LSFPKTVSLENCFLIRHPDLGNKSYSLHSLFVVGSGHLTLTVAPLDKCRGHCEMFKVDLEAGDLGYASMDYWMMSFV

SEQ ID NO: 336 - protein, 78 aa, Danio rerio
CPLLEIWSSTLQRCRLSSRRPQPSRVQVLGWMVVADGSPDVRLLPVQRCRKHCRSFSLRLEPGDMVFADSQIWLMELS

SEQ ID NO: 337 - protein, 9 aa, Homo sapiens
VVMSWAPPV

SEQ ID NO: 338 - DNA, 114 bp, Artificial Sequence
  source: /note=“Description of Artificial Sequence Synthetic polynucleotide”
caaggcttgacgactcggccgtgtatctctgtgccagcagcttagttttcgggacagggttcgtttcgggctacgagcagtacttcgggccgggcaccaggctcacggtcacag

(19) United States
(12) Patent Application Publication
(10) Pub. No.: US 2021/0220455 A1
(43) Pub. Date: Jul. 22, 2021

(54) COMPOSITIONS AND METHODS FOR PERSONALIZED NEOPLASIA VACCINES

(71) Applicants: The Broad Institute, Inc., Cambridge, MA (US); Dana Farber Cancer Institute, Inc., Boston, MA (US); The General Hospital Corporation, Boston, MA (US)

(72) Inventors: Nir Hacohen, Brookline, MA (US); Catherine Ju-Ying Wu, Brookline, MA (US); Edward F. Fritsch, Concord, MA (US)

(21) Appl. No.: 17/089,408

(22) Filed: Nov. 4, 2020

Related U.S. Application Data
(63) Continuation of application No. 14/877,125, filed on Oct. 7, 2015.
Application No. 14/877,125 is a continuation-in-part of application No. PCT/US14/33185, filed on Apr. 7, 2014.
(60) Provisional application No. 61/869,721, filed on Aug. 25, 2013.
(60) Provisional application No. 61/809,406, filed on Apr. 7, 2013.

(51) Int. Cl. A61K 39/00; A61K 39/39

(52) U.S. Cl. CPC A61K 39/0011; A61K 39/39; A61K 2039/80; A61K 2039/55561; A61K 2039/70; A61K 2039/545

(57) ABSTRACT

The invention provides a method of making a personalized neoplasia vaccine for a subject diagnosed as having a neoplasia, which includes identifying a plurality of mutations in the neoplasia, analyzing the plurality of mutations to identify a subset of at least five neo-antigenic mutations predicted to encode neo-antigenic peptides, the neo-antigenic mutations selected from the group consisting of missense mutations, neoORF mutations, and any combination thereof, and producing, based on the identified subset, a personalized neoplasia vaccine.

### RELATED APPLICATIONS

This application is a continuation application of U.S. patent application Ser. No. 14/877,125, filed on Oct. 7, 2015, now allowed, which is a continuation-in-part of International Application No. PCT/US2014/033185 filed Apr. 7, 2014 and which published as PCT Publication No. WO 2014/168874 on Oct. 16, 2014 and which claims the benefit of and priority to U.S. Provisional Patent Application No. 61/809,406, filed Apr. 7, 2013 and U.S. Provisional Patent Application No. 61/869,721, filed Aug. 25, 2013, the contents of which are incorporated herein by reference.

### STATEMENT AS TO FEDERALLY SPONSORED RESEARCH

This work was supported by the following grants from the National Institutes of Health, Grant No's: NIH/NCI-1R01CA155010-02 and NHLBI-5R01HL103532-03. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 28, 2015, is named 47608002002.txt and is 60,076 bytes in size.

### FIELD OF THE INVENTION

The present invention relates to personalized strategies for the treatment of neoplasia. More particularly, the present invention relates to the identification and use of a patient specific pool of tumor specific neo-antigens in a personalized tumor vaccine for treatment of the subject.

### BACKGROUND

Approximately 1.6 million Americans are diagnosed with neoplasia every year, and approximately 580,000 people in the United States are expected to die of the disease in 2013. Over the past few decades there been significant improvements in the detection, diagnosis, and treatment of neoplasia, which have significantly increased the survival rate for many types of neoplasia. However, only about 60% of people diagnosed with neoplasia are still alive 5 years after the onset of treatment, which makes neoplasia the second leading cause of death in the United States.

Currently, there are a number of different existing cancer therapies, including ablation techniques (e.g., surgical procedures, cryogenic/heat treatment, ultrasound, radiofrequency, and radiation) and chemical techniques (e.g., pharmaceutical agents, cytotoxic/chemotherapeutic agents, monoclonal antibodies, and various combinations thereof). Unfortunately, such therapies are frequently associated with serious risk, toxic side effects, and extremely high costs, as well as uncertain efficacy.

There is a growing interest in cancer therapies that seek to target cancerous cells with a patient's own immune system (e.g., cancer vaccines) because such therapies may mitigate/eliminate some of the above-described disadvantages. Cancer vaccines are typically composed of tumor antigens and immunostimulatory molecules (e.g., cytokines or TLR ligands) that work together to induce antigen-specific cytotoxic T cells that target and destroy tumor cells. Current cancer vaccines typically contain shared tumor antigens, which are native proteins (i.e.—proteins encoded by the DNA of all the normal cells in the individual) that are selectively expressed or over-expressed in tumors found in many individuals. While such shared tumor antigens are useful in identifying particular types of tumors, they are not ideal as immunogens for targeting a T-cell response to a particular tumor type because they are subject to the immune dampening effects of self-tolerance. Accordingly, there is a need for methods of identifying more effective tumor antigens that may be used for neoplasia vaccines.

### SUMMARY OF THE INVENTION

The present invention relates to a strategy for the personalized treatment of neoplasia, and more particularly to the identification and use of a personalized cancer vaccine consisting essentially of a pool of tumor-specific and patient-specific neo-antigens for the treatment of tumors in a subject. As described below, the present invention is based, at least in part, on the discovery that whole genome/exome sequencing may be used to identify all, or nearly all, mutated neo-antigens that are uniquely present in a neoplasia/tumor of an individual patient, and that this collection of mutated neo-antigens may be analyzed to identify a specific, optimized subset of neo-antigens for use as a personalized neoplasia vaccine for treatment of the patient's neoplasia/tumor.

In one aspect, the invention provides a method of making a personalized neoplasia vaccine for a subject diagnosed as having a neoplasia, which includes identifying a plurality of mutations in the neoplasia, analyzing the plurality of mutations to identify a subset of at least five neo-antigenic mutations predicted to encode neo-antigenic peptides, the neo-antigenic mutations selected from the group consisting of missense mutations, neoORF mutations, and any combination thereof, and producing, based on the identified subset, a personalized neoplasia vaccine.

In an embodiment, the invention provides that the identifying step further includes sequencing the genome, transcriptome, or proteome of the neoplasia.

In another embodiment, the analyzing step may further include determining one or more characteristics associated with the subset of at least five neo-antigenic mutations predicted to encode neo-antigenic peptides, the characteristics selected from the group consisting of molecular weight, cysteine content, hydrophilicity, hydrophobicity, charge, and binding affinity; and ranking, based on the determined characteristics, each of the neo-antigenic mutations within the identified subset of at least five neo-antigenic mutations. In an embodiment, the top 5-30 ranked neo-antigenic mutations are included in the personalized neoplasia vaccine. In another embodiment, the neo-antigenic mutations are ranked according to the order shown in FIG. 8.

In one embodiment, the personalized neoplasia vaccine comprises at least about 20 neo-antigenic peptides corresponding to the neo-antigenic mutations.

In another embodiment, the personalized neoplasia vaccine comprises one or more DNA molecules capable of expressing at least about 20 neo-antigenic peptides corresponding to the neo-antigenic mutations. In another embodiment, the personalized neoplasia vaccine comprises one or more RNA molecules capable of expressing at least 20 neo-antigenic peptides corresponding to the neo-antigenic mutations.

In embodiments, the personalized neoplasia vaccine comprises neoORF mutations predicted to encode a neoORF polypeptide having a Kd of ≤500 nM.

In another embodiment, the personalized neoplasia vaccine comprises missense mutations predicted to encode a polypeptide having a Kd of ≤150 nM, wherein the native cognate protein has a Kd of ≥1000 nM or ≤150 nM.

In another embodiment, the at least about 20 neo-antigenic peptides range from about 5 to about 50 amino acids in length. In another embodiment, the at least about 20 neo-antigenic peptides range from about 15 to about 35 amino acids in length. In another embodiment, the at least about 20 neo-antigenic peptides range from about 18 to about 30 amino acids in length. In another embodiment, the at least about 20 neo-antigenic peptides range from about 6 to about 15 amino acids in length. In yet another embodiment, the at least about 20 neo-antigenic peptides are 15, 16, 17, 18, 19, 20, 21, 22, 23, 24, or 25 amino acids in length.

In one embodiment, the personalized neoplasia vaccine further includes an adjuvant. In other embodiments, the adjuvant is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM, vector system, PLGA microparticles, resiquimod, SRL172, Virosomes and other Virus-like particles, YF-17D, VEGF trap, R848, beta-glucan, Pam3Cys, Aquila's QS21 stimulon, vadimezan, and/or AsA404 (DMXAA). In a preferred embodiment, the adjuvant is poly-ICLC.

In another aspect, the invention includes a method of treating a subject diagnosed as having a neoplasia with a personalized neoplasia vaccine, which includes identifying a plurality of mutations in the neoplasia; analyzing the plurality of mutations to identify a subset of at least five neo-antigenic mutations predicted to encode expressed neo-antigenic peptides, the neo-antigenic mutations selected from the group consisting of missense mutations, neoORF mutations, and any combination thereof; producing, based on the identified subset, a personalized neoplasia vaccine; and administering the personalized neoplasia vaccine to the subject, thereby treating the neoplasia.

In another embodiment, the identifying step may further include sequencing the genome, transcriptome, or proteome of the neoplasia.

In yet another embodiment, the analyzing step may further include determining one or more characteristics associated with the subset of at least five neo-antigenic mutations predicted to encode expressed neo-antigenic peptides, the characteristics selected from the group consisting of molecular weight, cysteine content, hydrophilicity, hydrophobicity charge, and binding affinity; and ranking, based on the determined characteristics, each of the neo-antigenic mutations within the identified subset of at least five neo-antigenic mutations.

In one embodiment, the top 5-30 ranked neo-antigenic mutations are included in the personalized neoplasia vaccine. In another embodiment, the neo-antigenic mutations are ranked according to the order shown in FIG. 8.

In one embodiment, the personalized neoplasia vaccine comprises at least 20 neo-antigenic peptides corresponding to the neo-antigenic mutations.

In another embodiment, the personalized neoplasia vaccine comprises one or more DNA molecules capable of expressing at least 20 neo-antigenic peptides corresponding to the neo-antigenic mutations.

In one embodiment, the personalized neoplasia vaccine comprises one or more RNA molecules capable of expressing at least 20 neo-antigenic peptides corresponding to the neo-antigenic mutations.

In one embodiment, the personalized neoplasia vaccine comprises neoORF mutations predicted to encode a neoORF polypeptide having a Kd of ≤500 nM.

In another embodiment, the personalized neoplasia vaccine comprises missense mutations predicted to encode a polypeptide having a Kd of ≤150 nM, wherein the native cognate protein has a Kd of ≥1000 nM or ≤150 nM.

In one embodiment, the at least 20 neo-antigenic peptides range from about 5 to about 50 amino acids in length. In one embodiment, the at least 20 neo-antigenic peptides range from about 15 to about 35 amino acids in length. In one embodiment, the at least 20 neo-antigenic peptides range from about 18 to about 30 amino acids in length. In one embodiment, the at least 20 neo-antigenic peptides range from about 6 to about 15 amino acids in length. In one embodiment, the at least 20 neo-antigenic peptides are 15, 16, 17, 18, 19, 20, 21, 22, 23, 24, or 25 amino acids in length.

In one embodiment, the administering further includes dividing the produced vaccine into two or more sub-pools; and injecting each of the sub-pools into a different location of the patient. In one embodiment, each of the sub-pools injected into a different location comprises neo-antigenic peptides such that the number of individual peptides in the sub-pool targeting any single patient HLA is one, or as few above one as possible.

In one embodiment, the administering step further includes dividing the produced vaccine into two or more sub-pools, wherein each sub-pool comprises at least five neo-antigenic peptides selected to optimize intra-pool interactions.

In one embodiment, optimizing comprises reducing negative interaction among the neo-antigenic peptides in the same pool.

In another aspect, the invention includes a personalize neoplasia vaccine prepared according to the above-described methods.

### Definitions

To facilitate an understanding of the present invention, a number of terms and phrases are defined below:

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease (e.g., a neoplasia, tumor, etc.).

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a tumor specific neo-antigen polypeptide analog retains the biological activity of a corresponding naturally-occurring tumor specific neo-antigen polypeptide, while having certain biochemical modifications that enhance the analog's function relative to a naturally-occurring polypeptide. Such biochemical modifications could increase the analog's protease resistance, membrane permeability, or half-life, without altering, for example, ligand binding. An analog may include an unnatural amino acid.

The phrase “combination therapy” embraces the administration of a pooled sample of neoplasia/tumor specific neo-antigens and one or more additional therapeutic agents as part of a specific treatment regimen intended to provide a beneficial (additive or synergistic) effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of tumor specific neo-antigens and at least one additional therapeutic agent (e.g., a chemotherapeutic agent, an anti-angiogenesis agent, an immunosuppressive agent, an anti-inflammatory agent, and the like) at the same or different times or they can be formulated as a single, co-formulated pharmaceutical composition comprising the two compounds. As another example, a combination of the present invention (e.g., a pooled sample of tumor specific neo-antigens and at least one additional therapeutic agent) may be formulated as separate pharmaceutical compositions that can be administered at the same or different time. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence.

The phrase “combination” embraces groups of compounds or non-drug therapies useful as part of a combination therapy.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “disease” is meant any condition or disorder that damages or interferes with the normal function of a cell, tissue, or organ.

By “effective amount” is meant the amount required to ameliorate the symptoms of a disease (e.g., a neoplasia/tumor) relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism—or in the genomic DNA of a neoplasia/tumor derived from the organism—the nucleic acid molecule of the invention is derived. The term therefore includes, for example, a recombinant DNA (e.g., DNA coding for a neoORF, read-through, or InDel derived polypeptide identified in a patient's tumor) that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

A “ligand” is to be understood as meaning a molecule which has a structure complementary to that of a receptor and is capable of forming a complex with the receptor. According to the invention, a ligand is to be understood as meaning a peptide or peptide fragment that has a suitable length and suitable binding motifs in its amino acid sequence, so that the peptide or peptide fragment is capable of forming a complex with proteins of MHC class I or MHC class II.

“Mutation” for the purposes of this document means a DNA sequence found in the tumor DNA sample of a patient that is not found in the corresponding normal DNA sample of that same patient. “Mutation” may also refer to patterns in the sequence of RNA from a patient that are not attributable to expected variations based on known information for an individual gene and are reasonably considered to be novel variations in, for example, the splicing pattern of one or more genes that has been specifically altered in the tumor cells of the patient.

“Neo-antigen” or “neo-antigenic” means a class of tumor antigens that arises from a tumor-specific mutation(s) which alters the amino acid sequence of genome encoded proteins.

By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

Unless specifically stated or obvious from context, as used herein, the term “or” is understood to be inclusive. Unless specifically stated or obvious from context, as used herein, the terms “a,” “an,” and “the” are understood to be singular or plural.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neo-antigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the pooled tumor specific neo-antigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

“Proteins or molecules of the major histocompatibility complex (MHC),” “MHC molecules,” “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them to specific cells there, in particular naïve T-cells, cytotoxic T-lymphocytes or T-helper cells. The major histocompatibility complex in the genome comprises the genetic region whose gene products are expressed on the cell surface and are important for binding and presenting endogenous and/or foreign antigens, and thus for regulating immunological processes. The major histocompatibility complex is classified into two gene groups coding for different proteins: molecules of MHC class I and MHC class II. The molecules of the two MHC classes are specialized for different antigen sources. The molecules of MHC class I typically present but are not restricted to endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles.

MHC molecules of class I consist of a heavy chain and a light chain and are capable of binding a peptide of about 8 to 11 amino acids, but usually 9 or 10 amino acids, if this peptide has suitable binding motifs, and presenting it to naïve and cytotoxic T-lymphocytes. The peptide bound by the MHC molecules of class I typically but not exclusively originates from an endogenous protein antigen. The heavy chain of the MHC molecules of class I is preferably an HLA-A, HLA-B or HLA-C monomer, and the light chain is β-2-microglobulin.

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular or exogenous protein antigen. The α-chain and the β-chain are in particular HLA-DR, HLA-DQ and HLA-DP monomers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

A “receptor/ligand complex” is also to be understood as meaning a “receptor/peptide complex” or “receptor/peptide fragment complex,” in particular a peptide- or peptide fragment-presenting MHC molecule of class I or of class II.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of, or the entirety of, a specified sequence; for example, a segment of a full-length cDNA or genomic sequence, or the complete cDNA or genomic sequence. For polypeptides, the length of the reference polypeptide sequence will generally be at least about 10-2,000 amino acids, 10-1,500, 10-1,000, 10-500, or 10-100. Preferably, the length of the reference polypeptide sequence may be at least about 10-50 amino acids, more preferably at least about 10-40 amino acids, and even more preferably about 10-30 amino acids, about 10-20 amino acids, about 15-25 amino acids, or about 20 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “specifically binds” is meant a compound or antibody that recognizes and binds a polypeptide of the invention, but which does not substantially recognize and bind other molecules in a sample, for example, a biological sample.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

A “T-cell epitope” is to be understood as meaning a peptide sequence that can be bound by MHC molecules of class I or II in the form of a peptide-presenting MHC molecule or MHC complex and then, in this form, be recognized and bound by naïve T-cells, cytotoxic T-lymphocytes or T-helper cells.

As used herein, the terms “treat,” “treated,” “treating,” “treatment,” and the like refer to reducing or ameliorating a disorder and/or symptoms associated therewith (e.g., a neoplasia or tumor). It will be appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The pharmaceutical compositions typically should provide a dosage of from about 0.0001 mg to about 200 mg of compound per kilogram of body weight per day. For example, dosages for systemic administration to a human patient can range from 0.01-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, of 200 mg/kg. Pharmaceutical dosage unit forms are prepared to provide from about 0.001 mg to about 5000 mg, for example from about 100 to about 2500 mg of the compound or a combination of essential ingredients per dosage unit form.

A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

The above-mentioned and other features and advantages of the present disclosure will be better understood when reading the following detailed description taken together with the following drawings in which:

FIG. 1 depicts a flow process for making a personalized cancer vaccine according to an exemplary embodiment of the invention.

FIG. 2 shows a flow process for pre-treatment steps for generating a cancer vaccine for a melanoma patient according to an exemplary embodiment of the invention.

FIG. 3 is a flowchart depicting an approach for addressing an initial patient population study according to an exemplary embodiment of the invention. Five patients may be treated in the first cohort at an anticipated safe dose level. If fewer than two of these five patients develop a dose limiting toxicity at, or prior to, the primary safety endpoint, then 10 more patients may be recruited at that dose level to expand the analysis of the patient population (e.g., to assess efficacy, safety, etc.). If two or more dose limiting toxicities (DLTs) are observed, then the dose of poly-ICLC may be reduced by 50% and five additional patients may be treated. If fewer than two of these five patients develop a dose limiting toxicity, then 10 more patients may be recruited at that dose level. However, if two or more patients at the reduced poly-ICLC level develop a DLT, then the study will be stopped.

FIGS. 4A and 4B show examples of different types of discrete mutations and neoORFs, respectively.

FIG. 5 illustrates an immunization schedule based on a prime boost strategy according to an exemplary embodiment of the present invention. Multiple immunizations may occur over the first ˜3 weeks to maintain an early high antigen exposure during the priming phase of immune response. Patients may then be rested for eight weeks to allow memory T cells to develop and these T cells will then be boosted in order to maintain a strong ongoing response.

FIG. 6 shows a time line indicating the primary immunological endpoint according to an exemplary aspect of the invention.

FIG. 7 illustrates a time line for administering a co-therapy with checkpoint blockade antibodies to evaluate the combination of relief of local immune suppression coupled with the stimulation of new immunity according to an exemplary embodiment of the invention. As shown in the scheme, patients who enter as appropriate candidates for checkpoint blockade therapy, e.g., anti-PDL1 as shown here, may be entered and immediately treated with antibody, while the vaccine is being prepared. Patients may then be vaccinated. Checkpoint blockade antibody dosing can be continued or possibly deferred while the priming phase of vaccination occurs.

FIG. 8 is a table that shows the ranking assignments for different neo-antigenic mutations according to an exemplary embodiment of the invention.

FIG. 9 shows a schematic depicting drug product processing of individual neo-antigenic peptides into pools of 4 subgroups according to an exemplary embodiment of the invention.

FIG. 10 shows a schematic representation of a strategy to systematically discover tumor neoantigens according to an exemplary embodiment of the invention. Tumor specific mutations in cancer samples may be detected using whole-exome (WES) or whole-genome sequencing (WGS) and identified through the application of mutation calling algorithms (e.g., Mutect). Subsequently, candidate neoepitopes may be predicted using well-validated algorithms (e.g., NetMHCpan) and their identification may be refined by experimental validation for peptide-HLA binding and by confirmation of gene expression at the RNA level. These candidate neoantigens may be subsequently tested for their ability to stimulate tumor-specific T cell responses.

FIGS. 11A-C show the frequency of classes of point mutations that have the potential to generate neoantigens in chronic lymphocytic leukemia (CLL). Analysis of WES and WGS data generated from 91 CLL cases reveals that (A) missense mutations are the most frequent class of the somatic alterations with the potential to generate neo-epitopes, while (B) frameshift insertions and deletions and (C) splice-site mutations constitute less common events.

FIGS. 12A-D depict the application of the NetMHCpan prediction algorithm to functionally-defined neoepitopes and CLL cases. FIG. 12 A shows the predicted binding (IC50) to their known restricting HLA allele of 33 functionally identified cancer neoepitopes reported in literature tested by NetMHCpan, sorted on the basis of predicted binding affinity. FIG. 12B shows the distribution of the number of predicted peptides with HLA binding affinity<150 nM (black) and 150-500 nM (grey) across 31 CLL patients with available HLA typing information. FIG. 12C shows a graph comparing the predicted binding (IC50<500 nM by NetMHCpan) of peptides from 4 patients with the experimentally determined binding affinity for HLA-A and -B allele binding using a competitive MHC I allele-binding assay with synthesized peptides. The percent of predicted peptides with evidence of experimental binding (IC50<500 nM) are indicated. FIG. 12D shows that from 26 CLL patients for which HLA typing and Affymetrix U133 2.0+ gene expression data were available, the distribution of gene expression was examined for all somatically mutated genes (n=347), and for the subset of gene mutations encoding neoepitopes with predicted HLA binding scores of IC50<500 nM (n=180). No-low: genes within the lowest quartile expression; medium: genes within the 2 middle quartiles of expression; and high: genes within the highest quartile of expression.

FIGS. 13A-B show the same data as in FIG. 12D but separately for 9-mer (FIG. 13A) and 10-mer peptides (FIG. 13B). In each case, percentages of peptides with predicted IC50<150 nM and 150-500 nM, with evidence of experimental binding are indicated.

FIGS. 14A-C depict that mutations in ALMS1 and C6ORF89 in Pt 1 generate immunogenic peptides. FIG. 14A shows that 25 missense mutations were identified in Pt 1 CLL cells from which 30 peptides from 13 mutations were predicted to bind to Pt 1's MEC class I alleles. A total of 14 peptides from 9 mutations were experimentally confirmed as HLA-binding. Post-transplant T cells (7 yrs) from Pt 1 were stimulated weekly ex vivo for 4 weeks with 5 pools of 6 mutated peptides with similar predicted HLA binding, per pool, and subsequently tested by IFN-γ ELISPOT assay. FIG. 14B shows that increased IFN-γ secretion by T cells was detected against Pool 2 peptides. Negative control—Irrelevant Tax peptide; positive control—PHA. FIG. 14C shows that of Pool 2 peptides, Pt 1 T cells were reactive to mutated ALMS1 and C6ORF89 peptides (right panel; averaged results from duplicate wells are displayed). Left panel-The predicted and experimental IC50 scores (nM) of mutated and wildtype ALMS1 and C6ORF89 peptides.

FIG. 15 illustrates that the sequence context around the sites of mutations in FNDC3B, C6orf89 and ALMS1 lack evolutionary conservation. The neoepitopes generated from each of the genes are boxed. Red—conserved amino acids (aa) in all 4 species; blue—conserved aa in at least 2 of 4 species; black—absent conservation across species.

FIG. 16 shows localization of somatic mutations reported in FNDC3B, C6orf89 and ALMS1 genes. Missense mutations identified in FNDC3B, C6orf89 and ALMS1 in CLL Pts 1 and 2 compared to previously reported somatic mutations in these genes (COSMIC database) across cancers.

FIG. 17A-G shows that mutated FNDC3B generates a naturally immunogenic neoepitope in Pt 2. FIG. 17A shows 26 missense mutations were identified in Pt 2 CLL cells from which 37 peptides from 16 mutations were predicted to bind to Pt 2's MHC class I alleles. A total of 18 peptides from 12 mutations were experimentally confirmed to bind. Post-transplant T cells (˜3 yrs) from Pt 2 were stimulated with autologous DCs or B cells pulsed with 3 pools of experimentally validated binding mutated peptides (18 peptides total) for 2 weeks ex vivo (See table S6). FIG. 17B shows increased IFN-γ secretion was detected by ELISPOT assay in T cells stimulated with Pool 1 peptides. FIG. 17C shows that of Pool 1 peptides, increased IFN-γ secretion was detected against the mut-FNDC3B peptide (bottom panel; averaged results from duplicated wells are displayed). Top panel—Predicted and experimental IC50 scores of mut- and wt-FNDC3B peptides. FIG. 17D illustrates that T cells reactive to mut-FNDC3B demonstrate specificity to the mutated epitope but not the corresponding wildtype peptide (concentrations: 0.1-10 μg/ml), and are polyfunctional, secreting IFN-γ, GM-CSF and IL-2 (Tukey post-hoc tests from two-way ANOVA modeling for comparisons between T cell reactivity against mut vs wt peptide). FIG. 17E shows that Mut-FNDC3B-specific T cells are reactive in a class I-restricted manner (left), and recognize an endogenously processed and presented form of mutated FNDC3B, since they recognized HLA-A2 APCs transfected with a plasmid encoding a minigene of 300 bp encompassing the FNDC3B mutation (right) (two-sided t test). Top right—Western blot analysis-confirming expression of minigenes encoding mut- and wt-FNDC3B. FIG. 17F shows that T cells recognizing the mut-FNDC3B epitope as detected by HLA-A2⁺/mut FNDC3B tetramers are more frequently detected in T cells in Pt 2 compared to T cells from a normal donor. FIG. 17G shows expression of FNDC3B (based on Affymetrix U133Plus2 array data) in Pt 2 (triangle), CLL-B cells (n=182) and normal CD19+ B cells from healthy adult volunteers (n=24).

FIG. 18 illustrates kinetics of the mut-FNDC3B specific T cell response in relation to the transplant course. FIG. 18 shows molecular tumor burden was measured in Pt 2 using a patient tumor-specific Taqman PCR assay based on the clonotypic IgH sequence at serial time points before and after HSCT (top panel). Middle panel—Detection of mut-FNDC3B reactive T cells in comparison to wt-FNDC3B or irrelevant peptides from peripheral blood before and after allo-HSCT by IFN-γ ELISPOT following stimulation with peptide-pulsed autologous B cells. The number of IFN-γ-secreting spots per cells at each time point was measured in triplicate (Welch t test; mut vs. wt). Inset—IFN-γ secretion of T cells from 6 months post-HSCT (purple) compared to 32 months post-HSCT (red) following exposure to APCs pulsed with 0.1-10 μg/ml (log scale) mut-FNDC3B peptide. Bottom panel—Detection of mut-FNDC3B-specific TCR Vβ11 cells by nested clone-specific CDR3 PCR before and after HSCT in peripheral blood of Pt 2 (See supplementary methods). Triangles—time points at which a sample was tested; NA—no amplification; black: amplification detected, where ‘+’ indicates detectable amplification up to 2-fold and ‘++’ indicates more than 2-fold greater amplification than the median level of all samples with detectable expression of the clone-specific Vβ11 sequence.

FIGS. 19A-D show the design of mut-FNDC3B specific TCR Vβ specific primers in Pt 2. FIG. 19A shows mut-FNDC3B specific T cells detected and isolated from Pt 2 PBMCs 6 months following HSCT using an IFN-γ catch assay. FIG. 19B shows RNA from FNDC3B-reactive T cells expressed TCR Vβ11, generating an amplicon of 350 bp in length. FIG. 19C shows Vβ11-specific real time primers were designed based on the sequence of the mut-FNDC3B clone-specific CDR3 rearrangement, such that the quantitative PCR probe was positioned in the region of junctional diversity (orange). FIG. 19D shows FNDC3B-reactive T cells were monoclonal for Vβ11, as detected by spectratyping.

FIGS. 20A-G illustrate the application of the neoantigen discovery pipeline across cancers. FIG. 20A shows the comparison of overall somatic mutation rate detected across cancers by massively parallel sequencing. Red-CLL; blue-clear cell renal carcinoma (RCC) and green—melanoma. LSCC: Lung squamous cell carcinoma, Lung AdCa: Lung adenocarcinoma, ESO AdCa: Esophageal adenocarcinoma, DLBCL: Diffused large B-cell lymphoma, GBM: Glioblastoma, Papillary RCC: Papillary renal cell carcinoma, Clear Cell RCC: Clear cell renal carcinoma, CLL: Chronic lymphocytic leukemia, AML: Acute myeloid leukemia. Distribution of FIG. 20B shows the number of missense, frameshift and splice-site mutations per case in melanoma, clear cell RCC and CLL, FIG. 20C shows the average neoORF length generated per sample and FIG. 20D shows predicted neopeptides with IC50<150 nM (dashed lines) and <500 nM (solid lines) generated from missense and frameshift mutations. FIG. 20E depicts the distributions (shown by box plot) of the number of missense, frameshift and splice-site mutations per case across 13 cancers. FIG. 20F shows the summed neoORF length generated per sample. 20G shows the predicted neopeptides with IC50<150 nM and with <500 nM generated from missense and frameshift mutations. For all box plots, the left and right ends of the boxes represent the 25th and 75th percentile values, respectively, while the segment in the middle is the median. The left and right extremes of the bars extend to the minimum and maximum values.

### DETAILED DESCRIPTION OF THE INVENTION

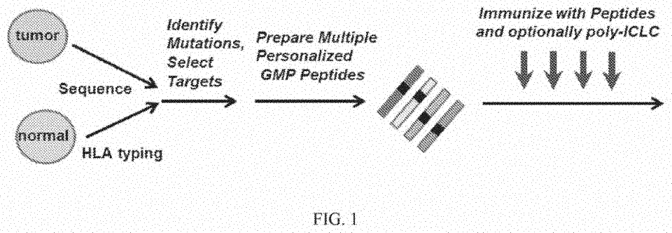

The present invention relates to personalized strategies for the treatment of neoplasia, and more particularly tumors, by administering a therapeutically effective amount of a pharmaceutical composition (e.g., a cancer vaccine) comprising a plurality of neoplasia/tumor specific neo-antigens to a subject (e.g., a mammal such as a human). As described in more detail below, the present invention is based, at least in part, on the discovery that whole genome/exome sequencing may be used to identify all, or nearly all, mutated neo-antigens that are uniquely present in a neoplasia/tumor of an individual patient, and that this collection of mutated neo-antigens may be analyzed to identify a specific, optimized subset of neo-antigens for use as a personalized cancer vaccine for treatment of the patient's neoplasia/tumor. For example, as shown in FIG. 1, a population of neoplasia/tumor specific neo-antigens may be identified by sequencing the neoplasia/tumor and normal DNA of each patient to identify tumor-specific mutations, and determining the patient's HLA allotype. The population of neoplasia/tumor specific neo-antigens and their cognate native antigens may then be subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, a plurality of peptides corresponding to a subset of these mutations may be designed and synthesized for each patient, and pooled together for use as a cancer vaccine in immunizing the patient. The neo-antigens peptides may be combined with an adjuvant (e.g., poly-ICLC) or another anti-neoplastic agent. Without being bound by theory, these neo-antigens are expected to bypass central thymic tolerance (thus allowing stronger anti-tumor T cell response), while reducing the potential for autoimmunity (e.g., by avoiding targeting of normal self-antigens).

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naïve or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines.

One of the critical barriers to developing curative and tumor-specific immunotherapy is the identification and selection of highly specific and restricted tumor antigens to avoid autoimmunity. Tumor neo-antigens, which arise as a result of genetic change (e.g., inversions, translocations, deletions, missense mutations, splice site mutations, etc.) within malignant cells, represent the most tumor-specific class of antigens. Neo-antigens have rarely been used in cancer vaccines due to technical difficulties in identifying them, selecting optimized neo-antigens, and producing neo-antigens for use in a vaccine. According to the present invention, these problems may be addressed by:
identifying all, or nearly all, mutations in the neoplasia/tumor at the DNA level using whole genome, whole exome (e.g., only captured exons), or RNA sequencing of tumor versus matched germline samples from each patient;
analyzing the identified mutations with one or more peptide-MHC binding prediction algorithms to generate a plurality of candidate neo-antigen T cell epitopes that are expressed within the neoplasia/tumor and may bind patient HLA alleles; and
synthesizing the plurality of candidate neo-antigen peptides selected from the sets of all neoORF peptides and predicted binding peptides for use in a cancer vaccine.

For example, translating sequencing information into a therapeutic vaccine may include:

(1) Prediction of personal mutated peptides that can bind to HLA molecules of the individual. Efficiently choosing which particular mutations to utilize as immunogen requires identification of the patient HLA type and the ability to predict which mutated peptides would efficiently bind to the patient's HLA alleles. Recently, neural network based learning approaches with validated binding and non-binding peptides have advanced the accuracy of prediction algorithms for the major HLA-A and -B alleles.

(2) Formulating the drug as a multi-epitope vaccine of long peptides. Targeting as many mutated epitopes as practically possible takes advantage of the enormous capacity of the immune system, prevents the opportunity for immunological escape by down-modulation of a particular immune targeted gene product, and compensates for the known inaccuracy of epitope prediction approaches. Synthetic peptides provide a particularly useful means to prepare multiple immunogens efficiently and to rapidly translate identification of mutant epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

(3) Combination with a strong vaccine adjuvant. Effective vaccines require a strong adjuvant to initiate an immune response. As described below, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4⁺ and CD8⁺ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4⁺ and CD8⁺ T cell, as well as antibody responses to the peptide in a recent phase 1 study. At the same time, poly-ICLC has been extensively tested in more than 25 clinical trials to date and exhibited a relatively benign toxicity profile.

The above-described advantages of the invention are described in further detail below.

### Identification of Tumor Specific Neo-Antigen Mutations

The present invention is based, at least in part, on the ability to identify all, or nearly all, of the mutations within a neoplasia/tumor (e.g., translocations, inversions, large and small deletions and insertions, missense mutations, splice site mutations, etc.). In particular, these mutations are present in the genome of neoplasia/tumor cells of a subject, but not in normal tissue from the subject. Such mutations are of particular interest if they lead to changes that result in a protein with an altered amino acid sequence that is unique to the patient's neoplasia/tumor (e.g., a neo-antigen). For example, useful mutations may include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence; and the like. Peptides with mutations or mutated polypeptides arising from, for example, splice-site, frameshift, read-through, or gene fusion mutations in tumor cells may be identified by sequencing DNA, RNA or protein in tumor versus normal cells.

Also within the scope of the inventions is personal neo-antigen peptides derived from common tumor driver genes and may further include previously identified tumor specific mutations. For example, known common tumor driver genes and tumor mutations in common tumor driver genes may be found on the world wide web at (www)sanger.ac.uk/cosmic.

A number of initiatives are currently underway to obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In a preferred embodiment, the DNA or RNA sample is obtained from a neoplasia/tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In one embodiment, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen et al. (French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA® is described in PCT Application No. WO1992/15712). GBA® uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. W01991/02087) the GBA® method is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88: 1143-1147 (1991); Prezant, T. R. et al., Hum. Mutat. 1: 159-164 (1992); Ugozzoli, L. et al., GATA 9: 107-112 (1992); Nyren, P. et al., Anal. Biochem. 208: 171-175 (1993)). These methods differ from GBA® in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C, et al., Amer. J. Hum. Genet. 52:46-59 (1993)).

An alternative method for identifying tumor specific neo-antigens is direct protein sequencing. Protein sequencing of enzymatic digests using multidimensional MS techniques (MSn) including tandem mass spectrometry (MS/MS)) can also be used to identify neo-antigens of the invention. Such proteomic approaches permit rapid, highly automated analysis (see, e.g., K. Gevaert and J. Vandekerckhove, Electrophoresis 21:1145-1154 (2000)). It is further contemplated within the scope of the invention that high-throughput methods for de novo sequencing of unknown proteins may be used to analyze the proteome of a patient's tumor to identify expressed neo-antigens. For example, meta shotgun protein sequencing may be used to identify expressed neo-antigens (see e.g., Guthals et al. (2012) Shotgun Protein Sequencing with Meta-contig Assembly, Molecular and Cellular Proteomics 11(10):1084-96).

Tumor specific neo-antigens may also be identified using MHC multimers to identify neo-antigen-specific T-cell responses. For example, highthroughput analysis of neo-antigen-specific T-cell responses in patient samples may be performed using MHC tetramer-based screening techniques (see e.g., Hombrink et al. (2011) High-Throughput Identification of Potential Minor Histocompatibility Antigens by MHC Tetramer-Based Screening: Feasibility and Limitations 6(8):1-11; Hadrup et al. (2009) Parallel detection of antigen-specific T-cell responses by multidimensional encoding of MHC multimers, Nature Methods, 6(7):520-26; van Rooij et al. (2013) Tumor exome analysis reveals neoantigen-specific T-cell reactivity in an Ipilimumab-responsive melanoma, Journal of Clinical Oncology, 31:1-4; and Heemskerk et al. (2013) The cancer antigenome, EMBO Journal, 32(2):194-203). It is contemplated within the scope of the invention that such tetramer-based screening techniques may be used for the initial identification of tumor specific neo-antigens, or alternatively as a secondary screening protocol to assess what neo-antigens a patient may have already been exposed to, thereby facilitating the selection of candidate neo-antigens for the vaccines of the invention.

### Design of Tumor Specific Neo-Antigens

The invention further includes isolated peptides (e.g., neo-antigenic peptides containing the tumor specific mutations identified by the methods of the invention, peptides that comprise know tumor specific mutations, and mutant polypeptides or fragments thereof identified by the method of the invention). These peptides and polypeptides are referred to herein as “neo-antigenic peptides” or “neo-antigenic polypeptides.” The term “peptide” is used interchangeably with “mutant peptide” and “neo-antigenic peptide” and “wildtype peptide” in the present specification to designate a series of residues, typically L-amino acids, connected one to the other, typically by peptide bonds between the alpha-amino and alpha-carboxyl groups of adjacent amino acids. The polypeptides or peptides can be of a variety of lengths and will minimally include the small region predicted to bind to the HLA molecule of the patient (the “epitope”) as well as additional adjacent amino acids extending in both the N- and C-terminal directions. The polypeptides or peptides can be either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. In certain embodiments the size of the at least one neo-antigenic peptide molecule may comprise, but is not limited to, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neo-antigenic peptide molecules are equal to or less than 50 amino acids. In a preferred embodiment, the neo-antigenic peptide molecules are equal to about 20 to about 30 amino acids.

A longer peptide may be designed in several ways. For example, when the HLA-binding regions (e.g., the “epitopes”) are predicted or known, a longer peptide may consist of either: individual binding peptides with an extension of 0-10 amino acids toward the N- and C-terminus of each corresponding gene product. A longer peptide may also consist of a concatenation of some or all of the binding peptides with extended sequences for each. In another case, when sequencing reveals a long (>10 residues) neo-epitope sequence present in the tumor (e.g. due to a frameshift, read-through or intron inclusion that leads to a novel peptide sequence), a longer peptide may consist of the entire stretch of novel tumor-specific amino acids. In both cases, use of a longer peptide requires endogenous processing by professional antigen presenting cells such as dendritic cells and may lead to more effective antigen presentation and induction of T cell responses. In some cases, it is desirable or preferable to alter the extended sequence to improve the biochemical properties of the polypeptide (properties such as solubility or stability) or to improve the likelihood for efficient proteasomal processing of the peptide (Zhang et al (2012) Aminopeptidase substrate preference affects HIV epitope presentation and predicts immune escape patterns in HIV-infected individuals. J. Immunol 188:5924-34; Hearn et al (2010) Characterizing the specificity and co-operation of aminopeptidases in the cytosol and ER during MHC Class I antigen presentation. J. Immunol 184(9):4725-32; Wiemerhaus et al (2012) Peptidases trimming MHC Class I ligands. Curr Opin Immunol 25:1-7).

The neo-antigenic peptides and polypeptides may bind an HLA protein. In preferred aspects, the neo-antigenic peptides and polypeptides may bind an HLA protein with greater affinity than the corresponding native/wild-type peptide. The neo-antigenic peptide or polypeptide may have an IC50 of about less than 1000 nM, about less than 500 nM, about less than 250 nM, about less than 200 nM, about less than 150 nM, about less than 100 nM, or about less than 50 nM.

In a preferred embodiment, the neo-antigenic peptides and polypeptides of the invention do not induce an autoimmune response and/or invoke immunological tolerance when administered to a subject.

The invention also provides compositions comprising a plurality of neo-antigenic peptides. In some embodiments, the composition comprises at least 5 or more neo-antigenic peptides. In some embodiments the composition contains at least about 6, about 8, about 10, about 12, about 14, about 16, about 18, or about 20 distinct peptides. In some embodiments the composition contains at least 20 distinct peptides. According to the invention, 2 or more of the distinct peptides may be derived from the same polypeptide. For example, if a preferred neo-antigenic mutation encodes a neoORF polypeptide, two or more of the neo-antigenic peptides may be derived from the neoORF polypeptide. In one embodiment, the two or more neo-antigenic peptides derived from the neoORF polypeptide may comprise a tiled array that spans the polypeptide (e.g., the neo-antigenic peptides may comprise a series of overlapping neo-antigenic peptides that spans a portion, or all, of the neoORF polypeptide). Without being bound by theory, each peptide is believed to have its own epitope; accordingly, a tiling array that spans one neoORF polypeptide may give rise to polypeptides that are targeted to different HLA molecules. Neo-antigenic peptides can be derived from any protein coding gene. Exemplary polypeptides from which the neo-antigenic peptides may be derived can be found for example at the COSMIC database (on the worldwide web at (www)sanger.ac.uk/cosmic). COSMIC curates comprehensive information on somatic mutations in human cancer. The peptide may contain the tumor specific mutation. In some aspects the tumor specific mutation is in a common driver gene or is a common driver mutation for a particular cancer type. For example, common driver mutation peptides may include, but are not limited to, the following: a SF3B1 polypeptide, a MYD88 polypeptide, a TP53 polypeptide, an ATM polypeptide, an Abl polypeptide, A FBXW7 polypeptide, a DDX3X polypeptide, a MAPK1 polypeptide, or a GNB1 polypeptide.

The neo-antigenic peptides, polypeptides, and analogs can be further modified to contain additional chemical moieties not normally part of the protein. Those derivatized moieties can improve the solubility, the biological half-life, absorption of the protein, or binding affinity. The moieties can also reduce or eliminate any desirable side effects of the proteins and the like. An overview for those moieties can be found in Remington's Pharmaceutical Sciences, 20^{th} ed., Mack Publishing Co., Easton, Pa. (2000).

For example, neo-antigenic peptides and polypeptides having the desired activity may be modified as necessary to provide certain desired attributes, e.g. improved pharmacological characteristics, while increasing or at least retaining substantially all of the biological activity of the unmodified peptide to bind the desired MHC molecule and activate the appropriate T cell. For instance, the neo-antigenic peptide and polypeptides may be subject to various changes, such as substitutions, either conservative or non-conservative, where such changes might provide for certain advantages in their use, such as improved MHC binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, III., Pierce), 2d Ed. (1984).

The neo-antigenic peptide and polypeptides may also be modified by extending or decreasing the compound's amino acid sequence, e.g., by the addition or deletion of amino acids. The neo-antigenic peptides, polypeptides, or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity. The non-critical amino acids need not be limited to those naturally occurring in proteins, such as L-a-amino acids, or their D-isomers, but may include non-natural amino acids as well, such as β-γ-δ-amino acids, as well as many derivatives of L-a-amino acids.

Typically, a neo-antigen polypeptide or peptide may be optimized by using a series of peptides with single amino acid substitutions to determine the effect of electrostatic charge, hydrophobicity, etc. on MHC binding. For instance, a series of positively charged (e.g., Lys or Arg) or negatively charged (e.g., Glu) amino acid substitutions may be made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place.

The neo-antigenic peptides and polypeptides may be modified to provide desired attributes. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the peptide may be linked to the T helper peptide without a spacer.

The neo-antigenic peptide may be linked to the T helper peptide either directly or via a spacer either at the amino or carboxy terminus of the peptide. The amino terminus of either the neo-antigenic peptide or the T helper peptide may be acylated. Exemplary T helper peptides include tetanus toxoid 830-843, influenza 307-319, malaria circumsporozoite 382-398 and 378-389.

### Production of Tumor Specific Neo-Antigens

The present invention is based, at least in part, on the ability to present the immune system of the patient with a pool of tumor specific neo-antigens. One of skill in the art will appreciate that there are a variety of ways in which to produce such tumor specific neo-antigens. In general, such tumor specific neo-antigens may be produced either in vitro or in vivo. Tumor specific neo-antigens may be produced in vitro as peptides or polypeptides, which may then be formulated into a personalized neoplasia vaccine and administered to a subject. As described in further detail below, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific neo-antigens may be produced in vivo by introducing molecules (e.g., DNA, RNA, viral expression systems, and the like) that encode tumor specific neo-antigens into a subject, whereupon the encoded tumor specific neo-antigens are expressed.

### In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

A further aspect of the invention provides a nucleic acid (e.g., a polynucleotide) encoding a neo-antigenic peptide of the invention, which may be used to produce the neo-antigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. A still further aspect of the invention provides an expression vector capable of expressing a polypeptide according to the invention. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

The invention further embraces variants and equivalents which are substantially homologous to the identified tumor specific neo-antigens described herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

The invention also includes expression vectors comprising the isolated polynucleotides, as well as host cells containing the expression vectors. It is also contemplated within the scope of the invention that the neo-antigenic peptides may be provided in the form of RNA or cDNA molecules encoding the desired neo-antigenic peptides. The invention also provides that the one or more neo-antigenic peptides of the invention may be encoded by a single expression vector. The invention also provides that the one or more neo-antigenic peptides of the invention may be encoded and expressed in vivo using a viral based system (e.g., an adenovirus system).

The term “polynucleotide encoding a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neo-antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neo-antigenic peptide fused in the same reading frame to a marker sequence that allows, for example, for purification of the encoded polypeptide, which may then be incorporated into the personalized neoplasia vaccine. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neo-antigenic peptides fused in the same reading frame to create a single concatamerized neo-antigenic peptide construct capable of producing multiple neo-antigenic peptides.

In embodiments, the present invention provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neo-antigenic peptide of the present invention.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neo-antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest will be inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific neo-antigenic peptides. Recombinant expression vectors are replicable DNA constructs which have synthetic or cDNA-derived DNA fragments encoding a tumor specific neo-antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### In Vivo Peptide/Polypeptide Synthesis

The present invention also contemplates the use of nucleic acid molecules as vehicles for delivering neo-antigenic peptides/polypeptides to the subject in vivo in the form of, e.g., DNA/RNA vaccines (see, e.g., WO2012/159643, and WO2012/159754, hereby incorporated by reference in their entirety).

In one embodiment, the personalized neoplasia vaccine may include separate DNA plasmids encoding, for example, one or more neo-antigenic peptides/polypeptides as identified in according to the invention. As discussed above, the exact choice of expression vectors will depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

In another embodiment, the personalized neoplasia vaccine may include separate RNA or cDNA molecules encoding neo-antigenic peptides/polypeptides of the invention.

In another embodiment the personalized neoplasia vaccine may include a viral based vector for use in a human patient such as, for example, and adenovirus system (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety).

### Pharmaceutical Compositions/Methods of Delivery

The present invention is also directed to pharmaceutical compositions comprising an effective amount of one or more compounds according to the present invention (including a pharmaceutically acceptable salt, thereof), optionally in combination with a pharmaceutically acceptable carrier, excipient or additive.

A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally or ocularly administered compound to be more readily absorbed into the blood) or which enhance delivery of the parent compound to a biological compartment (e.g., the retina) relative to the parent species.

While the tumor specific neo-antigenic peptides of the invention can be administered as the sole active pharmaceutical agent, they can also be used in combination with one or more other agents and/or adjuvants. When administered as a combination, the therapeutic agents can be formulated as separate compositions that are given at the same time or different times, or the therapeutic agents can be given as a single composition.

The tumor specific neo-antigenic peptides of the present invention may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

The pharmaceutically active compounds of this invention can be processed in accordance with conventional methods of pharmacy to produce medicinal agents for administration to patients, including humans and other mammals.

Modifications of the active compound can affect the solubility, bioavailability and rate of metabolism of the active species, thus providing control over the delivery of the active species. This can easily be assessed by preparing the derivative and testing its activity according to known methods well within the routine practitioner's skill in the art.

Pharmaceutical compositions based upon these chemical compounds comprise the above-described tumor specific neo-antigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art will recognize that a therapeutically effective amount of one of more compounds according to the present invention will vary with the infection or condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material-of the above type, a liquid carrier such as a fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations will be apparent to those skilled in the art.

A skilled artisan will recognize that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier will usually comprise sterile water or aqueous sodium chloride solution, though other ingredients including those which aid dispersion may be included. Of course, where sterile water is to be used and maintained as sterile, the compositions and carriers will also be sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific neo-antigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neo-antigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as Herceptin® and Rituxan®.

It should be understood that in addition to the ingredients particularly mentioned above, the formulations of the present invention may include other agents conventional in the art having regard to the type of formulation in question, for example, those suitable for oral administration may include flavoring agents.

In certain pharmaceutical dosage forms, the pro-drug form of the compounds may be preferred. One of ordinary skill in the art will recognize how to readily modify the present compounds to pro-drug forms to facilitate delivery of active compounds to a targeted site within the host organism or patient. The routine practitioner also will take advantage of favorable pharmacokinetic parameters of the pro-drug forms, where applicable, in delivering the present compounds to a targeted site within the host organism or patient to maximize the intended effect of the compound.

Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214. The prodrug forms may be active themselves, or may be those such that when metabolized after administration provide the active therapeutic agent in vivo.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The additional agents that may be included with the tumor specific neo-antigenic peptides of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Preferred unit dosage formulations are those containing a daily dose or unit, daily sub-dose, as hereinabove recited, or an appropriate fraction thereof, of the administered ingredient.

The dosage regimen for treating a disorder or a disease with the tumor specific neo-antigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject will depend on a number of factors, such as the mode of administration, the nature of the condition being treated, the body weight of the subject being treated and the judgment of the prescribing physician.

The amount of compound included within therapeutically active formulations according to the present invention is an effective amount for treating the disease or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound will depend on the condition being treated, the particular compound, and other clinical factors such as weight and condition of the patient and the route of administration of the compound. It is to be understood that the present invention has application for both human and veterinary use.

For oral administration to humans, a dosage of between approximately 0.1 to 100 mg/kg/day, preferably between approximately 1 and 100 mg/kg/day, is generally sufficient.

Where drug delivery is systemic rather than topical, this dosage range generally produces effective blood level concentrations of active compound ranging from less than about 0.04 to about 400 micrograms/cc or more of blood in the patient.

The compound is conveniently administered in any suitable unit dosage form, including but not limited to one containing 0.001 to 3000 mg, preferably 0.05 to 500 mg of active ingredient per unit dosage form. An oral dosage of 10-250 mg is usually convenient.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

In certain embodiments, the compound is administered once daily; in other embodiments, the compound is administered twice daily; in yet other embodiments, the compound is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compounds of the invention can be used to treat diseases and disease conditions that are acute, and may also be used for treatment of chronic conditions. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

The invention provides for pharmaceutical compositions containing at least one tumor specific neo-antigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, Transcutol®, and Azone®.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as Silastic™, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### Dosages

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with viral infection and/or autoimmune disease.

Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 μg/ml. The pharmaceutical compositions typically should provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about 1 μM of an agent is administered to a subject. In related embodiments, about 50-100 nM, 50-250 nM, 100-500 nM, 250-500 nM, 250-750 nM, 500-750 nM, 500 nM to 1 μM, or 750 nM to 1 μM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

### Combination Therapies

The tumor specific neo-antigen peptides and pharmaceutical compositions described herein can also be administered in combination with another therapeutic molecule. The therapeutic molecule can be any compound used to mitigate neoplasia, or symptoms thereof. Examples of such compounds include, but are not limited to, chemotherapeutic agents, anti-angiogenesis agents, checkpoint blockade antibodies or other molecules that reduce immune-suppression, and the like.

The tumor specific neo-antigen peptides can be administered before, during, or after administration of the additional therapeutic agent. In embodiments, the tumor specific neo-antigen peptides are administered before the first administration of the additional therapeutic agent. In embodiments, the tumor specific neo-antigen peptides are administered after the first administration of the additional therapeutic agent (e.g., 1, 2, 3, 4, 5, 6, 7, 8, 9, 10, 11, 12, 13, 14 days or more). In embodiments, the tumor specific neo-antigen peptides are administered simultaneously with the first administration of the additional therapeutic agent.

### Vaccines

In an exemplary embodiment, the present invention is directed to an immunogenic composition, e.g., a vaccine composition capable of raising a specific T-cell response. The vaccine composition comprises mutant neo-antigenic peptides and mutant neo-antigenic polypeptides corresponding to tumor specific neo-antigens identified by the methods described herein.

A suitable vaccine will preferably contain a plurality of tumor specific neo-antigenic peptides. In an embodiment, the vaccine will include between 1 and 100 sets peptides, more preferably between 1 and 50 such peptides, even more preferably between 10 and 30 sets peptides, even more preferably between 15 and 25 peptides. According to another preferred embodiment, the vaccine will include approximately 20 peptides, more preferably 5, 6, 7, 8, 9, 10, 11, 12, 13, 14, 15, 16, 17, 18, 19, 20, 21, 22, 23, 24, 25, 26, 27, 28, 29, or 30 different peptides, further preferred 6, 7, 8, 9, 10 11, 12, 13, 14, 15, 16, 17, 18, 19, 20, 21, 22, 23, 24, or 25 different peptides, and most preferably 18, 19, 20, 21, 22, 23, 24, or 25 different peptides.

In one embodiment of the present invention the different tumor specific neo-antigenic peptides and/or polypeptides are selected for use in the neoplasia vaccine so as to maximize the likelihood of generating an immune attack against the neoplasia/tumor of the patient. Without being bound by theory, it is believed that the inclusion of a diversity of tumor specific neo-antigenic peptides will generate a broad scale immune attack against a neoplasia/tumor. In one embodiment, the selected tumor specific neo-antigenic peptides/polypeptides are encoded by missense mutations. In a second embodiment, the selected tumor specific neo-antigenic peptides/polypeptides are encoded by a combination of missense mutations and neoORF mutations. In a third embodiment, the selected tumor specific neo-antigenic peptides/polypeptides are encoded by neoORF mutations.

In one embodiment in which the selected tumor specific neo-antigenic peptides/polypeptides are encoded by missense mutations, the peptides and/or polypeptides are chosen based on their capability to associate with the particular MHC molecules of the patient. Peptides/polypeptides derived from neoORF mutations can also be selected on the basis of their capability to associate with the particular MHC molecules of the patient, but can also be selected even if not predicted to associate with the particular MHC molecules of the patient.

The vaccine composition is capable of raising a specific cytotoxic T-cells response and/or a specific helper T-cell response.

The vaccine composition can further comprise an adjuvant and/or a carrier. Examples of useful adjuvants and carriers are given herein below. The peptides and/or polypeptides in the composition can be associated with a carrier such as, e.g., a protein or an antigen-presenting cell such as e.g. a dendritic cell (DC) capable of presenting the peptide to a T-cell.

Adjuvants are any substance whose admixture into the vaccine composition increases or otherwise modifies the immune response to the mutant peptide. Carriers are scaffold structures, for example a polypeptide or a polysaccharide, to which the neo-antigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM. vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co-stimulatory molecules in DCs. Furthermore, poly-ICLC was recently directly compared to CpG in non-human primates (rhesus macaques) as adjuvant for a protein vaccine consisting of human papillomavirus (HPV)16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4)).

CpG immuno stimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Thl cells and strong cytotoxic T-lymphocyte (CTL) generation, even in the absence of CD4 T-cell help. The Thl bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, June 2006, 471-484). U.S. Pat. No. 6,406,705 Bl describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8:1396-1401).

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the P1/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8⁺ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD8⁺ T-cell and antibody responses were markedly enhanced with the addition of poly-ICLC and Montanide compared to OLP alone or OLP and Montanide.

A vaccine composition according to the present invention may comprise more than one different adjuvant. Furthermore, the invention encompasses a therapeutic composition comprising any adjuvant substance including any of the above or combinations thereof. It is also contemplated that the peptide or polypeptide, and the adjuvant can be administered separately in any appropriate sequence.

A carrier may be present independently of an adjuvant. The function of a carrier can for example be to confer stability, to increase the biological activity, or to increase serum half-life. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diptheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail below, the MHC class I or II molecule may readily be loaded with the selected antigen in vitro.

Preferably, the antigen presenting cells are dendritic cells. Suitably, the dendritic cells are autologous dendritic cells that are pulsed with the neo-antigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278.

Thus, in one embodiment of the present invention the vaccine composition containing at least one antigen presenting cell is pulsed or loaded with one or more peptides of the present invention. Alternatively, peripheral blood mononuclear cells (PBMCs) isolated from a patient may be loaded with peptides ex vivo and injected back into the patient. As an alternative the antigen presenting cell comprises an expression construct encoding a peptide of the present invention. The polynucleotide may be any suitable polynucleotide and it is preferred that it is capable of transducing the dendritic cell, thus resulting in the presentation of a peptide and induction of immunity.

### Therapeutic Methods

The invention further provides a method of inducing a neoplasia/tumor specific immune response in a subject, vaccinating against a neoplasia/tumor, treating and or alleviating a symptom of cancer in a subject by administering the subject a neo-antigenic peptide or vaccine composition of the invention.

According to the invention, the above-described cancer vaccine may be used for a patient that has been diagnosed as having cancer, or at risk of developing cancer. In one embodiment, the patient may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

The peptide or composition of the invention is administered in an amount sufficient to induce a CTL response.

The neo-antigenic peptide, polypeptide or vaccine composition of the invention can be administered alone or in combination with other therapeutic agents. The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

In addition, the subject may be further administered an anti-immunosuppressive or immunostimulatory agent. For example, the subject is further administered an anti-CTLA antibody or anti-PD-1 or anti-PD-Ll. Blockade of CTLA-4 or PD-1/PD-L1 by antibodies can enhance the immune response to cancerous cells in the patient. In particular, CTLA-4 blockade has been shown effective when following a vaccination protocol (Hodi et al 2005).

The optimum amount of each peptide to be included in the vaccine composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 μg and 1.5 mg, preferably 10 μg to 500 μg, of peptide or DNA may be given and will depend from the respective peptide or DNA. Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine composition are known to those skilled in the art.

The inventive pharmaceutical composition may be compiled so that the selection, number and/or amount of peptides present in the composition is/are tissue, cancer, and/or patient-specific. For instance, the exact selection of peptides can be guided by expression patterns of the parent proteins in a given tissue to avoid side effects. The selection may be dependent on the specific type of cancer, the status of the disease, earlier treatment regimens, the immune status of the patient, and, of course, the HLA-haplotype of the patient. Furthermore, the vaccine according to the invention can contain individualized components, according to personal needs of the particular patient. Examples include varying the amounts of peptides according to the expression of the related neoantigen in the particular patient, unwanted side-effects due to personal allergies or other treatments, and adjustments for secondary treatments following a first round or scheme of treatment.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from usually less than about 0.1%, to at least about 2% to as much as 20% to 50% or more by weight, and will be selected primarily by fluid volumes, viscosities, etc., in accordance with the particular mode of administration selected.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant will, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963).

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO1996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest can also be used for delivery (see, e.g., Kiken et al, 2011; Su et al, 2011).

The peptides and polypeptides of the invention can also be expressed by attenuated viral hosts, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhi vectors and the like, will be apparent to those skilled in the art from the description herein.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA' vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients that do not respond to other conventional forms of therapy, or will not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they will destroy their specific target cell (i.e., a tumor cell). In order to optimize the in vitro conditions for the generation of specific cytotoxic T cells, the culture of stimulator cells is maintained in an appropriate serum-free medium.

Prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that will allow about 200, and preferably 200 or more, human Class I MHC molecules loaded with peptide to be expressed on the surface of each stimulator cell. Preferably, the stimulator cells are incubated with >2 μg/ml peptide. For example, the stimulator cells are incubates with >3, 4, 5, 10, 15, or more μg/ml peptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+ (effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte: stimulator cell ratio is in the range of about 30:1 to 300:1. The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses.

Since mutant cell lines do not exist for every human MHC allele, it is advantageous to use a technique to remove endogenous MHC-associated peptides from the surface of APC, followed by loading the resulting empty MHC molecules with the immunogenic peptides of interest. The use of non-transformed (non-tumorigenic), noninfected cells, and preferably, autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its al and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destabilize p2microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions do not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody-tagged molecules may then be extracted from the stimulator-effector cell admixture via appropriate means, e.g., via well-known immunoprecipitation or immunoassay methods.

Effective, cytotoxic amounts of the activated CD8+ cells can vary between in vitro and in vivo uses, as well as with the amount and type of cells that are the ultimate target of these killer cells. The amount will also vary depending on the condition of the patient and should be determined via consideration of all appropriate factors by the practitioner. Preferably, however, about 1×10⁶ to about 1×10¹², more preferably about 1×10⁸ to about 1×10¹¹, and even more preferably, about 1×10⁹ to about 1×10¹⁰ activated CD8+ cells are utilized for adult humans, compared to about 5×10⁶-5×10⁷ cells used in mice.

Preferably, as discussed above, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells is not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient's tumor. CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type 1 (TH1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells non-specifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD8+ cells; 2) an intermediate level in which native, tumor-associated CD4+ epitopes may be used to augment CD8+ cells; and 3) a patient specific level in which neoantigen CD4+ epitopes may be used to augment CD8+ cells in a patient specific manner.

CD8+ cell immunity may also be generated with neo-antigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gp100 melanoma antigen-derived peptides with autologous peptide pulsed CD40L/IFN-g-activated mature DCs via an IL-12p70-producing patient DC vaccine (see e.g., Carreno et al (2013) L-12p70-producing patient DC vaccine elicits Tc1-polarized immunity, Journal of Clinical Investigation, 123(8):3383-94 and Ali et al. (2009) In situ regulation of DC subsets and T cells mediates tumor regression in mice, Cancer Immunotherapy, 1(8):1-10). It is contemplated within the scope of the invention that neo-antigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-12), tumor necrosis factor (TNF), interferon gamma-induced protein 10 (IP-10), interleukin 1 (IL-1), and type I interferons (IFN), and minimal interleukin 10 (IL-10) production. DCs may be differentiated from frozen peripheral blood mononuclear cells (PBMCs) obtained by leukapheresis, while PBMCs may be isolated by Ficoll gradient centrifugation and frozen in aliquots.

Illustratively, the following 7 day activation protocol may be used. Day 1—PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. August 1; (78). doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact with T cells to generate a potent cytotoxic T lymphocyte response to the cancer neo-antigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1(8):1-10; Ali et al. (2009) Infection-mimicking materials to program dendritic cells in situ. Nat Mater 8:151-8), and such a vaccine is currently being tested in a Phase I clinical trial recently initiated at the Dana-Farber Cancer Institute. This approach has also been shown to lead to regression of glioblastoma, as well as the induction of a potent memory response to prevent relapse, using the C6 rat glioma model.24 In the current proposal. The ability of such an implantable, biomatrix vaccine delivery scaffold to amplify and sustain tumor specific dendritic cell activation may lead to more robust anti-tumor immunosensitization than can be achieved by traditional subcutaneous or intra-nodal vaccine administrations.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Cancer Vaccine Testing Protocol

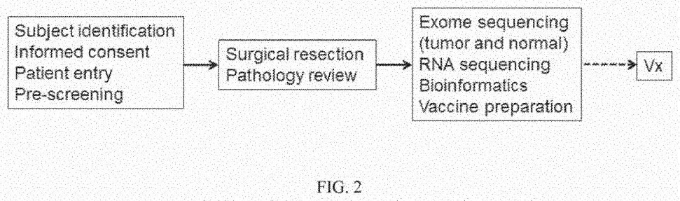

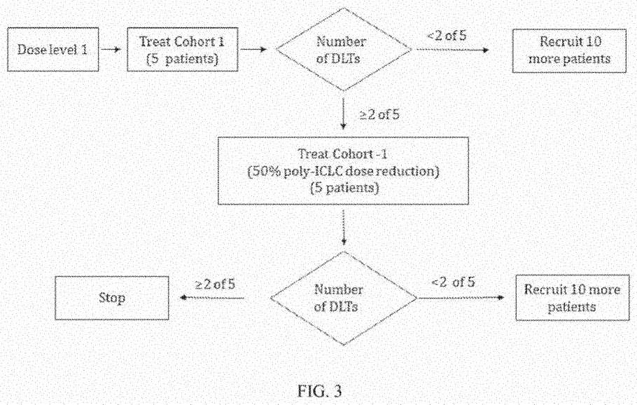

The above-described compositions and methods may be tested on 15 patients with high-risk melanoma (fully resected stages IIIB, IIIC and IVM1a,b) according to the general flow process shown in FIG. 2. Patients may receive a series of priming vaccinations with a mixture of personalized tumor-specific peptides and poly-ICLC over a 4 week period followed by two boosts during a maintenance phase. All vaccinations will be subcutaneously delivered. The vaccine will be evaluated for safety, tolerability, immune response and clinical effect in patients and for feasibility of producing vaccine and successfully initiating vaccination within an appropriate time frame. The first cohort will consist of 5 patients, and after safety is adequately demonstrated, an additional cohort of 10 patients may be enrolled (see, e.g., FIG. 3 depicting an approach for an initial population study). Peripheral blood will be extensively monitored for peptide-specific T-cell responses and patients will be followed for up to two years to assess disease recurrence.

As described above, there is a large body of evidence in both animals and humans that mutated epitopes are effective in inducing an immune response and that cases of spontaneous tumor regression or long term survival correlate with CD8⁺ T-cell responses to mutated epitopes (Buckwalter and Srivastava P K. “It is the antigen(s), stupid” and other lessons from over a decade of vaccitherapy of human cancer. Seminars in immunology 20:296-300 (2008); Karanikas et al, High frequency of cytolytic T lymphocytes directed against a tumor-specific mutated antigen detectable with HLA tetramers in the blood of a lung carcinoma patient with long survival. Cancer Res. 61:3718-3724 (2001); Lennerz et al, The response of autologous T cells to a human melanoma is dominated by mutated neo-antigens. Proc Natl Acad Sci USA. 102:16013 (2005)) and that “immunoediting” can be tracked to alterations in expression of dominant mutated antigens in mice and man (Matsushita et al, Cancer exome analysis reveals a T-cell-dependent mechanism of cancer immunoediting Nature 482:400 (2012); DuPage et al, Expression of tumor-specific antigens underlies cancer immunoediting Nature 482:405 (2012); and Sampson et al, Immunologic escape after prolonged progression-free survival with epidermal growth factor receptor variant III peptide vaccination in patients with newly diagnosed glioblastoma J Clin Oncol. 28:4722-4729 (2010)).

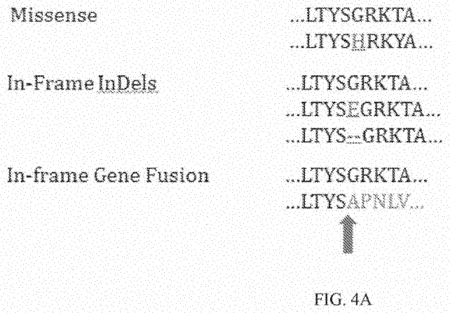

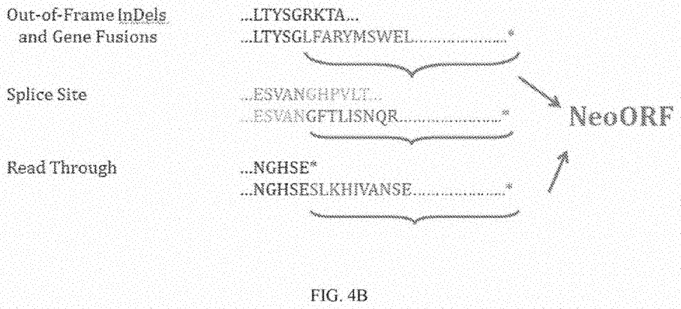

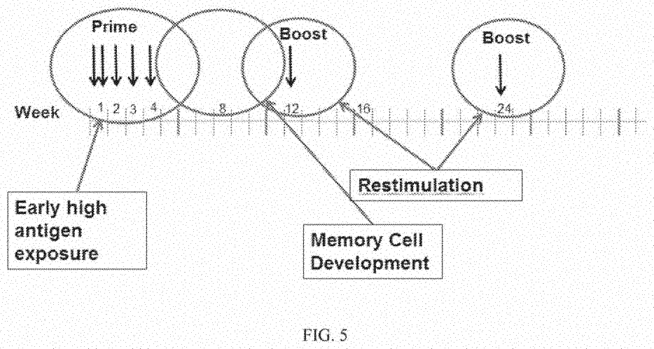

Next-generation sequencing can now rapidly reveal the presence of discrete mutations such as coding mutations in individual tumors, most commonly single amino acid changes (e.g., missense mutations; FIG. 4A) and less frequently novel stretches of amino acids generated by frame-shift insertions/deletions/gene fusions, read-through mutations in stop codons, and translation of improperly spliced introns (e.g., neoORFs; FIG. 4B). NeoORFs are particularly valuable as immunogens because the entirety of their sequence is completely novel to the immune system and so are analogous to a viral or bacterial foreign antigen. Thus, neoORFs: (1) are highly specific to the tumor (i.e. there is no expression in any normal cells); (2) can bypass central tolerance, thereby increasing the precursor frequency of neoantigen-specific CTLs. For example, the power of utilizing analogous foreign sequences in a therapeutic anti-cancer vaccine was recently demonstrated with peptides derived from human papilloma virus (HPV). ˜50% of the 19 patients with pre-neoplastic, viral-induced disease who received 3-4 vaccinations of a mix of HPV peptides derived from the viral oncogenes E6 and E7 maintained a complete response for ≥24 months (Kenter et a, Vaccination against HPV-16 Oncoproteins for Vulvar Intraepithelial Neoplasia NEJM 361:1838 (2009)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins, they are not subject to the immune-dampening effects of self-tolerance. Therefore, mutated proteins are more likely to be immunogenic and are also more specific for the tumor cells compared to normal cells of the patient.

Utilizing recently improved algorithms for predicting which missense mutations create strong binding peptides to the patient's cognate MHC molecules, a set of peptides representative of optimal mutated epitopes (both neoORF and missense) for each patient will be identified and prioritized and up to 20 or more peptides will be prepared for immunization (Zhang et al, Machine learning competition in immunology—Prediction of HLA class I binding peptides J Immunol Methods 374:1 (2011); Lundegaard et al Prediction of epitopes using neural network based methods J Immunol Methods 374:26 (2011)). Peptides ˜20-35 amino acids in length will be synthesized because such “long” peptides undergo efficient internalization, processing and cross-presentation in professional antigen-presenting cells such as dendritic cells, and have been shown to induce CTLs in humans (Melief and van der Burg, Immunotherapy of established (pre) malignant disease by synthetic long peptide vaccines Nature Rev Cancer 8:351 (2008)).

In addition to a powerful and specific immunogen, an effective immune response requires a strong adjuvant to activate the immune system (Speiser and Romero, Molecularly defined vaccines for cancer immunotherapy, and protective T cell immunity Seminars in Immunol 22:144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (2011)). Hiltonol®, a GMP preparation of poly-ICLC prepared by Oncovir, Inc, will be utilized as the adjuvant.

### Example 2

### Target Patient Population

Patients with stage IIIB, IIIC and IVM1a,b, melanoma have a significant risk of disease recurrence and death, even with complete surgical resection of disease (Balch et al, Final Version of 2009 AJCC Melanoma Staging and Classification J Clin Oncol 27:6199-6206 (2009)). An available systemic adjuvant therapy for this patient population is interferon-α (IFNα) which provides a measurable but marginal benefit and is associated with significant, frequently dose-limiting toxicity (Kirkwood et al, Interferon alfa-2b Adjuvant Therapy of High-Risk Resected Cutaneous Melanoma: The Eastern Cooperative Oncology Group Trial EST 1684 J Clin Oncol 14:7-17 (1996); Kirkwood et al, High- and Low-dose Interferon Alpha-2b in High-Risk Melanoma: First Analysis of Intergroup Trial E1690/S9111/C9190 J Clin Oncol 18:2444-2458 (2000)). These patients are not immuno-compromised by previous cancer-directed therapy or by active cancer and thus represent an excellent patient population in which to assess the safety and immunological impact of the vaccine. Finally, current standard of care for these patients does not mandate any treatment following surgery, thus allowing for the 8-10 week window for vaccine preparation.

The target population will be cutaneous melanoma patients with clinically detectable, histologically confirmed nodal (local or distant) or in transit metastasis, who have been fully resected and are free of disease (most of stage IIIB (because of the need to have adequate tumor tissue for sequencing and cell line development, patients with ulcerated primary tumor but micrometastatic lymph nodes (T1-4b, N1a or N2a) will be excluded.), all of stage IIIC, and stage IVM1a, b). These may be patients at first diagnosis or at disease recurrence after previous diagnosis of an earlier stage melanoma.

Tumor harvest: Patients will undergo complete resection of their primary melanoma (if not already removed) and all regional metastatic disease with the intent of rendering them free of melanoma. After adequate tumor for pathological assessment has been harvested, remaining tumor tissue will be placed in sterile media in a sterile container and prepared for disaggregation. Portions of the tumor tissue will be used for whole-exome and transcriptome sequencing and cell line generation and any remaining tumor will be frozen.

Normal tissue harvest: A normal tissue sample (blood or sputum sample) will be taken for whole exome sequencing.

Patients with clinically evident locoregional metastatic disease or fully resectable distant nodal, cutaneous or lung metastatic disease (but absence of unresectable distant or visceral metastatic disease) will be identified and enrolled on the study. Entry of patients prior to surgery is necessary in order to acquire fresh tumor tissue for melanoma cell line development (to generate target cells for in vitro cytotoxicity assays as part of the immune monitoring plan).

### Example 3

### Dose and Schedule

For patients who have met all pre-treatment criteria, vaccine administration will commence as soon as possible after the study drug has arrived and has met incoming specifications. For each patient, there will be four separate study drugs, each containing 5 of 20 patient-specific peptides. Immunizations may generally proceed according to the schedule shown in FIG. 5.

Patients will be treated in an outpatient clinic. Immunization on each treatment day will consist of four 1 ml subcutaneous injections, each into a separate extremity in order to target different regions of the lymphatic system to reduce antigenic competition. If the patient has undergone complete axillary or inguinal lymph node dissection, vaccines will be administered into the right or left midriff as an alternative. Each injection will consist of 1 of the 4 study drugs for that patient and the same study drug will be injected into the same extremity for each cycle. The composition of each 1 ml injection is:
0.75 ml study drug containing 300 μg each of 5 patient-specific peptides
0.25 ml (0.5 mg) of 2 mg/ml poly-ICLC (Hiltonol®)
   During the induction/priming phase, patients will be immunized on days 1, 4, 8, 15 and 22. In the maintenance phase, patients will receive booster doses at weeks 12 and 24.

Blood samples may be obtained at multiple time points: pre- (baseline; two samples on different days); day 15 during priming vaccination; four weeks after the induction/priming vaccination (week 8); pre- (week 12) and post- (week 16) first boost; pre- (week 24) and post- (week 28) second boost 50-150 ml blood will be collected for each sample (except week 16). The primary immunological endpoint will be at week 16, and hence patients will undergo leukapheresis (unless otherwise indicated based on patient and physician assessment).

### Example 4

### Immune Monitoring

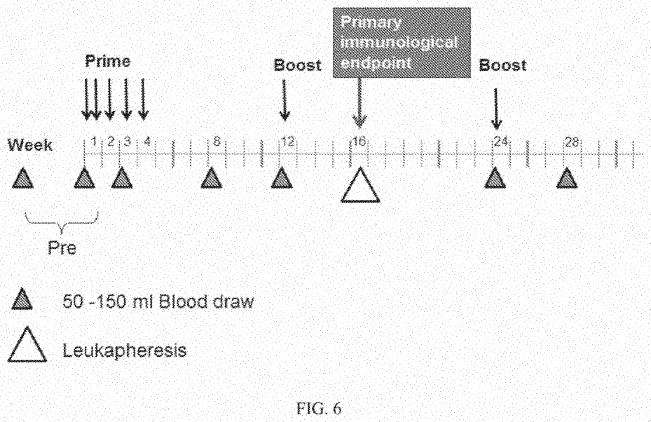

The immunization strategy is a “prime-boost” approach, involving an initial series of closely spaced immunizations to induce an immune response followed by a period of rest to allow memory T-cells to be established. This will be followed by a booster immunization, and the T-cell response 4 weeks after this boost is expected to generate the strongest response and will be the primary immunological endpoint. Global immunological response will be initially monitored using peripheral blood mononuclear cells from this time point in an 18 hr ex vivo ELISPOT assay, stimulating with a pool of overlapping 15mer peptides (11 aa overlap) comprising all the immunizing epitopes. Pre-vaccination samples will be evaluated to establish the baseline response to this peptide pool. As warranted, additional PBMC samples will be evaluated to examine the kinetics of the immune response to the total peptide mix. For patients demonstrating responses significantly above baseline, the pool of all 15mers will be de-convoluted to determine which particular immunizing peptide(s) were immunogenic. In addition, a number of additional assays will be conducted on a case-by-case basis for appropriate samples:
The entire 15mer pool or sub-pools will be used as stimulating peptides for intracellular cytokine staining assays to identify and quantify antigen-specific CD4+, CD8+, central memory and effector memory populations
Similarly, these pools will be used to evaluate the pattern of cytokines secreted by these cells to determine the T_{H}1 vs T_{H}2 phenotype
Extracellular cytokine staining and flow cytometry of unstimulated cells will be used to quantify Treg and myeloid-derived suppressor cells (MDSC).
If a melanoma cell line is successfully established from a responding patient and the activating epitope can be identified, T-cell cytotoxicity assays will be conducted using the mutant and corresponding wild type peptide
PBMC from the primary immunological endpoint will be evaluated for “epitope spreading” by using known melanoma tumor associated antigens as stimulants and by using several additional identified mutated epitopes that were not selected to be among the immunogens, as shown in FIG. 6.
   Immuno-histochemistry of the tumor sample will be conducted to quantify CD4+, CD8+, MDSC, and Treg infiltrating populations.

### Example 5

### Clinical Efficacy in Patients with Metastatic Disease

Vaccine treatment of patients with metastatic disease is complicated by their need for an effective therapy for the active cancer and the consequent absence of an off treatment time window for vaccine preparation. Furthermore, these cancer treatments may compromise the patient's immune system, possibly impeding the induction of an immune response. With these considerations in mind, settings may be chosen where timing of vaccine preparation fits temporally with other standard care approaches for the particular patient population and/or where such standard care is demonstrably compatible with an immunotherapeutic approach. There are two types of settings that may be pursued:

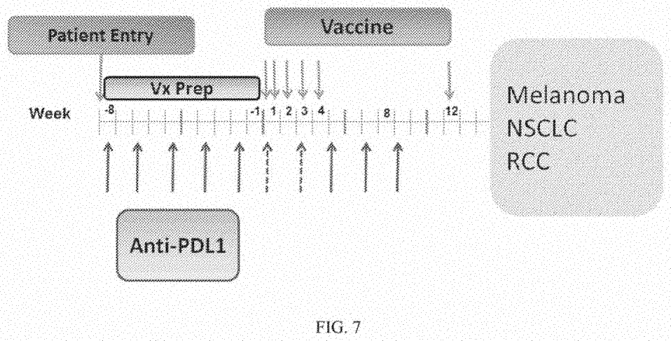

1. Combination with checkpoint blockade: Checkpoint blockade antibodies have emerged as an effective immunotherapy for metastatic melanoma (Hodi et al, Improved Survival with Ipilimumab in Patients with Metastatic Melanoma NEJM 363:711-723 (2010)) and are being actively pursued in other disease settings including non-small cell lung cancer (NSCLC) and renal cell carcinoma (Topalian et al, Safety, Activity, and Immune Correlates of Anti-PD-1 Antibody in Cancer NEJM 366:2443-2454 (2012); Brahmer et al, Safety and Activity of Anti-PD-L1 Antibody in Patients with Advanced Cancer NEJM 366:2455-2465(2012)). Although the mechanism of action is not proven, both reversal of relief from local immunosuppression and enhancement of an immune response are possible explanations. Integrating a powerful vaccine to initiate an immune response with checkpoint blockade antibodies may provide synergies, as observed in multiple animal studies (van Elsas et al Combination immunotherapy of B16 melanoma using anti-cytotoxic T lymphocyte-associated antigen 4 (CTLA-4)and granulocyte/macrophage colony-stimulating factor (GM-CSF)-producing vaccines induces rejection of subcutaneous and metastatic tumors accompanied by autoimmune depigmentation J Exp Med 190:35-366 (1999); Li et al, Anti-programmed death-1 synergizes with granulocyte macrophage colony-stimulating factor-secreting tumor cell immunotherapy providing therapeutic benefit to mice with established tumors Clin Cancer Res 15:1623-1634 (2009); Pardoll, D. M. The blockade of immune checkpoints in cancer immunotherapy Nature Reviews Cancer 12:252-264 (2012); Curran et al. PD-1 and CTLA-4 combination blockade expands infiltrating T cells and reduces regulatory T and myeloid cells within B16 melanoma tumors. Proc Natl Acad Sci USA. 2010 Mar. 2; 107(9):4275-80; Curran et al. Tumor vaccines expressing flt3 ligand synergize with ctla-4 blockade to reject preimplanted tumors. Cancer Res. 2009 Oct. 1; 69(19):7747-55). Patients can be immediately started on checkpoint blockade therapy while vaccine is being prepared and once prepared, the vaccine dosing can be integrated with antibody therapy, as illustrated in FIG. 7; and

2. Combination with standard treatment regimens exhibiting beneficial immune properties.

a) Renal cell carcinoma (RCC) patients who present with metastatic disease typically undergo surgical de-bulking followed by systemic treatment, which is commonly with one of the approved tyrosine kinase inhibitors (TKI) such as sunitinib, pazopanib and sorafenib. Of the approved TKIs, sunitinib has been shown to increase T_{H}1 responsiveness and decrease Treg and myeloid-derived suppressor cells (Finke et al, Sunitinib reverses Type-1 immune suppression and decreases T-regulatory cells in renal cell carcinoma patients Clin Can Res 14:6674-6682 (2008); Terme et al, VEGFA-VEGFR pathway blockade inhibits tumor-induced regulatory T cell proliferation in colorectal cancer (Cancer Research Author Manuscript published Online (2102)). The ability to immediately treat patients with an approved therapy that does not compromise the immune system provides the needed window to prepare the vaccine and could provide synergy with a vaccine therapy. In addition, cyclophosphamide (CTX) has been implicated in multiple animal and human studies to have an inhibitory effect on Treg cells and a single dose of CTX prior to a vaccine has been recently shown to improve survival in RCC patients who responded to the vaccine (Walter et al, Multipeptide immune response to a cancer vaccine IMA901 after single-dose cyclophosphamide associates with longer patient survival Nature Medicine 18:1254-1260 (2012)). Both of these immune-synergistic approaches have been utilized in a recently completed phase 3 study of a native peptide vaccine in RCC (ClinicalTrials.gov, NCT01265901 IMA901 in Patients Receiving Sunitinib for Advanced/Metastatic Renal Cell Carcinoma);

b) Alternatively, standard treatment of glioblastoma (GBM) involves surgery, recovery and follow-up radiation and low dose temozolomide (TMZ) followed by a four week rest period before initiating standard dose TMZ. This standard treatment provides a window for vaccine preparation followed by initiation of vaccination prior to starting standard dose TMZ. Interestingly, in a study in metastatic melanoma, peptide vaccination during standard dose TMZ treatment increased the measured immune responsiveness compared to vaccination alone, suggesting additional synergistic benefit (Kyte et al, Telomerase peptide vaccination combined with temozolomide: a clinical trial in stage IV melanoma patients Clin Cancer Res 17:4568 (2011)).

### Example 6

### Vaccine Preparation

Patient tumor tissue will be surgically resected, and tumor tissue will be disaggregated and separate portions used for DNA and RNA extraction and for patient-specific melanoma cell line development. DNA and/or RNA extracted from the tumor tissue will be used for whole-exome sequencing (e.g., by using the Illumina HiSeq platform) and to determine HLA typing information. It is contemplated within the scope of the invention that missense or neoORF neo-antigenic peptides may be directly identified by protein-based techniques (e.g., mass spectrometry).

Bioinformatics analysis will be conducted as follows. Sequence analysis of the Exome and RNA-SEQ fast Q files will leverage existing bioinformatic pipelines that have been used and validated extensively in large-scale projects such as the TCGA for many patient samples (e.g., Chapman et al, 2011, Stransky et al, 2011, Berger et al, 2012). There are two sequential categories of analyses: data processing and cancer genome analysis.
Data processing pipeline: The Picard data processing pipeline (picard.sourceforge.net/) was developed by the Sequencing Platform. Raw data extracted from (e.g., Illumina) sequencers for each tumor and normal sample is subjected to the following processes using various modules in the Picard pipeline:
   (i). Quality recalibration: Original base quality scores reported by the Illumina pipeline will be recalibrated based on the read-cycle, the lane, the flow cell tile, the base in question, and the preceding base.
   (ii). Alignment: BWA (Li and Durbin, 2009) will be used to align read pairs to the human genome (hg19).
   (iii). Mark duplicates: PCR and optical duplicates will be identified based on read pair mapping positions and marked in the final barn file.
The output of Picard is a bam file (Li et al, 2009) (samtools.sourceforge.net/SAM1.pdf) that stores the base sequences, quality scores, and alignment details for all reads for the given sample.
Cancer Mutation Detection Pipeline: Tumor and matched normal barn files from the Picard pipeline will be analyzed as described below:
   1. Quality Control
      (i). Sample mix-up during sequencing will be done by comparing initial SNP fingerprinting done on a sample at a few dozen sites with exome sequencing pileups at those sites.
      (ii). Intra-sample tumor/normal mixup will be checked by first comparing the insert size distribution of lanes that correspond to the same library for both tumor and normal samples, and discarding those lanes that have a different distribution. Bioinformatic analysis will be applied to tumor and matched normal exome samples to get the DNA copy number profiles. Tumor samples should also have more copy number variation than the corresponding normals. Lanes corresponding to normal samples that do not have flat profiles will be discarded, as will tumor lanes that don't have profiles consistent with other lanes from the same tumor sample will be discarded.
      (iii). Tumor purity and ploidy will be estimated based on the bioinformatic-generated copy number profiles.
      (iv). ContEst (Cibulskis et al, 2011) will be used to determine the level of cross-sample contamination in samples.
   2. Local realignment around putative indels
      True somatic and germline small indels with respect to the reference genome often result in misalignment and miscalls of missense mutations and indels. This will be corrected for by doing a local realignment using the GATK IndelRealigner module (on the worldwide web at (www)broadinstitute.org/gatk) (McKenna et al, 2010, Depristo et al, 2011) of all reads that map in the vicinity of putative indels and evaluating them comprehensively to ensure consistency and correctness of indel calls.
   3. Identification of somatic single nucleotide variations (SSNVs)
      Somatic base pair substitutions will be identified by analyzing tumor and matched normal samples from a patient using a Bayesian statistical framework called muTect (Cibulskis et al, 2013). In the preprocessing step, reads with a preponderance of low quality bases or mismatches to the genome are filtered out. Mutect then computes two log-odds (LOD) scores which encapsulate confidence in presence and absence of the variant in the tumor and normal samples respectively. In the post-processing stage candidate mutations are empirically filtered by various criteria to account for artifacts of capture, sequencing and alignment. One such filter, for example, tests for consistency between distributions of orientations of reads that harbor the mutation and the overall orientation distribution of reads that map to the locus to ensure that there is no strand bias. The final set of mutations will then be annotated with the Oncotator tool by several fields including genomic region, codon, cDNA and protein changes.
   4. Identification of somatic small insertions and deletions
      The local realignment output from section 2.2 will be used to predict candidate somatic and germline indels based on assessment of reads supporting the variant exclusively in tumor or both in tumor and normal bams respectively. Further filtering based on number and distribution of mismatches and base quality scores will be done (McKenna et al, 2010, DePristo et al, 2011). All indels will be manually inspected using the Integrated Genomics Viewer (Robinson et al, 2011) (on the worldwide web at (www)broadinstitute.org/igv) to ensure high-fidelity calls.
   5. Gene fusion detection
      The first step in the gene fusion detection pipeline is alignment of tumor RNA-Seq reads to a library of known gene sequences following by mapping of this alignment to genomic coordinates. The genomic mapping helps collapse multiple read pairs that map to different transcript variants that share exons to common genomic locations. The DNA aligned bam file will be queried for read pairs where the two mates map to two different coding regions that are either on different chromosomes or at least 1 MB apart if on the same chromosome. It will also be required that the pair ends aligned in their respective genes be in the direction consistent with coding→coding 5′→3′ direction of the (putative) fusion mRNA transcript. A list of gene pairs where there are at least two such ‘chimeric’ read pairs will be enumerated as the initial putative event list subject to further refinement. Next, all unaligned reads will be extracted from the original bam file, with the additional constraint that their mates were originally aligned and map into one of the genes in the gene pairs obtained as described above. An attempt will then be made to align all such originally unaligned reads to the custom “reference” built of all possible exon-exon junctions (full length, boundary-to-boundary, in coding 5′→3′ direction) between the discovered gene pairs. If one such originally unaligned read maps (uniquely) onto a junction between an exon of gene X and an exon of gene Y, and its mate was indeed mapped to one of the genes X or Y, then such a read will be marked as a “fusion” read. Gene fusion events will be called in cases where there is at least one fusion read in correct relative orientation to its mate, without excessive number of mismatches around the exon:exon junction and with a coverage of at least 10 bp in either gene. Gene fusions between highly homologous genes (ex. HLA family) are likely spurious and will be filtered out.
   6. Estimation of clonality
   Bioinformatic analysis may be used to estimate clonality of mutations. For example, the ABSOLUTE algorithm (Carter et al, 2012, Landau et al, 2013) may be used to estimate tumor purity, ploidy, absolute copy numbers and clonality of mutations. Probability density distributions of allelic fractions of each mutation will be generated followed by conversion to cancer cell fractions (CCFs) of the mutations. Mutations will be classified as clonal or subclonal based on whether the posterior probability of their CCF exceeds 0.95 is greater or lesser than 0.5 respectively.
   7. Quantification of expression
      The TopHat suite (Langmead et al, 2009) will be used to align RNA-Seq reads for the tumor and matched normal bams to the hg19 genome. The quality of RNA-Seq data will be assessed by the RNA-SeQC (DeLuca et al, 2012) package. The RSEM tool (Li et al, 2011) will then be used to estimate gene and isoform expression levels. The generated reads per kilobase per million and tau estimates will be used to prioritize neo-antigens identified in each patient as described elsewhere.
   8. Validation of mutations in RNA-Seq
      Mutations that will be identified by analysis of whole exome data (section 2.3) will be assessed for presence in the corresponding RNA-Seq tumor bam file of the patient. For each variant locus, a power calculation based on the beta-binomial distribution will be performed to ensure that there is at least 80% power to detect it in the RNA-Seq data. A capture identified mutation will be considered validated if there are at least 2 reads harboring the mutation for adequately powered sites.
Selection of Tumor-Specific Mutation-Containing Epitopes: All missense mutations and neoORFs will be analyzed for the presence of mutation-containing epitopes using the neural-network based algorithm netMHC, provided and maintained by the Center for Biological Sequence Analysis, Technical University of Denmark, Netherlands. This family of algorithms were rated the top epitope prediction algorithms based on a competition recently completed among a series of related approaches (ref). The algorithms were trained using an artificial neural network based approach on multiple different human HLA A and B alleles utilizing over 100,000 measured binding and non-binding interactions.

The accuracy of the algorithms were evaluated by conducting predictions from mutations found in CLL patients for whom the HLA allotypes were known. The included allotypes were A0101, A0201, A0310, A1101, A2402, A6801, B0702, B0801, B1501. Predictions were made for all 9mer and 10 mer peptides spanning each mutation using netMHCpan in mid-2011. Based on these predictions, seventy-four (74) 9mer peptides and sixty-three (63) 10mer peptides, most with predicted affinities below 500 nM, were synthesized and the binding affinity was measured using a competitive binding assay (Sette).

The predictions for these peptides were repeated in March 2013 using each of the most up to date versions of the netMHC servers (netMHCpan, netMHC and netMHCcons). These three algorithms were the top rated algorithms among a group of 20 used in a competition in 2012 (Zhang et al). The observed binding affinities were then evaluated with respect to each of the new predictions. For each set of predicted and observed values, the % of correct predictions for each range is given, as well as the number of samples. The definition for each range is as follows:
0-150 Predicted to have an affinity equal to or lower than 150 nM and measured to have an affinity equal to or lower than 150 nM.
0-150*: Predicted to have an affinity equal to or lower than 150 nM and measured to have an affinity equal to or lower than 500 nM.
151-500 nM: Predicted to have an affinity greater than 150 nM but equal to or lower than 500 nM and measured to have an affinity equal to or below 500 nM.
FN (>500 nM): False Negatives—Predicted to have an affinity greater than 500 nM but measured to have an affinity equal to or below 500 nM.
   For 9mer peptides (Table 1) , there was little difference between the algorithms, with the slightly higher value for the 151-500 nM range for netMHC cons not judged to be significant because of the low number of samples.

TABLE 1

| Range (nM) | 9mer PAN | 9mer netMHC | 9mer CONS |
|---|---|---|---|
| 0-150 | 76% | 78% | 76% |
| | (33) | (37) | (34) |
| 0-150* | 91% | 89% | 88% |
| | (33) | (37) | (34) |
| 151-500 | 50% | 50% | 62% |
| | (28) | (14) | (13) |
| FN (>500) | 38% | 39% | 41% |
| | (13) | (23) | (27) |

For 10mer peptides (Table 2), again there was little difference between the algorithms except that netMHC produced significantly more false positives than netMHCpan or netMMHCcons. However, the precision of the 10mer predictions is slightly lower in the 0-150 nM and 0-150* nM ranges and significantly lower in the 151-500 nM range, compared to the 9mers.

TABLE 2

| Range (nM) | 10mer PAN | 10mer netMHC | 10mer CONS |
|---|---|---|---|
| 0-150 | 53% | 50% | 59% |
| | (19) | (16) | (17) |
| 0-150* | 68% | 69% | 76% |
| | (19) | (16) | (17) |
| 151-500 | 35% | 42% | 35% |
| | (26) | (12) | (23) |
| FN (>500) | 11% | 23% | 13% |
| | (18) | (35) | (23) |

For 10mers, only predictions in the 0-150 nM range will be utilized due to the lower than 50% precision for binders in the 151-500 nM range.

The number of samples for any individual HLA allele was too small to draw any conclusions regarding accuracy of the prediction algorithm for different alleles. Data from the largest available subset (0-150* nM; 9mer) is shown in Table 3 as an example.

TABLE 3

| | Fraction |
|---|---|
| Allele | correct |
| A0101 | 2/2 |
| A0201 | 9/11 |
| A0301 | 5/5 |
| A1101 | 4/4 |
| A2402 | 0/0 |
| A6801 | ¾ |
| B0702 | 4/4 |
| B0801 | ½ |
| B1501 | 2/2 |

Only predictions for HLA A and B alleles will be utilized as there is little available data on which to judge accuracy of predictions for HLA C alleles (Zhang et al).

An evaluation of melanoma sequence information and peptide binding predictions was conducted using information from the TCGA database. Information from 220 melanomas from different patients revealed that on average there were approximately 450 missense and 5 neoORFs per patient. 20 patients were selected at random and the predicted binding affinities were calculated for all the missense mutations using netMHC (Lundegaard et al Prediction of epitopes using neural network based methods J Immunol Methods 374:26 (2011)). As the HLA allotypes were unknown for these patients, the number of predicted binding peptides per allotype was adjusted based on the frequency of that allotype (Bone Marrow Registry dataset for the expected affected dominant population in the geographic area [Caucasian for melanoma]) to generate a predicted number of actionable mutant epitopes per patient. For each of these mutant epitopes (MUT), the corresponding native (WT) epitope binding was also predicted. Utilizing a single peptide for predicted missense binders with Kd≤500 nM and a WT/MUT Kd ratio of >5× and over-lapping peptides spanning the full length of each neoORF, 80% (16 of 20) of patients were predicted to have at least 20 peptides appropriate for vaccination. For a quarter of the patients, neoORF peptides could constitute nearly half to all of the 20 peptides. Thus, there is an adequate mutational load in melanoma to expect a high proportion of patients to generate an adequate number of immunogenic peptides.

### Example 7

### Prioritization of Immunizing Peptides

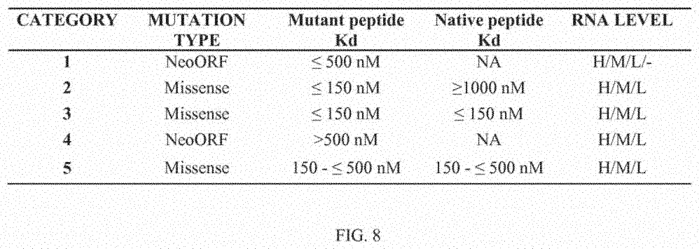

Peptides for immunization may be prioritized based on a number of criteria: neoORF vs. missense, predicted Kd for the mutated peptide, the comparability of predicted affinity for the native peptide compared to the mutated peptide, whether the mutation occurs in an oncogenic driver gene or related pathway, and # of RNA-Seq reads (see e.g., FIG. 8).

As shown in FIG. 8, peptides derived from segments of neoORF mutations that are predicted to bind (Kd<500 nM) may be given the highest priority based on the absence of tolerance for these entirely novel sequences and their exquisite tumor-specificity.

The similar class of missense mutations in which the native peptide is not predicted to bind (Kd>1000 nM) and the mutated peptide is predicted to bind with strong/moderate affinity (Kd<150 nM) may be given the next highest priority. This class (Group I discussed above) represents approximately 20% of naturally observed T-cell responses.

The third highest priority may be given to the more tightly binding (<150 nM) subset of the Group II class discussed above. This class is responsible for approximately almost ⅔ of naturally observed T-cell responses.

All the remaining peptides derived from the neoORF mutations may be given the fourth priority. Despite not being predicted to bind, these are included based on the known false negative rate, potential binding to HLA-C, potential for presence of Class II epitopes and the high value of utilizing totally foreign antigens.

The fifth priority may be given to the subset of Group II with lower predicted binding affinities (150-500 nM). This class is responsible for approximately 10% of the naturally observed T-cell responses.

As the predicted affinity decreases, higher stringency may be applied to expression levels. Within each grouping, peptides may be ranked based on binding affinity (e.g., the lowest Kd may have the highest priority). Within a given grouping of missense mutations, oncogenic driver mutations may be given higher priority. A normal human peptidome library of ˜12.6 million unique 9 and 10 mers curated from all known human protein sequences (HG19) has been created. Prior to final selection, any potential predicted epitopes derived from a missense mutation and all neoORF regions may be screened against this library, and perfect matches may be excluded. As discussed below, particular peptides predicted to have deleterious biochemical properties may be eliminated or modified.

According to the techniques herein, RNA levels may be analyzed to assess neoantigen expression. For example, RNA-Seq read-count may be used as a proxy to estimate neoantigen expression. However, there is no currently available information to assess the minimum RNA expression level required in a tumor cell needed to initiate cytolysis. Even the level of expression from “pioneer” translation of messages destined for nonsense mediated decay may be sufficient for target generation. Accordingly, the techniques herein initially set broad acceptance limits for RNA levels that may vary inversely with the priority group. As the predicted affinity decreases, higher stringency may be applied to expression levels. One of skill in the art will appreciate that as additional information becomes available, such limits may be adjusted.

Because of the high value of neoORFs as targets due to their novelty and exquisite tumor specificity, neoORFs with predicted binding epitopes (Kd≤500 nM) may be utilized even if there are no detectable mRNA molecules by RNA-Seq (Rank 1). Regions of neoORFs without predicted binding epitopes (>500 nM), may generally be utilized only if some level of RNA expression is detected (Rank 4). All missense mutations with strong to intermediate predicted MHC binding affinity (≤150 nM) may generally be utilized unless there were no RNA-Seq reads (Ranks 2 and 3). For missense mutations with lower predicted binding affinity (150-≤500 nM), these will likely be utilized only if a slightly higher level of RNA expression is detected (Rank 5).

Oncogenic drivers may represent a high priority group. For example, within a given grouping of missense mutations, oncogenic driver mutations may be of higher priority. This approach is based on the observed down-regulation of genes that are targeted by immune pressure (e.g., immunoediting). In contrast to other immune targets where down-regulation may not have a deleterious effect of cancer cell growth, continued expression of oncogenic driver genes may be crucial to cancer cell survival, thus shutting off a pathway of immune escape. Exemplary oncogenic drivers are listed in Table 3-1 (see e.g., Vogelstein et al; GOTERM_BP Assignment of genes to Gene Ontology Term—Biological Function on the worldwide web at (www)geneontology.org; BIOCARTA Assignment of genes to signaling pathways, on the worldwide web at (www)biocarta.com; KEGG Assignment of genes to pathways according to KEGG pathway database, on the worldwide web at (www)genome.jp/krgg/pathway.html; REACTOME Assignment of genes to pathways according to REACTOME pathways and gene interactions, on the worldwide web at (www)reactome.org).

TABLE 3-1
Exemplary Oncogenic Driver Genes

| | | # Mutated | | Tumor | | | |
|---|---|---|---|---|---|---|---|
| Gene | | Tumor | Oncogene | Suppressor | | Core | |
| Symbol | Gene Name | Samples** | score* | Gene score* | Classification* | pathway | Process |
| ABL1 | c-abl oncogene 1, | 851 | 93% | 0% | Oncogene | Cell | Cell |
| | receptor tyrosine | | | | | Cycle/Apoptosis | Survival |
| | kinase | | | | | | |
| AKT1 | v-akt murine | 155 | 93% | 1% | Oncogene | PI3K | Cell |
| | thymoma viral | | | | | | Survival |
| | oncogene homolog 1 | | | | | | |
| ALK | anaplastic lymphoma | 189 | 72% | 1% | Oncogene | PI3K; RAS | Cell |
| | receptor tyrosine | | | | | | Survival |
| | kinase | | | | | | |
| AR | androgen receptor | 23 | 54% | 0% | Oncogene | Transcriptional | Cell Fate |
| | | | | | | Regulation | |
| BCL2 | B-cell CLL/lymphoma | 45 | 27% | 1% | Oncogene | Cell | Cell |
| | 2 | | | | | Cycle/Apoptosis | Survival |
| BRAF | v-raf murine sarcoma | 24288 | 100% | 0% | Oncogene | RAS | Cell |
| | viral oncogene | | | | | | Survival |
| | homolog B1 | | | | | | |
| CARD11 | caspase recruitment | 74 | 30% | 1% | Oncogene | Cell | Cell |
| | domain family, | | | | | Cycle/Apoptosis | Survival |
| | member 11 | | | | | | |
| CBL | Cas-Br-M (murine) | 168 | 57% | 9% | Oncogene | PI3K; RAS | Cell |
| | ecotropic retroviral | | | | | | Survival |
| | transforming | | | | | | |
| | sequence | | | | | | |
| CRLF2 | cytokine receptor-like | 10 | 100% | 0% | Oncogene | STAT | Cell |
| | factor 2 | | | | | | Survival |
| CSF1R | colony stimulating | 48 | 50% | 15% | Oncogene | PI3K; RAS | Cell |
| | factor 1 receptor | | | | | | Survival |
| CTNNB1 | catenin (cadherin- | 3262 | 92% | 1% | Oncogene | APC | Cell Fate |
| | associated protein), | | | | | | |
| | beta 1, 88 kDa | | | | | | |
| DNMT1 | DNA (cytosine-5-)- | 22 | 36% | 5% | Oncogene | Chromatin | Cell Fate |
| | methyltransferase 1 | | | | | Modification | |
| DNMT3A | DNA (cytosine-5-)- | 788 | 74% | 12% | Oncogene | Chromatin | Cell Fate |
| | methyltransferase 3 | | | | | Modification | |
| | alpha | | | | | | |
| EGFR | epidermal growth | 10628 | 97% | 0% | Oncogene | PI3K; RAS | Cell |
| | factor receptor | | | | | | Survival |
| | (erythroblastic | | | | | | |
| | leukemia viral (v-erb- | | | | | | |
| | b) oncogene | | | | | | |
| | homolog, avian) | | | | | | |
| ERBB2 | v-erb-b2 | 164 | 67% | 3% | Oncogene | PI3K; RAS | Cell |
| | erythroblastic | | | | | | Survival |
| | leukemia viral | | | | | | |
| | oncogene homolog 2, | | | | | | |
| | neuro/glioblastoma | | | | | | |
| | derived oncogene | | | | | | |
| | homolog (avian) | | | | | | |
| EZH2 | enhancer of zeste | 276 | 67% | 12% | Oncogene | Chromatin | Cell Fate |
| | homolog 2 | | | | | Modification | |
| | (Drosophila) | | | | | | |
| FGFR2 | fibroblast growth | 121 | 49% | 6% | Oncogene | PI3K; RAS; STAT | Cell |
| | factor receptor 2 | | | | | | Survival |
| FGFR3 | fibroblast growth | 2948 | 99% | 0% | Oncogene | PI3K; RAS; STAT | Cell |
| | factor receptor 3 | | | | | | Survival |
| FLT3 | fms-related tyrosine | 11520 | 98% | 0% | Oncogene | RAS; PI3K; STAT | Cell |
| | kinase 3 | | | | | | Survival |
| FOXL2 | forkhead box L2 | 330 | 100% | 0% | Oncogene | TGF-β | Cell Fate |
| GATA2 | GATA binding protein | 45 | 53% | 4% | Oncogene | NOTCH, TGF-β | Cell Fate |
| | 2 | | | | | | |
| GNA11 | guanine nucleotide | 110 | 92% | 1% | Oncogene | PI3K; RAS; MAPK | Cell |
| | binding protein (G | | | | | | Survival |
| | protein), alpha 11 (Gq | | | | | | |
| | class) | | | | | | |
| GNAQ | guanine nucleotide | 245 | 95% | 1% | Oncogene | PI3K; RAS; MAPK | Cell |
| | binding protein (G | | | | | | Survival |
| | protein), q | | | | | | |
| | polypeptide | | | | | | |
| GNAS | GNAS complex locus | 422 | 93% | 2% | Oncogene | APC; PI3K; | Cell |
| | | | | | | TGF-β, RAS | Survival/ |
| | | | | | | | Cell Fate |
| H3F3A | H3 histone, family 3B | 122 | 93% | 0% | Oncogene | Chromatin | Cell Fate |
| | (H3.3B); H3 histone, | | | | | Modification | |
| | family 3A | | | | | | |
| | pseudogene; H3 | | | | | | |
| | histone, family 3A; | | | | | | |
| | similar to H3 histone, | | | | | | |
| | family 3B; similar to | | | | | | |
| | histone H3.3B | | | | | | |
| HIST1H3B | histone cluster 1, H3j; | 25 | 60% | 0% | Oncogene | Chromatin | Cell Fate |
| | histone cluster 1, H3i; | | | | | Modification | |
| | histone cluster 1, | | | | | | |
| | H3h; histone cluster | | | | | | |
| | 1, H3g; histone | | | | | | |
| | cluster 1, H3f; histone | | | | | | |
| | cluster 1, H3e; | | | | | | |
| | histone cluster 1, | | | | | | |
| | H3d; histone cluster | | | | | | |
| | 1, H3c; histone | | | | | | |
| | cluster 1, H3b; | | | | | | |
| | histone cluster 1, | | | | | | |
| | H3a; histone cluster | | | | | | |
| | 1, H2ad; histone | | | | | | |
| | cluster 2, H3a; | | | | | | |
| | histone cluster 2, | | | | | | |
| | H3c; histone cluster | | | | | | |
| | 2, H3d | | | | | | |
| HRAS | v-Ha-ras Harvey rat | 812 | 96% | 0% | Oncogene | RAS | Cell |
| | sarcoma viral | | | | | | Survival |
| | oncogene homolog | | | | | | |
| IDH1 | isocitrate | 4509 | 100% | 0% | Oncogene | Chromatin | Cell Fate |
| | dehydrogenase 1 | | | | | Modification | |
| | (NADP+), soluble | | | | | | |
| IDH2 | isocitrate | 1029 | 99% | 0% | Oncogene | Chromatin | Cell Fate |
| | dehydrogenase 2 | | | | | Modification | |
| | (NADP+), | | | | | | |
| | mitochondrial | | | | | | |
| JAK1 | Janus kinase 1 | 61 | 26% | 18% | Oncogene | STAT | Cell |
| | | | | | | | Survival |
| JAK2 | Janus kinase 2 | 32692 | 100% | 0% | Oncogene | STAT | Cell |
| | | | | | | | Survival |
| JAK3 | Janus kinase 3 | 89 | 60% | 6% | Oncogene | STAT | Cell |
| | | | | | | | Survival |
| KIT | similar to Mast/stem | 4720 | 90% | 0% | Oncogene | PI3K; RAS; STAT | Cell |
| | cell growth factor | | | | | | Survival |
| | receptor precursor | | | | | | |
| | (SCFR) (Proto- | | | | | | |
| | oncogene tyrosine- | | | | | | |
| | protein kinase Kit) (c- | | | | | | |
| | kit) (CD117 antigen); | | | | | | |
| | v-kit Hardy- | | | | | | |
| | Zuckerman 4 feline | | | | | | |
| | sarcoma viral | | | | | | |
| | oncogene homolog | | | | | | |
| KLF4 | Kruppel-like factor 4 | 61 | 80% | 4% | Oncogene | Transcriptional | Cell Fate |
| | | | | | | Regulation; WNT | |
| KRAS | v-Ki-ras2 Kirsten rat | 23261 | 100% | 0% | Oncogene | RAS | Cell |
| | sarcoma viral | | | | | | Survival |
| | oncogene homolog | | | | | | |
| MAP2K1 | mitogen-activated | 13 | 67% | 0% | Oncogene | RAS | Cell |
| | protein kinase kinase | | | | | | Survival |
| | 1 | | | | | | |
| MED12 | mediator complex | 337 | 84% | 0% | Oncogene | Cell | Cell |
| | subunit 12 | | | | | Cycle/Apoptosis; | Survival |
| | | | | | | TGF-β | |
| MET | met proto-oncogene | 159 | 61% | 4% | Oncogene | PI3K; RAS | Cell |
| | (hepatocyte growth | | | | | | Survival |
| | factor receptor) | | | | | | |
| MPL | myeloproliferative | 531 | 96% | 0% | Oncogene | STAT | Cell |
| | leukemia virus | | | | | | Survival |
| | oncogene | | | | | | |
| MYD88 | myeloid | 134 | 92% | 1% | Oncogene | Cell | Cell |
| | differentiation | | | | | Cycle/Apoptosis | Survival |
| | primary response | | | | | | |
| | gene (88) | | | | | | |
| NFE2L2 | nuclear factor | 102 | 74% | 1% | Oncogene | Cell | Cell |
| | (erythroid-derived 2)- | | | | | Cycle/Apoptosis | Survival |
| | like 2 | | | | | | |
| NRAS | neuroblastoma RAS | 2738 | 99% | 0% | Oncogene | RAS | Cell |
| | viral (v-ras) oncogene | | | | | | Survival |
| | homolog | | | | | | |
| PDGFRA | platelet-derived | 653 | 84% | 1% | Oncogene | PI3K; RAS | Cell |
| | growth factor | | | | | | Survival |
| | receptor, alpha | | | | | | |
| | polypeptide | | | | | | |
| PIK3CA | phosphoinositide-3- | 4560 | 95% | 1% | Oncogene | PI3K | Cell |
| | kinase, catalytic, | | | | | | Survival |
| | alpha polypeptide | | | | | | |
| PPP2R1A | protein phosphatase | 86 | 85% | 2% | Oncogene | Cell | Cell |
| | 2 (formerly 2A), | | | | | Cycle/Apoptosis | Survival |
| | regulatory subunit A, | | | | | | |
| | alpha isoform | | | | | | |
| PTPN11 | protein tyrosine | 410 | 90% | 0% | Oncogene | RAS | Cell |
| | phosphatase, non- | | | | | | Survival |
| | receptor type 11; | | | | | | |
| | similar to protein | | | | | | |
| | tyrosine phosphatase, | | | | | | |
| | non-receptor type 11 | | | | | | |
| RET | ret proto-oncogene | 500 | 86% | 1% | Oncogene | RAS; PI3K | Cell |
| | | | | | | | Survival |
| SETBP1 | SET binding protein 1 | 95 | 25% | 4% | Oncogene | Chromatin | Cell Fate |
| | | | | | | Modification; | |
| | | | | | | Replication | |
| SF3B1 | splicing factor 3b, | 516 | 91% | 0% | Oncogene | Transcriptional | Cell Fate |
| | subunit 1, 155 kDa | | | | | Regulation | |
| SMO | smoothened homolog | 34 | 51% | 3% | Oncogene | HH | Cell Fate |
| | (Drosophila) | | | | | | |
| SPOP | speckle-type POZ | 35 | 66% | 3% | Oncogene | Chromatin | Cell Fate |
| | protein | | | | | Modification; | |
| | | | | | | HH | |
| SRSF2 | SRSF2 | 273 | 95% | 2% | Oncogene | Transcriptional | Cell Fate |
| | serine/arginine-rich | | | | | Regulation | |
| | splicing factor 2 | | | | | | |
| TSHR | thyroid stimulating | 301 | 86% | 0% | Oncogene | PI3K; MAPK | Cell |
| | hormone receptor | | | | | | Survival |
| U2AF1 | U2 small nuclear RNA | 96 | 92% | 1% | Oncogene | Transcriptional | Cell Fate |
| | auxiliary factor 1 | | | | | Regulation | |

### Example 8

### Peptide Production and Formulation

GMP neo-antigenic peptides for immunization will be prepared by chemical synthesis Merrifield RB: Solid phase peptide synthesis. I. The synthesis of a tetrapeptide. J. Am. Chem. Soc. 85:2149-54, 1963) in accordance with FDA regulations. Three development runs have been conducted of 20 ˜20-30mer peptides each. Each run was conducted in the same facility and utilized the same equipment as will be used for the GMP runs, utilizing draft GMP batch records. Each run successfully produced >50 mg of each peptide, which were tested by all currently planned release tests (e.g., Appearance, Identify by MS, Purity by RP-HPLC, Content by Elemental Nitrogen, and TFA content by RP-HPLC) and met the targeted specification where appropriate. The products were also produced within the timeframe anticipated for this part of the process (approximately 4 weeks). The lyophilized bulk peptides were placed on a long term stability study and will be evaluated at various time points up to 12 months.

Material from these runs has been used to test the planned dissolution and mixing approach. Briefly, each peptide will be dissolved at high concentration (50 mg/ml) in 100% DMSO and diluted to 2 mg/ml in an aqueous solvent. Initially, it was anticipated that PBS would be used as a diluent, however, a salting out of a small number of peptides caused a visible cloudiness. D5W (5% dextrose in water) was shown to be much more effective; 37 of 40 peptides were successfully diluted to a clear solution. The only problematic peptides are very hydrophobic peptides. The predicted biochemical properties of planned immunizing peptides will be evaluated and synthesis plans may be altered accordingly (using a shorter peptide, shifting the region to be synthesized in the N- or C-terminal direction around the predicted epitope, or potentially utilizing an alternate peptide). Ten separate peptides in DMSO/D5W were subjected to two freeze/thaw cycles and showed full recovery. Two individual peptides were dissolved in DMSO/D5W and placed on stability at two temperatures (−20° C. and −80° C.). These peptides will be evaluated (RP-HPLC, MS and pH) for up to 6 months. To date, both peptides are stable at the 12 week time point with additional time points at 24 weeks to be evaluated.

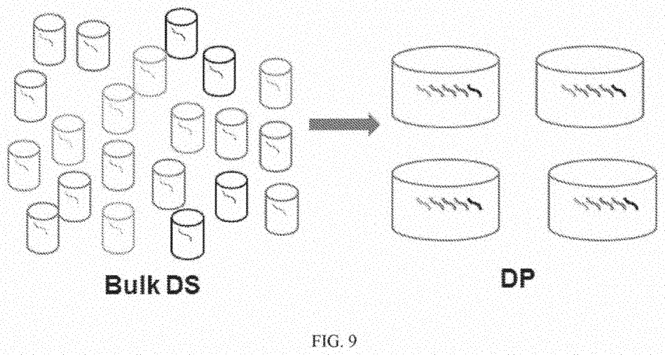

As shown in FIG. 9, the design of the dosage form process is to prepare 4 pools of patient-specific peptides consisting of 5 peptides each. A RP-HPLC assay has been prepared and qualified to evaluate these peptide mixes. This assay achieves good resolution of multiple peptides within a single mix and can also be used to quantitate individual peptides.

Membrane filtration (0.2 μm pore size) will be used to reduce bioburden and conduct final filter sterilization. Four different appropriately sized filter types were initially evaluated and the Pall, PES filter (#4612) was selected. To date, 4 different mixtures of 5 different peptides each have been prepared and individually filtered sequentially through two PES filters. Recovery of each individual peptide was evaluated utilizing the RP-HPLC assay. For 18 of the 20 peptides, the recovery after two filtrations was >90%. For two highly hydrophobic peptides, the recovery was below 60% when evaluated at small scale but were nearly fully recovered (87 and 97%) at scale. As stated above, approaches will be undertaken to limit the hydrophobic nature of the sequences selected.

GMP neo-antigenic peptides for immunization will be prepared by chemical synthesis Merrifield R B: Solid phase peptide synthesis. I. The synthesis of a tetrapeptide. J. Am. Chem. Soc. 85:2149-54, 1963) in accordance with FDA regulations.

### Example 9

### Endpoint Assessment

The primary immunological endpoint of this study will be the assessment of T cell response measured by ex vivo IFN-γ ELISPOT. IFN-γ secretion occurs as a result of the recognition of cognate peptides or mitogenic stimuli by CD4⁺ and/or CD8⁺ T-cells. A multitude of different CD4⁺ and CD8⁺ determinants will likely be presented to T cells in vivo since the 20-30mer peptides used for vaccination should undergo processing into smaller peptides by antigen presenting cells. Without being bound by theory, it is believed that the combination of personalized neo-antigen peptides, which are novel to the immune system and thus not subject to the immune-dampening effects of self-tolerance, and the powerful immune adjuvant poly-ICLC will induce strong CD4⁺ and/or CD8⁺ responses. The expectation is therefore that T cell responses are detectable ex vivo i.e. without the need for in vitro expansion of epitope specific T cells through short-term culture. Patients will initially be evaluated using the total pool of peptide immunogens as stimulant in the ELISPOT assay. For patients demonstrating a robust positive response, the precise immunogenic peptide(s) will be determined in follow-up analysis. The IFN-γ ELISPOT is generally accepted as a robust and reproducible assay to detect ex vivo T cell activity and determine specificity. In addition to the analysis of the magnitude and determinant mapping of the T cell response in peripheral blood monocytes, other aspects of the immune response induced by the vaccine are critical and will be assessed. These evaluations will be performed in patients who exhibit an ex vivo IFN-γ ELISPOT response in the screening assay. They include the evaluation of T cell subsets (Th1 versus Th2, T effector versus memory cells), analysis of the presence and abundance of regulatory cells such as T regulatory cells or myeloid derived suppressor cells, and cytotoxicity assays if patient-specific melanoma cells lines are successfully established.

### Example 10

### Peptide Synthesis

GMP peptides will be synthesized by standard solid phase synthetic peptide chemistry and purified by RP-HPLC. Each individual peptide will be analyzed by a variety of qualified assays to assess appearance (visual), purity (RP-HPLC), identity (by mass spectrometry), quantity (elemental nitrogen), and trifluoroacetate counterion (RP-HPLC) and released.

The personalized neoantigen peptides may be comprised of up to 20 distinct peptides unique to each patient. Each peptide may be a linear polymer of ˜20-˜30 L-amino acids joined by standard peptide bonds. The amino terminus may be a primary amine (NH2-) and the carboxy terminus is a carbonyl group (—COOH). The standard 20 amino acids commonly found in mammalian cells are utilized (alanine, arginine, asparagine, aspartic acid, cysteine, glutamine, glutamic acid , glycine, histidine, isoleucine, leucine lysine, methionine, phenylalanine, proline, serine, threonine, tryptophan, tyrosine, valine). The molecular weight of each peptide varies based on its length and sequence and is calculated for each peptide.

Personalized neoantigen peptides may be supplied as a box containing 2 ml Nunc Cryo vials with color-coded caps, each vial containing approximately 1.5 ml of a frozen DMSO/D5W solution containing up to 5 peptides at a concentration of 400 ug/ml. There may be 10-15 vials for each of the four groups of peptides. The vials are to be stored at −80 oC until use. Ongoing stability studies support the storage temperature and time.

Storage and Stability: The personalized neoantigen peptides are stored frozen at −80 oC. The thawed, sterile filtered, in process intermediates and the final mixture of personalized neoantigen peptides and poly-ICLC can be kept at room temperature but should be used within 4 hours .

Compatibility: The personalized neoantigen peptides will be mixed with ⅓ volume poly-ICLC just prior to use.

### Example 11

### Administration

Following mixing with the personalized neo-antigenic peptides/polypeptides, the vaccine (e.g., peptides+poly-ICLC) is to be administered subcutaneously.

Preparation of personalized neo-antigenic peptides/polypeptides pools: peptides will be mixed together in 4 pools of up to 5 peptides each. The selection criteria for each pool will be based on the particular MHC allele to which the peptide is predicted to bind.

Pool Composition: The composition of the pools will be selected on the basis of the particular HLA allele to which each peptide is predicted to bind. The four pools will be injected into anatomic sites that drain to separate lymph node basins. This approach was chosen in order to potentially reduce antigenic competition between peptides binding to the same HLA allele as much as possible and involve a wide subset of the patient's immune system in developing an immune response. For each patient, peptides predicted to bind up to four different HLA A and B alleles will be identified. Some neoORF derived peptides will not be associated with any particular HLA allele. The approach to distributing peptides to different pools will be to spread each set of peptides associated with a particular HLA allele over as many of the four pools as possible. It is highly likely there will be situations where there will be more than 4 predicted peptides for a given allele, and in these cases it will be necessary to allocate more than one peptide associated with a particular allele to the same pool. Those neoORF peptides not associated with any particular allele will be randomly assigned to the remaining slots. An example is shown below:

| | | |
|---|---|---|
| A1 | HLA A0101 | 3 peptides |
| A2 | HLA A1101 | 5 peptides |
| B1 | HLA B0702 | 2 peptides |
| B2 | HLA B6801 | 7 peptides |
| X | NONE (neoORF) | 3 peptides |

| Pool # | 1 | 2 | 3 | 4 |
|---|---|---|---|---|
| | B2 | B2 | B2 | B2 |
| | B2 | B2 | B2 | A2 |
| | A2 | A2 | A2 | A2 |
| | A1 | A1 | A1 | B1 |
| | B1 | X | X | X |

Peptides predicted to bind to the same MHC allele will be placed into separate pools whenever possible. Some of the neoORF peptides may not be predicted to bind to any MHC allele of the patient. These peptides will still be utilized however, primarily because they are completely novel and therefore not subject to the immune-dampening effects of central tolerance and therefore have a high probability of being immunogenic. NeoORF peptides also carry a dramatically reduced potential for autoimmunity as there is no equivalent molecule in any normal cell. In addition, there can be false negatives arising from the prediction algorithm and it is possible that the peptide will contain a HLA class II epitope (HLA class II epitopes are not reliably predicted based on current algorithms). All peptides not identified with a particular HLA allele will be randomly assigned to the individual pools. The amounts of each peptide are predicated on a final dose of 300 μg of each peptide per injection.

For each patient, four distinct pools (labeled “A”, “B”, “C” and “D”) of 5 synthetic peptides each will have been prepared manufacturer and stored at −80° C. On the day of immunization, the complete vaccine consisting of the peptide component(s) and poly-ICLC will be prepared in a laminar flow biosafety cabinet in the research pharmacy. One vial each (A, B, C and D) will be thawed at room temperature and moved into a biosafety cabinet for the remaining steps. 0.75 ml of each peptide pool will be withdrawn from the vial into separate syringes. Separately, four 0.25 ml (0.5 mg) aliquots of poly-ICLC will be withdrawn into separate syringes. The contents of each peptide-pool containing syringe will then be gently mixed with a 0.25 ml aliquot of poly-ICLC by syringe-to-syringe transfer. The entire one ml of the mixture will be used for injection. These 4 preparations will be labeled “study drug A”, “study drug B”, “study drug C”, and “study drug D”.

Injections: At each immunization, each of the 4 study drugs will be injected subcutaneously into one extremity. Each individual study drug will be administered to the same extremity at each immunization for the entire duration of the treatment (i.e. study drug A will be injected into left arm on day 1, 4, 8 etc., study drug B will be injected into right arm on days 1, 4, 8 etc.). Alternative anatomical locations for patients who are status post complete axillary or inguinal lymph node dissection are the left and right midriff, respectively.

Vaccine will be administered following a prime/boost schedule. Priming doses of vaccine will be administered on days 1, 4, 8, 15, and 22 as shown above. In the boost phase, vaccine will be administered on days 85 (week 13) and 169 (week 25).

All patients receiving at least one dose of vaccine will be evaluable for toxicity. Patients will be evaluable for immunologic activity if they have received all vaccinations during the induction phase and the first vaccination (boost) during the maintenance phase.

### Example 12

### Pharmacodynamic Studies

The immunization strategy is a “prime-boost” approach, involving an initial series of closely spaced immunizations to induce an immune response followed by a period of rest to allow memory T-cells to be established. This will be followed by a booster immunization, and the T-cell response 4 weeks after this boost (16 weeks after the first vaccination) is expected to generate the strongest response and will be the primary immunological endpoint. Immune monitoring will be performed in a step-wise fashion as outlined below to characterize the intensity and quality of the elicited immune responses. Peripheral blood will be collected and PBMC will be frozen at two separate time points prior to the first vaccination (baseline) and at different time points thereafter as illustrated in Schema B and specified in the study calendar. Immune monitoring in a given patient will be performed after the entire set of samples from the induction phase and the maintenance phase, respectively, have been collected. If sufficient tumor tissue is available, a portion of the tumor will be used to develop autologous melanoma cell lines for use in cytotoxic T-cell assays.

### Example 13

### Screening Ex Vivo IFN-γ ELISPOT

For each patient, a set of screening peptides will be synthesized. The screening peptides will be 15 amino acids in length (occasionally a 16mer or 17mer will be used), overlapping by 11 amino acids and covering the entire length of each peptide or the entire length of the neoORF for neoORF-derived peptides. The entire set of patient-specific screening peptides will be pooled together at approximately equal concentration and a portion of each peptide will also be stored individually. Purity of the peptide pool will be ascertained by testing PBMC from 5 healthy donors with established low background in ex vivo IFN-γ ELISPOTs. Initially, PBMC obtained at baseline and at week 16 (the primary immunological endpoint) will be stimulated for 18 hours with the complete pool of overlapping 15-mer peptides (11 amino acids overlap) to examine the global response to the peptide vaccine. Subsequent assays may utilize PBMC collected at other time points as indicated. If no response is identified at the primary immunological endpoint using the ex vivo IFN-γ ELISPOT assay, PBMC will be stimulated with the peptide pool for a longer time period (up to 10 days) and re-analyzed.

### Example 14

### Deconvolution of Epitopes in Follow-Up Ex Vivo IFN-γ ELISPOT Assays

Once an ex vivo IFN-γ ELISPOT response elicited by an overlapping peptide pool is observed (defined as at least 55 spot forming units/10⁶ PBMC or increased at least 3 times over baseline), the particular immunogenic peptide eliciting this response will be identified by de-convoluting the peptide pool based into sub-pools based on the immunizing peptides and repeating the ex vivo IFN-γ ELISPOT assays. For some responses, an attempt will be made to precisely characterize the stimulating epitope by utilizing overlapping 8-10 mer peptides derived from confirmed, stimulating peptides in IFN-γ ELISPOT assays. Additional assays may be conducted on a case-by case basis for appropriate samples. For example,
The entire 15mer pool or sub-pools will be used as stimulating peptides for intracellular cytokine staining assays to identify and quantify antigen-specific CD4+, CD8+, central memory and effector memory populations
Similarly, these pools will be used to evaluate the pattern of cytokines secreted by these cells to determine the T_{H}1 vs T_{H}2 phenotype
Extracellular cytokine staining and flow cytometry of unstimulated cells will be used to quantify Treg and myeloid-derived suppressor cells (MDSC).
If a melanoma cell line is successfully established from a responding patient and the activating epitope can be identified, T-cell cytotoxicity assays will be conducted using the mutant and corresponding wild type peptide
PBMC from the primary immunological endpoint will be evaluated for “epitope spreading” by using known melanoma tumor associated antigens as stimulants and by using several additional identified mutated epitopes that were not selected to be among the immunogens
Immuno-histochemistry of tumor samples will be conducted to quantify CD4+, CD8+, MDSC, and Treg infiltrating populations.

### Example 15

### Pipeline for the Systematic Identification of Tumor Neoantigens

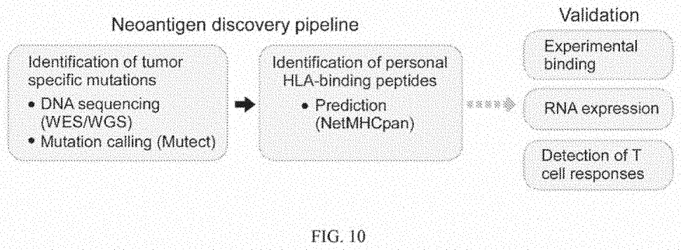

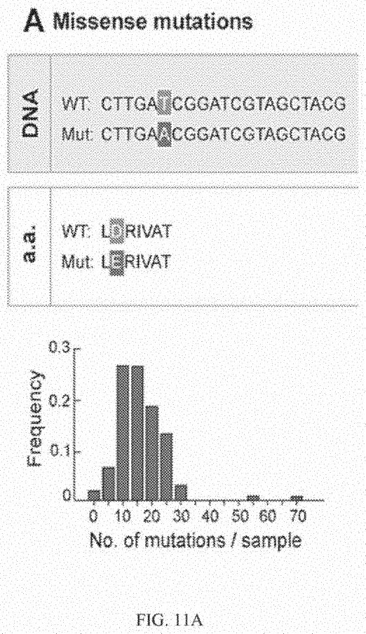

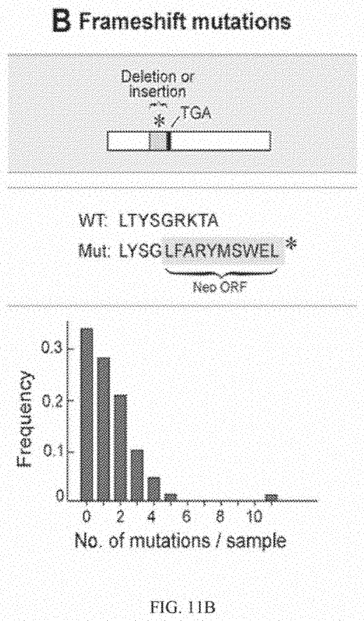

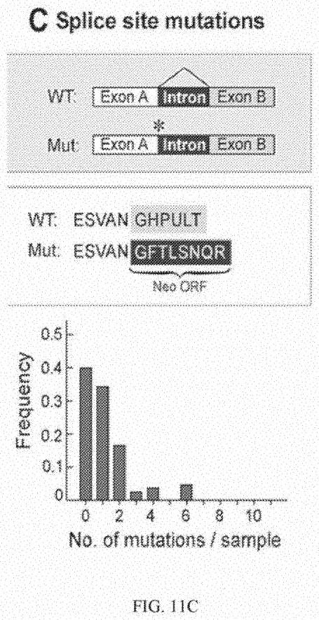

Recent advances in sequencing technologies and peptide epitope predictions were leveraged to generate a two-step pipeline to systematically discover candidate tumor-specific HLA-bound neoantigens. As depicted in FIG. 10, this approach starts with DNA sequencing of tumors (e.g., by either whole-exome (WES) or whole-genome sequencing (WGS)) in parallel with matched normal DNA to comprehensively identify non-synonymous somatic mutations (see e.g., Lawrence et al. 2013; Cibulski et al. 2012). Next, candidate tumor specific mutated peptides generated by tumor mutations with the potential to bind personal class I HLA proteins, and hence be presented to CD8⁺ T cells, may be predicted using prediction algorithms such as, for example, NetMHCpan (see e.g., Lin 2008; Zhang 2011). Candidate peptide antigens were further evaluated based on experimental validation of their binding to HLA and expression cognate mRNAs in autologous leukemia cells.

This pipeline was applied to a large dataset of sequenced CLL samples (see e.g., Wang et al. 2011). From 91 cases that were sequenced by either WES or WGS, a total of 1838 non-synonymous mutations were discovered in protein-coding regions, corresponding to a mean somatic mutation rate of 0.72 (±0.36 s.d.) per megabase (range, 0.08 to 2.70), and a mean of 20 non-synonymous mutations per patient (range, 2 to 76) (see e.g., Wang et al. 2011). Three general classes of mutations were identified that would be expected to generate regions of amino acid changes and hence potentially be recognized immunologically. The most abundant class included missense mutation that cause single amino acid (aa) changes, representing 90% of somatic mutations per CLL. Of 91 samples, 99% harbored missense mutations and 69% had between 10-25 missense mutations (see e.g., FIG. 2A). The other two classes of mutations, frameshifts and splice-site mutations (mutations at exon-intron junctions) have the potential to generate longer stretches of novel amino acid sequences entirely specific to the tumor (neo-open reading frames, or neoORFs), with a higher number of neoantigen peptides per given alteration (compared to missense mutations). However, consistent with data from other cancer types, neoORF-generating mutations were approximately 10 fold less abundant than missense mutations in CLL (see e.g., FIGS. 2B-C). Given the prevalence of missense mutations, subsequent experimental studies was focused on the analysis of neoepitopes generated by missense mutations.

### Example 16

### Somatic Missense Mutations Generate Neopeptides Predicted to Bind to Personal HLA Class I Alleles

T cell recognition of peptide epitopes by the T cell receptor (TCR) requires the display of peptides bound within the binding groove of HLA molecules on the surface of antigen-presenting cells. Recent comparative studies across the >30 available class I prediction algorithms have shown NetMHCpan to consistently perform with high sensitivity: and specificity across HLA alleles (see e.g., Zhang et al. 2011).

The NetMHCpan algorithm was tested against a set of 33 known mutated epitopes that were originally identified in the literature on the basis of their functional activity (i.e., ability to stimulate antitumor cytolytic T cell responses) or were characterized as immunogenic minor histocompatibility antigens to determine whether the algorithm would correctly predict binding for the 33 known mutated epitopes (see e.g., Tables 4 and 5). Tables 4 and 5 show HLA-peptide binding affinities of known functionally derived immunogenic mutated epitopes across human cancers using NetMHCpan. Table 4 shows epitopes from missense mutations (NSCLC: non-small cell lung cancer; MEL: melanoma; CLL: chronic lymphocytic leukemia; RCC: clear cell renal carcinoma; BLD: bladder cancer; NR: not reported; ). bolded underlined font: IC50<150 nM, italicized font: IC50 150-500 nM and italicized and underlined font: IC50>500 nM.

TABLE 4

| | | | | T cell | | Mutated | | Wildtype | | | |
|---|---|---|---|---|---|---|---|---|---|---|---|
| | | | Clini- | re- | | | Pre- | | Pre- | | |
| | | | cal | sponse | | | dicted | | dicted | W/ | Ref- |
| | | Dis- | Re- | MUT>>> | HLA | Observed | IC50 | Observed | IC50 | MUT | er- |
| Group | Gene | ease | sponse | WT | Allele | epitope | (nM) | epitope | (nM) | IC50 | ence |
| 1 | ME-1 | NSCLC | Yes | Yes | A*02:01 | FLDEFMEGV | 3 | FLDEFMEAV | 2 | 0.7 | (50) |
| | PLEKHM2 | MEL | Yes | Yes | A*01:01 | LTDDRLFTCY | 3 | LTDDRLFTCH | 97 | 32 | (36) |
| | PRDX5 | MEL | NR | Yes | A*02:01 | LLLDDLLVSI | 5 | LLLDDSLVSI | 7 | 1.4 | (51) |
| | MATN2 | MEL | Yes | Yes | A*11:01 | KTLTSVFQK | 5 | ETLTSVFQK | 20 | 4 | (36) |
| | DDX21 | MEL | Yes | Yes | A*68:01 | EAFIQPITR | 10 | EASIQPITR | 29 | 3 | (52) |
| | RBAF | MEL | Yes | Yes | B*07:02 | RPHVPESAF | 10 | GPHVPESAF | 68 | 7 | (13) |
| | GAS7 | MEL | Yes | Yes | A*02:01 | SLADEAEVYL | 12 | SLADEAEVHL | 39 | 3 | (14) |
| | SIRT2 | MEL | Yes | Yes | A*03:01 | KIFSEVTLK | 14 | KIFSEVTPK | 16 | 1.1 | (13) |
| | EF2 | NSCLC | NR | Yes | A*68:02 | ETVSEQSNV | 16 | ETVSEESNV | 27 | 2 | (53) |
| | GAPDH | MEL | Yes | Yes | A*02:01 | GIVEGLITTV | 21 | GIVEGLMTTV | 27 | 1.3 | (14) |
| | HSP 70 | RCC | NR | Yes | A*02:01 | SLFEGIDIYT | 23 | SLFEGIDFYT | 7 | 0.3 | (54) |
| | ACTININ | NSCLC | Yes | Yes | A*02:01 | FIASNGVKLV | 29 | FIASKGVKLV | 44 | 2 | (55) |
| | CDK12 | MEL | Yes | Yes | A*11:01 | CILGKLFTK | 33 | CILGELFTK | 42 | 1.3 | (36) |
| | KIAA1440 | RCC | Yes | Yes | A*01:01 | QTACEVLDY | 33 | QTTCEVLDY | 78 | 2 | (14) |
| | HAUS3 | MEL | Yes | Yes | A*02:01 | ILNAMIAKI | 34 | ILNAMITKI | 36 | 1.1 | (36) |
| | PPP1R3B | MEL | Yes | Yes | A*01:01 | YTDFHCQYV | 49 | YTDFPCQYV | 72 | 1.5 | (36) |
| | MUM-2 | MEL | Yes | Yes | B*44:02 | SELFRSGLDSY | 184 | SELFRSRLDSY | 182 | 1 | (56) |
| 2 | KIAA0205 | BLD | NR | Yes | B*44:03 | AEPIDIQTW | 258 | AEPINIQTVV | 288 | 1.1 | (57) |
| | GPNMB | MEL | Yes | Yes | A*03:01 | TLDWLLQTPK | 282 | TLGWLLQTPK | 179 | 0.6 | (13) |
| | CSNK1A1 | MEL | Yes | Yes | A*02:01 | GLFGDIYLAI | 6 | GSFGDIYLAI | 1312 | 219 | (36) |
| 3 | CLPP | MEL | Yes | Yes | A*02:01 | ILDKVLVHL | 32 | ILDKVLVHP | 7566 | 236 | (58) |
| | CTNNB1 | MEL | Yes | Yes | A*24:02 | SYLDSGIHF | 41 | SYLDSGIHS | 18746 | 457 | (59) |
| | SNRP | MEL | Yes | Yes | A*03:01 | KILDAVVAQK | 48 | KILDAVVAQE | 14976 | 312 | (13) |
| | OS9 | MEL | NR | Yes | B*44:03 | KELEGILLL | 60 | KELEGILLP | 1161 | 19 | (60) |
| | MYH2 | MEL | Yes | Yes | A*03:01 | KINKNPKYK | 141 | EINKNPKYK | 4960 | 35 | (61) |
| | MART-2 | MEL | Yes | Yes | A*01:01 | FLEGNEVGKTY | 1115 | FLGGNEVGKTY | 4504 | 4 | (62) |
| 4 | NFYC | NSCLC | NR | Yes | B*52:01 | AQQITKTEV | 7314 | AQQITQTEV | 5701 | 0.8 | (63) |
| | CDK4 | MEL | NR | Yes | A*02:01 | ACDPHSGHFV | 11192 | ARDPHSGHFV | 25222 | 2 | (64) |

Table 5 shows epitopes from minor histocompatibility antigens (MM: multiple myeloma; HM: hematological malignancy; B-ALL: B cell acute lymphocytic leukemia).

TABLE 5

| | | | | | | Mutated | | | | | |
|---|---|---|---|---|---|---|---|---|---|---|---|
| | | | | T cell | | Ob- | Pre- | Ob- | Pre- | | |
| | | | Clinical | response | | served | dicted | served | dicted | WT/ | Ref- |
| | | | | MUT>>> | | epi- | IC50 | epi- | IC50 | MUT | er- |
| Group | Gene | Disease | sReponse | WT | Allele | tope | (nM) | tope | (nM) | IC50 | ence |
| 1 | ECGF- | MM | Yes | Yes | B*07:02 | RPHAIR | 3 | RPRAIR | 2 | 0.7 | (65) |
| | 1 | | | | | RPLAL | | RPLAL | | | |
| 1 | KIAA | HM | Yes | NR | A*02:01 | VLHDD | 17 | VLRDD | 140 | 8 | (66) |
| | 0223 | | | | | LLEA | | LLEA | | | |
| | (HA- | | | | | | | | | | |
| | 1) | | | | | | | | | | |
| 1 | BCL2 | HM | NR | Yes | A*24:02 | DYLQY | 22 | DYLQC | 34 | 2 | (67) |
| | A1 | | | | | VLQI | | VLQI | | | |
| 1 | BCL2 | HM | NR | Yes | A*24:02 | KEFED | 36 | KEFED | 27 | 0.8 | (67) |
| | A1 | | | | | DIINW | | GIINW | | | |
| 1 | HB-1 | B- | NR | Yes | B*44:03 | EEKRG | 81 | EEKRG | 67 | 1 | (68) |
| | | ALL | | | | SLHVW | | SLYVW | | | |

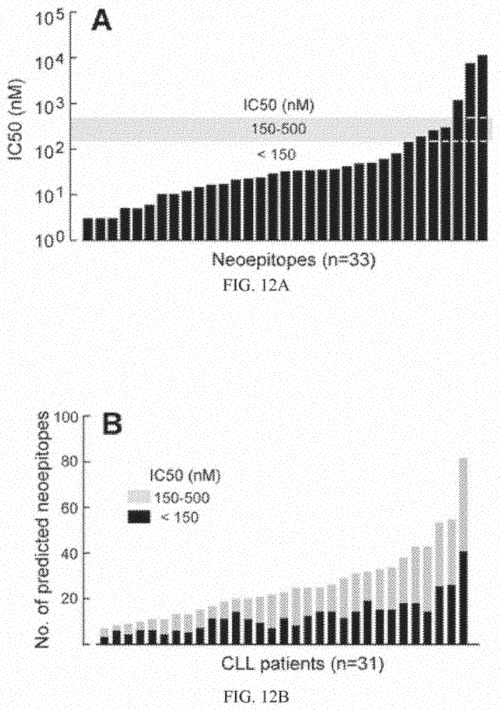

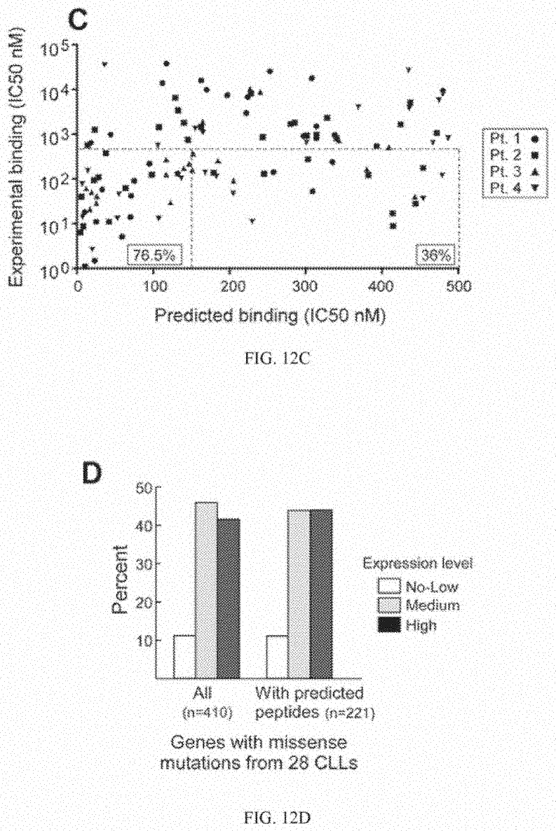

Among all tiled 9-mer and 10-mer possibilities, NetMHCpan identified all 33 functionally validated mutated epitopes as the best binding peptide among the possible choices for the given mutation. The median predicted binding affinity (IC50) to the known reported HLA restricting elements of each of the 33 mutated epitopes was 32 nM (range, 3-11, 192 nM). By setting the predicted IC50 cut-offs to 150 and 500 nM, 82 and 91% of the functionally validated peptides, respectively, were captured (see e.g., Tables 4 and 5 and FIG. 12A).

On the basis of its high degree of sensitivity and specificity, NetMHCpan was then applied to the 31 of 91 CLL cases for which HLA typing information was available. By convention, peptides with IC50<150 nM were considered as strong to intermediate binders, IC50 150-500 nM as weak binders, and IC50>500 nM as non-binders, respectively (see e.g., Cai et al. 2012). For all 91 CLL cases, a median of 10 strong binding peptides (range, 2-40) and 12 intermediate to weak binding peptides (range, 2-41) was found. In total, a median of 22 (range, 6-81) peptides per case was predicted with IC50<500 nM (see e.g., FIG. 12B and Table 6). In particular, Table 6 shows that the numbers and affinity distributions of peptides predicted from 31 CLL cases with available HLA typing. Patients expressing the 8 most common HLA-A, -B alleles in the Caucasian population are marked with an “x”.

TABLE 6

| | | | | | | | | | | # of predicted | |
|---|---|---|---|---|---|---|---|---|---|---|---|
| | | | | | | | | | | neopeptides | |
| Pts | HLA-A alleles | | | | HLA-B alleles | | | | Uncommon | <150 | 150-500 |
| (1) | *01:01 | *02:01 | *24:02 | *03:01 | *07:02 | *08:01 | *15:01 | *38:01 | alleles | (nM) | (nM) |
| P46 | x | | | x | | x | x | | | 10 | 12 |
| P91 | x | x | | | x | x | | | | 17 | 20 |
| P32 | x | x | | | | x | | | B*44:02 | 13 | 6 |
| P60 | x | | | | x | x | | | A*68:01 | 14 | 19 |
| P5 | | x | | | | | x | | A*32:01; | 6 | 15 |
| | | | | | | | | | B*44:02 | | |
| P88 | | x | | | x | | | | A*29:02; | 17 | 25 |
| | | | | | | | | | B*44:03 | | |
| P7 | | x | | x | | | | | B*35:01; | 8 | 12 |
| | | | | | | | | | *57:01 | | |
| P8 | | | | x | | | | x | A*31:01; | 6 | 8 |
| | | | | | | | | | B*14:02 | | |
| P14 | | x | | x | | | | | B*51:01; | 5 | 4 |
| | | | | | | | | | *62.01 | | |
| P28 | | x | | | | | | x | A*26:01; | 10 | 6 |
| | | | | | | | | | B*48:01 | | |
| P34 | | | x | x | | | | | B*18:01; | 5 | 7 |
| | | | | | | | | | *39:06 | | |
| P35 | | x | | | | | | x | B*51:01 | 11 | 13 |
| P37 | x | | | | | x | | | A*03:02; | 2 | 4 |
| | | | | | | | | | B*44:03 | | |
| P49 | | x | | | x | | | | A*11:01; | 25 | 29 |
| | | | | | | | | | B*35:03 | | |
| P42 | | | x | | | | | x | A*26:01; | 10 | 18 |
| | | | | | | | | | B*35:02 | | |
| P66 | x | | | | | x | | | A*23:01; | 4 | 8 |
| | | | | | | | | | B*49:01 | | |
| P82 | | x | | | | | | x | A*26:01; | 13 | 29 |
| | | | | | | | | | B*35:08 | | |
| P83 | x | x | | | | | | | B*06:01; | 5 | 2 |
| | | | | | | | | | *04.01 | | |
| P87 | | | | | | | x | x | A*26:01; | 24 | 29 |
| | | | | | | | | | *31:01 | | |
| P47 | | | | x | x | | | | A*11:01; | 10 | 9 |
| | | | | | | | | | B*51:01 | | |
| P48 | | | x | | | | x | | A*11:01; | 13 | 17 |
| | | | | | | | | | B*51:01 | | |
| P16 | | x | | | | | | | A*11:01; | 13 | 12 |
| | | | | | | | | | B*44:02; | | |
| | | | | | | | | | *51:01 | | |
| P36 | | | x | | | | | | A*68:01; | 3 | 5 |
| | | | | | | | | | B*14:02 | | |
| P39 | | | | x | | | | | A*32:01; | 40 | 41 |
| | | | | | | | | | B*35:01; | | |
| | | | | | | | | | *44:01 | | |
| P53 | | | | | | | | x | A*26:01; | 13 | 11 |
| | | | | | | | | | B*35:01 | | |
| P73 | | | | | | | x | | A*31:01; | 18 | 13 |
| | | | | | | | | | *68:01; | | |
| | | | | | | | | | B*49:01 | | |
| P90 | | x | | | | | | | A*29:02; | 3 | 7 |
| | | | | | | | | | B*45:01; | | |
| | | | | | | | | | *55:01 | | |
| P45 | | x | | | | | | | A*68:02; | 5 | 5 |
| | | | | | | | | | B*44:02; | | |
| | | | | | | | | | *15:03 | | |
| P1 | | | | | | | | | A*11:01; | 7 | 17 |
| | | | | | | | | | *23:01; | | |
| | | | | | | | | | B*35:01; | | |
| | | | | | | | | | *51:01 | | |
| P40 | | | | | | | | | A*11:01; | 14 | 18 |
| | | | | | | | | | *32:01; | | |
| | | | | | | | | | B*40:01; | | |
| | | | | | | | | | *44:03 | | |
| P41 | | | | | | | | | A*29:03; | 10 | 8 |
| | | | | | | | | | *32:01; | | |
| | | | | | | | | | B*44:03 | | |

### Example 17

### More than Half of Predicted HLA-Binding Neopeptides Showed Direct Binding to HLA Proteins In Vitro

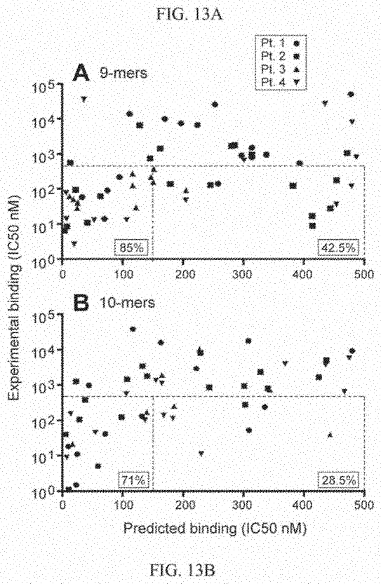

As shown in Table 7, IC50 nM scores generated by HLA-peptide binding predictions were validated using a competitive MHC I allele binding assay and focused on class I-A and -B alleles. To this end, 112 mutated peptides (9 or 10-mer mutated peptides) with predicted IC50 scores of less than 500 nM that were identified from 4 CLL cases (Pt 1-4) were synthesized. The experimental results correlated with the binding predictions. Experimental binding (defined as IC 50<500 NM) was confirmed in 76.5% and 36% of peptides predicted with IC50 of <150 nM or 150-500 nM, respectively (see e.g., FIG. 12C). In total, ˜54.5% (61/112) of predicted peptides were experimentally validated as binders to personal HLA alleles. Overall, the predictions for 9-mer peptides were more sensitive than for 10-mer peptides, as 60% vs 44.5% of predicted peptides (IC50<500 nM) could be experimentally validated, respectively, as shown in (FIG. 13).

TABLE 7
Predicted and experimental HLA-binding
results of candidate neoepitopes generated
from 4 CLL cases.

| | | | | | Candidate | |
|---|---|---|---|---|---|---|
| | | | | | neoepitopes | |
| | | | | | IC50 (nM) | |
| | | | | HLA | Pre- | Experi- |
| Pt | Gene | Sequence | Length | allele | dicted | mental |
| 1 | THOC6 | ELWCRQPPYR | 10 | A*33:01 | 10 | 18 |
| 1 | THOC6 | ELWCRQPPYR | 10 | A*68:12 | 59 | 5.1 |
| 1 | CDC25A | QSYCEPSSYR | 10 | A*68:12 | 23 | 1.5 |
| 1 | ALMS1 | TVPSSSFSHR | 10 | A*68:12 | 25 | 11 |
| 1 | WHSC1L1 | EVQASKHTK | 9 | A*68:12 | 33 | 58 |
| 1 | CRYBA1 | VVVCYQYSGYR | 10 | A*33:01 | 44 | 972 |
| 1 | CDC25A | SYCEPSSYR | 9 | A*33:01 | 70 | 14 |
| 1 | THNSL2 | ATIESVQGAK | 10 | A*68:12 | 71 | 42 |
| 1 | ALMS1 | TPTVPSSSF | 9 | B*35:01 | 75 | 91 |
| 1 | RALGAPB | WIMVLVLPK | 9 | A*68:12 | 95 | 218 |
| 1 | THOC6 | ELWCRQPPY | 9 | B*35:01 | 112 | 13776 |
| 1 | RALGAPB | DWIMVLVLPK | 10 | A*33:01 | 117 | 37826 |
| 1 | C6orf89 | MPIEPGDIGC | 10 | B*35:01 | 132 | 131 |
| 1 | STRAP | LISACKDGKR | 10 | A*68:12 | 163 | 15845 |
| 1 | CRYBA1 | YQYSGYRGY | 9 | B*35:01 | 170 | 9851 |
| 1 | WHSC1L1 | LLNEVQASK | 9 | A*68:12 | 197 | 7440 |
| 1 | RALGAPB | DWIMVLVLPK | 10 | A*68:12 | 222 | 2956 |
| 1 | STRAP | ISACKDGKR | 9 | A*68:12 | 224 | 6671 |
| 1 | XPO1 | KTVVNKLFK | 9 | A*68:12 | 253 | 25393 |
| 1 | HMGN2 | NSAENGDAK | 9 | A*68:12 | 258 | 141 |
| 1 | THOC6 | LWCRQPPYR | 9 | A*33:01 | 297 | 915 |
| 1 | POLR2A | VQKIFHINPR | 10 | A*33:01 | 308 | 17699 |
| 1 | CDC25A | QSYCEPSSYR | 10 | A*33:01 | 309 | 53 |
| 1 | ALMS1 | SSSFSHREK | 9 | A*68:12 | 314 | 1496 |
| 1 | CDC25A | SYCEPSSYR | 9 | A*68:12 | 314 | 812 |
| 1 | ALMS1 | TVPSSSFSHR | 10 | A*33:01 | 335 | 237 |
| 1 | THNSL2 | TIESVQGAK | 9 | A*68:12 | 338 | 953 |
| 1 | POLR2A | MIWNVQKIF | 9 | B*35:01 | 393 | 541 |
| 1 | CDC25A | QSYCEPSSY | 9 | B*35:01 | 478 | 50000 |
| 1 | DSCAML1 | SSIRSFVLQY | 10 | B*35:01 | 480 | 9195 |
| 2 | NIN | FLQEETLTQM | 10 | A*02:01 | 10.63 | 1.1 |
| 2 | FNDC3B | VVMSWAPPV | 9 | A*02:01 | 4.21 | 6.4 |
| 2 | SLC46A1 | CSDSKLIGY | 9 | A*01:01 | 8.13 | 8.5 |
| 2 | SYT15 | EMLIKPKEL | 9 | B*08:01 | 414.37 | 8.9 |
| 2 | F2R | ILLMTVTSI | 9 | A*02:01 | 41.91 | 11 |
| 2 | ACSM2A | SLMEHWALG | 9 | A*02:01 | 413.95 | 17 |
| 2 | C16orf57 | LLRVHTEHV | 9 | B*08:01 | 443.97 | 28 |
| 2 | ACSM2A | SLMEHWALGA | 10 | A*02:01 | 5.67 | 40 |
| 2 | TBC1D9B | KMTFLFPNL | 9 | A*02:01 | 63.7 | 62 |
| 2 | SF381 | GLVDEQQEV | 9 | A*02:01 | 22.26 | 94 |
| 2 | LRRC41 | ALPDPILQSI | 10 | A*02:01 | 28.18 | 107 |
| 2 | LRRC41 | GVWALPDPI | 9 | A*02:01 | 382.07 | 122 |
| 2 | FNDC3B | AVVMSWAPPV | 10 | A*02:01 | 98.15 | 123 |
| 2 | F2R | TSIDRFLAV | 9 | B*08:01 | 245.43 | 130 |
| 2 | KIAA0467 | GPSWGLSLM | 9 | B*07:02 | 179.31 | 137 |
| 2 | C16orf57 | LLRVHTEHV | 9 | A*02:01 | 454.23 | 175 |
| 2 | C22orf28 | VVVNCSSMTFL | 10 | A*02:01 | 302.94 | 274 |
| 2 | FNDC3B | VMSWAPPVGL | 10 | A*02:01 | 37.77 | 378 |
| 2 | GDF2 | ILYKDDMGV | 9 | A*02:01 | 13.74 | 567 |
| 2 | FNDC3B | NIQARAVVM | 9 | B*08:01 | 145.51 | 743 |
| 2 | C16orf57 | HVRCKSGNKF | 10 | B*08:01 | 340.37 | 803 |
| 2 | LRRC41 | LPDPILQSIL | 10 | B*07:02 | 243.46 | 855 |
| 2 | F2R | SILLMTVTSI | 10 | A*02:01 | 301.24 | 929 |
| 2 | ACSM2A | LMEHWALGA | 9 | A*02:01 | 314.16 | 968 |
| 2 | LRRC41 | LPDPILQSI | 9 | B*07:02 | 471.62 | 1056 |
| 2 | C16orf57 | VLLRVHTEHV | 10 | A*02:01 | 23.04 | 1252 |
| 2 | TBC1D9B | FPNLKDRDFL | 10 | B*07:02 | 107.39 | 1423 |
| 2 | SYT15 | MLIKPKELV | 9 | A*02:01 | 162.61 | 1442 |
| 2 | ACSM2A | ILCSLMEHWA | 10 | A*02:01 | 424.59 | 1651 |
| 2 | TBC1D9B | FPNLKDRDF | 9 | B*07:02 | 280.32 | 1687 |
| 2 | GDF2 | SILYKDDMGV | 10 | A*02:01 | 140.39 | 1775 |
| 2 | TP53 | NTFRHRVVV | 9 | B*08:01 | 285.7 | 1789 |
| 2 | SF381 | EVRTISALAI | 10 | B*08:01 | 327.97 | 2322 |
| 2 | GDF2 | VPTKLSPISI | 10 | B*07:02 | 132.77 | 3416 |
| 2 | ELK3 | LLLQDSECKA | 10 | A*02:01 | 437.05 | 5074 |
| 2 | KIAA0467 | SQPGPSWGL | 9 | A*02:01 | 128.72 | 6511 |
| 2 | RNF150 | KPAVSSDSDI | 10 | B*07:02 | 228.47 | 8085 |
| 3 | ZNF182 | ITHTGEKPY | 9 | B*15:01 | 205.26 | 92 |
| 3 | ZNF182 | ITHTGEKPYK | 10 | A*03:01 | 443.32 | 40 |
| 3 | ZNF253 | KFSNSNIYK | 9 | A*03:01 | 116.69 | 273 |
| 3 | IREB2 | LTRGTFANIK | 10 | A*01:01 | 343.52 | 739 |
| 3 | TLK2 | LTDFGLSKIM | 10 | A*03:01 | 164.9 | 1897 |
| 3 | TLK2 | LTDFGLSKI | 10 | A*01:01 | 227 | 10452 |
| 3 | TLK2 | KLTDFGLSK | 9 | A*03:01 | 26 | 41 |
| 3 | MYD88 | SLSLGAHQK | 9 | A*03:01 | 122.42 | 30 |
| 3 | PATE2 | FLKHKQSCAV | 10 | B*08:01 | 17 | 21 |
| 3 | PATE2 | GVMTSCFLK | 9 | A*03:01 | 25 | 29 |
| 3 | PATE2 | FLKHKQSCA | 9 | B*08:01 | 19 | 51 |
| 3 | JTB | GLLCAFTLK | 9 | A*03:01 | 12 | 62 |
| 3 | JTB | HLCGLLCAF | 9 | B*15:01 | 117 | 125 |
| 3 | OR13C5 | LSIFKISSL | 9 | B*08:01 | 151 | 158 |
| 3 | PATE2 | VMTSCFLKHK | 10 | A*03:01 | 140 | 174 |
| 3 | PATE2 | MTSCFLKHK | 9 | A*03:01 | 147 | 218 |
| 3 | OR13C5 | KISSLEGRSK | 10 | A*03:01 | 185 | 257 |
| 3 | OR13C5 | LSIFKISSL | 9 | B*15:01 | 152 | 368 |
| 4 | MAPK14 | RPTFYRQGL | 9 | B*07:02 | 6.7 | 76 |
| 4 | SCYL2 | EVAGFVFDK | 9 | A*68:01 | 7.3 | 14 |
| 4 | SCYL2 | EVAGFVFDKK | 10 | A*68:01 | 7.4 | 8.8 |
| 4 | COL5A3 | FTAGGEPCLY | 10 | A*01:01 | 14 | 153 |
| 4 | MPDZ | FSIVGGYGR | 9 | A*68:01 | 20 | 2.6 |
| 4 | CUL1 | YMKKAEAPL | 9 | B*08:01 | 36 | 34841 |
| 4 | MUC2 | APITTTTTV | 9 | B*07:02 | 53 | 13 |
| 4 | KDM5D | HSIPLRQSVK | 10 | A*68:01 | 55 | 45 |
| 4 | TBC1D25 | ISYLGRDRLR | 10 | A*68:01 | 106 | 556 |
| 4 | NUP98 | APGFNTTPA | 9 | B*07:02 | 107 | 13 |
| 4 | ZNF330 | KAFFCDDHTR | 10 | A*68:01 | 137 | 102 |
| 4 | MPDZ | RPHGDLPIYV | 10 | B*07:02 | 155 | 1321 |
| 4 | TBC1D25 | RLRQEVYLSL | 10 | B*08:01 | 165 | 1084 |
| 4 | CUL1 | YMKKAEAPLL | 10 | B*08:01 | 168 | 138 |
| 4 | TBC1D25 | RLRQEVYLSL | 10 | B*07:02 | 183 | 114 |
| 4 | LANCL1 | CLTKRSIAF | 9 | B*08:01 | 205 | 47 |
| 4 | COL5A3 | FTAGGEPCLY | 10 | A*68:01 | 230 | 11 |
| 4 | SF381 | EYVLNTTAR | 9 | A*68:01 | 301 | 651 |
| 4 | CNN1 | DPKLGTAQPL | 10 | B*07:02 | 369 | 3974 |
| 4 | PPP2R2C | QTHEPEFDY | 9 | A*01:01 | 435 | 26184 |
| 4 | MUC2 | APITTTTTVT | 10 | B*07:02 | 436 | 3731 |
| 4 | CUL1 | EAPLLEEQR | 9 | A*68:01 | 454 | 36 |
| 4 | LANCL1 | CLTKRSIAFL | 10 | B*08:01 | 467 | 640 |
| 4 | NUP98 | APGFNTTPAT | 10 | B*07:02 | 475 | 5744 |
| 4 | MUC2 | TTAPITTTT | 9 | A*68:01 | 479 | 118 |
| 4 | CUL1 | YMKKAEAPL | 9 | B*07:02 | 480 | 7927 |
| 4 | LOXL2 | IPGFKFDNL | 9 | B*07:02 | 487 | 809 |

** An experimental binding assay for A*68:12 was not available. Because A*68:12 and A*68:01 have identical primary structures in the B and F main peptide binding pockets and have been predicted to have similar binding specificity (Sidney and Sette, 2007), experimental binding for peptides predicted to bind A*68:12 were assayed against A*68:01.

### Example 18

### Neoantigens are Expressed in CLL Tumors

CTL responses against an epitope would only be useful if the gene encoding the epitope is expressed in the target cells. Of the 31 patient samples sequenced and typed for HLA, 26 were subjected to genome-wide expression profiling (see e.g., Brown et al. 2012). The expression level of 347 genes with mutations in CLL samples was classified as having low/absent (lowest quartile), medium (middle two quartiles), or high (highest quartile) expression. As shown in FIG. 12D, 80% of the 347 mutated genes (or 79% of the 180 mutations with predicted HLA-binding) were expressed at medium or high expression levels. A similar high frequency of expression was observed among the subset of 221 mutated genes (88.6%) with predicted class I binding epitopes.

RNA levels may be determined based on the number of reads per gene product, and ranked by quartiles. “H”—Top quartile; “M”—Middle two quartiles; “L”—Lowest quartile (excluding genes with no reads; “-”—no reads detectable. As the predicted affinity decreases, higher stringency may be applied to expression levels. NeoORFs with predicted binders were utilized even if there was no detectable mRNA molecules by RNA-Seq. There is no data currently available to assess what, if any, the minimum expression level required in a tumor cell would be for a neoORF to be useful as a target for activated T-cells. Even the level of expression of “pioneer” translation of messages destined for nonsense mediated decay may be sufficient for target generation ((Chang Y F, Imam J S, Wilkinson M F: The nonsense-mediated decay RNA surveillance pathway. Annu Rev Biochem 76:51-74, 2007). Therefore, because of the high value of neoORFs as targets due to their novelty and exquisite tumor specificity, neoORFs may be utilized as immunogens even if expression at the RNA level is low or undetectable.

### Example 19

### T Cells Targeting Candidate Neoepitopes were Detected in CLL Patient 1 Following HSCT

The post-allogeneic hematopoietic stem cell transplantation (HSCT) setting in CLL was analyzed to determine whether an immune response against the predicted mutated peptides could develop in patients. Reconstitution of T cells from a healthy donor following HSCT can overcome endogenous immune defects of the host, and also allow priming against leukemia cells in the host in vivo. Analysis focused on two patients who had both undergone unrelated reduced intensity conditioning allo-HSCT for advanced CLL and had achieved continuous remission for greater than 4 years following HSCT (see e.g., Table 8). Post-transplant T cells were collected 7 years (Patient 1) and 4 years (Patient 2) from the time of transplant.

Table 8 shows the clinical characteristics of CLL Pts 1 and 2. Both patients have achieved ongoing continuous remission following HSCT of greater than 7 (Pt 1) and 4 years (Pt 2). M: male; HSCT: hematopoietic stem cell transplantation; RIC: reduced intensity conditioning; Flu/Bu: Fludarabine/Busulfan; GvHD: graft vs host disease; URD: unrelated donor; Mis: missense; FS: frameshift.

TABLE 8

| | | | Allogeneic HSCT | | | | | | | | | |
|---|---|---|---|---|---|---|---|---|---|---|---|---|
| | | | | Stem | Days to | | Number of Mutations | | | | Neoepitopes | |
| | HLA | Age/ | Conditioning | cell | cGvHD | GvHD | | | | Putative | (IC50 < 500 nM) | |
| Pt | typing | Sex | regimen | source | Onset | meds | Total | Mis | FS | drivers | Predicted | Experimental |
| 1 | A*33:01/ | 51/M | RIC | URD | 448 | Imatinib/ | 33 | 25 | 8 | XPO1 | 30 | 14 |
| | *68:12 | | Flu/Bu | PBSC | | Prednisone | | | | | | |
| | B*35:01/ | | | | | | | | | | | |
| | *14:01 | | | | | | | | | | | |
| 2 | A*01:01/ | 72/M | RIC | URD | 208 | Imatinib | 27 | 26 | 1 | TP53, | 37 | 18 |
| | *02:01 | | Fu/Bu | PBSC | | | | | | SF3B1 | | |
| | B*07:02/ | | | | | | | | | | | |
| | *08:01 | | | | | | | | | | | |

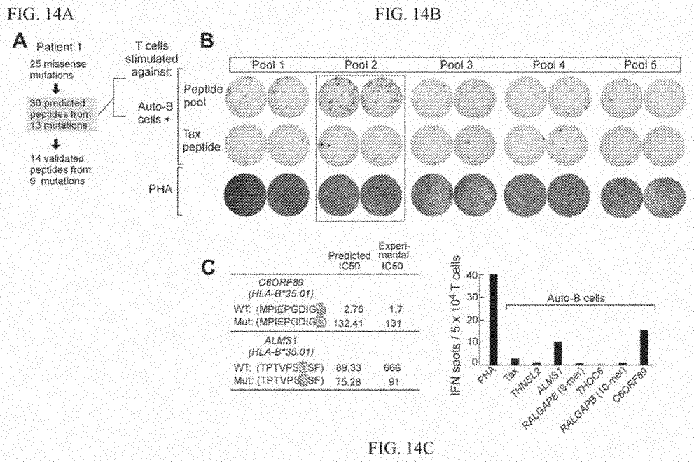

For Patient (Pt 1), 25 missense mutations were identified by WES. In total, 30 peptides from 13 mutations were predicted to bind to personal HLA (13 peptides with IC50<150; 17 peptides with IC50 150-500 nM). As shown in FIG. 14A, experimental validation of peptide predictions confirmed HLA binding for 14 peptides derived from 9 mutations. All 30 predicted HLA binding peptides were selected for T cell priming studies, and were organized into 5 pools of 6 peptides/pool (see e.g., Table 9). Peptides with similar predicted binding scores were put together within the same pool.

Table 9 provides a summary of peptides from Pt 1 missense mutations that were included in peptide pools for T cell stimulation studies. In Pt 1, all predicted peptides with IC50<500 nM binding to HLA -A and -B alleles were used. 5 pools of mutated peptides with 6 peptides/pool listed in decreasing order of predicted binding affinities to MHC class I alleles. The corresponding experimental HLA-peptide binding affinities, wildtype peptides and their predicted IC50 scores are included in the far right columns.

TABLE 9

| | | | | MUT peptide | | | WT peptide | |
|---|---|---|---|---|---|---|---|---|
| | | | | | Pre- | Experi- | | Pre- |
| | | | | | dicted | mental | | dicted |
| | | | HLA | | IC50 | IC50 | | IC50 |
| Pool | Gene | Length | allele | Sequence | (nM) | (nM) | Sequence | (nM) |
| 1 | THOC6 | 10 | A*33:01 | ELWCRQPPYR | 10 | 18 | ELWRRQPPYR | 11 |
| | THOC6 | 10 | A*68:12 | ELWCRQPPYR | 59 | 5.1 | ELWRRQPPYR | 61 |
| | CDC25A | 10 | A*68:12 | QSYCEPSSYR | 23 | 1.5 | QSYCEPPSYR | 37 |
| | ALMS1 | 10 | A*68:12 | TVPSSSFSHR | 25 | 11 | TVPSGSFSHR | 35 |
| | WHSC1L1 | 9 | A68:12 | EVQASKHTK | 33 | 58 | EVQASEHTK | 34 |
| | CRYBA1 | 10 | A*33:01 | WVCYQYSGYR | 44 | 972 | WVCYQYPGYR | 50 |
| | CDC25A | 9 | A*33:01 | SYCEPSSYR | 70 | 14 | SYCEPPSYR | 61 |
| 2 | THNSL2 | 10 | A*68:12 | ATIESVQGAK | 71 | 42 | AAIESVQGAK | 470 |
| | ALMS1 | 9 | B*35:01 | TPTVPSSSF | 75 | 91 | TPTVPSGSF | 89 |
| | RALGAPB | 9 | A*68:12 | WIMVLVLPK | 95 | 218 | WIMALVLPK | 46 |
| | THOC6 | 9 | B*35:01 | ELWCRQPPY | 112 | 13776 | ELWRRQPPY | 126 |
| | RALGAPB | 10 | A*33:01 | DWIMVLVLPK | 117 | 37826 | DWIMALVLPK | 171 |
| | C6orf89 | 10 | B*35:01 | MPIEPGDIGC | 132 | 131 | MPIEPGDIGY | 3 |
| | STRAP | 10 | A*68:12 | LISACKDGKR | 163 | 15845 | LISACKDGKP | 38499 |
| 3 | CRYBA1 | 9 | B*35:01 | YQYSGYRGY | 170 | 9851 | YQYPGYRGY | 171 |
| | WHSC1L1 | 9 | A68:12 | LLNEVQASK | 197 | 7440 | LLNEVQASE | 21454 |
| | RALGAPB | 10 | A*68:12 | DWI MVLVLPK | 222 | 2956 | DWIMALVLPK | 299 |
| | STRAP | 9 | A*68:12 | ISACKDGKR | 224 | 6671 | ISACKDGKP | 39393 |
| 4 | XPO1 | 9 | A68:12 | KTVVNKLFK | 253 | 25393 | KTVVNKLFE | 18346 |
| | HMGN2 | 9 | A68:12 | NSAENGDAK | 258 | 141 | NPAENGDAK | 3679 |
| | THOC6 | 9 | A*33:01 | LWCRQPPYR | 297 | 915 | LWRRQPPYR | 222 |
| | POLR2A | 10 | A*33:01 | VQKIFHINPR | 308 | 17699 | AQKIFHINPR | 738 |
| | CDC25A | 10 | A*33:01 | QSYCEPSSYR | 309 | 53 | QSYCEPPSYR | 398 |
| | ALMS1 | 9 | A68:12 | SSSFSHREK | 314 | 1496 | SGSFSHREK | 3554 |
| 5 | CDC25A | 9 | A*68:12 | SYCEPSSYR | 314 | 812 | SYCEPPSYR | 597 |
| | ALMS1 | 10 | A*33:01 | TVPSSSFSHR | 335 | 237 | TVPSGSFSHR | 378 |
| | THNSL2 | 9 | A*68:12 | TIESVQGAK | 338 | 953 | AIESVQGAK | 3861 |
| | POLR2A | 9 | B*35:01 | MIWNVQKIF | 393 | 541 | MIWNAQKIF | 294 |
| | CDC25A | 9 | B*35:01 | QSYCEPSSY | 478 | 50000 | QSYCEPPSY | 472 |
| | DSCAML1 | 10 | B*35:01 | SSIRSFVLQY | 480 | 9195 | SSIRGFVLQY | 391 |

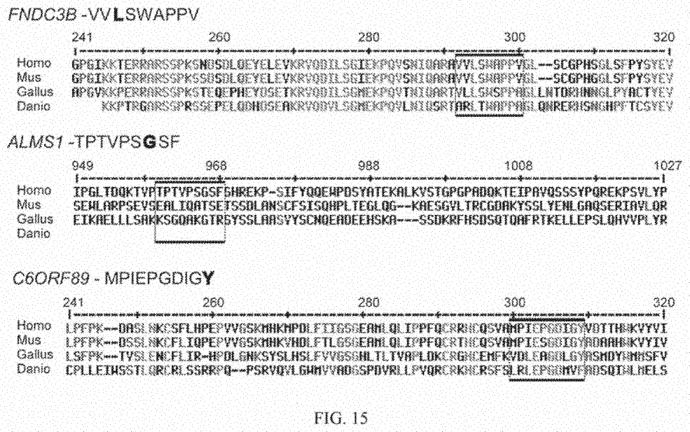

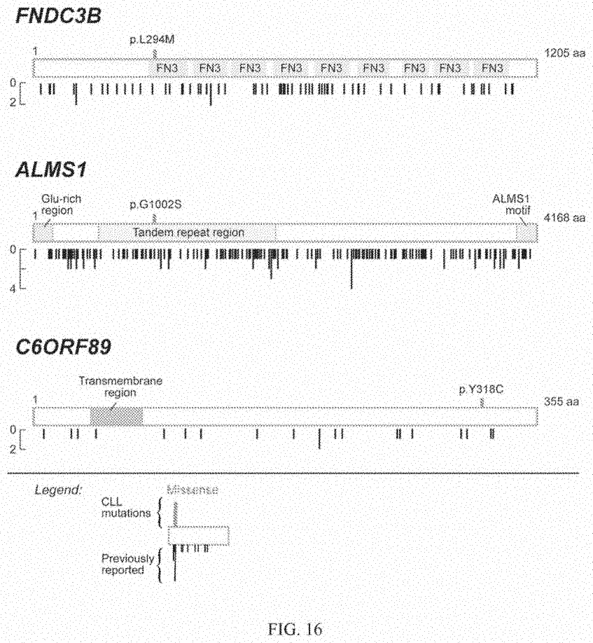

T cells were tested for neoantigen reactivity by expanding them using autologous antigen presenting cells (APCs) pulsed with candidate neoantigen peptide pools (once per week×4 weeks). As shown in FIG. 14B, reactivity in a IFN-γ ELISPOT assay was detected against Pool 2, but not against an irrelevant peptide (Tax peptide). Deconvolution of the pool revealed that the mutated (mut) ALMS1 and C6orf89 peptides within Pool 2 were immunogenic. ALMS1 plays a role in ciliary function, cellular quiescence and intracellular transport, and mutations in this gene have been implicated in type II diabetes. C6orf89 encodes a protein that interacts with bombesin receptor subtype-3, which is involved in cell cycle progression and wound repair of bronchial epithelial cells. Both mutated sites were not in conserved regions of the gene, and were not within genes previously reported to be mutated in cancer. Both of the target peptides were among the subset of 14 predicted peptides that could be experimentally confirmed to bind Pt 1's HLA alleles. The experimental binding scores of mut and wildtype (wt) ALMS1 were 91 and 666 nM, respectively; and of mut- and wt-C6ORF89, 131 and 1.7 nM, respectively (see e.g., FIG. 14C and Table 9). Both mutated genes localized to poorly conserved regions and did not localize to previously reported mutation sites in cancers (see e.g., FIGS. 15-16).

### Example 20

### CLL Patient 2 Exhibited Immunity Against a Mutated FNDC3B Peptide That is Naturally Processed

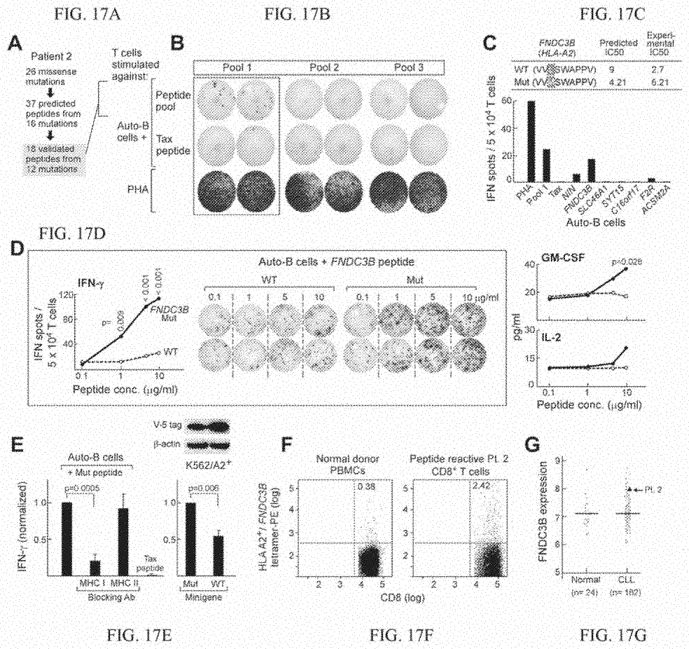

In Patient 2, the ability personal neoantigens to contribute to memory T responses in the setting of long-lived remission was tested. From this individual, 26 non-synonymous missense mutations were identified. In total, 37 peptides from 16 mutations were predicted to bind to personal HLA alleles, of which 18 peptides from 12 mutations could be experimentally validated (15 with IC50<150; 3 with IC50 150-500 nM) (see e.g., FIG. 17A). In Pt 2, all 18 experimentally validated HLA-binding peptides were studied. T cell stimulations were performed using 3 pools of 6 peptides/pool (see e.g., Table 10). Table 10 shows a summary of peptides from Pt 2 missense mutations that were included in peptide pools for T cell stimulation studies. In Pt 2, all peptides that were experimentally confirmed to bind to HLA-A and -B alleles were used. 3 pools of peptides with 6 peptides/pool listed in decreasing order of experimental binding affinity of mutated peptides. The corresponding wildtype peptides and their predicted IC50 scores are included in the far right columns.

TABLE 10

| | | | | MUT peptide | | | WT peptide | |
|---|---|---|---|---|---|---|---|---|
| | | | | | Pre- | Experi- | | Pre- |
| | | | | | dicted | mental | | dicted |
| | | | HLA | | IC50 | IC50 | | IC50 |
| Pool | Gene | Length | allele | Sequence | (nM) | (nM) | Sequence | (nM) |
| 1 | NIN | 10 | A*02:01 | FLQEETLTQM | 10.63 | 1.1 | FLQEERLTQM | 45 |
| | FNDC3B | 9 | A*02:01 | VVMSWAPPV | 4.21 | 6.2 | VVLSWAPPV | 9 |
| | SLC46A1 | 9 | A*01:01 | CSDSKLIGY | 8.13 | 8.5 | CWDSKLIGY | 1778 |
| | SYT15 | 9 | B*080:1 | EMLIKPKEL | 414.37 | 8.9 | EMLSKPKEL | 785 |
| | F2R | 9 | A*02:01 | ILLMTVTSI | 41.91 | 11 | ILLMTVISI | 53 |
| | ACSM2A | 9 | A*02:01 | SLMEHWALG | 413.95 | 17 | SLMEPWALG | 1313 |
| 2 | C16orf57 | 9 | B*080:1 | LLRVHTEHV | 443.97 | 28 | LLRVHTEQV | 498.35 |
| | ACSM2A | 10 | A*02:01 | SLMEHWALGA | 5.67 | 40 | SLMEPWALGA | 9.8 |
| | TBC1D9B | 9 | A*02:01 | KMTFLFPNL | 63.7 | 62 | KMTFLFANL | 93 |
| | SF3B1 | 9 | A*02:01 | GLVDEQQEV | 22.26 | 94 | GLVDEQQKV | 51 |
| | LRRC41 | 10 | A*02:01 | ALPDPILQSI | 28.18 | 107 | ALPGPILQSI | 99 |
| | LRRC41 | 9 | A*02:01 | GVWALPDPI | 382.07 | 122 | GVWALPGPI | 963 |
| 3 | FNDC3B | 10 | A*02:01 | AVVMSWAPPV | 98.15 | 123 | AVVLSWAPPV | 89 |
| | F2R | 9 | B*080:1 | TSIDRFLAV | 245.43 | 130 | ISIDRFLAV | 252 |
| | KIAA0467 | 9 | B*07:02 | GPSWGLSLM | 179.31 | 137 | GPSRGLSLM | 39 |
| | C16orf57 | 9 | A*02:01 | LLRVHTEHV | 454.23 | 175 | LLRVHTEQV | 433.02 |
| | C22orf28 | 10 | A*02:01 | VVVNCSSMTFL | 302.94 | 274 | VVVNRSSMTFL | 835 |
| | FNDC3B | 10 | A*02:01 | VMSWAPPVGL | 37.77 | 378 | VLSWAPPVGL | 48 |

Peptides with similar experimental binding scores were combined within the same pool. Responses were assessed after 2 rounds of weekly stimulations of T cells against mutated peptide pool-pulsed autologous APCs, and T cells were found to be reactive against Pool 1, as shown in FIG. 17B. Deconvolution of the pool revealed mut-FNDC3B to be the dominant immunogenic peptide among others within this pool (experimental IC50 of mut- and wt-FNDC3B were 6.2 and 2.7 nM, respectively; see e.g., FIG. 17C). The function of FNDC3B in blood malignancies is unclear, although down-regulation of FNDC3B expression is known to upregulate miR-143 expression, which has been shown to differentiate prostate cancer stem cells and promote prostate cancer metastasis. Similar to ALMS1 and C6orf89, the mutation in FNDC3B neither localized to evolutionarily conserved regions nor was it previously reported in other cancers (see e.g., FIGS. 15 and 16).

T cell reactivity against mut-FNDC3B was polyfunctional (secreting GM-CSF, IFN-γ and IL-2), and specific to the mut-FNDC3B peptide but not its wildtype counterpart. Testing T cell reactivity against different concentrations of mut- and wt-FNDC3B peptides revealed a high avidity and specificity of mut-FNDC3B reactive T cells. T cell reactivity was abrogated by the presence of class I blocking antibody (W6/32), indicating that T cell reactivity was class I restricted (see e.g., FIGS. 17D-E). Moreover, the mut-FNDC3B peptide appeared to be a naturally processed and presented peptide since T cell reactivity was detected against HLA-A2-expressing APCs that were transfected with a 300 basepair minigene encompassing the region of gene mutation but not the wildtype minigene, as shown in FIG. 17E, right panel.

Using a mut-FNDC3B/A2⁺-specific tetramer, a discrete population of mut-FNDC3B-reactive CD8⁺ T cells was detected within Pool 1-stimulated T cells (2.42% of the population) compared to control PBMCs from a healthy adult HLA-A2+ volunteer (0.38%), as shown in FIG. 17F. Gene expression analysis of FNDC3B in a large dataset of 182 CLL cases (including Pt 2) and 24 CD19⁺ B cells collected from normal volunteers revealed this gene to be relatively overexpressed in Patient 2 compared to other CLLs and normal B cells, as shown in FIG. 17G. Accordingly, it is clear that long-lived neoantigen-specific T cells could be tracked in CLL Patient 2.

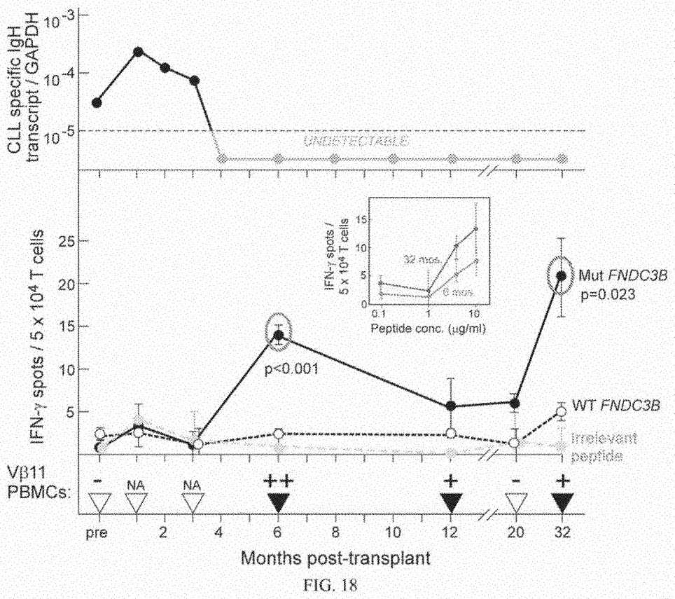

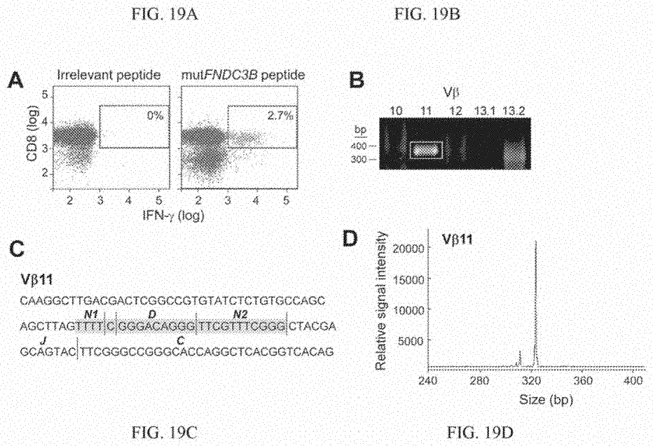

To define the kinetics of mut-FNDC3B specific T cells in relationship to post-HSCT course, Pt 2 T cells isolated from different time points before and after HSCT were stimulated for 2 weeks and then tested for IFN-γ reactivity on ELISPOT. The emergence of mut-FNDC3B-specific T cells coincided with molecular remission and was sustained over time with continuous remission. As shown in FIG. 18 (top and middle panel), mut-FNDC3B T cell responses were not detected before or up to 3 months following HSCT. Molecular remission was first achieved 4 months following HSCT, and mut-FNDC3B-specific T cells were then first detected 6 months following HSCT. Antigen-specific reactivity subsequently waned (between 12 and 20 months post-HSCT), but was again strongly detected at 32 months post-HSCT. Based on molecular analysis of the TCR of the mut-FNDC3B-specific T cells, Vβ11 was identified as the predominant CDR3 Vβ subfamily used by the reactive T cells, as shown in FIG. 19 and Table 11). Table 11 shows primers used for amplification of the TCR Vβ subfamily.

TABLE 11

| | | Amplicon |
|---|---|---|
| | Forward primer | size |
| Name | sequence (5′-3′) | (bp) |
| Vβ1 | GCACAACAGTTCCCTGACTTGCAC | 346 |
| Vβ2 | TCATCAACCATGCAAGCCTGACCT | 349 |
| Vβ3 | GTCTCTAGAGAGAAGAAGGAGCGC | 346 |
| Vβ4 | ACATATGAGAGTGGATTTGTCATT | 378 |
| Vβ5.1 | ATACTTCAGTGAGACACAGAGAAAC | 396 |
| Vβ5.2 | TTCCCTAACTATAGCTCTGAGCTG | 343 |
| Vβ6 | AGGCCTGAGGGATCCGTCTC | 340 |
| Vβ7 | CCTGAATGCCCCAACAGCTCTC | 347 |
| Vβ8 | ATTTACTTTAACAACAACGTTCCG | 404 |
| Vβ9 | CCTAAATCTCCAGACAAAGCTCAC | 348 |
| Vβ10 | CCACGGAGTCAGGGGACACAGCAC | 313 |
| Vβ11 | TCCAACCTGCAAAGCTTGAGGACT | 312 |
| Vβ12 | CATGGGCTGAGGCTGATC | 417 |
| Vβ13.1 | CAAGGAGAAGTCCCCAAT | 372 |
| Vβ13.2 | GGTGAGGGTACAACTGCC | 390 |
| Vβ14 | GTCTCTCGAAAAGAGAAGAGGAAT | 349 |
| Vβ15 | AGTGTCTCTCGACAGGCACAGGCT | 352 |
| Vβ16 | AAAGAGTCTAAACAGGATGAGTCC | 395 |
| Vβ17 | GGAGATATAGCTGAAGGGTA | 372 |
| Vβ18 | GATGAGTCAGGAATGCCAAAGGAA | 380 |
| Vβ19 | TCCTCTCACTGTGACATCGGCCCA | 322 |
| Vβ20 | AGCTCTGAGGTGCCCCAGAATCTC | 370 |
| Vβ22 | AAGTGATCTTGCGCTGTGTCCCCA | 490 |
| Vβ23 | AGGACCCCCAGTTCCTCATTTC | 435 |
| Vβ24 | CCCAGTTTGGAAAGCCAGTGACCC | 509 |
| Vβ25 | TCAACAGTCTCCAGAATAAGGACG | 352 |
| | Reverse primer | |
| Name | sequence (5′-3′) | |
| External Cβ | GACAGCGGAAGTGGTTGCGGGGT | |
| Internal Cβ | FAM-CGGGCTGCTCC | |
| | TTGAGGGGCTGCG | |

This molecular information was used to develop a clone-specific nested PCR assay. Applying this assay, it was observed that T cells with the same specificity for mut-FNDC3B were not detected in PBMCs (n=3) and CD8⁺ T cells of normal healthy volunteers (see e.g., Table 12), but could be detected with similar kinetics as detection of IFN-γ secretion following HSCT in the patient as shown in FIG. 18, bottom panel. Although relative numbers of clone-specific T cells declined over time, lower concentrations of peptide antigen could stimulate T cell reactivity at 32 months compared to 6 months post-HSCT, indicating the emergence of potentially more antigen-sensitive memory T cells over time (see e.g., FIG. 18, inset).

Table 12 shows detection of mut-FNDC3B specific TCR Vβ11, using T cell receptor-specific primers in Pt 2. A real-time PCR assay was designed to detect the mut-FNDC3B-specific TCR Vβ11 clone. This clone was not detectable in healthy donor PBMCs (n=3) or CD8 T cells, but clearly detectable in cDNA from mut-FNDC3B reactive T cells from Pt 2 (at 6 months post-HSCT). The PCR products were normalized over 18S ribosomal RNA. −, negative: no amplification; +, positive: amplification detected; ++, double positive: amplification detected and amplification level is more than median level of all positive samples.

TABLE 12

| | Vβ11 Clone specific | |
|---|---|---|
| cDNA | PCR | 18s ribosomal RNA |
| T cell clone | ++ | + |
| Healthy donor PBMCs | − | + |
| (n = 3) | | |
| Healthy donor CD8 T | − | + |
| cells | | |

### Example 21

### Large Numbers of Candidate Neoantigens were Predicted Across Diverse Cancers

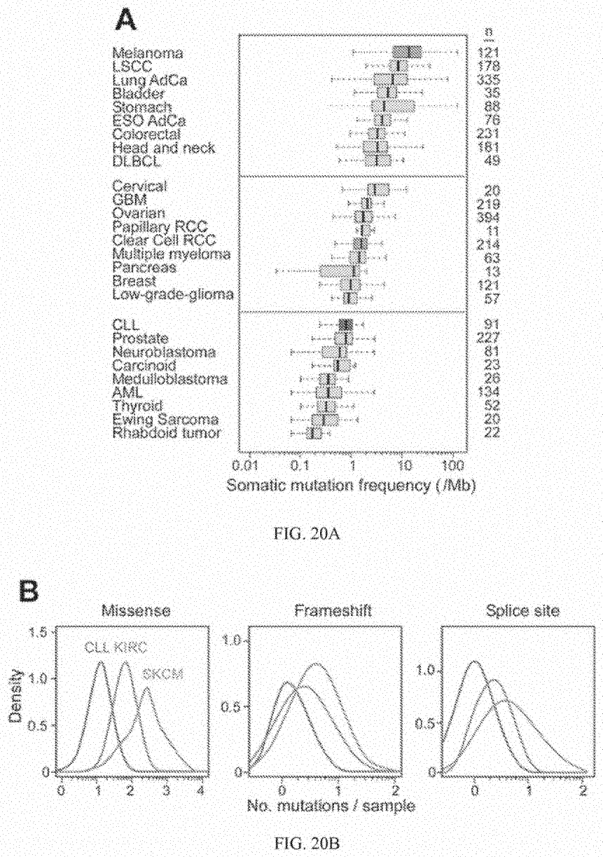

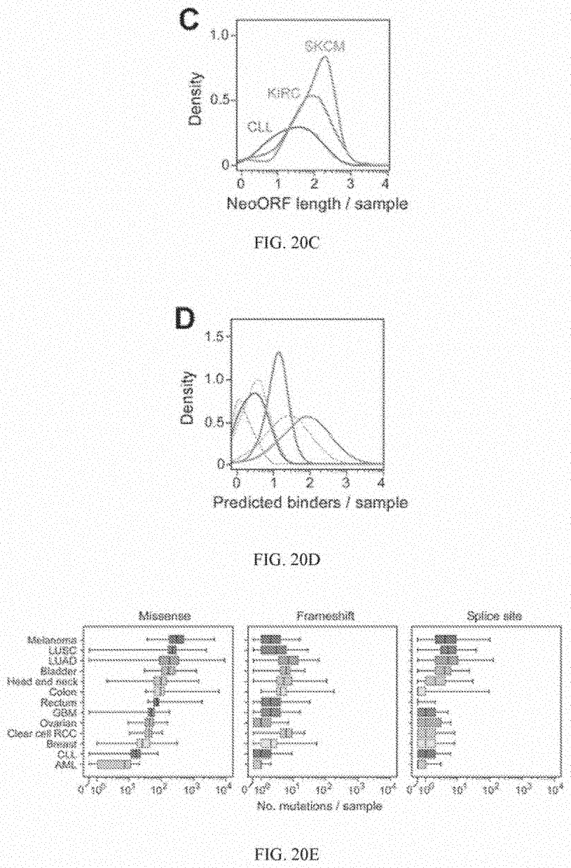

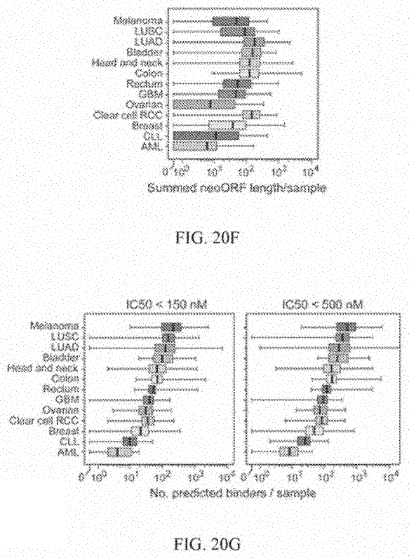

The overall somatic mutation rate of CLL is similar to other blood malignancies, but low in comparison to solid tumor malignancies (see e.g., FIG. 20A). To examine how tumor type and mutation rate impacts the abundance and quality of candidate neoantigens, the pipeline was applied to publicly available WES data from 13 malignancies—including high (melanoma (MEL)), lung squamous (LUSC) and adeno (LUAD) carcinoma, head and neck cancer (HNC), bladder cancer, colon and rectum adenocarcinoma, medium (glioblastoma (GBM), ovarian, clear cell renal carcinoma (clear cell RCC), and breast cancer) and low (CLL and acute myeloid leukemia (AML) cancers. To perform this analysis, a recently described algorithm that enables inference of HLA typing from the WES data was also implemented (Liu et al. 2013).

The overall mutation rate in these solid malignancies was an order of magnitude higher than for CLL and was associated with an increased median number of missense mutations. For example, melanoma displayed a median of 300 (range, 34-4276) missense mutations per case, while RCC had 41 (range, 10-101), respectively. Frameshift and splice-site mutations in RCC and melanoma were increased by only 2-3 fold in frequency as compared to CLL and summed neoORF length per sample were increased only moderately (by 5-13 fold). Overall, the median number of predicted neopeptides with IC50<500 nM generated from missense and frameshift events per sample was proportional to the mutation rate; this was approximately 20- and 4-fold higher for melanoma (488; range, 18-5811) and RCC (80; range, 6-407)), respectively, compared to CLL (24; range 2-124). With a more stringent threshold of IC50<150 nM, the corresponding numbers of predicted neopeptides were 212, 35 and 10 for melanoma, RCC and CLL, respectively, as shown in FIG. 20B and Table 13).

Table 13 shows the distribution of mutation classes, summed neoORF sizes and number of predicted binding peptides across 13 cancers. MEL: melanoma, LUSC: lung squamous cell carcinoma, LUAD: lung adenocarcinoma, BLCA: bladder, HNSC: head and neck cancer, COAD: colon adenocarcinoma, READ: renal adenocarcinoma, GBM: glioblastoma, OV: ovarian, RCC: clear cell renal carcinoma, BRCA: breast, CLL: chronic lymphocytic leukemia, AML: acute myeloid leukemia. *-predicted number of peptides based on missense and frameshift mutations.

TABLE 13

| | # of mutations/sample | | | | | | | | # of predicted peptides | | | |
|---|---|---|---|---|---|---|---|---|---|---|---|---|
| | median (range) | | | | | | Summed | | median (range)* | | | |
| Cancer | | | Frame | | Splice | | NeoORF | | IC50 < | | IC50 | |
| type | Missense | | shift | | site | | length/Sample | | 150 (nM) | | 150-500 (nM) | |
| MEL | 300 | (34-4276) | 2 | (0-16) | 4 | (0-101) | 48 | (0-425) | 212 | (10-2566) | 488 | (18-5811) |
| LUSC | 212 | (0-2397) | 3 | (0-28) | 5 | (0-37) | 86.5 | (0-975) | 149.5 | (0-1320) | 351.5 | (0-2946) |
| LUAD | 172.5 | (0-8971) | 7 | (0-61) | 5 | (0-127) | 173.5 | (0-2137) | 122 | (0-6999) | 269.5 | (1-16360) |
| BLCA | 161.5 | 28-1194) | 6 | (0-22) | 4 | (0-22) | 152 | (0-780) | 97 | 19-1073) | 232.5 | (59-2337) |
| HNSC | 95 | (2-1400) | 5 | (0-106) | 2 | (0-29) | 124.5 | (0-2585) | 66.5 | (2-1139) | 159.5 | (3-2916) |
| COAD | 93 | (32-5902) | 4 | (1-182) | 0 | (0-96) | 121 | (9-4794) | 68 | (15-2155) | 172 | (40-5199) |
| READ | 72.5 | (37-1837) | 2 | (0-31) | 0 | (0-2) | 51 | (0-929) | 52 | (14-1215) | 114 | (38-2750) |
| GBM | 47 | (0-169) | 2 | (0-16) | 1 | (0-5) | 47 | (0-539) | 39 | (0-166) | 90 | (0-332) |
| OV | 42 | (9-149) | 1 | (0-7) | 1 | (0-6) | 7.5 | (0-328) | 30 | (3-181) | 70 | (13-420) |
| RCC | 41 | (10-101) | 6 | (0-22) | 1 | (0-8) | 143 | (0-813) | 35 | (2-223) | 80 | (6-407) |
| BRCA | 25 | (1-300) | 2 | (0-54) | 1 | (0-8) | 37 | (0-1415) | 21 | (0-346) | 47 | (0-781) |
| CLL | 16 | (0-75) | 1 | (0-9) | 1 | (0-6) | 11 | (0-427) | 10 | (0-50) | 24 | (2-124) |
| AML | 7 | (0-20) | 1 | (0-2) | 0 | (0-3) | 6 | (0-160) | 4 | (0-19) | 8 | (0-41) |

*Refers only to predicted epitopes arising from missense mutations.

### Example 22

### Clinical Strategies for Addressing Clonal Mutations

“Clonal” mutations are those that are found in all cancer cells within a tumor, while “subclonal” mutations are those that statistically are not in all cancer cells and therefore are derived from a sub population within the tumor.

According to the techniques herein, bioinformatic analysis may be used to estimate clonality of mutations. For example, the ABSOLUTE algorithm (Carter et al, 2012, Landau et al, 2013) may be used to estimate tumor purity, ploidy, absolute copy numbers and clonality of mutations. Probability density distributions of allelic fractions of each mutation may be generated followed by conversion to cancer cell fractions (CCFs) of the mutations. Mutations may be classified as clonal or subclonal based on whether the posterior probability of their CCF exceeds 0.95 is greater or lesser than 0.5 respectively.

It is contemplated within the scope of the disclosure that a neoantigen vaccine may include peptides to clonal, sub-clonal or both types of mutations. The decision may depend on the disease stage of the patient and the tumor sample(s) sequenced. For an initial clinical study in the adjuvant setting, it may not be necessary to distinguish between the two mutations types during peptide selection, however, one of skill in the art will appreciate that such information may be useful in guiding future studies for a number of reasons.

First, subject tumor cells may be genetically heterogeneous. Multiple studies have been published in which tumors representing different stages of disease progression have been evaluated for heterogeneity. These include examining the evolution from a pre-malignant disease (Myelodysplastic syndrome) to leukemia (secondary acute myelogenous leukemia [AML]) (Walter et al 2012), relapse following therapy-induced remission of AML(Ding et al 2012), evolution from primary to metastatic breast cancer and medulloblastomas (Ding et al 2012; Wu et al Nature 2012), and evolution from primary to highly metastatic pancreatic and renal cancers (Yachida et al 2012; Gerlinger et al 2012). Most studies utilized genome or exome sequencing but one study also evaluated copy number variations and CpG methylation pattern variations. These studies have shown that genetic events are acquired during cancer cell growth which alter the profile of mutations. Many, and usually most (40%-90%), of the earliest detectable mutations (“founder mutations”) persist in all evolved variants but new mutations unique to evolved clones do arise and these may be distinct between different evolved clones. These changes can be driven by host/cancer cell “environmental” pressures and/or therapeutic intervention and thus more highly metastatic disease or prior therapeutic intervention generally lead to more significant heterogeneity.

Second, it is contemplated that a single tumor for each patient may be initially sequenced, which may provide a snapshot of the profile of genetic variation for that particular point in time. The sequenced tumor may be derived from a clinically evident lymph node, in transit/satellite metastasis, or resectable visceral metastasis. None of the initially tested patients will have disease that has clinically progressed to multiple sites; however, it is contemplated that the techniques described herein in will be broadly applicable to patients have cancer that has progressed to multiple sites. Within this tumor cell population, “clonal mutations” may be comprised of both founder mutations and any novel mutations present in the cell that seeded the resected tumor and sub-clonal mutations represent those that evolved during growth of the resected tumor.

Third, the clinically important tumor cells for the vaccine induced T-cells to target are frequently not the resected tumor cells but rather other currently undetectable tumor cells within a given patient. These cells may have spread directly from the primary tumor or from the resected tumor, may have derived from a dominant or sub-dominant population within the seeding tumor and may have genetically evolved further at the surgically resected site. These events are currently unpredictable.

Thus, for the surgically resected adjuvant setting, there is no a priori way to decide whether mutations found in the resected tumor that are clonal or subclonal represent the optimal choice for targeting other non-resected cancer cells. For example, mutations that are subclonal within the resected tumor may be clonal at other sites if those other sites were seeded from a subpopulation of cells containing the sub-clonal mutation within the resected tumor.

In other disease settings however, such as settings in which patients carry multiple and metastatic lesions, sequencing of more than one lesion (or parts of lesion) or lesions from different time points may provide more information relative to effective peptide selection. Clonal mutations may typically be prioritized in the design of neo-antigen epitopes for the vaccine. In some instances, especially as the tumor evolves and sequencing details from metastatic lesions are evaluated for an individual patient, certain subclonal mutations may be prioritized for consideration as part of peptide selection.

### Example 23

### Personalized Cancer Vaccines Stimulate Immunity Against Tumor Neoantigens

The above-described detailed integration of comprehensive bioinformatics with functional data in CLL and other cancers provides several novel biological insights. First, although CLL is a relatively low mutation rate cancer, it was nonetheless possible to identify epitopes generated by somatic mutations that elicited long-term T cell responses. Whole-exome sequencing data from 31 CLL samples revealed that per case, a median of 22 peptides (range, 6-81) were predicted to bind to personal HLA-A and -B alleles with IC50<500 nM originating from a median of 16 (range, 2-75) missense mutations. Approximately 75% and half (54.5%) of predicted peptides with IC50<150 nM and 500 nM, respectively, were experimentally validated to bind to the patient's HLA alleles. RNA expression analysis showed that nearly 90% of the cognate genes corresponding to the predicted mutated peptides were confirmed to be expressed in CLL cells and expression of a transcript from the mutated allele was detected in each of the three (data not shown) examples tested. Only a fraction of all neoepitopes had generated a spontaneous T-cell response although this response was still detectable years after transplant; ˜6% (3/48) of all predicted and tested mutated peptides or 9% (3/32) of experimentally validated and tested mutated peptides stimulated IFN-γ secretion responses from patient T cells. This rate of neo-epitope discovery in CLL, a low mutation rate tumor, is remarkably similar to the rates recently reported in melanoma (4.5%, or 11/247 peptides; Robbins P F, Lu Y C, El-Gamil M, et al: Mining exomic sequencing data to identify mutated antigens recognized by adoptively transferred tumor-reactive T cells. Nat Med, 2013), a high mutation rate cancer. Hence, functional neoepitopes can be systematically discovered across the broad range of cancers including low mutation rate tumors.

A second key finding is that T cell responses against CLL neoepitopes were long-lived (on the order of several years), associated with continuous disease remission and were generated during in vitro stimulation in a timeframe consistent with memory T cell responses. These studies add to the growing literature that responses against tumor neoantigens contribute to efficacious immune responses. Thus, although approximately 5% of predicted peptides generated from missense mutations yielded detectable T cell responses, the kinetics of the response suggest a possible role in ongoing anti-leukemia surveillance functions. The functional impact of neoantigen-directed T-cell responses is supported by a recent study from Castle et al. (Castle J C, Kreiter S, Diekmann J, et al: Exploiting the mutanome for tumor vaccination. Cancer Res 72:1081-1091, 2012) who identified candidate neoepitopes by WES of B16 murine melanoma and prediction of peptide-HLA allele binders. A subset of these predicted epitopes not only elicited immune responses that were specific to the mutated peptide and not the wildtype counterpart, but could also control the disease both therapeutically and prophylactically. While it was difficult to directly compare the relative contributions of tumor neoantigens versus other types of CLL antigens such as overexpressed or shared native antigens (in contrast to melanoma, CLL tumor antigens are not well characterized) or to the GvL response, prior characterization of antigen-specific T cell responses from a melanoma patient with prolonged survival suggest that anti-neoantigen immunity is more prolonged and sustained over time than that against shared overexpressed tumor antigens.

Third, these results highlight the concept that targeting tumor-specific “trunk” mutations can be impactful from the immunologic standpoint. All three of the immunogenic neoantigens (mutated FND3CB, ALMS1, C6orf89) in the two patients appeared to be passenger mutations, not directly contributory to the oncogenic process, and were clonal, affecting the bulk of the cancer mass. Several features of these immunogenic mutations suggest them to be passenger mutations: lack of sequence conservation around the mutation and lack of previously reported mutations in other cancers at the observed sites. Because clonal evolution is a fundamental feature of cancer, it has been posited that immunologic targeting of cancer drivers would have the advantage of minimal antigenic drift, given their essentiality in tumor function that would require them to be maintained in the face of selective pressure. Although such an advantage may be possible, it is apparently not a requirement. Additionally, driver mutations may not necessarily generate immunogenic peptides. For example, the TP53-S83R mutation in Patient 2 did not generate a predicted epitope of <500 nM against any of its class I HLA-A or -B alleles.

Finally, analysis of the binding characteristics of the neoantigen data from the literature (Table 4) as well as the candidate neoepitopes from the data in CLL revealed conceptual insights into the types of point mutations most likely to effectively create a T cell response. It was found that a consistent feature of immunogenic neoepitopes was a predicted binding affinity<500 nM (3 of 3 of immunogenic CLL peptides and 30 of 33 [91%] of the historical functional neoepitopes) and the majority of these (92%) displayed predicted affinities<150 nM. Unexpectedly however, in most cases (3 of 3 immunogenic CLL peptides and 27 of 33 [82%] historical functional epitopes), the corresponding wild type epitopes were also predicted to bind with comparable strong/intermediate (<150 nM, Group 1 in Table 4) or weak (150-500 nM, Group 2 in Table 4) affinity. The data support the idea that two types of mutations are commonly observed among naturally occurring T-cell responses to neoantigens: (1) mutations at positions that lead to substantially better binding to the MHC allele (mutated ALMS1 as well as 6 of 33 (18%) of the historical functionally-identified neoepitopes [‘Group 3’, Table 4]), presumably due to improved interaction with MHC, or (2) mutations at positions that do not significantly interact with MHC but instead presumably alter the T cell receptor binding ((2 of 3 CLL epitopes [FNDC3B and C6orf89] and 24 of 33 (73%) naturally immunogenic neoepitopes [‘Group 1’ and ‘Group 2’, Table 4]). The distinction between these two types of mutations fits with the concept that the peptide can be considered as a “key’, which must fit both the MHC and the TCR “locks” in order to stimulate cytolysis, allowing mutations to independently vary MHC or TCR binding. Excepting the contribution of minor histocompatiblility antigens to graft-vs-host disease, there are no reports of auto-immune sequelae linked to neoantigens in these patients, even in those patients where a reaction occurs to a mutated peptide and the cognate native peptide is predicted to be a tight binder. This result is consistent with the idea that MHC-binding native peptides are normally involved in the negative selection process in which T cells bearing TCRs reactive to these native peptides are thymically deleted or rendered anergic, and yet the T cell repertoire can accommodate the development of a specific immune response to a neoeptiope peptide due to an altered presentation of the mutated peptide to the T cell receptor. It is clear that each individual tumor in a patient may harbor a broad spectrum of both shared and personal genetic alterations that may continue to evolve in response to the environment, and that this progression may often lead to resistance to therapy. Given the uniqueness and plasticity of tumors, an optimal therapy may need to be customized based on the exact mutations present in each tumor, and may need to target multiple nodes to avoid resistance. The vast repertoire of human CTLs has the potential to create such a therapy that targets multiple, personalized tumor antigens. As discussed above, the present disclosure shows that it is possible to systematically identify CTL target antigens harboring tumor-specific mutations by using massively parallel sequencing in combination with algorithms that effectively predict HLA-binding peptides. Advantageously, the present disclosure allows tumor neoantigens in a variety of low and high mutation rate cancers to be predicted, and experimentally identifies long-lived CTLs that target leukemia neoantigens in CLL patients. The present disclosure supports the existence of protective immunity targeting tumor neoantigens, and provides a method for selecting neoantigens for personalized tumor vaccines.

As discussed in detail above, the techniques described herein were applied to a unique group of CLL patients who developed clinically evident durable remission associated with anti-tumor immune responses following allogeneic-HSCT. These graft-versus-leukemia responses have typically been attributed to allo-reactive immune responses targeting hematopoietic cells. However, the above described results indicate that the GvL response is also associated with CTLs that recognize personal leukemia neoantigens. These results are consistent with data indicating that the existence of GvL-associated CTLs with specificity for tumor, rather than allo-antigens. It has been postulated that neoantigen-reactive CTLs are important in cancer surveillance because the study of a long-term melanoma survivor found that CTLs targeting neoantigens are significantly more abundant and sustained than those against non-mutated overexpressed tumor antigens (Lennerz V, Fatho M, Gentilini C, et al: The response of autologous T cells to a human melanoma is dominated by mutated neoantigens. Proc Natl Acad Sci USA 102:16013-8, 2005). The data presented above is consistent with this melanoma study because neoantigen-specific T cell responses in CLL patients were found to be long-lived (on the order of several years) memory T cells (based on their rapid stimulation kinetics in vitro) and associated with continuous disease remission. Accordingly, neoantigen-reactive CTLs likely play an active role in controlling leukemia in transplanted CLL patients.

More generally, the abundance of neoantigens across many tumors was estimated and found to be ˜1.5 HLA-binding peptides with IC50<500 nM per point mutation and ˜4 binding peptides per frameshift mutation. As expected, the rate of predicted HLA binding peptides mirrored the somatic mutation rate per tumor type (see e.g., FIG. 20). Two approaches were used to study the relationship between predicted binding affinity and immunogenic neoantigens that induce CTLs. The above-described techniques were applied to published immunogenic tumor neoantigens (i.e. in which reactive CTLs were observed in patients) demonstrated that the vast majority (91%) of functional neoantigens are predicted to bind HLA with IC50<500 nM (with ˜70% of wild type counterpart epitopes predicted to bind at a similar affinity) (see e.g., Table 4). This test used a gold standard set of neoantigens confirmed that the techniques described herein correctly classify true positives. A prospective prediction of neoepitopes followed by functional validation showed that 6% (3/48) of predicted epitopes were associated with neoantigen-specific T cell responses in patients—comparable to the rate of 4.8% found recently for melanoma. The low proportion does not necessarily imply low prediction accuracy for the algorithm. Rather, the number of true neoantigens is greatly underestimated because: (i) allo-HSCT is a general cellular therapy likely to induce only a small number of neoantigen-specific T cell memory clones; and (ii) standard T cell expansion methods are not sensitive enough to detect naïve T cells that represent a much larger part of the repertoire but with much lower precursor frequencies. Although the frequency of CTLs that target neoORFs has yet to be measured, it is specifically contemplated within the scope of the invention that this class of neoantigens may be an excellent candidate neoepitope because it is likely to be more specific (for lack of a wild type counterpart) and immunogenic (as a result of bypassing thymic tolerance).

With the ongoing development of highly powerful vaccination reagents, the present disclosure provides techniques that make it feasible to generate personalized cancer vaccines that effectively stimulate immunity against tumor neoantigens.

### Materials and Methods

Patient samples: Heparinized blood was obtained from patients enrolled on clinical research protocols at the Dana-Farber Cancer Institute (DFCI). All clinical protocols were approved by the DFCI Human Subjects Protection Committee. Peripheral blood mononuclear cells (PBMCs) from patient samples were isolated by Ficoll/Hypaque density-gradient centrifugation, cryopreserved with 10% DMSO, and stored in vapor-phase liquid nitrogen until the time of analysis. For a subset of patients, HLA typing was performed by either molecular or serological typing (Tissue Typing Laboratory, Brigham and Women's Hospital, Boston, Mass.).

Whole exome capture sequencing data for CLL and other cancers: The list for melanoma was obtained from dbGaP database (phs000452.v1.p1) and for the 11 other cancers, through TCGA (available through the Sage Bionetworks' Synapse resource (on the worldwide web at (www)synapse.org/#!Synapse:syn1729383)). The HLA-A, HLA-B and HLA-C loci in 2488 samples across these 13 tumor types were sequenced using a two-stage likelihood based approach, and this data is summarized in Table 14. Briefly, a dedicated sequence library consisting of all known HLA alleles (6597 unique entries), based on the IMGT database, was constructed. From this resource, a secondary library of 38-mers was generated, and putative reads emanating from the HLA locus were extracted from total sequence reads based on perfect matches against it. The extracted reads were then aligned to the IMGT-based HLA sequence library using the Novoalign software (on the worldwide web at (www)novocraft.com), and HLA alleles were inferred through a two-stage likelihood calculation. In the first stage, population-based frequencies were used as priors for each allele and the posterior likelihoods were calculated based on quality and insert size distributions of aligned reads. Alleles with the highest likelihoods for each of HLA-A, B and C genes were identified as the first set of alleles. A heuristic weighting strategy of the computed likelihoods in conjunction with the first set of winners were then used to identify the second set of alleles.

Table 14 shows TCGA patient IDs for neoantigen load estimates across cancers. LUSC (lung squamous carcinoma), LUAD (lung adeno carcinoma), BLCA (bladder), HNSC (head and neck), COAD (colon) and READ (rectum), GBM (glioblastoma), OV (ovarian), RCC (clear cell renal carcinoma), AML (acute myeloid leukemia) and BRCA (breast),

TABLE 14

| TCGA Barcodes | Disease | UUID |
|---|---|---|
| TCGA-BL-A0C8-01A-11D-A10S-08 | BLCA | 134b0a5e-a0ba-444d-bc4b-bdceb02d5b04 |
| TCGA-BL-A13I-01A-11D-A13W-08 | BLCA | aa490522-7bb9-4f82-8f19-eaf63f719bfe |
| TCGA-BL-A13J-01A-11D-A10S-08 | BLCA | 0c7aca3f-e006-4de3-afc2-20b4f727d4fd |
| TCGA-BL-A3JM-01A-12D-A21A-08 | BLCA | b181ba68-f50f-4faf-b7b5-356e119b5f04 |
| TCGA-BT-A0S7-01A-11D-A10S-08 | BLCA | b2e5d244-94c1-4dbf-8d33-34b595903310 |
| TCGA-BT-A0YX-01A-11D-A10S-08 | BLCA | d61ccd8c-b798-46e0-aeed-f95b4f3ba4ff |
| TCGA-BT-A20J-01A-11D-A14W-08 | BLCA | 1d3c0ff9-d149-4d21-8955-5fb849fc5462 |
| TCGA-BT-A20N-01A-11D-A14W-08 | BLCA | 341bbffe-7587-4ad0-b3b4-68e64080e216 |
| TCGA-BT-A20O-01A-21D-A14W-08 | BLCA | 7df63263-de4e-4ed8-804f-9e8fee3be2d5 |
| TCGA-BT-A20P-01A-11D-A14W-08 | BLCA | e6c78a98-f45b-482b-a551-4f11b8c1ff8b |
| TCGA-BT-A20Q-01A-11D-A14W-08 | BLCA | 8c619cbc-9e91-4716-9711-5236e55d8f46 |
| TCGA-BT-A20R-01A-12D-A16O-08 | BLCA | e9bbbfc3-0beb-4f91-92a1-081bff7c4a07 |
| TCGA-BT-A20T-01A-11D-A14W-08 | BLCA | 301d6ce3-4099-4c1d-8e50-c04b7ce91450 |
| TCGA-BT-A20U-01A-11D-A14W-08 | BLCA | 4576527b-b288-4f50-a9ea-5d5dede22561 |
| TCGA-BT-A20V-01A-11D-A14W-08 | BLCA | 973d0577-8ca4-44a1-817f-1d3c1bada151 |
| TCGA-BT-A20W-01A-21D-A14W-08 | BLCA | 85ccdf9b-f787-4701-822f-ae0fce5b4fc5 |
| TCGA-BT-A20X-01A-11D-A16O-08 | BLCA | 9b4586ee-4091-484f-8be8-5a5196fe7b6f |
| TCGA-BT-A2LB-01A-11D-A18F-08 | BLCA | e7aea186-f13b-43b1-8693-f90f51e005dd |
| TCGA-BT-A2LD-01A-12D-A20D-08 | BLCA | cc95719c-7fcc-4ed7-837e-1840c0a6bc27 |
| TCGA-BT-A3PH-01A-11D-A21Z-08 | BLCA | cda1a403-16b6-487c-a82a-c377d1d0f89d |
| TCGA-BT-A3PJ-01A-21D-A21Z-08 | BLCA | b73523d7-f5a5-4140-8537-4df4d1ecf465 |
| TCGA-BT-A3PK-01A-21D-A21Z-08 | BLCA | 4ad38e8e-e63e-41d9-9216-617be7fa1d75 |
| TCGA-C4-A0EZ-01A-21D-A10S-08 | BLCA | b01a7081-8eb5-4728-a517-52156cdfe7ed |
| TCGA-C4-A0F0-01A-12D-A10S-08 | BLCA | 612fd956-9a41-4201-9d74-6ab50f6ae987 |
| TCGA-C4-A0F1-01A-11D-A10S-08 | BLCA | 9377460a-8497-41b8-b2c2-5f50cfeda1fe |
| TCGA-C4-A0F6-01A-11D-A10S-08 | BLCA | 608f8c75-40e4-44f2-bdde-5f07aa6b4bee |
| TCGA-C4-A0F7-01A-11D-A10S-08 | BLCA | f389176f-d8f3-45c2-aae4-7378a3d6fc7f |
| TCGA-CF-A1HR-01A-11D-A13W-08 | BLCA | 69acf4f1-063f-453d-b148-681518c0bc39 |
| TCGA-CF-A1HS-01A-11D-A13W-08 | BLCA | b36e672b-c5d8-4481-bbb3-7be805215212 |
| TCGA-CF-A27C-01A-11D-A16O-08 | BLCA | acc629cb-ad03-4cec-9b21-922e4932ef3e |
| TCGA-CF-A3MF-01A-12D-A21A-08 | BLCA | c66c92d5-df65-46e6-861d-d8a98808e6a3 |
| TCGA-CF-A3MG-01A-11D-A20D-08 | BLCA | 4c89ce08-ed24-4179-8884-4706660b7da8 |
| TCGA-CF-A3MH-01A-11D-A20D-08 | BLCA | 8867b16f-cd05-41e9-b3ca-4c72a1ebeb70 |
| TCGA-CF-A3MI-01A-11D-A20D-08 | BLCA | 0afabd62-8454-41b4-9b02-386681589688 |
| TCGA-CU-A0YN-01A-21D-A10S-08 | BLCA | 803ab221-b813-4bcc-95a9-1f686d172d3c |
| TCGA-CU-A0YO-01A-11D-A10S-08 | BLCA | e80278f9-2059-4e98-92b2-3e9868fc5818 |
| TCGA-CU-A0YR-01A-12D-A10S-08 | BLCA | 31382822-3792-47bc-99e8-8a1ee1e4e58b |
| TCGA-CU-A3KJ-01A-11D-A21A-08 | BLCA | e22c6a44-4f8e-44eb-8ca8-dff0f2fc5575 |
| TCGA-DK-A1A3-01A-11D-A13W-08 | BLCA | 2322f7cd-7d55-4a9f-b7f3-da3068089383 |
| TCGA-DK-A1A5-01A-11D-A13W-08 | BLCA | 448fe471-3f4e-4dc8-a4e0-6f147dc93abe |
| TCGA-DK-A1A6-01A-11D-A13W-08 | BLCA | df8a913c-5160-4fc5-950d-7c890e24e820 |
| TCGA-DK-A1A7-01A-11D-A13W-08 | BLCA | 91f458e6-64b7-454d-a542-b0aa23638fd8 |
| TCGA-DK-A1AA-01A-11D-A13W-08 | BLCA | 804ffa2e-158b-447d-945c-707684134c87 |
| TCGA-DK-A1AB-01A-11D-A13W-08 | BLCA | 5f0fb2ba-0351-4ce0-8b74-31aa3deecae1 |
| TCGA-DK-A1AC-01A-11D-A13W-08 | BLCA | a5dc17f5-abda-4534-b0f8-34b59ed4faa3 |
| TCGA-DK-A1AD-01A-11D-A13W-08 | BLCA | 32398d56-8668-41b1-9c0b-c6aea6e3e787 |
| TCGA-DK-A1AE-01A-11D-A13W-08 | BLCA | abd2d959-d5ed-4eb3-9759-67eb1aa23325 |
| TCGA-DK-A1AF-01A-11D-A13W-08 | BLCA | fbdcd7f9-1901-4e90-8e3c-71b05dc96da1 |
| TCGA-DK-A1AG-01A-11D-A13W-08 | BLCA | 7d2a22eb-7344-4cba-ad7d-94c3f9ef3d7c |
| TCGA-DK-A2HX-01A-12D-A18F-08 | BLCA | a8f0d416-2102-43ea-9cf1-465c37f9642a |
| TCGA-DK-A2I1-01A-11D-A17V-08 | BLCA | f350676a-e308-42fe-8297-9d18ba7027b1 |
| TCGA-DK-A2I2-01A-11D-A17V-08 | BLCA | 537e0d59-dd1c-479e-877f-eb9523c0967e |
| TCGA-DK-A2I4-01A-11D-A21A-08 | BLCA | d68074b8-ce96-4dc5-b14c-3bbc7ba92ad9 |
| TCGA-DK-A2I6-01A-12D-A18F-08 | BLCA | 97a755af-ca00-4116-8a32-0984dbfb1585 |
| TCGA-DK-A3IK-01A-32D-A21A-08 | BLCA | f730e341-8102-4405-95e2-46a3455a35cc |
| TCGA-DK-A3IL-01A-11D-A20D-08 | BLCA | 4838b5a9-968c-4178-bffb-3fafe1f6dc09 |
| TCGA-DK-A3IM-01A-11D-A20D-08 | BLCA | 780f4201-4e59-47b8-b3b7-d322a6162b2d |
| TCGA-DK-A3IN-01A-11D-A20D-08 | BLCA | 173c1518-6bcb-4e25-a119-de32dab91286 |
| TCGA-DK-A3IQ-01A-31D-A20D-08 | BLCA | c3da3cc2-2299-4a3e-9de8-7a1d0a10345d |
| TCGA-DK-A3IS-01A-21D-A21A-08 | BLCA | 92a59313-da12-4896-b164-fd2d50684638 |
| TCGA-DK-A3IT-01A-31D-A20D-08 | BLCA | 07db4596-cb49-4a32-bc99-3b202ffe61a2 |
| TCGA-DK-A3IU-01A-11D-A20D-08 | BLCA | 52de410f-3ce3-4ee6-87f3-8ec2e829962f |
| TCGA-DK-A3IV-01A-22D-A21A-08 | BLCA | 7cecfbbc-5fe4-4413-95fd-07533aacbb73 |
| TCGA-E5-A2PC-01A-11D-A202-08 | BLCA | 62b9f71c-2dab-455a-a454-579e8843f712 |
| TCGA-FD-A3B3-01A-12D-A202-08 | BLCA | 8e9fb61d-c90d-440b-857a-12e1048435ea |
| TCGA-FD-A3B4-01A-12D-A202-08 | BLCA | df922c85-5a10-487f-a9d5-220d5090e2e4 |
| TCGA-FD-A3B5-01A-11D-A20D-08 | BLCA | d05f9b81-7ba9-4231-aae6-1d2c14df22d7 |
| TCGA-FD-A3B6-01A-21D-A20D-08 | BLCA | 36524c53-ac54-4a42-a982-bed2e4354268 |
| TCGA-FD-A3B7-01A-31D-A20D-08 | BLCA | fc76c5bd-315d-4981-ae53-705f40d2c078 |
| TCGA-FD-A3B8-01A-31D-A20D-08 | BLCA | 7957bb77-8329-43a0-b1a8-140f2cb6b91b |
| TCGA-FD-A3N5-01A-11D-A21A-08 | BLCA | 418a3dec-96ff-4719-becb-e1a8260cce2f |
| TCGA-FD-A3N6-01A-11D-A21A-08 | BLCA | d4615ca0-b5c7-4a5c-8593-bd50034a78ae |
| TCGA-FD-A3NA-01A-11D-A21A-08 | BLCA | d079a32c-270b-4c43-8372-884e8d0c48ed |
| TCGA-G2-A2EC-01A-11D-A17V-08 | BLCA | 1376c881-cea5-4470-8dc1-63c69f201570 |
| TCGA-G2-A2EF-01A-12D-A18F-08 | BLCA | 4e5917bd-2cb1-438c-a46c-5d8ca5b2fd0e |
| TCGA-G2-A2EJ-01A-11D-A17V-08 | BLCA | 82f98ff9-7161-45c3-8107-033b47e25f21 |
| TCGA-G2-A2EK-01A-22D-A18F-08 | BLCA | eb73bb35-af99-47b8-8bbb-33b5374e5c74 |
| TCGA-G2-A2EL-01A-12D-A18F-08 | BLCA | 56924619-0724-4b3e-9c53-27c27d3789d6 |
| TCGA-G2-A2EO-01A-11D-A17V-08 | BLCA | ebb5cdb6-df4a-436d-b4a6-1655d263e3dd |
| TCGA-G2-A2ES-01A-11D-A17V-08 | BLCA | 5c628df6-a848-4177-87b8-714788118980 |
| TCGA-G2-A3IE-01A-11D-A20D-08 | BLCA | ebacd09f-c204-4cd2-a087-07bc4f2c5b74 |
| TCGA-GC-A3I6-01A-11D-A20D-08 | BLCA | 372feefe-ee84-4833-8651-8f023f38a56a |
| TCGA-GC-A3RB-01A-12D-A21Z-08 | BLCA | eaf54383-4286-4416-9b18-be1081797df2 |
| TCGA-GD-A2C5-01A-12D-A17V-08 | BLCA | 2b142863-b963-4cc9-8f8f-c72503c93390 |
| TCGA-GD-A3OP-01A-21D-A21Z-08 | BLCA | 3e02d723-691a-448c-85e2-4e39a3696ba5 |
| TCGA-GD-A3OQ-01A-32D-A21Z-08 | BLCA | fb985b3d-b0f7-42a0-bc3c-f71d9c5f78d8 |
| TCGA-GD-A3OS-01A-12D-A21Z-08 | BLCA | 9b3e164d-aaa0-4bb5-b7b8-6264b2746a47 |
| TCGA-GV-A3JV-01A-11D-A21Z-08 | BLCA | 5fed4b8a-4b59-4424-bbf1-bc73ce041361 |
| TCGA-GV-A3JW-01A-11D-A20D-08 | BLCA | 4534413b-d0d0-4b34-a3d4-f821705485ae |
| TCGA-GV-A3JX-01A-11D-A20D-08 | BLCA | 21525d6f-4222-4e0a-9f07-8adbbd55c54f |
| TCGA-GV-A3JZ-01A-11D-A21A-08 | BLCA | 074fc904-0a0e-4114-b569-89d51e7a89db |
| TCGA-GV-A3QG-01A-11D-A21Z-08 | BLCA | 90534196-b1d8-4054-b4d5-1d29943b52bc |
| TCGA-GV-A3QI-01A-11D-A21Z-08 | BLCA | 33a9da52-5471-456f-84cb-13c5de5b0994 |
| TCGA-H4-A2HO-01A-11D-A17V-08 | BLCA | 2e327841-eef0-42dd-883e-7d5b5a0d3a93 |
| TCGA-H4-A2HQ-01A-11D-A17V-08 | BLCA | 94108975-b7a0-40ba-ad39-e44cc62e8cc0 |
| TCGA-HQ-A2OE-01A-11D-A202-08 | BLCA | 61324839-e90a-49f2-a9c9-629d7b125fe9 |
| TCGA-A1-A0SB-01A-11D-A142-09 | BRCA | db9d40fb-bfce-4c3b-a6c2-41c5c88982f1 |
| TCGA-A1-A0SD-01A-11D-A10Y-09 | BRCA | 1847727f-ea57-4e2e-84e5-a10e764c9096 |
| TCGA-A1-A0SE-01A-11D-A099-09 | BRCA | 0539776c-3943-41d0-972c-8dc833a603e5 |
| TCGA-A1-A0SF-01A-11D-A142-09 | BRCA | b291200e-3c22-411a-85d0-fbe1570acda2 |
| TCGA-A1-A0SG-01A-11D-A142-09 | BRCA | 39642c6d-9191-4746-8a9d-62d437bfdce8 |
| TCGA-A1-A0SH-01A-11D-A099-09 | BRCA | 473d6ae4-162a-4136-b44f-fad42529a31a |
| TCGA-A1-A0SI-01A-11D-A142-09 | BRCA | e218c272-a7e1-4bc9-b8c5-d2d1c903550f |
| TCGA-A1-A0SJ-01A-11D-A099-09 | BRCA | a55c6a44-c0f5-4300-8df4-4a70befe2d3b |
| TCGA-A1-A0SK-01A-12D-A099-09 | BRCA | d1b43161-cbc1-4bf6-b8bb-a72a2e5e1150 |
| TCGA-A1-A0SM-01A-11D-A099-09 | BRCA | 2057b341-ff5c-45ef-83bb-005e29b2e740 |
| TCGA-A1-A0SN-01A-11D-A142-09 | BRCA | 1b8d93f4-acc2-48ee-9ca8-a327eb0463c2 |
| TCGA-A1-A0SO-01A-22D-A099-09 | BRCA | b3568259-c63c-4eb1-bbc7-af711ddd33db |
| TCGA-A1-A0SP-01A-11D-A099-09 | BRCA | d3ae9617-b6cd-4d98-b631-39bd4afd3c4e |
| TCGA-A1-A0SQ-01A-21D-A142-09 | BRCA | 9055ddce-a0ff-4980-af86-c07f949acbc3 |
| TCGA-A2-A04N-01A-11D-A10Y-09 | BRCA | 389dd52b-a7b7-46f0-83ae-308e485466a8 |
| TCGA-A2-A04P-01A-31D-A128-09 | BRCA | a85cf239-ff51-46e7-9688-4c2cb49c66b9 |
| TCGA-A2-A04Q-01A-21W-A050-09 | BRCA | 02eb17d4-9e9e-4e32-96b0-90ccdda3f167 |
| TCGA-A2-A04R-01A-41D-A117-09 | BRCA | 1f8e4326-dfc7-4635-a9b7-a9207a392748 |
| TCGA-A2-A04U-01A-11D-A10Y-09 | BRCA | f819433a-44db-4022-abdb-d6123cfa30b2 |
| TCGA-A2-A04V-01A-21W-A050-09 | BRCA | 89501861-2778-4b88-9a44-939fed99850d |
| TCGA-A2-A04W-01A-31D-A10Y-09 | BRCA | 7822a6b1-68c8-4675-993c-c4b54a510c09 |
| TCGA-A2-A04X-01A-21W-A050-09 | BRCA | 66a73891-2fea-450c-8224-0865d98b4346 |
| TCGA-A2-A04Y-01A-21W-A050-09 | BRCA | 3669bbbd-2e75-4b57-a5a8-8eebc25a97c2 |
| TCGA-A2-A0CL-01A-11D-A10Y-09 | BRCA | a630ed59-dd23-45e1-aa16-4f7a98e32728 |
| TCGA-A2-A0CM-01A-31W-A050-09 | BRCA | fe8023d4-5476-4c58-bf70-cbf65cdd4327 |
| TCGA-A2-A0CP-01A-11W-A050-09 | BRCA | a776e274-fe9f-49a9-83ab-95ca6819c96b |
| TCGA-A2-A0CQ-01A-21W-A050-09 | BRCA | fa0d7183-8757-4f95-87b2-2366a1dbd508 |
| TCGA-A2-A0CS-01A-11D-A10Y-09 | BRCA | fe96b832-cb86-4499-948a-5124a43d5c95 |
| TCGA-A2-A0CT-01A-31W-A071-09 | BRCA | 2b412ad8-abda-4cf8-8f68-59dbce80031e |
| TCGA-A2-A0CU-01A-12W-A050-09 | BRCA | a9aa68af-f5fe-4ac0-987f-8af49b85c231 |
| TCGA-A2-A0CV-01A-31D-A10Y-09 | BRCA | 5d1dead5-d9a5-42d3-a703-4c38ad6e8f57 |
| TCGA-A2-A0CW-01A-21D-A10Y-09 | BRCA | da4f0f85-b16f-40fa-95c6-524d70d7ac4d |
| TCGA-A2-A0CX-01A-21W-A019-09 | BRCA | 975adb76-3561-41a0-959a-68da470816c7 |
| TCGA-A2-A0CZ-01A-11W-A050-09 | BRCA | 95d5c606-367a-46b5-b663-dcea3f42e2a2 |
| TCGA-A2-A0D0-01A-11W-A019-09 | BRCA | 3f20d0fe-aaa1-40f1-b2c1-7f070f93aef5 |
| TCGA-A2-A0D1-01A-11W-A050-09 | BRCA | a762809c-15c9-485e-ad7a-ef28427750e9 |
| TCGA-A2-A0D2-01A-21W-A050-09 | BRCA | 05656575-69e7-4745-a89d-ca0568eb5559 |
| TCGA-A2-A0D3-01A-11D-A10Y-09 | BRCA | 8183420e-7f44-4024-b3db-6b53ad293988 |
| TCGA-A2-A0D4-01A-11W-A019-09 | BRCA | f3accede-1716-4d44-bad4-5427a9ebd675 |
| TCGA-A2-A0EM-01A-11W-A050-09 | BRCA | 0e01c6b8-9edd-4965-b247-ee7e68124f48 |
| TCGA-A2-A0EN-01A-13D-A099-09 | BRCA | 12362ad7-6866-4e7a-9ec6-8a0a68df8896 |
| TCGA-A2-A0EO-01A-11W-A050-09 | BRCA | 8e2f9eb7-0660-47ae-b86e-652e99fa69ca |
| TCGA-A2-A0EQ-01A-11W-A050-09 | BRCA | 2c449ea9-c3ff-4726-8566-5933e2b7056d |
| TCGA-A2-A0ER-01A-21W-A050-09 | BRCA | 31ed187e-9bfe-4ca3-8cbb-10c1e0184331 |
| TCGA-A2-A0ES-01A-11D-A10Y-09 | BRCA | 64d42c62-5c2d-49f5-856e-72beef88044d |
| TCGA-A2-A0ET-01A-31D-A045-09 | BRCA | f7b40023-4adc-4c7d-ae73-5c10ddcbc0fb |
| TCGA-A2-A0EU-01A-22W-A071-09 | BRCA | de30da8f-903f-428e-a63d-59625fc858a9 |
| TCGA-A2-A0EV-01A-11W-A050-09 | BRCA | 9433bf4f-23ba-4fe7-9503-1ad243d74225 |
| TCGA-A2-A0EW-01A-21D-A10Y-09 | BRCA | a045a04e-4f7b-4f9a-a733-47ad24475496 |
| TCGA-A2-A0EX-01A-21W-A050-09 | BRCA | 9308f50c-1320-4c45-acc7-38f43b6f9a36 |
| TCGA-A2-A0EY-01A-11W-A050-09 | BRCA | a8cde596-e3f5-4b20-9e7f-45d079893176 |
| TCGA-A2-A0ST-01A-12D-A099-09 | BRCA | dd669f44464d-4afc-a5ac-5f7769d1db43 |
| TCGA-A2-A0SU-01A-11D-A099-09 | BRCA | 6ceaf20f-1458-4f7f-954a-e2f58ed163bf |
| TCGA-A2-A0SV-01A-11D-A099-09 | BRCA | 6d3206c6-0ca8-4b2b-a160-b1719217f9c7 |
| TCGA-A2-A0SW-01A-11D-A099-09 | BRCA | 7fbd2807-a5bb-4030-a299-524ec3ab4543 |
| TCGA-A2-A0SX-01A-12D-A099-09 | BRCA | b54bc31e-bdcc-4ad5-998e-5a9c542f83bb |
| TCGA-A2-A0SY-01A-31D-A099-09 | BRCA | efaa9c0b-c14b-4141-b48c-cc2c6b89ab73 |
| TCGA-A2-A0T0-01A-22D-A099-09 | BRCA | 3c107ce4-a6ac-469b-b1c0-cd86674b5766 |
| TCGA-A2-A0T1-01A-21D-A099-09 | BRCA | 9515373a-d982-45fa-b8f9-363f9ba8649f |
| TCGA-A2-A0T2-01A-11W-A097-09 | BRCA | c7918143-dbce-45b3-8d24-2993a9e2b7f4 |
| TCGA-A2-A0T3-01A-21D-A10Y-09 | BRCA | 0ca029bb-3b3a-48ec-8ade-5591e8e8629f |
| TCGA-A2-A0T4-01A-31D-A099-09 | BRCA | 0f1b1fda-4956-498a-b8ff-e98b5d64e509 |
| TCGA-A2-A0T6-01A-11D-A099-09 | BRCA | e4dcb280-c309-4ebb-a58d-e6389a0306ee |
| TCGA-A2-A0T7-01A-21D-A099-09 | BRCA | 3ea4d98d-f8d9-433e-94f1-b0199bfdb198 |
| TCGA-A2-A0YC-01A-11D-A117-09 | BRCA | 4cccf7dc-7c53-409f-a6b1-f86e0f07250b |
| TCGA-A2-A0YD-01A-11D-A10G-09 | BRCA | 30c9f9e5-90b2-4c73-bce5-eb6a3d31f496 |
| TCGA-A2-A0YF-01A-21D-A10G-09 | BRCA | 11571107-fe70-4140-afff-f4792a4fd473 |
| TCGA-A2-A0YG-01A-21D-A10G-09 | BRCA | bf82035c-9cd1-4355-acdd-8a007708e976 |
| TCGA-A2-A0YH-01A-11D-A10G-09 | BRCA | e5558a39-eab2-4216-ba88-b63c2de48b01 |
| TCGA-A2-A0YI-01A-31D-A10M-09 | BRCA | 6d2ae968-c977-4b65-869a-5e96ff3216e9 |
| TCGA-A2-A0YJ-01A-11D-A10G-09 | BRCA | 3fe8e99f-dce5-4df9-983e-efe63d56bdd5 |
| TCGA-A2-A0YK-01A-22D-A117-09 | BRCA | 7c27f81e-62fb-478c-9cee-8e20db9300f2 |
| TCGA-A2-A0YL-01A-21D-A10G-09 | BRCA | 3cc80b41-603d-4735-85c7-71f540dc6e5c |
| TCGA-A2-A0YM-01A-11D-A10G-09 | BRCA | 1125ec93-6d24-4537-9c89-526f2d6b2299 |
| TCGA-A2-A0YT-01A-11D-A10G-09 | BRCA | 827c6a2f-fb1b-4845-9cb1-11013a16da3f |
| TCGA-A2-A1FV-01A-11D-A13L-09 | BRCA | 51b7064c-d9fc-4312-ad25-b014ef81c821 |
| TCGA-A2-A1FW-01A-11D-A13L-09 | BRCA | 6ccdb42e-1ad1-4175-b83a-a24b019dc640 |
| TCGA-A2-A1FX-01A-11D-A13L-09 | BRCA | 0d3dd7a0-ad8d-46cc-86c4-c1994a7b4b74 |
| TCGA-A2-A1FZ-01A-51D-A17G-09 | BRCA | 0f7038bb-fd25-468e-8bd9-dcd4312d13cb |
| TCGA-A2-A1G0-01A-11D-A13L-09 | BRCA | f7eacf95-478d-4d81-a5e3-f5a8938c83ec |
| TCGA-A2-A1G1-01A-21D-A13L-09 | BRCA | afe70076-1044-4fdd-bebc-14a97b1a8363 |
| TCGA-A2-A1G4-01A-11D-A13L-09 | BRCA | 420a4771-6376-4b52-a2e3-e62aaf4d4ed6 |
| TCGA-A2-A1G6-01A-11D-A13L-09 | BRCA | c012bce9-de13-4e32-a29e-8ab64e16ea96 |
| TCGA-A2-A259-01A-11D-A16D-09 | BRCA | 93febb0a-587c-47f2-9a59-117f7aa475c5 |
| TCGA-A2-A25A-01A-12D-A16D-09 | BRCA | 5739a7e1-7fa3-434c-b1c3-c0a9e570c858 |
| TCGA-A2-A25B-01A-11D-A167-09 | BRCA | 6e839eaf-1dbb-43f5-8846-c980e05540c7 |
| TCGA-A2-A25C-01A-11D-A167-09 | BRCA | 2411fc4a-c0d7-4a60-a861-f4d954ef1ed5 |
| TCGA-A2-A25D-01A-12D-A16D-09 | BRCA | 56b152c3-9de5-4b1c-b6b4-0116cb7ce097 |
| TCGA-A2-A25E-01A-11D-A167-09 | BRCA | 8dce6a9d-ecb7-4699-9fda-1b09b1b1de43 |
| TCGA-A2-A25F-01A-11D-A167-09 | BRCA | 1ed40576-4f1c-4cf6-8eea-e816c5d73d90 |
| TCGA-A7-A0CD-01A-11W-A019-09 | BRCA | d29ba065-28ca-4dfb-9588-06be857f67b2 |
| TCGA-A7-A0CG-01A-11W-A019-09 | BRCA | 351275c7-70ca-4ddc-be76-a6ff4dc7655e |
| TCGA-A7-A0CJ-01A-21W-A019-09 | BRCA | c9f6a65e-ae20-410d-a397-34aef0818ff3 |
| TCGA-A7-A0DA-01A-31D-A10Y-09 | BRCA | 878337fe-9f41-44f5-9760-3977e7d75308 |
| TCGA-A7-A13D-01A-13D-A12Q-09 | BRCA | 418e916b-7a4e-4fab-8616-15dcec4d79f8 |
| TCGA-A7-A13G-01A-11D-A13L-09 | BRCA | ef847b83-eb88-435b-bcfd-4b51d4dfa5fe |
| TCGA-A7-A26E-01A-11D-A167-09 | BRCA | 73651880-cfbd-4f8d-8031-a14b3ac65454 |
| TCGA-A7-A26F-01A-21D-A167-09 | BRCA | fc73db72-d0ac-48d0-b809-2f7540482ec5 |
| TCGA-A7-A26G-01A-21D-A167-09 | BRCA | 36d1a85e-a09b-4537-86e0-eaf1eb03aed8 |
| TCGA-A7-A26H-01A-11D-A167-09 | BRCA | fbeade79-28ef-4e85-8282-67e691630ca3 |
| TCGA-A7-A26I-01A-11D-A167-09 | BRCA | 81fff2d1-d6ed-4963-a5f6-5899cde6b359 |
| TCGA-A7-A26J-01A-11D-A167-09 | BRCA | be2ca34f-5c15-4b38-a207-52df296a98ee |
| TCGA-A8-A06N-01A-11W-A019-09 | BRCA | 03d266a3-eb3e-4893-af6b-cb70d197d98f |
| TCGA-A8-A06O-01A-11W-A019-09 | BRCA | 29cd408e-a04b-418a-85e2-6ef95840ddbc |
| TCGA-A8-A06P-01A-11W-A019-09 | BRCA | 239b3d55-c5d6-4478-967b-1cbad3c03c81 |
| TCGA-A8-A06Q-01A-11W-A050-09 | BRCA | 473d5422-978a-48be-be32-2b7516d6d2d5 |
| TCGA-A8-A06R-01A-11D-A015-09 | BRCA | c6b00eff-6c4e-4d79-a9b1-8fb1f3090816 |
| TCGA-A8-A06T-01A-11W-A019-09 | BRCA | 11ec4a6f-f2dc-4b0b-9ba5-6fea8222e2d7 |
| TCGA-A8-A06U-01A-11W-A019-09 | BRCA | 277c2e8a-dd28-4b8f-96d3-ea790a1986b6 |
| TCGA-A8-A06X-01A-21W-A019-09 | BRCA | dc306402-3a55-4996-b786-f3f738f13dd3 |
| TCGA-A8-A06Y-01A-21W-A019-09 | BRCA | 3bede568-d8b6-44c0-99e0-a9b6c7d4ce80 |
| TCGA-A8-A06Z-01A-11W-A019-09 | BRCA | f540c4f8-75b3-47d7-a7cf-53cbf7a2c814 |
| TCGA-A8-A075-01A-11D-A099-09 | BRCA | 085dd125-1f95-46aa-a480-2965090e8591 |
| TCGA-A8-A076-01A-21W-A019-09 | BRCA | dfa06058-320b-4cc6-ac18-a42e59019b1c |
| TCGA-A8-A079-01A-21W-A019-09 | BRCA | 06221ce8-ab65-4694-945b-059b9c15ede4 |
| TCGA-A8-A07B-01A-11W-A019-09 | BRCA | 734421b9-ed55-45b0-9ad5-51bc754ebe90 |
| TCGA-A8-A07C-01A-11D-A045-09 | BRCA | 6ab33f67-b69d-4a2d-a424-841f5fbf1ee7 |
| TCGA-A8-A07E-01A-11W-A050-09 | BRCA | fa018a20-2c26-4d47-831f-75280b6464df |
| TCGA-A8-A07F-01A-11W-A019-09 | BRCA | 73d907e6-4ba0-431f-a009-8366644ffaf0 |
| TCGA-A8-A07G-01A-11W-A050-09 | BRCA | 49f77aa5-446b-49f6-bd1b-02d3ff7b9dfc |
| TCGA-A8-A07I-01A-11W-A019-09 | BRCA | 7718c3f0-1c90-4940-bc30-ea4f417851bb |
| TCGA-A8-A07J-01A-11W-A019-09 | BRCA | c8eac36c-c3a7-4c88-b928-832ab279045b |
| TCGA-A8-A07L-01A-11W-A019-09 | BRCA | 4cc86f29-061e-4058-8e8f-4c48191f52aa |
| TCGA-A8-A07O-01A-11W-A019-09 | BRCA | 4574b64d-8848-46e4-913e-5d318c1f6162 |
| TCGA-A8-A07P-01A-11W-A019-09 | BRCA | 2b88ff64-bf43-43e8-9ea9-0de571520d72 |
| TCGA-A8-A07R-01A-21W-A050-09 | BRCA | f377217c-399f-4b3f-9090-fa5189b2bfc6 |
| TCGA-A8-A07U-01A-11W-A050-09 | BRCA | e6409415-8453-489d-a731-49257cade2a3 |
| TCGA-A8-A07W-01A-11W-A019-09 | BRCA | 9bc8dbab-c700-498c-8ff7-ccc62c911349 |
| TCGA-A8-A07Z-01A-11W-A019-09 | BRCA | e4af33f9-f5fe-4e52-8ca0-991bbce2270d |
| TCGA-A8-A081-01A-11W-A019-09 | BRCA | d29c3a5b-aab5-4d1b-bdaf-eb6fa405bc80 |
| TCGA-A8-A082-01A-11W-A019-09 | BRCA | 575d25ea-eae7-423a-9464-d3b2806bf9eb |
| TCGA-A8-A083-01A-21W-A019-09 | BRCA | 1904e458-1a6c-4e91-88cc-10ee154ded5b |
| TCGA-A8-A084-01A-21W-A019-09 | BRCA | 6f6f7048-5b7a-4827-af2b-cfecc4a60025 |
| TCGA-A8-A085-01A-11W-A019-09 | BRCA | cbdea951-3dc9-42c2-bfdd-3796c30e928e |
| TCGA-A8-A086-01A-11W-A019-09 | BRCA | 13d89926-9e4c-434f-80b4-4fb15e4426f6 |
| TCGA-A8-A08A-01A-11W-A019-09 | BRCA | 0257d030-6d78-452c-9dcc-79fe50533543 |
| TCGA-A8-A08B-01A-11W-A019-09 | BRCA | 267a951b-2967-4849-9ea7-d2205838fcc7 |
| TCGA-A8-A08F-01A-11W-A019-09 | BRCA | 4975eeda-984e-4a7a-8193-43d8b6e0271c |
| TCGA-A8-A08G-01A-11W-A019-09 | BRCA | 8da61928-e935-4a33-8e46-840e637163d7 |
| TCGA-A8-A08H-01A-21W-A019-09 | BRCA | 26161c06-f816-489a-8800-e0a68a4ce78a |
| TCGA-A8-A08I-01A-11W-A019-09 | BRCA | 4525400d-0a2c-4cc7-9c71-9ad6d9faf93f |
| TCGA-A8-A08J-01A-11W-A019-09 | BRCA | ae458901-e900-4aaa-bde6-3eda8912fbd5 |
| TCGA-A8-A08L-01A-11W-A019-09 | BRCA | 8b819a59-f0c1-456a-9e81-64b5bed025c1 |
| TCGA-A8-A08O-01A-21W-A071-09 | BRCA | bc1398b9-d4ec-43e8-86bc-7025afaf93d5 |
| TCGA-A8-A08P-01A-11W-A019-09 | BRCA | 2fbe3da3-ce62-4edf-933b-367f983e221a |
| TCGA-A8-A08R-01A-11W-A050-09 | BRCA | 05362091-8e04-46e2-81e7-1efddc0d8c63 |
| TCGA-A8-A08S-01A-11W-A050-09 | BRCA | 9c981525-80af-4f79-b94a-be00131ab872 |
| TCGA-A8-A08T-01A-21W-A019-09 | BRCA | af5f43d9-5ff3-4fd8-9c1c-30a88d2bab8e |
| TCGA-A8-A08X-01A-21W-A019-09 | BRCA | 67c7d350-5c82-49b0-a7eb-6ca829ffcbc9 |
| TCGA-A8-A08Z-01A-21W-A019-09 | BRCA | 96afb6d0-29ea-4bd5-8a9d-130e42954707 |
| TCGA-A8-A090-01A-11W-A019-09 | BRCA | 783e4c13-8fa5-4591-9453-1e59ca167e10 |
| TCGA-A8-A091-01A-11W-A019-09 | BRCA | 6618f367-c782-43a0-b5c8-a53d9bda6722 |
| TCGA-A8-A092-01A-11W-A019-09 | BRCA | 732dd0ab-c869-4d35-973f-9db064680fb1 |
| TCGA-A8-A093-01A-11W-A019-09 | BRCA | 8f64ba22-0958-4fdb-8161-f83cfe57c95d |
| TCGA-A8-A094-01A-11W-A019-09 | BRCA | ab9bf7a6-688e-4388-9682-6b1616723fde |
| TCGA-A8-A095-01A-11W-A019-09 | BRCA | d16f025a-4187-4632-b833-02a3ffa54210 |
| TCGA-A8-A096-01A-11W-A019-09 | BRCA | 8a411a0a-ec66-4d9f-b0e4-f1c1f969d605 |
| TCGA-A8-A097-01A-11W-A050-09 | BRCA | 15ca7c47-131a-4dd7-b0a7-584577b4b02c |
| TCGA-A8-A099-01A-11W-A019-09 | BRCA | 1066cb38-e051-42fa-a8bc-20b659c17a13 |
| TCGA-A8-A09A-01A-11W-A019-09 | BRCA | ecfedc29-5c31-4d3d-b599-fc0a1c0beafa |
| TCGA-A8-A09B-01A-11W-A019-09 | BRCA | a8be37d2-2743-4fde-9aae-2623b5a03b60 |
| TCGA-A8-A09C-01A-11W-A019-09 | BRCA | b56cf2cb-bb2a-46b6-b3b4-84dd8b364984 |
| TCGA-A8-A09D-01A-11W-A019-09 | BRCA | d0ef396f-4e9f-40ba-a09c-0a96832cabf9 |
| TCGA-A8-A09E-01A-11W-A019-09 | BRCA | d6465963-5ea6-44a5-96b0-dff0b0fae4c4 |
| TCGA-A8-A09G-01A-21W-A019-09 | BRCA | 3bd68e94-d902-4079-8fdb-16edcc90de1c |
| TCGA-A8-A09I-01A-22W-A050-09 | BRCA | 96d5070d-1fa9-4fa5-b2c9-472240dfd3b9 |
| TCGA-A8-A09K-01A-11W-A019-09 | BRCA | d8cd75f2-5ee5-4296-a781-a6a16ee94506 |
| TCGA-A8-A09M-01A-11W-A019-09 | BRCA | 8e92515a-8049-4ebb-9117-a137c06e5d04 |
| TCGA-A8-A09N-01A-11W-A019-09 | BRCA | 304a2945-f134-45c7-9eaa-c6c9c2435552 |
| TCGA-A8-A09Q-01A-11W-A019-09 | BRCA | 51a8ac83-bafa-4df7-a52d-a1e1fb45799d |
| TCGA-A8-A09R-01A-11W-A019-09 | BRCA | 35ebf91d-6fec-4d28-9b21-493d0e14f8db |
| TCGA-A8-A09T-01A-11W-A019-09 | BRCA | e565da2b-4a3f-4be1-9cf7-2845145d1dbc |
| TCGA-A8-A09V-01A-11D-A045-09 | BRCA | 818f1a34-17c5-409a-b5f5-4a8576db0d44 |
| TCGA-A8-A09W-01A-11W-A019-09 | BRCA | 9a2690ce-485f-4d4f-9673-d86f91be27a4 |
| TCGA-A8-A09X-01A-11W-A019-09 | BRCA | 48e532ea-2af5-427a-a784-781e208cced6 |
| TCGA-A8-A0A1-01A-11W-A019-09 | BRCA | 73aa20fe-b74b-41ae-88d3-2d5a66908c25 |
| TCGA-A8-A0A2-01A-11W-A050-09 | BRCA | b681dba3-a608-47c2-9ae8-5d761d1e800e |
| TCGA-A8-A0A4-01A-11W-A019-09 | BRCA | 1fc4d542-86ac-42bc-9fbb-272c23e6aa72 |
| TCGA-A8-A0A7-01A-11W-A019-09 | BRCA | 28be7b14-730d-44f7-bf93-a7590b4a08f8 |
| TCGA-A8-A0A9-01A-11W-A019-09 | BRCA | 228e66eb-1dc6-4c01-8252-c557a8f53916 |
| TCGA-A8-A0AB-01A-11W-A050-09 | BRCA | ad2a2f5d-dad6-4c03-b235-20810d6d34dc |
| TCGA-A8-A0AD-01A-11W-A071-09 | BRCA | 6e6511fa-4f6e-4184-84b8-9e9e7a863632 |
| TCGA-AC-A23C-01A-12D-A167-09 | BRCA | 91766158-e175-4270-bc01-8e853fc9f391 |
| TCGA-AC-A23E-01A-11D-A159-09 | BRCA | 137cb73f-394a-459a-83e6-0b3c85c955cd |
| TCGA-AN-A03X-01A-21W-A019-09 | BRCA | f177234e-e0a7-4f85-b73d-48e0080c805d |
| TCGA-AN-A03Y-01A-21W-A019-09 | BRCA | f4849adc-b6e8-40bd-9de4-dc5bb37d2a79 |
| TCGA-AN-A041-01A-11W-A050-09 | BRCA | f18c7389-6c8d-485f-a7f7-a450a42e3719 |
| TCGA-AN-A049-01A-21W-A019-09 | BRCA | 1d0c87ef-6840-4051-85d5-7fc2c544578c |
| TCGA-AN-A04A-01A-21W-A050-09 | BRCA | 7e8f250c-6162-4049-8559-5bfdf054b021 |
| TCGA-AN-A04C-01A-21W-A050-09 | BRCA | c1302f79-cc50-487a-9db5-016df85e67d7 |
| TCGA-AN-A04D-01A-21W-A050-09 | BRCA | 9407735f-19e3-49d0-b783-cd9672dfa6a9 |
| TCGA-AN-A0AJ-01A-11W-A019-09 | BRCA | 97fbce82-0eed-4d70-9af2-57918a4ea8da |
| TCGA-AN-A0AL-01A-11W-A019-09 | BRCA | 47849ee3-b59e-4ccf-a261-65f7e252b885 |
| TCGA-AN-A0AM-01A-11W-A050-09 | BRCA | a238f21f-ca46-4759-b5b7-f8c3810dfbdb |
| TCGA-AN-A0AR-01A-11W-A019-09 | BRCA | a2d77acd-89db-4d2d-89d7-d1cc58cf576b |
| TCGA-AN-A0AS-01A-11W-A019-09 | BRCA | 2257c942-1274-47e7-86ad-b92ecfafc205 |
| TCGA-AN-A0AT-01A-11D-A045-09 | BRCA | f848b66f-bd9e-4fba-afd4-eb58848d1ef4 |
| TCGA-AN-A0FD-01A-11W-A050-09 | BRCA | abae6f4c-2378-4fbd-adea-f739e6629b22 |
| TCGA-AN-A0FF-01A-11W-A050-09 | BRCA | cd45e46c-50bf-449e-bb40-29ccffbbd49c |
| TCGA-AN-A0FJ-01A-11W-A019-09 | BRCA | 6b988737-0504-42bb-8c75-d70d7a312e68 |
| TCGA-AN-A0FK-01A-11W-A050-09 | BRCA | a765959e-b234-427d-aade-855d6d4981d9 |
| TCGA-AN-A0FL-01A-11W-A050-09 | BRCA | 18ee29ae-fe36-49a3-9843-e0757c69a7dd |
| TCGA-AN-A0FN-01A-11W-A050-09 | BRCA | 8f583981-b257-43ee-9c9e-71a192a49d38 |
| TCGA-AN-A0FS-01A-11W-A050-09 | BRCA | 9bb76d20-cefb-4f7a-80c2-aa2178e302a9 |
| TCGA-AN-A0FT-01A-11W-A050-09 | BRCA | 0598fc5f-9651-4ace-bf4e-56759d544e52 |
| TCGA-AN-A0FV-01A-11W-A019-09 | BRCA | c70259c1-f561-43d7-9829-6852815baa87 |
| TCGA-AN-A0FW-01A-11W-A050-09 | BRCA | 5afde43a-194c-4876-b244-2132aef2f505 |
| TCGA-AN-A0FX-01A-11W-A050-09 | BRCA | 2523cf22-1a16-42be-8560-833ed2031e3c |
| TCGA-AN-A0FY-01A-11W-A050-09 | BRCA | a6a8bd08-0e60-442d-adce-de020177f82c |
| TCGA-AN-A0FZ-01A-11W-A050-09 | BRCA | d77f59f7-8cff-41f3-a1bb-0de14524d4f4 |
| TCGA-AN-A0G0-01A-11W-A050-09 | BRCA | 9eb55dd2-a956-4dfe-8631-04722c49819f |
| TCGA-AN-A0XL-01A-11D-A10M-09 | BRCA | 1b08a181-a73b-4506-aaa3-3521f2c57207 |
| TCGA-AN-A0XN-01A-21D-A10G-09 | BRCA | 94a6c172-25e2-4438-945c-9b310f89ae22 |
| TCGA-AN-A0XO-01A-11D-A10G-09 | BRCA | f63863f5-cb60-4961-a5b4-ed5ea1fb3dc8 |
| TCGA-AN-A0XP-01A-11D-A117-09 | BRCA | 6179b498-2cea-4f7a-82a8-b7ec71431ea8 |
| TCGA-AN-A0XR-01A-11D-A10G-09 | BRCA | e7dc7492-3a84-49c7-8dea-8f508b53dc40 |
| TCGA-AN-A0XS-01A-22D-A10G-09 | BRCA | f1b5268d-556f-404f-a956-770df4a1e7aa |
| TCGA-AN-A0XT-01A-11D-A10G-09 | BRCA | 353d9161-95fd-4bec-abb7-859d9ee19785 |
| TCGA-AN-A0XU-01A-11D-A10G-09 | BRCA | 537c5818-eb89-4b46-8915-2bb2b9e4545f |
| TCGA-AN-A0XV-01A-11D-A10G-09 | BRCA | 6f0e5a39-e2c7-4a93-bd63-f1bab1e7c16e |
| TCGA-AN-A0XW-01A-11D-A10G-09 | BRCA | 200dba9e-201b-4634-a2cf-666e1f6710dc |
| TCGA-AO-A03L-01A-41W-A071-09 | BRCA | 743a29c4-e1cc-457a-8406-765f1a1bc114 |
| TCGA-AO-A03N-01B-11D-A10M-09 | BRCA | ef5987f1-46ac-430a-b94a-49afa0e286d4 |
| TCGA-AO-A03O-01A-11W-A019-09 | BRCA | 1578b356-7f42-4722-bc54-cd5f37954f6a |
| TCGA-AO-A03P-01A-11W-A019-09 | BRCA | 185c5e15-c068-4a72-8d5e-468624bf958a |
| TCGA-AO-A03R-01A-21W-A050-09 | BRCA | 6d2dc4e3-f1ed-4ef0-ae83-e09c87756d56 |
| TCGA-AO-A03T-01A-21W-A050-09 | BRCA | cbea866d-da66-4f7c-994b-c1ec35aa2d4b |
| TCGA-AO-A03U-01B-21D-A10M-09 | BRCA | 1e0ecd57-5c7d-4495-874d-9e286c999c22 |
| TCGA-AO-A03V-01A-11D-A10Y-09 | BRCA | d88c365f-366a-49d5-9860-b930aab3eb1b |
| TCGA-AO-A0J2-01A-11W-A050-09 | BRCA | 84b66e02-1b37-4424-b752-363f7861fe74 |
| TCGA-AO-A0J3-01A-11W-A050-09 | BRCA | ff706355-867e-4968-99ad-0af4e24ece51 |
| TCGA-AO-A0J4-01A-11W-A050-09 | BRCA | 7667f49c-449d-44ce-bab8-02a491bb6775 |
| TCGA-AO-A0J5-01A-11W-A050-09 | BRCA | 93ae73f6-c355-47be-a355-faa78c0632d4 |
| TCGA-AO-A0J6-01A-11W-A050-09 | BRCA | 7d21a0c4-03c7-4641-8b4d-7a5877960360 |
| TCGA-AO-A0J7-01A-11W-A050-09 | BRCA | a53056d9-e8bd-4cb1-ad67-85879ccc925d |
| TCGA-AO-A0J8-01A-21D-A045-09 | BRCA | 24ba5501-8097-4af6-b12c-bb6dcbe10cac |
| TCGA-AO-A0J9-01A-11W-A050-09 | BRCA | 9932232f-a7b0-4962-9b14-adb8316a4661 |
| TCGA-AO-A0JA-01A-11W-A071-09 | BRCA | 0215d4f1-6697-4e8f-afc4-ff7c6439e56d |
| TCGA-AO-A0JB-01A-11W-A071-09 | BRCA | 8f4f06be-2a16-4ae2-9dd4-5d87f480810b |
| TCGA-AO-A0JC-01A-11W-A071-09 | BRCA | 120f55df-5d1d-4073-a21a-632c892d3da9 |
| TCGA-AO-A0JD-01A-11W-A071-09 | BRCA | 9d3ad8d0-ddd3-44d2-ba0e-0b283a4fbf32 |
| TCGA-AO-A0JE-01A-11W-A071-09 | BRCA | 4f311714-ebb4-47fb-b471-62c6951d9066 |
| TCGA-AO-A0JF-01A-11W-A071-09 | BRCA | 191caa1a-5ab8-4db5-b42a-f1c5964b0b0d |
| TCGA-AO-A0JG-01A-31D-A099-09 | BRCA | cf7ec093-5040-43db-949c-f426795a7488 |
| TCGA-AO-A0JI-01A-21W-A100-09 | BRCA | 861297ec-2c88-4717-ae63-eb8e21fe8c52 |
| TCGA-AO-A0JJ-01A-11W-A071-09 | BRCA | 812191d1-6711-4efd-8932-c76159b60ffb |
| TCGA-AO-A0JL-01A-11W-A071-09 | BRCA | 56a22648-be92-402c-a225-bcaa44a7e612 |
| TCGA-AO-A0JM-01A-21W-A071-09 | BRCA | f070142b-f44e-4264-8919-dde7d02ad835 |
| TCGA-AO-A124-01A-11D-A10M-09 | BRCA | 987528ac-437a-4eb8-a335-4f2076d5c006 |
| TCGA-AO-A125-01A-11D-A10M-09 | BRCA | 17669c6d-2eeb-4d56-ac72-f06bfafb7e42 |
| TCGA-AO-A126-01A-11D-A10M-09 | BRCA | 85b39644-6f19-40dc-94c1-0afc93ee4981 |
| TCGA-AO-A129-01A-21D-A10M-09 | BRCA | cdf43c25-3ba7-4073-a92d-4a97f651f4a8 |
| TCGA-AO-A12A-01A-21D-A10Y-09 | BRCA | 77e7b41a-d4c8-42ee-ae6e-da15ea3634d9 |
| TCGA-AO-A12B-01A-11D-A10M-09 | BRCA | 865ebd77-7b7d-4a27-b945-df5ec8d1f86a |
| TCGA-AO-A12D-01A-11D-A10Y-09 | BRCA | b3065cfe-3067-4f08-8c82-46f10c1ec279 |
| TCGA-AO-A12E-01A-11D-A10M-09 | BRCA | b3990b59-e2f4-4759-8eb0-11ad3c34ac50 |
| TCGA-AO-A12F-01A-11D-A10Y-09 | BRCA | d1617673-57c2-40c1-a97043692ee13cf3 |
| TCGA-AO-A12G-01A-11D-A10M-09 | BRCA | 5b9d3741-2aa3-489b-93e6-3b5376b80d48 |
| TCGA-AO-A12H-01A-11D-A10Y-09 | BRCA | 5a535c49-d42e-43c6-9d32-dc76f28d4f0f |
| TCGA-AO-A1KO-01A-31D-A188-09 | BRCA | 2cdecb2b-40b1-4419-bcd9-101cee78966c |
| TCGA-AO-A1KP-01A-11D-A13L-09 | BRCA | bc36db60-3f6b-42c4-b03e-b7c74c3dda5c |
| TCGA-AO-A1KR-01A-12D-A142-09 | BRCA | d3b598d8-8a3b-4506-aa98-9fbc5b51afd4 |
| TCGA-AO-A1KS-01A-11D-A13L-09 | BRCA | 21074661-4b0f-4adc-b406-5801688a3ae9 |
| TCGA-AO-A1KT-01A-11D-A13L-09 | BRCA | 97b33dc3-6a62-419a-aa6c-cb84c9f92102 |
| TCGA-AQ-A04H-01B-11D-A10M-09 | BRCA | 73c13e04-1400-4ebb-aa80-f54becbe036c |
| TCGA-AQ-A04J-01A-02W-A050-09 | BRCA | cce21f2b-784b-4fa0-9809-ae532c528f8e |
| TCGA-AQ-A04L-01B-21D-A10M-09 | BRCA | e8d7feb0-981b-4ba0-b4d4-fa985064444b |
| TCGA-AQ-A0Y5-01A-11D-A14K-09 | BRCA | 4aa80fbd-a337-49b6-9371-223cbcfbc85d |
| TCGA-AQ-A1H2-01A-11D-A13L-09 | BRCA | 1ab2dc63-51ce-4a96-b7ad-f0d9eb198d10 |
| TCGA-AQ-A1H3-01A-31D-A13L-09 | BRCA | 1fa2017e-ce08-4a16-bdf6-f9bf1296c834 |
| TCGA-AR-A0TP-01A-11D-A099-09 | BRCA | bee5b9c8-739e-4530-b140-cd2b898d7afd |
| TCGA-AR-A0TQ-01A-11D-A099-09 | BRCA | b266fffc-263d-4b0f-a781-7437e41061b2 |
| TCGA-AR-A0TR-01A-11D-A099-09 | BRCA | 58ca11bf-17b0-4cff-b210-5b85d8e66ef5 |
| TCGA-AR-A0TS-01A-11D-A10Y-09 | BRCA | c9253ecc-cfac-4cc5-8dab-1e502d34d103 |
| TCGA-AR-A0TT-01A-31D-A099-09 | BRCA | 29cfdc11-2f20-436e-8913-340909684c06 |
| TCGA-AR-A0TU-01A-31D-A10G-09 | BRCA | 31922dbe-3b4a-4ac1-98fc-db88ae851462 |
| TCGA-AR-A0TV-01A-21D-A099-09 | BRCA | 0ec80200-12fe-479c-8ea0-982a9995f55a |
| TCGA-AR-A0TW-01A-11D-A099-09 | BRCA | b40d49ed-bc30-4656-9f36-ffc280de2fb8 |
| TCGA-AR-A0TX-01A-11D-A099-09 | BRCA | 63d635fa-d136-4e8a-a534-966ee678bb66 |
| TCGA-AR-A0TY-01A-12W-A12T-09 | BRCA | f915733b-aaf4-406d-af52-00de113e8e0c |
| TCGA-AR-A0TZ-01A-12D-A099-09 | BRCA | 90a26d5e-356b-424c-80bc-4723d24c594f |
| TCGA-AR-A0U0-01A-11D-A10G-09 | BRCA | 79e2c073-7727-4c34-ac28-5d7895144743 |
| TCGA-AR-A0U1-01A-11D-A10Y-09 | BRCA | 265ceec6-e9a8-499e-adf6-0c18c598532e |
| TCGA-AR-A0U2-01A-11D-A10G-09 | BRCA | f0194733-2347-43c4-a4a3-131642c27798 |
| TCGA-AR-A0U3-01A-11D-A10G-09 | BRCA | c8251555-77d3-4a20-9cc0-f7df0fda5955 |
| TCGA-AR-A0U4-01A-11D-A117-09 | BRCA | ed064e31-8fae-4f9c-8455-d7517f94e16b |
| TCGA-AR-A1AH-01A-11D-A12B-09 | BRCA | ff4a0f5a-9f30-4a2b-9915-62f2df5ad155 |
| TCGA-AR-A1AI-01A-11D-A12Q-09 | BRCA | 842846ea-881c-4d79-88d2-fc1703c58350 |
| TCGA-AR-A1AJ-01A-21D-A12Q-09 | BRCA | 4e1f9084-4729-4b3f-b036-6226d64fd25b |
| TCGA-AR-A1AK-01A-21D-A12Q-09 | BRCA | 52f7c22f-84cb-4263-93bf-1ae8cf8abbd2 |
| TCGA-AR-A1AL-01A-21D-A12Q-09 | BRCA | 8495c66e-dc95-4eae-909b-b51b8bc84889 |
| TCGA-AR-A1AN-01A-11D-A12Q-09 | BRCA | 9c879ced-92e8-4292-9b24-46005acab0f4 |
| TCGA-AR-A1AO-01A-11D-A12Q-09 | BRCA | b841db95-2eff-4181-8d44-3cde2f2f9e70 |
| TCGA-AR-A1AP-01A-11D-A12Q-09 | BRCA | 597e37c9-f0c9-4839-800e-6e9519ec3add |
| TCGA-AR-A1AQ-01A-11D-A12Q-09 | BRCA | 88ff7728-ecc9-4ec5-817e-4793619ab5a4 |
| TCGA-AR-A1AR-01A-31D-A135-09 | BRCA | 008ba655-a0a3-42c4-8c72-f1341365ef02 |
| TCGA-AR-A1AS-01A-11D-A12Q-09 | BRCA | 3f26f93c-e11a-4ec9-b73b-98fcadc209f4 |
| TCGA-AR-A1AT-01A-11D-A12Q-09 | BRCA | 7e00d4fa-b951-44d8-8fbf-fc7b9f19772e |
| TCGA-AR-A1AU-01A-11D-A12Q-09 | BRCA | d7cfeb04-ce20-4aab-8e5b-8a1483bcaaa5 |
| TCGA-AR-A1AV-01A-21D-A12Q-09 | BRCA | 0a0dd89c-5ec8-4015-9616-733e41361a64 |
| TCGA-AR-A1AW-01A-21D-A12Q-09 | BRCA | 33c6b6b5-1484-4002-8f84-ba67525a8777 |
| TCGA-AR-A1AX-01A-11D-A12Q-09 | BRCA | 71a3cf72-3539-4ade-97d1-6a1bd1ee4205 |
| TCGA-AR-A1AY-01A-21D-A12Q-09 | BRCA | 15f90ef0-831b-40a3-98bd-ec226a9e8b26 |
| TCGA-AR-A24H-01A-11D-A167-09 | BRCA | 6bb61dce-289d-4e39-8298-df5abe8049a2 |
| TCGA-AR-A24K-01A-11D-A167-09 | BRCA | df692383-1d6d-4caa-b44c-7a133ec4b7ee |
| TCGA-AR-A24L-01A-11D-A167-09 | BRCA | 2a93298a-d272-487c-ae4a-ec385844536e |
| TCGA-AR-A24M-01A-11D-A167-09 | BRCA | 722a8960-3a69-4f66-b972-74e6de94a1e8 |
| TCGA-AR-A24N-01A-11D-A167-09 | BRCA | b85b311c-1b29-44e3-8585-6995f9259221 |
| TCGA-AR-A24O-01A-11D-A167-09 | BRCA | 2c9fc77f-951b-4764-911a-f0cff3174fb1 |
| TCGA-AR-A24P-01A-11D-A167-09 | BRCA | dbdcf82a-3d37-4cfb-a70b-9b69ada0e732 |
| TCGA-AR-A24Q-01A-12D-A167-09 | BRCA | a9d691f2-ad2a-4a3b-ae30-ed4af96d75f2 |
| TCGA-AR-A24R-01A-11D-A167-09 | BRCA | baf43433-0001-4495-a37f-9132eb213157 |
| TCGA-AR-A24S-01A-11D-A167-09 | BRCA | aad32a56-5b98-433e-bb6e-48e09a027db6 |
| TCGA-AR-A24T-01A-11D-A167-09 | BRCA | 09991de6-2e8e-476f-987b-98d9a85dac7d |
| TCGA-AR-A24U-01A-11D-A167-09 | BRCA | 567cdc6c-df03-4642-8cbc-a269769ce1a1 |
| TCGA-AR-A24V-01A-21D-A167-09 | BRCA | bb77af66-bb8f-4590-9be8-5f729373c555 |
| TCGA-AR-A24W-01A-11D-A17G-09 | BRCA | 454e7cd4-8424-4cad-8fbb-f69affa5d1bf |
| TCGA-AR-A24X-01A-11D-A167-09 | BRCA | 53d55f5a-df86-44d7-a3a2-2dccc2557b7b |
| TCGA-AR-A24Z-01A-11D-A167-09 | BRCA | c11f2060-d3fb-4e3d-8058-b8cce44af519 |
| TCGA-AR-A250-01A-31D-A167-09 | BRCA | f7d9a372-fcd1-4462-9e0b-7eb46ddb68fd |
| TCGA-AR-A251-01A-12D-A167-09 | BRCA | 68b4de6d-352d-44e8-911a-f4541f28fc78 |
| TCGA-AR-A252-01A-11D-A167-09 | BRCA | e800d9b3-32a1-48eb-840b-9a3bec9d1f6e |
| TCGA-AR-A254-01A-21D-A167-09 | BRCA | fe2bdac0-832e-4268-bd8f-5dcfffda1979 |
| TCGA-AR-A255-01A-11D-A167-09 | BRCA | 505f1398-0bd8-4f1c-a142-651605158bf3 |
| TCGA-AR-A256-01A-11D-A167-09 | BRCA | ea43434b-197e-48ac-ae2e-46bc7f3776de |
| TCGA-B6-A0I2-01A-11W-A050-09 | BRCA | a9cae7c8-a62b-46ad-a98b-82e6b5fddf00 |
| TCGA-B6-A0I5-01A-11W-A100-09 | BRCA | f1139266-fade-4d27-ac67-60870e666295 |
| TCGA-B6-A0I6-01A-11D-A128-09 | BRCA | a876398c-5b1d-444f-a360-5fe2db697480 |
| TCGA-B6-A0I8-01A-11W-A050-09 | BRCA | ba80b13a-e20a-441b-b845-b617cc861ce7 |
| TCGA-B6-A0I9-01A-11W-A050-09 | BRCA | d2291482-9bbb-4f8f-a65b-c0737cf3acea |
| TCGA-B6-A0IA-01A-11W-A050-09 | BRCA | f7e5ada6-8f53-4765-a874-5ee9d258ad6a |
| TCGA-B6-A0IB-01A-11W-A050-09 | BRCA | ff80d5cd-7aed-499f-a472-153cc40f65de |
| TCGA-B6-A0IC-01A-11W-A050-09 | BRCA | f23fd730-0a18-4e3b-a2ed-f1a4231c2b53 |
| TCGA-B6-A0IE-01A-11W-A050-09 | BRCA | 4cb39f50-5031-4b08-baa3-1a366ada6514 |
| TCGA-B6-A0IG-01A-11W-A050-09 | BRCA | e8046519-d928-4fd3-b3e2-84585aa4f022 |
| TCGA-B6-A0IH-01A-11D-A10Y-09 | BRCA | 4a4488b9-74d9-4eb1-a7ef-c894c32db942 |
| TCGA-B6-A0IJ-01A-11W-A050-09 | BRCA | c63f9ddb-6301-400e-a0e8-197eea2efe75 |
| TCGA-B6-A0IK-01A-12W-A071-09 | BRCA | c5b1f426-562e-44e4-bcce-ce2ff6d969c8 |
| TCGA-B6-A0IM-01A-11W-A050-09 | BRCA | e99a4753-10db-4823-953d-e878a90e6b01 |
| TCGA-B6-A0IN-01A-11W-A050-09 | BRCA | ee2c9198-cea3-4a54-b96b-834a70c30d2f |
| TCGA-B6-A0IO-01A-11W-A050-09 | BRCA | 648cee86-f2e7-45a0-abf2-0ab0037e2eee |
| TCGA-B6-A0IP-01A-11D-A045-09 | BRCA | 94250f1c-d514-4dd2-b488-a93fbf111784 |
| TCGA-B6-A0IQ-01A-11W-A050-09 | BRCA | 583964cf-84ad-4ef1-90d1-2f6bfbeb245a |
| TCGA-B6-A0RE-01A-11W-A071-09 | BRCA | db2bd5cf-f0a7-4874-89eb-15029447dae1 |
| TCGA-B6-A0RG-01A-11W-A071-09 | BRCA | 9431c642-610e-4325-97b8-8b4c5c81cacd |
| TCGA-B6-A0RH-01A-21D-A10Y-09 | BRCA | 6e59b987-b4f0-4078-af2d-482c299103b6 |
| TCGA-B6-A0RI-01A-11W-A071-09 | BRCA | 50d83050-b98c-4a1a-a673-91dbc67c37c6 |
| TCGA-B6-A0RL-01A-11D-A099-09 | BRCA | 0d28966d-e03b-4b2a-ba07-b8f195efc29b |
| TCGA-B6-A0RM-01A-11D-A099-09 | BRCA | 3e03385e-f0fa-4e11-8bed-c6316802e1a9 |
| TCGA-B6-A0RN-01A-12D-A099-09 | BRCA | bbbcb493-2937-4a7b-8454-0abbbb379927 |
| TCGA-B6-A0RO-01A-22D-A099-09 | BRCA | 05e12ff8-023b-4ac1-b35d-f97b42e3da7a |
| TCGA-B6-A0RP-01A-21D-A099-09 | BRCA | efbdb449-b885-44bb-9054-9e97d6603cad |
| TCGA-B6-A0RQ-01A-11D-A10Y-09 | BRCA | f425edf3-0d08-49bf-94f6-f03343873a6c |
| TCGA-B6-A0RS-01A-11D-A099-09 | BRCA | 6b3ff733-402d-4390-8f57-57a9ad9b9969 |
| TCGA-B6-A0RT-01A-21D-A099-09 | BRCA | e1a297ed-1951-4d97-978c-56b452111ba5 |
| TCGA-B6-A0RU-01A-11D-A099-09 | BRCA | 251371ac-ef46-4e11-b45e-a2aaa986a2d2 |
| TCGA-B6-A0RV-01A-11D-A099-09 | BRCA | 39b0b605-29ae-4e2c-81dc-319446c807dd |
| TCGA-B6-A0WS-01A-11D-A10Y-09 | BRCA | 271d1985-1b15-4828-8261-4415ab048de9 |
| TCGA-B6-A0WT-01A-11D-A10G-09 | BRCA | 5fb780fb-12bc-4195-8f0c-2c6e3cc36b49 |
| TCGA-B6-A0WV-01A-11D-A10G-09 | BRCA | b92107c5-c46f-4606-b4e9-2dab55ca4e9c |
| TCGA-B6-A0WW-01A-11D-A10G-09 | BRCA | e9d6f59d-7d87-4fda-ab6f-e9c2501b8600 |
| TCGA-B6-A0WX-01A-11D-A10G-09 | BRCA | 47b5d831-5287-4f62-b17a-6e5eff2e4184 |
| TCGA-B6-A0WY-01A-11D-A10G-09 | BRCA | c973a902-abdf-41a3-8250-57011dfef1f4 |
| TCGA-B6-A0WZ-01A-11D-A10G-09 | BRCA | f6b8b1a9-370c-4023-b8bd-934e2a3d913a |
| TCGA-B6-A0X0-01A-21D-A10Y-09 | BRCA | 264fb6ef-65be-48fd-8216-6c493b620ad8 |
| TCGA-B6-A0X1-01A-11D-A10G-09 | BRCA | a492abf9-0cd3-402c-89e2-c49d650ef540 |
| TCGA-B6-A0X4-01A-11D-A10G-09 | BRCA | edbe95af-e727-4d0f-a2a4-a3c9f2afa901 |
| TCGA-B6-A0X5-01A-21D-A10G-09 | BRCA | da42f10b-d515-4678-a038-ed9c92a8b56b |
| TCGA-B6-A0X7-01A-11D-A10M-09 | BRCA | be5f93af-844a-4adb-ad89-05bfeefa58cd |
| TCGA-B6-A1KC-01B-11D-A159-09 | BRCA | fc3e822f-150d-47a7-a346-10919b42aa8c |
| TCGA-B6-A1KF-01A-11D-A13L-09 | BRCA | fbfbcb76-0524-4772-b918-1e8599a09d7f |
| TCGA-B6-A1KI-01A-11D-A14K-09 | BRCA | d9374702-8fc6-48c0-bec5-5c1105e641dc |
| TCGA-B6-A1KN-01A-11D-A13L-09 | BRCA | c1ad09c8-4237-48f0-b04c-7ee8ccaf8cf1 |
| TCGA-BH-A0AU-01A-11D-A12Q-09 | BRCA | d06209b8-8aba-44d8-b94a-990861c2324a |
| TCGA-BH-A0AV-01A-31D-A10Y-09 | BRCA | 9032b7fe-e38a-4641-a45e-67041668adc4 |
| TCGA-BH-A0AW-01A-11W-A071-09 | BRCA | 82057159-dd32-49fd-9ee7-82b4668f39c3 |
| TCGA-BH-A0AZ-01A-21D-A12Q-09 | BRCA | e6d90bb8-ad96-4cb8-a96f-a8202fcbc58f |
| TCGA-BH-A0B0-01A-21D-A10Y-09 | BRCA | 4680fd93-33c8-4aee-942b-5c616acd02cf |
| TCGA-BH-A0B1-01A-12W-A071-09 | BRCA | de20290a-1560-41fd-896b-a3ae1103423e |
| TCGA-BH-A0B4-01A-11W-A019-09 | BRCA | 83bee702-eb97-4216-a47e-d4e4eece279a |
| TCGA-BH-A0B5-01A-11D-A12Q-09 | BRCA | dfa0f8ea-ae94-4673-9751-f6cdad26022a |
| TCGA-BH-A0B9-01A-11W-A071-09 | BRCA | c57595bb-7953-4611-b0d1-3c2c40feb3b9 |
| TCGA-BH-A0BD-01A-11W-A050-09 | BRCA | eba2178f-6235-49c1-a49e-98de8ffdc6a0 |
| TCGA-BH-A0BF-01A-21D-A12Q-09 | BRCA | 39221056-704b-4a23-968d-3178dd9e790d |
| TCGA-BH-A0BG-01A-11D-A10Y-09 | BRCA | 923ee16a-2c42-46ee-b2cb-82075f2dd603 |
| TCGA-BH-A0BP-01A-11D-A10Y-09 | BRCA | 51405cf1-e844-4316-be17-85e8ad1de4a3 |
| TCGA-BH-A0BR-01A-21W-A12T-09 | BRCA | df82226e-2242-418b-9f5f-0a5e531826a4 |
| TCGA-BH-A0BS-01A-11D-A12Q-09 | BRCA | 81e4b7a4-8d94-4d31-9c08-325ee04f5f36 |
| TCGA-BH-A0BT-01A-11D-A12Q-09 | BRCA | 2299036e-7099-4b53-9143-5935442c3310 |
| TCGA-BH-A0BZ-01A-31D-A12Q-09 | BRCA | 1f07765a-3f2b-4b6f-88ef-0d7aab17a758 |
| TCGA-BH-A0C1-01B-11D-A12B-09 | BRCA | adebc709-8059-43c3-ad0e-a102fa1536ff |
| TCGA-BH-A0C3-01A-21D-A12Q-09 | BRCA | ec57ee0f-949e-4eee-91c2-dd129d657065 |
| TCGA-BH-A0C7-01B-11D-A10Y-09 | BRCA | ba3b30c5-8179-49bd-aacd-53326bf356f8 |
| TCGA-BH-A0DD-01A-31D-A12Q-09 | BRCA | 1a59cd97-2ee8-4f82-b542-e2f35171bc01 |
| TCGA-BH-A0DG-01A-21D-A12Q-09 | BRCA | ec4d4cbc-d5d1-418d-a292-cad9576624fd |
| TCGA-BH-A0DI-01A-21D-A12Q-09 | BRCA | 3777748c-5614-4826-8cde-eb7ecefb8101 |
| TCGA-BH-A0DO-01B-11D-A12B-09 | BRCA | 14649437-79a6-40bd-87b1-a278bfb2dcda |
| TCGA-BH-A0DS-01A-11W-A071-09 | BRCA | 6cfb5de9-ef59-4bc0-9ec2-f9bd5a9f2aee |
| TCGA-BH-A0DT-01A-21D-A12B-09 | BRCA | 30dbe353-86d5-40ed-84c2-dbddf7beb17b |
| TCGA-BH-A0DV-01A-21D-A12Q-09 | BRCA | 24ee6b1d-3594-4d12-91b3-8ad1b3c98f28 |
| TCGA-BH-A0DX-01A-11D-A10Y-09 | BRCA | bca403d9-48ff-4534-ba33-94b8fb9fee0f |
| TCGA-BH-A0E2-01A-11W-A071-09 | BRCA | 2703ce22-3ffa-4094-b3f1-1f573b5204a9 |
| TCGA-BH-A0E6-01A-11W-A050-09 | BRCA | 1c55939a-ae58-4ed9-8a6e-01bae8ac12f7 |
| TCGA-BH-A0E7-01A-11W-A050-09 | BRCA | 1ddc3a98-e0b9-4b8e-b3d3-9d39eb7d8264 |
| TCGA-BH-A0E9-01B-11D-A10Y-09 | BRCA | 48ccd30d-0c71-4117-8ccb-013986f14e95 |
| TCGA-BH-A0EA-01A-11D-A10Y-09 | BRCA | 561b8777-801a-49ed-a306-e7dafeb044b6 |
| TCGA-BH-A0EB-01A-11W-A050-09 | BRCA | 3861ca01-bcc3-42a9-835d-1ef9f1a053bd |
| TCGA-BH-A0EE-01A-11W-A050-09 | BRCA | 68d16e6a-20a5-428f-89d0-a8a0deda80cc |
| TCGA-BH-A0EI-01A-11D-A10Y-09 | BRCA | ee8e93e0-d08c-400e-8ed7-ae56d7aefbec |
| TCGA-BH-A0GY-01A-11W-A071-09 | BRCA | db589949-1630-45b2-b09b-0312d3efd60b |
| TCGA-BH-A0GZ-01A-11W-A071-09 | BRCA | 068bd892-6fee-46c2-945f-34a6c6804070 |
| TCGA-BH-A0H0-01A-11W-A071-09 | BRCA | 69110467-4cf5-4b5d-a2dd-b1c91e786959 |
| TCGA-BH-A0H3-01A-11D-A12Q-09 | BRCA | 12d7dc75-2e4f-42f6-a067-fe6d7118a0b6 |
| TCGA-BH-A0H6-01A-21W-A071-09 | BRCA | bbed00d2-9791-464d-a1ba-28fd56a0504e |
| TCGA-BH-A0HA-01A-11D-A12Q-09 | BRCA | 95f2ee35-a485-4995-8205-01623d97da2d |
| TCGA-BH-A0HB-01A-11W-A071-09 | BRCA | ed5f1077-62c1-43d8-8a27-56521bbdd8a5 |
| TCGA-BH-A0HI-01A-11D-A099-09 | BRCA | 507213d0-ef1c-400c-8724-24cd6a39feb8 |
| TCGA-BH-A0HL-01A-11W-A050-09 | BRCA | 1fd1db26-79e0-4018-8548-8fd20a96c479 |
| TCGA-BH-A0HN-01A-11D-A099-09 | BRCA | ada199c5-8015-481f-a46e-46fa42646cd8 |
| TCGA-BH-A0HO-01A-11W-A050-09 | BRCA | 354172e7-3e54-4ec4-88fa-fd7781cc86ae |
| TCGA-BH-A0HP-01A-12D-A099-09 | BRCA | ad52a8fb-7a76-4aa0-95fb-d6edab0fe2b2 |
| TCGA-BH-A0HQ-01A-11W-A050-09 | BRCA | f03af67f-3119-4ee4-a4b0-227d36f493ba |
| TCGA-BH-A0HU-01A-11W-A050-09 | BRCA | b46f2619-5937-4847-bb38-fe6022225ab9 |
| TCGA-BH-A0HW-01A-11W-A050-09 | BRCA | 706ec3be-bd65-4f42-b5cc-603f7f62c91a |
| TCGA-BH-A0HX-01A-21W-A071-09 | BRCA | 27df78cd-1f39-42f3-92e6-56664d4c472c |
| TCGA-BH-A0HY-01A-11W-A071-09 | BRCA | a63c2000-9e41-4897-8b01-4723c382096e |
| TCGA-BH-A0RX-01A-21D-A099-09 | BRCA | 48115e9a-5027-455a-a88e-c3d991dbf966 |
| TCGA-BH-A0W3-01A-11D-A10G-09 | BRCA | 3fa14183-e0c5-4dc2-bb4a-d8dd42f6578b |
| TCGA-BH-A0W4-01A-11D-A10G-09 | BRCA | fdafddde-aff1-42b4-bf94-a95861eacf53 |
| TCGA-BH-A0W5-01A-11D-A10G-09 | BRCA | aca1d737-c24c-49fd-86c0-ab2b29cd28de |
| TCGA-BH-A0W7-01A-11D-A10Y-09 | BRCA | 7d20774c-6aac-4eb0-a876-1be14e0f3004 |
| TCGA-BH-A0WA-01A-11D-A10G-09 | BRCA | 4076f947-a1f0-4101-9a79-79828eb3bbe3 |
| TCGA-BH-A18F-01A-11D-A12B-09 | BRCA | d414b3fe-b768-4a98-b285-5284bffa66f9 |
| TCGA-BH-A18H-01A-11D-A12B-09 | BRCA | d3c1b990-aae2-45f8-be28-8ccd192a0fab |
| TCGA-BH-A18I-01A-11D-A12B-09 | BRCA | f0ca4831-d56d-4bae-b304-bb43c5d2f09b |
| TCGA-BH-A18J-01A-11D-A12B-09 | BRCA | fd9923db-2a27-432e-a0c6-4c44e6ee1f53 |
| TCGA-BH-A18K-01A-11D-A12B-09 | BRCA | f75de986-bc8a-4ffe-9b35-011eee3a1446 |
| TCGA-BH-A18L-01A-32D-A12B-09 | BRCA | 883cd3c9-2681-4822-8b22-29149a027514 |
| TCGA-BH-A18M-01A-11D-A12B-09 | BRCA | 0e548c1e-cbb7-4432-8112-bb262a1ef9d9 |
| TCGA-BH-A18N-01A-11D-A12B-09 | BRCA | 13c38ac4-c410-4602-83e3-9b80b4f93839 |
| TCGA-BH-A18P-01A-11D-A12B-09 | BRCA | add624a3-57e9-46be-9bcc-3e53d7c2dfb7 |
| TCGA-BH-A18Q-01A-12D-A12B-09 | BRCA | a4de6680-33c3-4f6f-8696-453470a00bcb |
| TCGA-BH-A18R-01A-11D-A12B-09 | BRCA | 42facac2-81d9-4a9f-b4f6-1de89a7662fc |
| TCGA-BH-A18S-01A-11D-A12B-09 | BRCA | a01c12fc-a33e-4a06-8b69-ebe6d4f59c2b |
| TCGA-BH-A18T-01A-11D-A12B-09 | BRCA | 4e0ddfcb-e847-4132-bdce-aaee2e027b28 |
| TCGA-BH-A18U-01A-21D-A12B-09 | BRCA | a8400863-c145-4c6c-bcf3-e4cc4d816d22 |
| TCGA-BH-A18V-01A-11D-A12B-09 | BRCA | 6150dd25-a8f4-4d9f-9da0-f956855ab67d |
| TCGA-BH-A1EN-01A-11D-A17G-09 | BRCA | ca100ef0-be45-415f-909d-7172261d0084 |
| TCGA-BH-A1EO-01A-11D-A135-09 | BRCA | 20131381-8a11-425d-8954-980e6ec7c427 |
| TCGA-BH-A1ES-01A-11D-A135-09 | BRCA | 7ecda44b-e942-4077-9d18-2a844ec53c9d |
| TCGA-BH-A1ET-01A-11D-A135-09 | BRCA | 9bd66613-68ad-42c1-ab43-dac1386027f9 |
| TCGA-BH-A1EU-01A-11D-A135-09 | BRCA | dc578e75-e63c-4bdf-abfa-e2d063c9cd6d |
| TCGA-BH-A1EV-01A-11D-A135-09 | BRCA | 43fbe2a9-078a-4be2-b67c-b855329091f0 |
| TCGA-BH-A1EW-01A-11D-A135-09 | BRCA | c6f4b1b6-a8dd-4a9a-a500-b14a738fe18f |
| TCGA-BH-A1EX-01A-11D-A13L-09 | BRCA | 537b1685-0882-48ee-a38a-a05b5d1c8ba1 |
| TCGA-BH-A1EY-01A-11D-A13L-09 | BRCA | 7c035023-8ea9-4504-8f03-9573745cb6ef |
| TCGA-BH-A1F0-01A-11D-A135-09 | BRCA | 3903b485-366d-4318-b17d-a0194f032bd8 |
| TCGA-BH-A1F2-01A-31D-A13L-09 | BRCA | a5c67494-d843-4b14-ba9c-d077396ed2dc |
| TCGA-BH-A1F5-01A-12D-A13L-09 | BRCA | 82121518-98d6-4db6-8be4-74bbe232a9ed |
| TCGA-BH-A1F6-01A-11D-A13L-09 | BRCA | 34eb095d-3d44-4c59-9ef5-94592ba97900 |
| TCGA-BH-A1F8-01A-11D-A13L-09 | BRCA | 030cfc8a-7b43-4d73-8bfa-b68a47749e49 |
| TCGA-BH-A1FC-01A-11D-A13L-09 | BRCA | 84c77098-03d0-4b22-afb1-797703e85c6c |
| TCGA-BH-A1FD-01A-11W-A14Q-09 | BRCA | b372b5cd-4c38-4cd3-95e0-8708ce5437e7 |
| TCGA-BH-A1FE-01A-11D-A13L-09 | BRCA | 5e71fc3a-a2f4-4899-9c1f-8fee1ef29e2e |
| TCGA-BH-A1FG-01A-11D-A13L-09 | BRCA | 311f2f1a-75c8-4fee-b31d-0815d71a3173 |
| TCGA-BH-A1FH-01A-12D-A13L-09 | BRCA | fd6bd486-6371-4892-863e-64838fcea624 |
| TCGA-BH-A1FJ-01A-11D-A13L-09 | BRCA | dc62eafd-b5ad-42b4-9665-11ba6b22cff5 |
| TCGA-BH-A1FL-01A-11D-A13L-09 | BRCA | bb84cbb1-7244-4d92-8977-a37dbafc47b4 |
| TCGA-BH-A1FM-01A-11D-A13L-09 | BRCA | 7cb17736-03da-4f77-8397-145585a25b1e |
| TCGA-BH-A1FN-01A-11D-A13L-09 | BRCA | bf92d76e-31ff-4273-82ea-982c4c26394b |
| TCGA-BH-A1FR-01A-11D-A13L-09 | BRCA | a589f5ac-105c-45d6-96e1-55e3080f999c |
| TCGA-BH-A1FU-01A-11D-A14G-09 | BRCA | 9efd4bfb-d4e4-487e-8d1c-a19c2d62e3cf |
| TCGA-BH-A201-01A-11D-A14K-09 | BRCA | df6e619f-67a5-49f3-9768-4826aa2c9d1b |
| TCGA-BH-A202-01A-11D-A14K-09 | BRCA | e6feb69a-8827-4d43-94aa-036cf5150549 |
| TCGA-BH-A203-01A-12D-A167-09 | BRCA | 128b9209-2201-428c-87e7-65690bfe3875 |
| TCGA-BH-A204-01A-11D-A159-09 | BRCA | 2454d30f-1ca5-4f01-bfce-6ae10e84e75a |
| TCGA-BH-A208-01A-11D-A159-09 | BRCA | ae749fbb-6de7-4c51-b9d6-80a2ce7b5a29 |
| TCGA-BH-A209-01A-11D-A17G-09 | BRCA | 4eaf8116-4733-4865-8e22-5d03887bbc9b |
| TCGA-BH-A28Q-01A-11D-A16D-09 | BRCA | 0698379c-8f4e-460d-b7da-d3f6179dafd7 |
| TCGA-C8-A12K-01A-21D-A10Y-09 | BRCA | bcf92c27-3aa7-4449-9c7a-fc715789788f |
| TCGA-C8-A12L-01A-11D-A10Y-09 | BRCA | 998a465a-d084-4d7f-8c02-8c5be1e1ee27 |
| TCGA-C8-A12M-01A-11D-A135-09 | BRCA | 9a0a7b93-da6e-45b7-9a6f-190d79552b49 |
| TCGA-C8-A12N-01A-11D-A10Y-09 | BRCA | e2af7f0c-3cf4-4ffe-b764-b4fd83bf7694 |
| TCGA-C8-A12O-01A-11D-A10Y-09 | BRCA | 51dbda2a-106b-4597-aa49-609b677866c8 |
| TCGA-C8-A12P-01A-11D-A10Y-09 | BRCA | 540fe594-0186-40d3-b519-c1ccebe82247 |
| TCGA-C8-A12Q-01A-11D-A10Y-09 | BRCA | b6b4af38-7ebb-4fa8-9876-6d88d2b1e7e4 |
| TCGA-C8-A12T-01A-11D-A10Y-09 | BRCA | 961fae8a-d944-4866-b198-ea6f1e59a979 |
| TCGA-C8-A12U-01A-11D-A10Y-09 | BRCA | 444a1ef9-819a-41dc-baef-22057225efcd |
| TCGA-C8-A12V-01A-11D-A10Y-09 | BRCA | b8728982-8254-4aa8-baa5-aaeb6d852260 |
| TCGA-C8-A12W-01A-11D-A10Y-09 | BRCA | 5fb924d9-3201-491b-90b1-fe8a6320b2d7 |
| TCGA-C8-A12X-01A-11D-A10Y-09 | BRCA | f133a2e3-73a2-40b8-855f-e819e4d11630 |
| TCGA-C8-A12Y-01A-11D-A12B-09 | BRCA | d5c0a1a0-3d38-497b-9f47-107f06659cb1 |
| TCGA-C8-A12Z-01A-11D-A10Y-09 | BRCA | ae68cac5-e561-4094-98fa-2303cdaa6dbb |
| TCGA-C8-A130-01A-31D-A10Y-09 | BRCA | da70101d-10c2-47ab-bce1-7757dcbb08a2 |
| TCGA-C8-A131-01A-11D-A10Y-09 | BRCA | df8c72f3-ca4f-4a15-8d58-976d9c796570 |
| TCGA-C8-A132-01A-31D-A10Y-09 | BRCA | c038ab30-af2f-4771-bf82-dcf19f32efab |
| TCGA-C8-A133-01A-32D-A12B-09 | BRCA | 641e848d-e3e2-46a7-ad42-5e5672639816 |
| TCGA-C8-A134-01A-11D-A10Y-09 | BRCA | a3e8738b-2456-4f08-bb3d-5debb4265f85 |
| TCGA-C8-A135-01A-11D-A10Y-09 | BRCA | 6b47c22f-8b4e-40fd-9a12-18b539521224 |
| TCGA-C8-A137-01A-11D-A10Y-09 | BRCA | 08778f40-d895-46f1-8e7b-122fc598418b |
| TCGA-C8-A138-01A-11D-A10Y-09 | BRCA | f3474e56-8457-4f0b-8a2f-58fdd8f58607 |
| TCGA-C8-A1HE-01A-11D-A188-09 | BRCA | 8314bada-5bd3-4cd2-b308-4cb2db64de94 |
| TCGA-C8-A1HF-01A-11D-A135-09 | BRCA | 508a26f2-d117-44aa-b579-00a119b8bcc4 |
| TCGA-C8-A1HG-01A-11D-A135-09 | BRCA | ba937e3d-30b7-4446-84fb-5f77831a4843 |
| TCGA-C8-A1HI-01A-11D-A135-09 | BRCA | 75dc3bff-75da-4734-b930-a18fd3d1ebfe |
| TCGA-C8-A1HJ-01A-11D-A13L-09 | BRCA | a62c3601-b90f-402f-8212-ffdfde3c6df8 |
| TCGA-C8-A1HK-01A-21D-A13L-09 | BRCA | 357e0b08-fa33-4f58-92b0-d7293b63c01d |
| TCGA-C8-A1HL-01A-11D-A135-09 | BRCA | 88c9ef88-5d85-4a4b-9c68-d9ec709a1f07 |
| TCGA-C8-A1HM-01A-12D-A135-09 | BRCA | a2f9165d-9fe7-492e-9b4c-3cb4200c6e85 |
| TCGA-C8-A1HN-01A-11D-A135-09 | BRCA | a2576147-28eb-460f-9b97-916892d801e2 |
| TCGA-C8-A1HO-01A-11D-A13L-09 | BRCA | c6fb921c-78fe-4852-b2a5-edd5a02ae923 |
| TCGA-C8-A26V-01A-11D-A16D-09 | BRCA | 6c5a83f5-983f-434c-ac29-ddb84a7f1019 |
| TCGA-C8-A26W-01A-11D-A16D-09 | BRCA | d3db354e-f22c-4576-a7d7-6515f1c11002 |
| TCGA-C8-A26X-01A-31D-A16D-09 | BRCA | a5bc549a-1a1f-41b4-b548-14c448fed6c7 |
| TCGA-C8-A26Z-01A-11D-A16D-09 | BRCA | fa4f7af6-380f-4dbd-ba6a-8c0d22f56a9c |
| TCGA-C8-A273-01A-11D-A16D-09 | BRCA | c5e6f325-5fd0-4cff-8eaf-6e23e016f605 |
| TCGA-C8-A274-01A-11D-A16D-09 | BRCA | 5e6e7c20-47b3-4f0e-a3c7-8293993e39cf |
| TCGA-C8-A275-01A-21D-A16D-09 | BRCA | 7751a837-2656-4e3b-9182-556314c4f6a3 |
| TCGA-C8-A278-01A-11D-A167-09 | BRCA | 7bc48524-1f69-4d85-9d16-6db7844543bd |
| TCGA-C8-A27A-01A-11D-A167-09 | BRCA | d0fd3dcc-4ac7-4fe9-9fb8-c0676b6faabb |
| TCGA-C8-A27B-01A-11D-A167-09 | BRCA | 11e43e41-54b8-4232-b078-5062288d3868 |
| TCGA-D8-A13Y-01A-11D-A10Y-09 | BRCA | 8bb90325-028e-491a-bbaf-2cf4b3b87cd6 |
| TCGA-D8-A13Z-01A-11D-A10Y-09 | BRCA | c3722c97-80f5-4eea-bf50-5a214134bbcc |
| TCGA-D8-A140-01A-11D-A10Y-09 | BRCA | 795f051e-01c4-4b49-b179-bd18ba24433c |
| TCGA-D8-A141-01A-11D-A10Y-09 | BRCA | 807791d8-b6c0-4722-bf5c-d5fa3Obaffc6 |
| TCGA-D8-A143-01A-11D-A10Y-09 | BRCA | db1763d1-fcae-4a01-a0cb-3019e292aa10 |
| TCGA-D8-A145-01A-11D-A10Y-09 | BRCA | af6ca646-499a-4e0a-a194-cacf72e5810b |
| TCGA-D8-A146-01A-31D-A10Y-09 | BRCA | 9a7548dc-fc79-4ad4-a324-0e9f63c91a20 |
| TCGA-D8-A147-01A-11D-A10Y-09 | BRCA | 1f292323-cafc-4e45-bb4e-f5428e1a3276 |
| TCGA-D8-A1J9-01A-11D-A13L-09 | BRCA | 6627e4b1-b34c-4aa2-836e-093061442a6d |
| TCGA-D8-A1JB-01A-11D-A13L-09 | BRCA | 54621c54-b7ef-48e4-aa68-e2fe10bf0afb |
| TCGA-D8-A1JC-01A-11D-A13L-09 | BRCA | 63a9d14f-d91a-47af-8ef6-8124193aa110 |
| TCGA-D8-A1JD-01A-11D-A13L-09 | BRCA | 7df92725-fa63-494d-af9d-65c6ed76e023 |
| TCGA-D8-A1JE-01A-11D-A13L-09 | BRCA | bb34512b-2432-4256-968c-d7fdf38f126a |
| TCGA-D8-A1JF-01A-11D-A13L-09 | BRCA | d31358da-639c-4fe5-9f7c-c17c31fd2865 |
| TCGA-D8-A1JG-01B-11D-A13L-09 | BRCA | 0b15c6f7-8e3e-48ad-a4a2-97d2ada56c44 |
| TCGA-D8-A1JH-01A-11D-A188-09 | BRCA | 9f59481d-be89-4361-8cc3-3f1d46702016 |
| TCGA-D8-A1JI-01A-11D-A13L-09 | BRCA | 2c6a885b-0452-492c-8829-13ba4b2ac455 |
| TCGA-D8-A1JJ-01A-31D-A14K-09 | BRCA | 412f96a6-6599-40a6-9dd2-afba8c643910 |
| TCGA-D8-A1JK-01A-11D-A13L-09 | BRCA | fadaa39d-ebd2-4887-ae54-1fca12287fcf |
| TCGA-D8-A1JL-01A-11D-A13L-09 | BRCA | 425dbc9f-6bee-412a-b77a-22a2724ea4c6 |
| TCGA-D8-A1JM-01A-11D-A13L-09 | BRCA | f66d4178-34f3-4f5d-aa0a-7fdd03801033 |
| TCGA-D8-A1JN-01A-11D-A13L-09 | BRCA | c83c7d48-8671-4f27-b3dd-05411fa2f784 |
| TCGA-D8-A1JP-01A-11D-A13L-09 | BRCA | 1e21a355-0cb6-4a43-b134-50ff88dacf92 |
| TCGA-D8-A1JS-01A-11D-A13L-09 | BRCA | 4a9181d0-d3df-4791-99f0-4db076c22a3a |
| TCGA-D8-A1JT-01A-31D-A13L-09 | BRCA | 3be3972f-4125-44c3-94d6-0ddba2008fcf |
| TCGA-D8-A1JU-01A-11D-A13L-09 | BRCA | 7bff4f75-749d-4a63-9a64-0bcf1cd615ea |
| TCGA-D8-A1X5-01A-11D-A14G-09 | BRCA | db4526d4-e344-4b5a-bb66-fd43b41764ca |
| TCGA-D8-A1X6-01A-11D-A14K-09 | BRCA | 1951aa38-481b-464c-9a78-0819312a0a93 |
| TCGA-D8-A1X7-01A-11D-A14K-09 | BRCA | 7acb4232-db95-4889-942e-f1be897b4f2a |
| TCGA-D8-A1X8-01A-11D-A14K-09 | BRCA | 78c3c787-5731-4c38-8d7a-e5b503b11c36 |
| TCGA-D8-A1X9-01A-12D-A159-09 | BRCA | b5f65c3a-b922-4a81-863d-59b72b08d1bf |
| TCGA-D8-A1XA-01A-11D-A14G-09 | BRCA | a362780b-8917-4438-9693-ec9fa84c352a |
| TCGA-D8-A1XB-01A-11D-A14G-09 | BRCA | e5ca0f82-6fa9-4d54-adc7-385721f351f3 |
| TCGA-D8-A1XC-01A-11D-A14G-09 | BRCA | 68fd3045-073d-4242-8a41-41b707fca625 |
| TCGA-D8-A1XF-01A-11D-A14G-09 | BRCA | e1587f32-2ff9-40f3-97dd-b45b0f14be46 |
| TCGA-D8-A1XG-01A-11D-A14G-09 | BRCA | 800ff536-a1d2-4213-b85e-7780851c6378 |
| TCGA-D8-A1XJ-01A-11D-A14K-09 | BRCA | a37b27a2-c3b0-4f62-82a2-94e9205b1d6e |
| TCGA-D8-A1XL-01A-11D-A14K-09 | BRCA | 28d44e6e-c73f-4788-8ad4-2bd6572f643d |
| TCGA-D8-A1XM-01A-21D-A14K-09 | BRCA | 07418962-0a82-43a2-a66f-614903ea8380 |
| TCGA-D8-A1XO-01A-11D-A14K-09 | BRCA | b5ff68a2-da74-4608-941e-dbac40153077 |
| TCGA-D8-A1XR-01A-11D-A14K-09 | BRCA | 5913c8ff-26ce-4f26-909e-3ed292d3c538 |
| TCGA-D8-A1XS-01A-11D-A14K-09 | BRCA | 5d302c04-302e-4040-9429-37cd672e8d53 |
| TCGA-D8-A1XT-01A-11D-A14K-09 | BRCA | bc13601e-3e03-4d7d-8e6e-5b05ff500ea3 |
| TCGA-D8-A1XU-01A-11D-A14K-09 | BRCA | 55c547ee-7cc9-4b7a-aaca-22f2a8c8c3a4 |
| TCGA-D8-A1XV-01A-11D-A14K-09 | BRCA | a76adfd1-8c89-4c13-b570-5ccc47043a70 |
| TCGA-D8-A1XW-01A-11D-A14K-09 | BRCA | f29405cc-d712-4562-ac02-ca3c89fb82af |
| TCGA-D8-A1XY-01A-11D-A14K-09 | BRCA | edb6d161-8f50-4c11-8246-487c4ea9a55d |
| TCGA-D8-A1XZ-01A-11D-A14K-09 | BRCA | 381a9211-1f2b-4c14-895b-ee7fb6eb8c7f |
| TCGA-D8-A1Y0-01A-11D-A14K-09 | BRCA | 33ff7870-fa76-4e48-a223-a8e2441d8f53 |
| TCGA-D8-A1Y1-01A-21D-A14K-09 | BRCA | 2ea6e540-6e2f-48a5-99e3-27a0107d07b7 |
| TCGA-D8-A1Y2-01A-11D-A159-09 | BRCA | 9dbf62eb-0de7-4410-b44b-fdf59026d8e6 |
| TCGA-D8-A1Y3-01A-11D-A159-09 | BRCA | 64fa29ff-534f-4b22-b0c4-513e8657edb1 |
| TCGA-D8-A27E-01A-11D-A16D-09 | BRCA | eab47cbb-eab0-4dd6-9cd0-f2700e5b6227 |
| TCGA-D8-A27F-01A-11D-A16D-09 | BRCA | fc6d77a9-121b-48ab-a899-713c3d1319a2 |
| TCGA-D8-A27H-01A-11D-A16D-09 | BRCA | 78e51220-c9f8-44b2-bc1c-b34a56af3b54 |
| TCGA-D8-A27I-01A-11D-A16D-09 | BRCA | 47c0db0a-fc37-4fa0-832c-e67f089d3889 |
| TCGA-D8-A27K-01A-11D-A16D-09 | BRCA | 09fa0bc7-acb3-45b0-b687-977869c31d12 |
| TCGA-D8-A27L-01A-11D-A16D-09 | BRCA | 10666107-dffb-4c51-b3ee-71e70cde7c88 |
| TCGA-D8-A27M-01A-11D-A16D-09 | BRCA | cb9257f9-ca3f-4c14-a680-6632175dd526 |
| TCGA-D8-A27N-01A-11D-A16D-09 | BRCA | 6a411174-582a-4c68-bb04-5ea2e504bf7c |
| TCGA-D8-A27P-01A-11D-A16D-09 | BRCA | 94011b46-74e3-41c1-a3f6-6db1821d1778 |
| TCGA-D8-A27R-01A-11D-A16D-09 | BRCA | 27741c13-8d5f-43b8-8651-caf69acef0e4 |
| TCGA-D8-A27T-01A-11D-A16D-09 | BRCA | ecabcc6a-2767-4ad8-ac4f-54cc3d081b6e |
| TCGA-D8-A27W-01A-11D-A16D-09 | BRCA | b045d675-286b-4cf8-aed4-c7ff81a78919 |
| TCGA-E2-A105-01A-11D-A10M-09 | BRCA | 2441f3e0-2016-4313-8c05-486759f5dd0f |
| TCGA-E2-A107-01A-11D-A10M-09 | BRCA | 5804fc1c-063b-429d-a652-22b0de416bd6 |
| TCGA-E2-A108-01A-13D-A10M-09 | BRCA | e3e394d4-2593-4bf9-86e4-2e79d8cb8dab |
| TCGA-E2-A109-01A-11D-A10M-09 | BRCA | 3585e133-b3c1-4d90-b5f2-2b867e0ae0ec |
| TCGA-E2-A10A-01A-21D-A10Y-09 | BRCA | cd49ccc5-a776-4307-930c-298ba6cfdf79 |
| TCGA-E2-A10B-01A-11D-A10M-09 | BRCA | 9d712002-74cb-459a-b350-e9a4b49aac13 |
| TCGA-E2-A10C-01A-21D-A10M-09 | BRCA | 2750ed41-0bd4-4cf4-98f5-762957cf80b7 |
| TCGA-E2-A10E-01A-11D-A10M-09 | BRCA | 530b7e22-e70a-46ef-a0e8-bf2ef814850a |
| TCGA-E2-A14N-01A-31D-A135-09 | BRCA | 00c8d151-2223-4e36-8c66-6c09e42d8777 |
| TCGA-E2-A14O-01A-31D-A10Y-09 | BRCA | d6ab6f8d-0e65-40a3-bf98-7249e4075395 |
| TCGA-E2-A14P-01A-31D-A12B-09 | BRCA | 35a96eee-113b-45cb-a999-81c13545b104 |
| TCGA-E2-A14Q-01A-11D-A12B-09 | BRCA | ee51cf6d-351f-48f8-ab93-639c27c50e9f |
| TCGA-E2-A14R-01A-11D-A10Y-09 | BRCA | c7212115-1007-40cf-b9b5-7b25e2f5f2a4 |
| TCGA-E2-A14S-01A-11D-A12B-09 | BRCA | 78f39325-e1d0-4181-87f4-cb7f00e886d7 |
| TCGA-E2-A14T-01A-11D-A10Y-09 | BRCA | 14c1c6b6-575e-416b-b219-15552b62ea74 |
| TCGA-E2-A14V-01A-11D-A12B-09 | BRCA | 703314fe-bfd5-45d5-9ed5-fcdce8a19fd6 |
| TCGA-E2-A14W-01A-11D-A12B-09 | BRCA | fbdc8659-e9cc-483f-bd0a-1a24b5ada1cf |
| TCGA-E2-A14X-01A-11D-A10Y-09 | BRCA | 74039acd-5aca-4c65-818c-3b577d295be0 |
| TCGA-E2-A14Z-01A-11D-A10Y-09 | BRCA | c83eaaca-ced5-4630-abb5-ef34db888753 |
| TCGA-E2-A150-01A-11D-A12B-09 | BRCA | 446064de-ff64-4113-9080-360e5bf6d5e4 |
| TCGA-E2-A152-01A-11D-A12B-09 | BRCA | b266b370-425c-4146-8b72-59248436618e |
| TCGA-E2-A154-01A-11D-A10Y-09 | BRCA | 336e39fb-d407-4ced-b7bb-e8ff5329abdb |
| TCGA-E2-A155-01A-11D-A12B-09 | BRCA | a966904f-e8dd-473c-8626-84c25d7e0d6c |
| TCGA-E2-A156-01A-11D-A12B-09 | BRCA | 26003dce-0fc6-4538-a392-c80e1ebaa1e4 |
| TCGA-E2-A159-01A-11D-A10Y-09 | BRCA | 757c8a2d-90cf-4dab-a4dd-45f3cbdeaeeb |
| TCGA-E2-A15A-01A-11D-A12B-09 | BRCA | b7e3eff1-65d5-491f-a726-35dc6752b370 |
| TCGA-E2-A15C-01A-31D-A12B-09 | BRCA | 10c594a1-0843-4740-9d96-00211a9509fb |
| TCGA-E2-A15D-01A-11D-A10Y-09 | BRCA | 891295d6-4dd0-4ab4-bbce-13da7f3c30d0 |
| TCGA-E2-A15E-01A-11D-A12B-09 | BRCA | c6f107df-1186-4d6d-b5b5-2393e9369dd1 |
| TCGA-E2-A15F-01A-11D-A10Y-09 | BRCA | 33edf937-b09f-49ec-8f4c-e05dee7ece1f |
| TCGA-E2-A15G-01A-11D-A12B-09 | BRCA | d45bb60a-e73b-4b95-8637-e8d17fcca745 |
| TCGA-E2-A15H-01A-11D-A12B-09 | BRCA | 7875c5b3-ced2-4669-a3d5-45739b850af7 |
| TCGA-E2-A15I-01A-21D-A135-09 | BRCA | 9bec02b4-7cf0-4797-b1ac-253ef78a34af |
| TCGA-E2-A15J-01A-11D-A12Q-09 | BRCA | e5fd7cbd-8fce-49e9-8d2c-d2a2e61367a5 |
| TCGA-E2-A15O-01A-11D-A10Y-09 | BRCA | 39c1df91-b670-4f6b-b5ff-dbb6b66d30af |
| TCGA-E2-A15P-01A-11D-A10Y-09 | BRCA | 5f1853c2-6579-42d0-adc2-636b5de543e4 |
| TCGA-E2-A15R-01A-11D-A10Y-09 | BRCA | 11799240-0275-48fe-84ef-85e188839bbe |
| TCGA-E2-A15S-01A-11D-A10Y-09 | BRCA | 01f78efa-ba0b-4263-81fd-d3d8ea1bc5fd |
| TCGA-E2-A15T-01A-11D-A10Y-09 | BRCA | eff74709-36af-4da4-91c1-01100ddc7735 |
| TCGA-E2-A1AZ-01A-11D-A12Q-09 | BRCA | f961c932-cf37-47c8-8520-8d0d444dc94f |
| TCGA-E2-A1B0-01A-11D-A12Q-09 | BRCA | 14e3b00c-cbec-4733-8fa4-829b8e7d9808 |
| TCGA-E2-A1B1-01A-21D-A12Q-09 | BRCA | a6e77a14-e5e5-452e-a46f-5629ee8228e3 |
| TCGA-E2-A1B4-01A-11D-A12Q-09 | BRCA | a6aa4529-7996-4b66-9632-2559293db35d |
| TCGA-E2-A1B6-01A-31D-A12Q-09 | BRCA | 1aaf88fc-f7cb-4239-a420-224352194160 |
| TCGA-E2-A1BD-01A-11D-A12Q-09 | BRCA | f5ac1986-272b-48d2-9a73-4a550e38a997 |
| TCGA-E2-A1IE-01A-11D-A188-09 | BRCA | e416f05b-c7d2-479b-8068-803492e86d86 |
| TCGA-E2-A1IF-01A-11D-A142-09 | BRCA | 7751c2d5-e548-4439-aac1-e7b9dce97583 |
| TCGA-E2-A1IG-01A-11D-A142-09 | BRCA | 84da47a3-49e1-4f94-bea9-dd20b6627adb |
| TCGA-E2-A1IH-01A-11D-A188-09 | BRCA | cd886e35-4201-4732-90c6-142d8fe309b1 |
| TCGA-E2-A1II-01A-11D-A142-09 | BRCA | 698c8a73-c6b6-45bd-82fc-9bd0f140729d |
| TCGA-E2-A1IJ-01A-11D-A142-09 | BRCA | 3aff2da1-1647-4b95-abdb-c9db923cfc22 |
| TCGA-E2-A1IK-01A-11D-A17G-09 | BRCA | 8577ac01-1274-4bd5-ab04-380eaa78d95b |
| TCGA-E2-A1IL-01A-11D-A14G-09 | BRCA | 1540ae03-7bb4-418b-afbc-44bf3ad60a31 |
| TCGA-E2-A1IN-01A-11D-A13L-09 | BRCA | 9e85559f-098e-4b0f-8034-4798789e710b |
| TCGA-E2-A1IO-01A-11D-A142-09 | BRCA | 986e9b9f-ae15-4743-a150-d6ee11f3c077 |
| TCGA-E2-A1IU-01A-11D-A14G-09 | BRCA | 7fcd5fda-8155-4b48-afb9-9e7958627113 |
| TCGA-E2-A1L6-01A-11D-A13L-09 | BRCA | f610239f-5610-4d7b-bc31-ae3ccb9c425d |
| TCGA-E2-A1L7-01A-11D-A142-09 | BRCA | 33a09072-6554-4d46-b738-0852624940af |
| TCGA-E2-A1L8-01A-11D-A13L-09 | BRCA | 04a7762f-2cbb-498b-ab4e-921406c1aec0 |
| TCGA-E2-A1L9-01A-11D-A13L-09 | BRCA | a50cd2b2-913d-41bf-94ad-45464547b348 |
| TCGA-E2-A1LA-01A-11D-A142-09 | BRCA | bdcd4800-3258-446f-b6e5-3c8e2f46c656 |
| TCGA-E2-A1LB-01A-11D-A142-09 | BRCA | 377b1816-61e1-431a-9952-71e4d58bbd48 |
| TCGA-E2-A1LG-01A-21D-A14K-09 | BRCA | 7cdbe0e8-f614-4f54-b864-fd6b39e8ef1c |
| TCGA-E2-A1LH-01A-11D-A14G-09 | BRCA | 605f1d27-db45-449a-a68f-4888b8c786a1 |
| TCGA-E2-A1LI-01A-12D-A159-09 | BRCA | c812374c-8bc9-4ccf-9157-fbd9d162ee1e |
| TCGA-E2-A1LK-01A-21D-A14G-09 | BRCA | 4e84eed6-82a8-4e91-b0fd-61ec6ef69ce9 |
| TCGA-E2-A1LL-01A-11D-A142-09 | BRCA | 47312f61-5ef4-4f25-9320-8fbb4758790e |
| TCGA-E2-A1LS-01A-12D-A159-09 | BRCA | 40087f80-85f6-4cc4-95c9-0639153dd3f4 |
| TCGA-E9-A1N3-01A-12D-A159-09 | BRCA | 6c3891a9-baa9-4309-9974-d82fd5f97417 |
| TCGA-E9-A1N4-01A-11D-A14K-09 | BRCA | a3784a48-47a7-4587-91dd-5b8873a24ca9 |
| TCGA-E9-A1N5-01A-11D-A14G-09 | BRCA | 432a9f5e-0f2a-4cd2-a910-ee9ee30c1ff3 |
| TCGA-E9-A1N8-01A-11D-A142-09 | BRCA | cac57844-0e46-489b-8d94-ceea5788c050 |
| TCGA-E9-A1N9-01A-11D-A14G-09 | BRCA | 2aa7a1db-40a5-421b-97ab-1031e6fa7f04 |
| TCGA-E9-A1NA-01A-11D-A142-09 | BRCA | a3d223eb-20e6-40b9-9f07-e5f865bd2439 |
| TCGA-E9-A1NC-01A-12W-A16L-09 | BRCA | 2ba4c398-b94b-49f8-bb88-9d0cb3347d2c |
| TCGA-E9-A1ND-01A-11D-A142-09 | BRCA | 8e72652d-3b99-47b2-87fe-04b96b243722 |
| TCGA-E9-A1NE-01A-21D-A14K-09 | BRCA | dbd34322-ac40-41f0-acc7-7bfd06afdf67 |
| TCGA-E9-A1NF-01A-11D-A14G-09 | BRCA | cd428bec-fc31-4d2d-9e6c-c8f30608d797 |
| TCGA-E9-A1NG-01A-21D-A14K-09 | BRCA | 1cbf389d-1ec8-4543-880f-4ef64c55a44b |
| TCGA-E9-A1NH-01A-11D-A14G-09 | BRCA | 13c312ec-0add-4758-ab8d-c193e2e08c6d |
| TCGA-E9-A1NI-01A-11W-A16H-09 | BRCA | 3bf0b169-f870-4887-be06-414f20f1dcf0 |
| TCGA-E9-A1QZ-01A-21D-A167-09 | BRCA | 2d47b244-e5e4-4645-91cb-71de1d685a95 |
| TCGA-E9-A1R0-01A-22D-A16D-09 | [symbol] | c09eaa03-c14c-4a96-a505-4d999e45270e |
| TCGA-E9-A1R2-01A-11D-A14G-09 | BRCA | b321a2d9-5345-4891-b450-bfd696c6cfb0 |
| TCGA-E9-A1R3-01A-31D-A14K-09 | BRCA | ba6af877-7a23-4738-a867-01a5dd8a8050 |
| TCGA-E9-A1R4-01A-21D-A14G-09 | BRCA | 15d9c916-a12e-48a0-8a0f-8c240c54bd37 |
| TCGA-E9-A1R5-01A-11D-A14K-09 | BRCA | a04ba6e9-2bc4-4cab-96d8-0820e0390d84 |
| TCGA-E9-A1R6-01A-11D-A14G-09 | BRCA | b8a1805d-a43a-4433-a90b-01715e8cc554 |
| TCGA-E9-A1R7-01A-11D-A14K-09 | BRCA | b3991854-6634-4428-bef7-a7d9ad9cca30 |
| TCGA-E9-A1RA-01A-11D-A14G-09 | BRCA | 6d067461-2002-468e-934d-2721f6cb97ff |
| TCGA-E9-A1RB-01A-11D-A17G-09 | BRCA | 2ce0333c-deca-4199-a06c-ede43c5575fc |
| TCGA-E9-A1RC-01A-11D-A159-09 | BRCA | 5b5e7eb2-8efc-4681-ab8c-49a9cc4ac6d6 |
| TCGA-E9-A1RD-01A-11D-A159-09 | BRCA | 23f7a698-eab1-40f1-926c-c95d4ed8213d |
| TCGA-E9-A1RE-01A-11D-A159-09 | BRCA | 4a9c0873-f496-48a4-853c-2b41b2dbaa9e |
| TCGA-E9-A1RF-01A-11D-A159-09 | BRCA | 43983619-d863-4816-a334-445f6ca36541 |
| TCGA-E9-A1RG-01A-11D-A14G-09 | BRCA | 81896525-0e3f-47ff-9b0d-95b45aef718c |
| TCGA-E9-A1RH-01A-21D-A167-09 | BRCA | 2ecb84c0-c307-4fa9-85e3-2f722dd365a3 |
| TCGA-E9-A1RI-01A-11D-A167-09 | BRCA | 661c0074-dac9-44c6-bebc-202cfb9fb735 |
| TCGA-E9-A226-01A-21D-A159-09 | BRCA | 866e5e9b-4e6c-49e2-9ea6-560f9bd99c2b |
| TCGA-E9-A227-01A-11D-A159-09 | BRCA | 15eb25c4-f4a7-446e-b654-ae39ccd2cf00 |
| TCGA-E9-A228-01A-31D-A159-09 | BRCA | 4a804a8d-7dc8-4b5b-9537-b7f8f7133bda |
| TCGA-E9-A229-01A-31D-A17G-09 | BRCA | a27fa57d-d1ad-4534-a933-0fdcc5f06a8c |
| TCGA-E9-A22A-01A-11D-A159-09 | BRCA | 25bf7831-6878-4bac-b23d-e94a555b2232 |
| TCGA-E9-A22B-01A-11D-A159-09 | BRCA | e46a5d19-2dd7-4c34-8fff-6276278c58b3 |
| TCGA-E9-A22D-01A-11D-A159-09 | BRCA | 3dfdc7fd-3f69-4297-a4cf-1a05b75d302f |
| TCGA-E9-A22E-01A-11D-A159-09 | BRCA | a1d7dafc-a755-44a6-b45b-dc6aae309d3e |
| TCGA-E9-A22G-01A-11D-A159-09 | BRCA | 2be1b92a-6041-4d2b-9cf8-b9723921987f |
| TCGA-E9-A22H-01A-11D-A159-09 | BRCA | 42993dbb-b99b-4b48-8038-05cf14fec886 |
| TCGA-E9-A243-01A-21D-A167-09 | BRCA | c6bb16c6-cb0f-44c6-93e7-6c55d0958f82 |
| TCGA-E9-A244-01A-11D-A167-09 | BRCA | 9edf63e8-ae94-4b2f-8521-b56dadc21cd5 |
| TCGA-E9-A245-01A-22D-A16D-09 | BRCA | bdd591f9-21d1-4ce5-bfde-30e7ac3d440a |
| TCGA-E9-A247-01A-11D-A167-09 | BRCA | 7c184a2b-d857-444a-936c-43e38a196df9 |
| TCGA-E9-A248-01A-11D-A167-09 | BRCA | fee90b4e-f005-4b40-a9af-d1e590b1e8a8 |
| TCGA-E9-A249-01A-11D-A167-09 | BRCA | 2799ad7e-d6f0-4919-b7f6-1c957b4c74f8 |
| TCGA-E9-A24A-01A-11D-A167-09 | BRCA | d11d3770-a4f4-4d15-94f4-149cca27d391 |
| TCGA-E9-A295-01A-11D-A16D-09 | BRCA | f3d5e986-046f-4f75-8abc-67a3b99f742d |
| TCGA-EW-A1IW-01A-11D-A13L-09 | BRCA | 8b8732c3-78b1-409b-bc8c-c482575361bb |
| TCGA-EW-A1IX-01A-12D-A142-09 | BRCA | 01ea194f-dc06-4e15-9b9e-1c73668040e0 |
| TCGA-EW-A1IY-01A-11D-A188-09 | BRCA | 01d3fddf-b447-4925-a5cb-c5fd70c97278 |
| TCGA-EW-A1IZ-01A-11D-A188-09 | BRCA | 18db4143-48cc-424c-8d23-46cf23056528 |
| TCGA-EW-A1J1-01A-11D-A188-09 | BRCA | 4b8d51b3-8393-45d4-a73d-3c22c561d6f3 |
| TCGA-EW-A1J2-01A-21D-A13L-09 | BRCA | c906931e-dc1a-434c-96cd-58088762f1e7 |
| TCGA-EW-A1J3-01A-11D-A13L-09 | BRCA | ac13b81a-ca05-432c-918a-0c9c8170bf46 |
| TCGA-EW-A1J5-01A-11D-A13L-09 | BRCA | 98bb3025-0637-4106-8621-12df7b5d662f |
| TCGA-EW-A1J6-01A-11D-A188-09 | BRCA | d95c5cb1-d081-47fa-8ac0-1ade7652a0af |
| TCGA-EW-A1OV-01A-11D-A142-09 | BRCA | e27ca8f5-3f76-4531-87ea-ba3a44f6830d |
| TCGA-EW-A1OX-01A-11D-A142-09 | BRCA | 7828f9cf-aa93-44a0-8070-efdf90a677f0 |
| TCGA-EW-A1OY-01A-11D-A142-09 | BRCA | 925323a2-ca03-48f4-8c37-1a8a6f8a6daa |
| TCGA-EW-A1OZ-01A-11D-A142-09 | BRCA | a73152be-2293-403d-940b-74ac05810808 |
| TCGA-EW-A1P0-01A-11D-A142-09 | BRCA | 6475f4dd-782c-411a-b7ce-9c9ebd0753b8 |
| TCGA-EW-A1P1-01A-31D-A14G-09 | BRCA | 28a56927-bab8-4a8c-be11-f46e37ea34c1 |
| TCGA-EW-A1P3-01A-11D-A142-09 | BRCA | e783933d-1c24-4cd5-82b7-0d680f9c3c22 |
| TCGA-EW-A1P4-01A-21D-A142-09 | BRCA | 204e4ef3-e6b8-469f-9024-56c6f6f07afd |
| TCGA-EW-A1P5-01A-11D-A142-09 | BRCA | 84b4da42-9673-4448-9185-a12857ab422f |
| TCGA-EW-A1P6-01A-11D-A142-09 | BRCA | eef5cea9-82f6-4001-8e2c-701e43a9787a |
| TCGA-EW-A1P7-01A-21D-A142-09 | BRCA | 402abf40-5a01-467d-a5be-b9101743f34b |
| TCGA-EW-A1P8-01A-11D-A142-09 | BRCA | e55f338f-97e2-4394-ae23-c92606069485 |
| TCGA-EW-A1PA-01A-11D-A142-09 | BRCA | 56c8aca4-b3bd-4791-b05d-0b2338b6346d |
| TCGA-EW-A1PB-01A-11D-A142-09 | BRCA | 9ddf2119-a222-4fa5-a9f3-0bec7eeea36b |
| TCGA-EW-A1PD-01A-11D-A142-09 | BRCA | 5a288561-bf14-4cb9-b2f5-9ece0e038319 |
| TCGA-EW-A1PE-01A-11D-A142-09 | BRCA | 54377bac-8f52-4116-b7e5-b71a8a721ac4 |
| TCGA-EW-A1PG-01A-11D-A142-09 | BRCA | bd3801e2-c5bb-4116-9ce3-97903fc6956e |
| TCGA-EW-A1PH-01A-11D-A14K-09 | BRCA | ce860c6f-c87a-4a45-92df-ca34bfb2e8b2 |
| TCGA-GI-A2C8-01A-11D-A16D-09 | BRCA | 535a899d-67ca-4500-8dda-63a331a3611c |
| TCGA-AA-3664-01A-01W-0900-09 | COAD | 9cff122a-9960-4f2e-ba5b-94736bad7f2b |
| TCGA-AA-3666-01A-02W-0900-09 | COAD | d7065ea5-88b0-4b56-a367-5defa0d9ed27 |
| TCGA-AA-3667-01A-01W-0900-09 | COAD | c2799cdc-c6f7-44ba-a72c-e1632b434575 |
| TCGA-AA-3672-01A-01W-0900-09 | COAD | 04dc0b16-834c-4351-b3b9-58fe558c634d |
| TCGA-AA-3673-01A-01W-0900-09 | COAD | 7952f001-8901-44b4-833e-824282967118 |
| TCGA-AA-3678-01A-01W-0900-09 | COAD | 968fea30-df40-425f-87ba-935942dbd450 |
| TCGA-AA-3679-01A-02W-0900-09 | COAD | 94cfbc05-df22-4db0-9aa0-808faab01c61 |
| TCGA-AA-3680-01A-01W-0900-09 | COAD | 20dd1d44-2321-4a84-b8b9-894073c6acd3 |
| TCGA-AA-3681-01A-01W-0900-09 | COAD | e5fea94c-f2ab-4476-b641-f2764eb0d026 |
| TCGA-AA-3684-01A-02W-0900-09 | COAD | 6ecc0812-6ce3-4569-9868-6c4936236682 |
| TCGA-AA-3685-01A-02W-0900-09 | COAD | db8d5d6c-c200-4ffc-a1bb-8465044cefad |
| TCGA-AA-3688-01A-01W-0900-09 | COAD | 7224118e-b762-4e72-8bee-9e87c37aac7f |
| TCGA-AA-3692-01A-01W-0900-09 | COAD | 6e2f4d01-6413-473e-98f4-9256ca4285d5 |
| TCGA-AA-3693-01A-01W-0900-09 | COAD | 45ea6cb9-8d5e-4470-bd07-a2c59ddc5cf0 |
| TCGA-AA-3695-01A-01W-0900-09 | COAD | db143a45-b2c5-4dce-98d4-d15dccc5b757 |
| TCGA-AA-3696-01A-01W-0900-09 | COAD | 9e1f1824-12e2-42be-aa57-e0d0b4079a4c |
| TCGA-AA-3715-01A-01W-0900-09 | COAD | 554258ce-99c3-49a3-bfbf-131ec867a0e9 |
| TCGA-AA-3812-01A-01W-0900-09 | COAD | 28087364-af53-4ac4-b1b2-bbe54b71c040 |
| TCGA-AA-3814-01A-01W-0900-09 | COAD | 733e8b21-718b-405d-b860-ed36c70a8411 |
| TCGA-AA-3818-01A-01W-0900-09 | COAD | 9ddb06a8-300e-40d2-8f6a-c851e2f90d90 |
| TCGA-AA-3819-01A-01W-0900-09 | COAD | 0192a572-a235-400d-8fb1-af81e40d3763 |
| TCGA-AA-3831-01A-01W-0900-09 | COAD | 7843d5c1-373d-4a55-82b8-db2f8ead890c |
| TCGA-AA-3833-01A-01W-0900-09 | COAD | 9ea5c555-6e44-4313-8572-779a099efaaa |
| TCGA-AA-3837-01A-01W-0900-09 | COAD | 888c1825-a44b-49cb-bed1-09db01e54b75 |
| TCGA-AA-3848-01A-01W-0900-09 | COAD | 729fbad4-0152-44e5-b26b-dffc1f7dcf70 |
| TCGA-AA-3852-01A-01W-0900-09 | COAD | 1ee1ab0a-cd8c-49d5-ab8c-0d2a2f94724f |
| TCGA-AA-3854-01A-01W-0900-09 | COAD | 2a7ecd84-d49c-484c-a918-381769835ebc |
| TCGA-AA-3856-01A-01W-0900-09 | COAD | 7a07d137-7936-486d-aeb5-6d9598fe4660 |
| TCGA-AA-3858-01A-01W-0900-09 | COAD | 99e41f17-b760-4b34-8230-39aa42db46fd |
| TCGA-AA-3860-01A-02W-0900-09 | COAD | 57869735-96fd-4439-ba2d-583df6fc32a0 |
| TCGA-AA-3875-01A-01W-0900-09 | COAD | 06e6b2e8-634e-4b03-989e-0d192b60b64a |
| TCGA-AA-3966-01A-01W-1073-09 | COAD | 689f1a40-4315-48bc-8b05-75d800e17b44 |
| TCGA-AA-3994-01A-01W-1073-09 | COAD | 4348f66a-e104-4fdd-bdee-2f346832835d |
| TCGA-AA-A004-01A-01W-A00E-09 | COAD | 0b856311-aa63-44b7-a191-9d6d8308c3d0 |
| TCGA-AA-A00N-01A-02W-A00E-09 | COAD | dfb1aec9-d196-49e6-bdb1-9318222b8121 |
| TCGA-AA-A00O-01A-02W-A00E-09 | COAD | 0328eea5-c89c-4462-8af8-48a28ed38537 |
| TCGA-AA-A010-01A-01W-A00E-09 | COAD | 77cdcb19-16fa-4330-921c-e21f17c2298e |
| TCGA-AA-A017-01A-01W-A00E-09 | COAD | a0ad6347-d20c-494a-a094-b816c4fec5de |
| TCGA-AA-A01D-01A-01W-A00E-09 | COAD | e00404be-0bea-4893-89cf-cc24073f10b1 |
| TCGA-AA-A01I-01A-02W-A00E-09 | COAD | ee78a7e5-6ddb-4d06-8fb1-ba7300af59e1 |
| TCGA-AA-A01K-01A-01W-A00E-09 | COAD | 7b7c405e-65c8-4633-ac54-0a112fb478ac |
| TCGA-AA-A024-01A-02W-A00E-09 | COAD | 45a6b8e2-a4a7-400e-ba7a-f93c29f50fe4 |
| TCGA-AA-A029-01A-01W-A00E-09 | COAD | 41be5565-479e-4c56-b48b-1de52dad2299 |
| TCGA-AA-A02F-01A-01W-A00E-09 | COAD | 68c4226b-dfbd-4130-b50e-94839bcb1b0f |
| TCGA-AA-A02H-01A-01W-A00E-09 | COAD | 1cbf3771-fb49-4517-83ba-8e112fcb1d00 |
| TCGA-AA-A02J-01A-01W-A00E-09 | COAD | 5d03450f-b249-4dcd-927b-713158acc8b2 |
| TCGA-AA-A02W-01A-01W-A00E-09 | COAD | 2104138f-b09d-4452-91e1-c4a10382f009 |
| TCGA-AY-4070-01A-01W-1073-09 | COAD | a7a74785-31cf-4527-bae2-991d7df97b5f |
| TCGA-AY-4071-01A-01W-1073-09 | COAD | 80aa3f17-b072-4e59-a6fc-1afe016fa477 |
| TCGA-02-0003-01A-01D-1490-08 | GBM | 458f13e0-34f3-4a92-b3b3-9a3c2ee3ef23 |
| TCGA-02-0033-01A-01D-1490-08 | GBM | 39d1f122-31d0-4e1c-95a7-0e65e75b1457 |
| TCGA-02-0047-01A-01D-1490-08 | GBM | ce03026e-b756-43a2-972d-b3a4dcda5491 |
| TCGA-02-0055-01A-01D-1490-08 | GBM | 9cd89af4-5118-4adb-aa1d-fbd03bf42a33 |
| TCGA-02-2470-01A-01D-1494-08 | GBM | 0b35f2ff-2a08-4585-a1a9-cfc6a9f5b224 |
| TCGA-02-2483-01A-01D-1494-08 | GBM | 4d7f2c74-862b-4aad-98e1-fa831f14a905 |
| TCGA-02-2485-01A-01D-1494-08 | GBM | 0332b017-17d5-4083-8fc4-9d6f8fdbbbde |
| TCGA-02-2486-01A-01D-1494-08 | GBM | 3331813c-f538-4833-b5eb-a214b7d52334 |
| TCGA-06-0119-01A-08D-1490-08 | GBM | 0cda6181-c62b-4ced-a543-d6138fd2e94a |
| TCGA-06-0122-01A-01D-1490-08 | GBM | 08c54819-32fa-455d-a443-fc71dfd3f03a |
| TCGA-06-0124-01A-01D-1490-08 | GBM | 6ae82bf8-7076-43fb-a541-4c7db5d49280 |
| TCGA-06-0125-02A-11D-2280-08 | GBM | 96e3db14-2bb1-4f68-aed6-5e794750c96e |
| TCGA-06-0126-01A-01D-1490-08 | GBM | c3c3059d-e2fb-45ea-80b5-99fb040cba29 |
| TCGA-06-0128-01A-01D-1490-08 | GBM | c5688535-bda4-4831-aaba-e0c19101d7b0 |
| TCGA-06-0129-01A-01D-1490-08 | GBM | 73e7aa35-91b4-4392-bbb9-9ec21f30250c |
| TCGA-06-0130-01A-01D-1490-08 | GBM | c09f0ebd-d604-49a3-9738-0c65fd47fbf9 |
| TCGA-06-0132-01A-02D-1491-08 | GBM | 53c2e159-5774-499f-b0d1-e04fa3faf5c3 |
| TCGA-06-0137-01A-01D-1490-08 | GBM | 37c11dfc-c37c-4cb6-bd81-9e0a7789b0f1 |
| TCGA-06-0139-01A-01D-1490-08 | GBM | c84ff17d-436d-49c1-aef2-b998ffe4a693 |
| TCGA-06-0140-01A-01D-1490-08 | GBM | 18c94086-d2cc-45cd-9bad-f8968a042d5e |
| TCGA-06-0141-01A-01D-1490-08 | GBM | 5af251d5-e76b-480c-8142-6d6fbfce0b2a |
| TCGA-06-0142-01A-01D-1490-08 | GBM | 4bce79ce-c59c-4d86-b25f-28c8edda1651 |
| TCGA-06-0145-01A-01W-0224-08 | GBM | 8f904068-2967-4b38-8813-3ad0a99e4af8 |
| TCGA-06-0151-01A-01D-1491-08 | GBM | 5fea9ebc-8c1b-4078-af87-79c7f5b5470b |
| TCGA-06-0152-01A-02W-0323-08 | GBM | 79062efd-2b09-4798-a504-0a18ca30ef2d |
| TCGA-06-0154-01A-03D-1491-08 | GBM | f5045707-3ddd-4ade-959a-b368437752fb |
| TCGA-06-0155-01B-01D-1492-08 | GBM | 2dc59e9b-3a60-4178-9fa0-81cf5171622d |
| TCGA-06-0157-01A-01D-1491-08 | GBM | b1e62d8e-24d2-4118-8cd0-3142acebdd5b |
| TCGA-06-0158-01A-01D-1491-08 | GBM | 14580533-4a0c-47ca-bb51-c233700de35c |
| TCGA-06-0165-01A-01D-1491-08 | GBM | 1728988e-0877-4194-92c5-92c1ee6c5f5b |
| TCGA-06-0166-01A-01D-1491-08 | GBM | 70157018-a3c5-4ef8-9314-f8715a3438a4 |
| TCGA-06-0167-01A-01D-1491-08 | GBM | d530c696-235d-4a41-944d-e7f7ae21aa17 |
| TCGA-06-0168-01A-01D-1491-08 | GBM | 2b3bab1e-dddd-4c2c-b5ec-7bb6e700e070 |
| TCGA-06-0169-01A-01D-1490-08 | GBM | 06053a14-2d9a-4df0-a79b-81bda36bf3c3 |
| TCGA-06-0171-02A-11D-2280-08 | GBM | 39520be3-a2af-4189-acf4-9d239363333a |
| TCGA-06-0173-01A-01D-1491-08 | GBM | 0908aac1-d3b7-4eec-96f2-a28c3738388c |
| TCGA-06-0174-01A-01D-1491-08 | GBM | 017c9167-0354-41e4-ad50-fb38fcb5668c |
| TCGA-06-0178-01A-01D-1491-08 | GBM | a4fa779b-d116-4696-b170-60f3e215e9fb |
| TCGA-06-0184-01A-01D-1491-08 | GBM | a5a2e50f-dc7e-44cc-bffe-b675a707bf53 |
| TCGA-06-0185-01A-01W-0254-08 | GBM | bc62d57d-b536-41ab-a344-e765fd3f7439 |
| TCGA-06-0188-01A-01W-0254-08 | GBM | cc0c78e7-1d76-45e6-b043-dc209bb9a32a |
| TCGA-06-0189-01A-01D-1491-08 | GBM | 25c64c53-746c-4e92-976a-8bd947fb9c7f |
| TCGA-06-0190-02A-01D-2280-08 | GBM | c065761d-f775-457f-bda0-4c7c257a701e |
| TCGA-06-0192-01B-01W-0348-08 | GBM | 43d7bc6f-be9b-4d5e-bcec-4fb30b0d9b65 |
| TCGA-06-0195-01B-01D-1491-08 | GBM | 2a2fac52-44aa-41f7-ae27-de6b7eba8ff1 |
| TCGA-06-0209-01A-01D-1491-08 | GBM | b4a7de67-14b6-4b8c-abbe-9eaa990d905e |
| TCGA-06-0210-02A-01D-2280-08 | GBM | b60392fb-43d9-4c9c-b91b-ded40492e61c |
| TCGA-06-0211-02A-02D-2280-08 | GBM | 3914c02e-44ad-4c96-8464-61aa95b42c49 |
| TCGA-06-0213-01A-01D-1491-08 | GBM | 885f9df7-fc27-43c2-9acc-833c410b2db1 |
| TCGA-06-0214-01A-02D-1491-08 | GBM | 08ac57ec-0036-4134-a9bb-f22eaa27ab0d |
| TCGA-06-0216-01B-01D-1492-08 | GBM | eac73a02-b2e0-4601-9bd6-aceb07594fe8 |
| TCGA-06-0219-01A-01D-1491-08 | GBM | a6c6c454-058f-41ec-93c3-3cff44bed149 |
| TCGA-06-0221-02A-11D-2280-08 | GBM | b2d17671-d2e1-4c97-8b01-a976d5abe1d6 |
| TCGA-06-0237-01A-02D-1491-08 | GBM | a50b5271-484a-436e-ac6f-6074071015fd |
| TCGA-06-0238-01A-02D-1492-08 | GBM | 7e8c6b9f-0fec-49ea-9ecb-c9ba1fb4cb74 |
| TCGA-06-0240-01A-03D-1491-08 | GBM | 20f74001-1cb8-451d-8173-5795fa93432b |
| TCGA-06-0241-01A-02D-1491-08 | GBM | 4dd4035a-c800-41b0-85c9-02531d2910ed |
| TCGA-06-0644-01A-02D-1492-08 | GBM | 2553c4d2-5f6a-4eba-84b6-04c4761ebf5c |
| TCGA-06-0645-01A-01D-1492-08 | GBM | 3f458a3c-baac-427d-b3d6-6f15104a8886 |
| TCGA-06-0646-01A-01D-1492-08 | GBM | 89742b5d-0256-48c7-8d8f-41b6e5e5b561 |
| TCGA-06-0648-01A-01W-0323-08 | GBM | 33f8304e-11c3-4a9d-ad21-ffea555309dc |
| TCGA-06-0649-01B-01W-0348-08 | GBM | 27af6a5f-993d-41f0-a9af-65e5a8cc41d4 |
| TCGA-06-0650-01A-02D-1696-08 | GBM | 89af56db-b7f9-41d2-af62-c9b2ee7b540f |
| TCGA-06-0686-01A-01W-0348-08 | GBM | 4af220fa-c00b-40b1-ae82-b2c256a3d3fe |
| TCGA-06-0743-01A-01D-1492-08 | GBM | 430e6ca1-d678-4373-8d8d-9d93412c8012 |
| TCGA-06-0744-01A-01W-0348-08 | GBM | d80afd62-48a6-4da4-8026-e6384e86cf62 |
| TCGA-06-0745-01A-01W-0348-08 | GBM | 188c837e-6389-48eb-8b77-91c8a2f099ac |
| TCGA-06-0747-01A-01W-0348-08 | GBM | 7773738f-f5dd-48ae-870c-aa89aea77450 |
| TCGA-06-0749-01A-01W-0348-08 | GBM | 1121aced-04ae-4ba2-a467-c5b8445a0a76 |
| TCGA-06-0750-01A-01W-0348-08 | GBM | fc15ced3-5ed1-4f88-8789-09ec713bd613 |
| TCGA-06-0875-01A-01W-0424-08 | GBM | 862cc896-a0dc-4f02-9940-8c9a5016027b |
| TCGA-06-0876-01A-01W-0424-08 | GBM | c2f27319-4e84-4b12-bce1-623ea20722be |
| TCGA-06-0877-01A-01W-0424-08 | GBM | dda2b842-fd8b-4d14-9aa5-3cd3abc0a0e1 |
| TCGA-06-0878-01A-01W-0424-08 | GBM | 07869e29-9ced-4be5-9a6c-8fd3c29ae487 |
| TCGA-06-0879-01A-01W-0424-08 | GBM | f96b8966-e0c2-4fb6-b3f6-e76d7953d537 |
| TCGA-06-0881-01A-02W-0424-08 | GBM | 1069a9d0-9978-4c01-8516-947200264314 |
| TCGA-06-0882-01A-01W-0424-08 | GBM | 385a3692-3208-479f-9f39-37fb65501b80 |
| TCGA-06-1804-01A-01D-1696-08 | GBM | d9a1ff46-8d28-451e-937f-bdad42bddd64 |
| TCGA-06-1806-01A-02D-1845-08 | GBM | beb40d7c-3861-4efe-9b1d-34ba68a66c9d |
| TCGA-06-2557-01A-01D-1494-08 | GBM | c27290e4-6835-448a-abdc-df8ddd5f4630 |
| TCGA-06-2558-01A-01D-1494-08 | GBM | 19f41e2f-cff9-4f04-ba65-6d945bf05edd |
| TCGA-06-2559-01A-01D-1494-08 | GBM | 8df5560b-9f8f-4636-bdb2-1af8b45df1ba |
| TCGA-06-2561-01A-02D-1494-08 | GBM | f9898ad3-f9b6-4061-90ef-30e0eab0a706 |
| TCGA-06-2562-01A-01D-1494-08 | GBM | 6cb3467e-0ad8-4dd9-8b9b-9103629fd16f |
| TCGA-06-2563-01A-01D-1494-08 | GBM | 1d81086c-bf8b-4459-abcf-1ff905c6bf74 |
| TCGA-06-2564-01A-01D-1494-08 | GBM | 9225f366-b08b-4c43-a09f-a16b3bcfb5aa |
| TCGA-06-2565-01A-01D-1494-08 | GBM | c866726d-2d95-4d23-b3d4-0e28a0b3da00 |
| TCGA-06-2567-01A-01D-1494-08 | GBM | d40a4861-b8c4-4fb8-815a-4e82801eedca |
| TCGA-06-2569-01A-01D-1494-08 | GBM | 617eec0b-78e9-4663-946c-c01e7e00a7de |
| TCGA-06-2570-01A-01D-1495-08 | GBM | 04339769-517c-448d-a7ca-951f83608c60 |
| TCGA-06-5408-01A-01D-1696-08 | GBM | ed8ca267-0153-475b-9154-361af62ff767 |
| TCGA-06-5410-01A-01D-1696-08 | GBM | 67244284-dc40-46cb-a2ac-3f4a38f7bbe4 |
| TCGA-06-5411-01A-01D-1696-08 | GBM | 2fdab641-d73b-4f9a-aa4c-c1944f131a69 |
| TCGA-06-5412-01A-01D-1696-08 | GBM | b6be0866-b8ae-4767-8cdc-e1dd4f78f440 |
| TCGA-06-5413-01A-01D-1696-08 | GBM | 72c13e51-0dd2-4e96-af37-aa471407436f |
| TCGA-06-5414-01A-01D-1486-08 | GBM | 7aa16ff4-169a-4206-83d1-a2495fb56f62 |
| TCGA-06-5415-01A-01D-1486-08 | GBM | fca08ee9-b480-4dc7-be56-f1eb03b56f7c |
| TCGA-06-5417-01A-01D-1486-08 | GBM | 66350d36-6662-4d4c-9cf8-e052a17cddba |
| TCGA-06-5418-01A-01D-1486-08 | GBM | ae28fd78-d254-46fa-aba1-1353931aa414 |
| TCGA-06-5856-01A-01D-1696-08 | GBM | 0bd9b573-712b-4da1-9c33-7b7f43d4af31 |
| TCGA-06-5858-01A-01D-1696-08 | GBM | 951799e6-12f0-4cf6-8732-f2e044db7210 |
| TCGA-06-5859-01A-01D-1696-08 | GBM | bb404507-ab63-4d82-99c6-f3297bffc46f |
| TCGA-06-6388-01A-12D-1845-08 | GBM | c9214f8b-6684-4e29-812c-2a44963e8914 |
| TCGA-06-6389-01A-11D-1696-08 | GBM | 10911471-5404-42d5-817e-f9616e7dacfc |
| TCGA-06-6390-01A-11D-1696-08 | GBM | f04b6bde-63e0-41c9-89f7-07673f9de0f6 |
| TCGA-06-6391-01A-11D-1696-08 | GBM | 40fc77dc-46df-4487-925f-1d87c5326661 |
| TCGA-06-6693-01A-11D-1845-08 | GBM | 45ca8f53-6d0e-4659-a81f-258184b7a70e |
| TCGA-06-6694-01A-12D-1845-08 | GBM | b5a5717d-0e3d-4b44-82f3-5b68187beb52 |
| TCGA-06-6695-01A-11D-1845-08 | GBM | 13817acd-8c1e-4154-8b88-7cdc5f2660a7 |
| TCGA-06-6697-01A-11D-1845-08 | GBM | 7d947ed1-1315-459e-b973-f3dd624d9e39 |
| TCGA-06-6698-01A-11D-1845-08 | GBM | d605a279-c0ea-467c-a423-cdf21547f87e |
| TCGA-06-6699-01A-11D-1845-08 | GBM | 90ba858d-e3bb-40d8-98ee-eeb127c58409 |
| TCGA-06-6700-01A-12D-1845-08 | GBM | 6da42a38-94dd-49b7-8a03-df0f7174ca6f |
| TCGA-06-6701-01A-11D-1845-08 | GBM | fad178f1-385b-4f94-bd29-567c1aa0a8fc |
| TCGA-08-0386-01A-01D-1492-08 | GBM | 90bf7f8f-4b8c-410f-afa6-2b439ec82f97 |
| TCGA-12-0615-01A-01D-1492-08 | GBM | a6068793-51e4-4762-9150-cdfb030e8ade |
| TCGA-12-0616-01A-01D-1492-08 | GBM | b0e2fed7-38bd-48d8-a786-ac574c9fa5be |
| TCGA-12-0618-01A-01D-1492-08 | GBM | 390fc5e9-787e-4a3f-86c8-e3e0e7e43824 |
| TCGA-12-0619-01A-01D-1492-08 | GBM | 79c65ab5-1924-4710-96e4-31e9a615a53e |
| TCGA-12-0688-01A-02D-1492-08 | GBM | 143dc738-1694-4105-8115-9cc0902ef35b |
| TCGA-12-0692-01A-01W-0348-08 | GBM | 937fb2a6-3856-4086-a327-8d8e593b7b7b |
| TCGA-12-0821-01A-01W-0424-08 | GBM | 357e3a3c-cceb-4b38-bc35-6fe8f5be5ac8 |
| TCGA-12-1597-01B-01D-1495-08 | GBM | 7d35c610-cc06-4aa5-8c96-2f7b7465069f |
| TCGA-12-3649-01A-01D-1495-08 | GBM | 2580567a-8f51-4cb7-9525-bba987c55e36 |
| TCGA-12-3650-01A-01D-1495-08 | GBM | 8b1d52e2-489b-4972-9bef-1690ccd2bac9 |
| TCGA-12-3652-01A-01D-1495-08 | GBM | ab460bc2-e504-4b7f-8533-ab06448a55bc |
| TCGA-12-3653-01A-01D-1495-08 | GBM | fdc52d48-828e-481f-ba1c-0264f1da38a5 |
| TCGA-12-5295-01A-01D-1486-08 | GBM | 796f5741-3b2d-46e5-b74f-e5a76604a401 |
| TCGA-12-5299-01A-02D-1486-08 | GBM | a44954fc-49f2-489a-8593-7de98963e4f8 |
| TCGA-12-5301-01A-01D-1486-08 | GBM | 891fc6bc-d0a7-4064-842c-43d500b4ef5d |
| TCGA-14-0740-01B-01D-1845-08 | GBM | f49859c4-adf9-4c53-8288-8a7ad65a940d |
| TCGA-14-0781-01B-01D-1696-08 | GBM | 13878ec6-fce7-423e-b545-6656145e9d2c |
| TCGA-14-0786-01B-01D-1492-08 | GBM | 75fa4de1-29fd-4b54-b63a-add459f1d69c |
| TCGA-14-0787-01A-01W-0424-08 | GBM | 184b240c-ebf1-4ecf-87eb-aae0718cd81f |
| TCGA-14-0789-01A-01W-0424-08 | GBM | 3462087f-f791-43b4-b9d9-b11cc48eaf9e |
| TCGA-14-0790-01B-01D-1494-08 | GBM | d63d49a0-9413-4583-a7a5-cb2c202cc085 |
| TCGA-14-0813-01A-01W-0424-08 | GBM | 754cd19e-a319-4ddf-887b-ddca4914cdf9 |
| TCGA-14-0817-01A-01W-0424-08 | GBM | a5f06dfc-e9b2-46a6-bee5-604d2839baad |
| TCGA-14-0862-01B-01D-1845-08 | GBM | f0b7d451-8190-45a4-8242-bf698f05243d |
| TCGA-14-0871-01A-01W-0424-08 | GBM | 0cc45f48-0967-42dc-8035-e76c6bd0a3fd |
| TCGA-14-1034-02B-01D-2280-08 | GBM | 7cae6c0b-36fe-411b-bbba-093a4c846d84 |
| TCGA-14-1043-01B-11D-1845-08 | GBM | a439c422-8728-42f5-8dda-6e9e1590478c |
| TCGA-14-1395-01B-11D-1845-08 | GBM | 8825b7a5-dfac-4e21-b4ec-05161b1341e9 |
| TCGA-14-1450-01B-01D-1845-08 | GBM | 7ec7f174-13f6-44b1-83e3-6f35a244f00e |
| TCGA-14-1456-01B-01D-1494-08 | GBM | e525e774-f925-41cd-9822-15aeeee29190 |
| TCGA-14-1823-01A-01W-0643-08 | GBM | 1c3ddf6a-e496-4b87-833b-084d814b6876 |
| TCGA-14-1825-01A-01W-0643-08 | GBM | f0d7cb8b-995c-419b-a366-aadb156879bc |
| TCGA-14-1829-01A-01W-0643-08 | GBM | c69ca476-9e11-4f6e-a4f5-6952f792a580 |
| TCGA-14-2554-01A-01D-1494-08 | GBM | 53dec97d-0464-4ffd-8e2e-95b2b9a03af0 |
| TCGA-15-0742-01A-01W-0348-08 | GBM | 3c015456-02f0-4473-be25-b53166da41ea |
| TCGA-15-1444-01A-02D-1696-08 | GBM | cbd4d4e7-f1c4-446c-8dbc-ce06c872ec14 |
| TCGA-16-0846-01A-01W-0424-08 | GBM | cf3eb226-36c2-4498-a5c1-3f161de6fa3f |
| TCGA-16-0861-01A-01W-0424-08 | GBM | deab6efd-8213-4f35-a897-060c605ce58b |
| TCGA-16-1045-01B-01W-0611-08 | GBM | c92c1d87-0df9-4c5a-baef-2dd26ad6d75a |
| TCGA-19-1390-01A-01D-1495-08 | GBM | d7e8e408-0a8f-4177-ad38-08c5da484ed0 |
| TCGA-19-2619-01A-01D-1495-08 | GBM | b765a4c7-4fe8-444c-95bd-6a4d03af1432 |
| TCGA-19-2620-01A-01D-1495-08 | GBM | 6de41ac1-229b-40b9-a494-5588c284351d |
| TCGA-19-2623-01A-01D-1495-08 | GBM | a14ae5c3-fee0-4ed7-9080-51056ce62ef2 |
| TCGA-19-2624-01A-01D-1495-08 | GBM | a8f86b64-914c-4d89-897b-33bcdd1759f7 |
| TCGA-19-2625-01A-01D-1495-08 | GBM | b0833912-0cb6-4d2a-bd18-9fc211793b30 |
| TCGA-19-2629-01A-01D-1495-08 | GBM | 56ffaa35-814c-4c0b-b3c6-d4514d34fec2 |
| TCGA-19-5947-01A-11D-1696-08 | GBM | d5e7dd90-ead0-40fe-94c5-bc740cb509ab |
| TCGA-19-5950-01A-11D-1696-08 | GBM | 8d6626e2-ea32-4b1d-8f2b-389294121692 |
| TCGA-19-5951-01A-11D-1696-08 | GBM | 57cf584c-8c95-42ec-9cb0-707228b70010 |
| TCGA-19-5952-01A-11D-1696-08 | GBM | 483cad63-ca73-4b31-b4c7-9d73f2cb4186 |
| TCGA-19-5953-01B-12D-1845-08 | GBM | a0180465-3685-4735-a76e-acbeebfa635a |
| TCGA-19-5954-01A-11D-1696-08 | GBM | cfd4e06e-203f-4a6f-8aa9-60828e0d4d68 |
| TCGA-19-5955-01A-11D-1696-08 | GBM | c8abde95-f4d7-4d48-879b-bd584eaf8a25 |
| TCGA-19-5958-01A-11D-1696-08 | GBM | fd385a8e-d6dc-4e65-a023-ce485793c410 |
| TCGA-19-5959-01A-11D-1696-08 | GBM | dd3e4733-7154-4162-9a61-a3a685e5f561 |
| TCGA-19-5960-01A-11D-1696-08 | GBM | b8151614-b08f-49a3-ab6f-2e780f765a17 |
| TCGA-26-1442-01A-01D-1696-08 | GBM | 17e25583-886e-4dc9-802b-35e67971073d |
| TCGA-26-5132-01A-01D-1486-08 | GBM | d1132127-1250-43af-9c16-425798a3d1a7 |
| TCGA-26-5133-01A-01D-1486-08 | GBM | 533051f3-5ea5-41a4-8727-11dc6d786607 |
| TCGA-26-5134-01A-01D-1486-08 | GBM | 11956d98-4ba5-486f-ae79-05aacebe0631 |
| TCGA-26-5135-01A-01D-1486-08 | GBM | 2ce48f01-2f61-49d9-a56a-7438bf4a37d7 |
| TCGA-26-5136-01B-01D-1486-08 | GBM | 39e0587b-1b04-4c68-8ae4-3ae7781e8017 |
| TCGA-26-5139-01A-01D-1486-08 | GBM | 8199001b-a3c9-47e1-97cf-943fa8030f46 |
| TCGA-26-6173-01A-11D-1845-08 | GBM | af373e42-cbbf-4a89-8479-bdd413011885 |
| TCGA-26-6174-01A-21D-1845-08 | GBM | 3ba04f15-48f4-4851-a21f-8fa7cc9eac6b |
| TCGA-27-1830-01A-01W-0643-08 | GBM | b391392a-9865-4bf4-b5f1-fa4fb2ad1343 |
| TCGA-27-1831-01A-01D-1494-08 | GBM | 9880c3c9-5685-42a7-8fe9-7585ea1a1d37 |
| TCGA-27-1832-01A-01W-0643-08 | GBM | 7ea7ee22-55a6-4748-9607-d93a6a367122 |
| TCGA-27-1833-01A-01W-0643-08 | GBM | 4d8d34d9-7069-436c-84d6-ace5760c2aec |
| TCGA-27-1834-01A-01W-0643-08 | GBM | a6c0824e-3d2a-498a-af77-44ea96ba5ce4 |
| TCGA-27-1835-01A-01D-1494-08 | GBM | 6d5fd73b-4cad-44ae-8c79-67f2b9d30328 |
| TCGA-27-1836-01A-01D-1494-08 | GBM | 8c58f090-31a3-4b2f-93e7-1ae6f6d73350 |
| TCGA-27-1837-01A-01D-1494-08 | GBM | 61ad1d55-21a9-49c4-925b-54a24703afda |
| TCGA-27-1838-01A-01D-1494-08 | GBM | 881af1d2-3fbc-44dd-8362-e6c386345cf6 |
| TCGA-27-2518-01A-01D-1494-08 | GBM | dae099ff-330f-492b-a06d-6f975e9e5aea |
| TCGA-27-2519-01A-01D-1494-08 | GBM | b0daafab-b783-4cfc-9f7d-8017d98e80bb |
| TCGA-27-2521-01A-01D-1494-08 | GBM | 3678d5f3-9a29-4750-b0a9-20e971ff6aa4 |
| TCGA-27-2523-01A-01D-1494-08 | GBM | d60f54f5-b154-42c4-99fb-cea4e7a33dc7 |
| TCGA-27-2524-01A-01D-1494-08 | GBM | ce679bfd-fbf9-4c78-822e-37d2322d544b |
| TCGA-27-2526-01A-01D-1494-08 | GBM | bc1abcb7-b4e9-4447-b0c5-0fc09401eec0 |
| TCGA-27-2527-01A-01D-1494-08 | GBM | b8b00995-ada6-493b-bafc-0f6c9def41c9 |
| TCGA-27-2528-01A-01D-1494-08 | GBM | 374cbd87-428e-4509-85c1-b7d3302c30a0 |
| TCGA-28-1747-01C-01D-1494-08 | GBM | 7c746081-ac14-4ae2-9564-d67d52f2627c |
| TCGA-28-1753-01A-01D-1494-08 | GBM | c7143f1e-458c-4129-aa91-61b8e4b90e53 |
| TCGA-28-2499-01A-01D-1494-08 | GBM | 28583f40-c3fc-4213-91c1-99d7d536551e |
| TCGA-28-2501-01A-01D-1696-08 | GBM | 2a2cb25d-4069-4824-b09d-2d49634ed284 |
| TCGA-28-2502-01B-01D-1494-08 | GBM | 707466c8-138a-4ed0-b806-6579464595cb |
| TCGA-28-2509-01A-01D-1494-08 | GBM | f4a62fe0-cee2-487a-9a8a-4cd98d8380df |
| TCGA-28-2510-01A-01D-1696-08 | GBM | 5f2dc303-9859-4b63-8aab-c387da4b2cc1 |
| TCGA-28-2513-01A-01D-1494-08 | GBM | 52dd150e-abd7-4fd2-abe9-09428c5a610c |
| TCGA-28-2514-01A-02D-1494-08 | GBM | 6eef4a0e-3fef-4529-8193-21b380d96344 |
| TCGA-28-5204-01A-01D-1486-08 | GBM | e9590ee4-92d8-4afb-908e-0c816d2b82f3 |
| TCGA-28-5207-01A-01D-1486-08 | GBM | 2d795a16-bdc3-44f0-8c01-6eeec0e1a0b1 |
| TCGA-28-5208-01A-01D-1486-08 | GBM | 76209124-b3f0-4bb2-8b2c-e268abdefe2b |
| TCGA-28-5209-01A-01D-1486-08 | GBM | ef8b63f3-b820-46ac-a99c-3d401a6203d7 |
| TCGA-28-5211-01C-11D-1845-08 | GBM | f8dc846b-1b17-4699-9dc5-3f79e21eee94 |
| TCGA-28-5213-01A-01D-1486-08 | GBM | b866e742-5ed0-4d7d-b96c-52f8f6f37142 |
| TCGA-28-5214-01A-01D-1486-08 | GBM | c992e603-30c9-4e30-a425-8050189db4f8 |
| TCGA-28-5215-01A-01D-1486-08 | GBM | 34c77b5d-c3a6-4e83-96f4-fadd729362d9 |
| TCGA-28-5216-01A-01D-1486-08 | GBM | cde8518a-ce8e-4b54-ab21-5ad4171ab1b3 |
| TCGA-28-5218-01A-01D-1486-08 | GBM | 68008a98-3889-4dd2-bcf9-f1f6cbca6355 |
| TCGA-28-5219-01A-01D-1486-08 | GBM | f016e9f7-66a3-4f50-b9cd-58b1c8a955e9 |
| TCGA-28-5220-01A-01D-1486-08 | GBM | f7b80486-fa19-49c7-8ace-ea61338677d7 |
| TCGA-28-6450-01A-11D-1696-08 | GBM | 5f10d0c5-05b8-44bb-98ce-bbea41820850 |
| TCGA-32-1970-01A-01D-1494-08 | GBM | 65723119-bdfe-46f0-b629-c171023abd71 |
| TCGA-32-1979-01A-01D-1696-08 | GBM | 0c81ebb9-20a6-40c1-9be2-17b99517e988 |
| TCGA-32-1980-01A-01D-1696-08 | GBM | 9b267205-1994-46ff-8d0f-56625dae7c1b |
| TCGA-32-1982-01A-01D-1494-08 | GBM | 9cf7c4cb-ce19-4b79-9163-b74369603e22 |
| TCGA-32-1986-01A-01D-1494-08 | GBM | 5afe3ffc-ba3a-49bb-9837-091b600cbb35 |
| TCGA-32-2615-01A-01D-1495-08 | GBM | 65e3c804-b1a3-4e21-9407-90a6edc4e290 |
| TCGA-32-2632-01A-01D-1495-08 | GBM | 27203e18-af27-478c-a224-8bca77a81c90 |
| TCGA-32-2634-01A-01D-1495-08 | GBM | 52b2a114-4f8c-4e02-af9d-24c4a05d4ca0 |
| TCGA-32-2638-01A-01D-1495-08 | GBM | 1e103221-ab46-4a5c-9b96-5e34f0d49fc2 |
| TCGA-32-5222-01A-01D-1486-08 | GBM | f48abf4d-f1fb-48bf-97a1-0c38435b6af7 |
| TCGA-41-2571-01A-01D-1495-08 | GBM | 36349a22-17eb-48d8-9b69-1921ee7576ff |
| TCGA-41-2573-01A-01D-1495-08 | GBM | fadc9e2a-d97d-4e86-a814-4f32f8cfd7a5 |
| TCGA-41-2575-01A-01D-1495-08 | GBM | 4943e80a-d098-49cd-8261-1d53d42f8223 |
| TCGA-41-3392-01A-01D-1495-08 | GBM | c08b37a5-9938-4ab0-8183-d73b01cb9a89 |
| TCGA-41-5651-01A-01D-1696-08 | GBM | 5fd77ba9-5015-4d8b-86a0-582e5c76bdd6 |
| TCGA-41-6646-01A-11D-1845-08 | GBM | 6272bb0c-c47b-4cd2-9f59-398f1a75f020 |
| TCGA-74-6573-01A-12D-1845-08 | GBM | 0941e50e-1205-49ed-8735-1f86eaf87718 |
| TCGA-74-6575-01A-11D-1845-08 | GBM | f4ec96d6-d7fc-4892-9a36-80802f387a12 |
| TCGA-74-6577-01A-11D-1845-08 | GBM | 5be142d5-b6f7-4e1e-ae75-49b302b332a2 |
| TCGA-74-6578-01A-11D-1845-08 | GBM | a2ae2128-4d95-4261-a30d-bd6be58de8e0 |
| TCGA-74-6584-01A-11D-1845-08 | GBM | cedd2d49-371b-4b12-8aac-6a9bd38f2ccb |
| TCGA-76-4925-01A-01D-1486-08 | GBM | ca2fa3da-18d6-4e8b-8081-b07022ead6a8 |
| TCGA-76-4926-01B-01D-1486-08 | GBM | 3c93cb58-d39b-4a5e-907a-8b5438630d21 |
| TCGA-76-4927-01A-01D-1486-08 | GBM | 2dc69425-dbfd-4228-ab78-541062b5c445 |
| TCGA-76-4928-01B-01D-1486-08 | GBM | 6e30f277-875e-4ab8-bc7c-0a5121cde6d1 |
| TCGA-76-4929-01A-01D-1486-08 | GBM | af4f8b89-837a-48b7-b0e7-12aec23fc285 |
| TCGA-76-4931-01A-01D-1486-08 | GBM | d4a27742-ca69-4f54-9bce-ec33d8481fed |
| TCGA-76-4932-01A-01D-1486-08 | GBM | 81656daa-af7c-430c-afa3-0eb10eb9a695 |
| TCGA-76-4934-01A-01D-1486-08 | GBM | e9bc4701-562e-4d35-a949-53a61fd96651 |
| TCGA-76-4935-01A-01D-1486-08 | GBM | c8d06abf-437d-4bc9-804b-44345af74f36 |
| TCGA-76-6191-01A-12D-1696-08 | GBM | 4dbf66ef-4108-4a86-a8eb-6ba8cdefb4a2 |
| TCGA-76-6192-01A-11D-1696-08 | GBM | c29754bc-44e8-4980-98a1-b8d69700f4a3 |
| TCGA-76-6193-01A-11D-1696-08 | GBM | 6a751d65-5fcf-4c03-8253-8f1b8faccab2 |
| TCGA-76-6280-01A-21D-1845-08 | GBM | 9096e339-7730-4d7a-acab-a6c4d26c52c3 |
| TCGA-76-6282-01A-11D-1696-08 | GBM | 1c7f63d2-a2a4-42c3-928b-319695a66443 |
| TCGA-76-6283-01A-11D-1845-08 | GBM | a4083f8b-0c39-4d65-a372-b494caf84f8d |
| TCGA-76-6285-01A-11D-1696-08 | GBM | 28380a2f-d302-45fb-a4c5-31b2fd150bc3 |
| TCGA-76-6286-01A-11D-1845-08 | GBM | 45d03116-6cff-4074-9c26-2e5f1a8854d3 |
| TCGA-76-6656-01A-11D-1845-08 | GBM | fe66f11a-e03d-49c5-befe-db74ef55ce61 |
| TCGA-76-6657-01A-11D-1845-08 | GBM | 6ba47878-126c-420d-b3c1-ca7ea8c182d0 |
| TCGA-76-6660-01A-11D-1845-08 | GBM | f4960945-c464-49c2-8ad6-d73a6fa47b20 |
| TCGA-76-6661-01B-11D-1845-08 | GBM | 8329c910-7ccf-4e84-b468-bd6cf23327a2 |
| TCGA-76-6662-01A-11D-1845-08 | GBM | 7f7c80ca-6ad9-4820-83ca-5248b3873eea |
| TCGA-76-6663-01A-11D-1845-08 | GBM | 624864ad-3178-4a6d-a0cf-7fa3e9bdf8da |
| TCGA-76-6664-01A-11D-1845-08 | GBM | 6a8f17c6-060d-492e-8a39-53d9ac7035a4 |
| TCGA-81-5910-01A-11D-1696-08 | GBM | bcf79a66-30e6-4554-982e-38d8eab46114 |
| TCGA-81-5911-01A-12D-1845-08 | GBM | a501e01b-249c-43cb-aee24355c3c697dd |
| TCGA-87-5896-01A-01D-1696-08 | GBM | 640c33a6-a7df-4dba-9c21-367a9a839f0f |
| TCGA-BA-4074-01A-01D-1434-08 | HNSC | 2c84e904-0cbc-4645-b7e5-94ec45e61268 |
| TCGA-BA-4075-01A-01D-1434-08 | HNSC | 5b3fec35-d127-4cb5-859b-edac003acdf3 |
| TCGA-BA-4076-01A-01D-1434-08 | HNSC | 93dda6a6-907d-4dc2-9391-36dd09c767c6 |
| TCGA-BA-4077-01B-01D-1434-08 | HNSC | 9b37211a-2150-4d33-bc6a-9d6a0a429708 |
| TCGA-BA-4078-01A-01D-1434-08 | HNSC | f02d0332-d7c8-4d2a-98ca-dbe7826437ae |
| TCGA-BA-5149-01A-01D-1512-08 | HNSC | 6e98841c-ce33-4b7e-882d-ce65707d4c10 |
| TCGA-BA-5151-01A-01D-1434-08 | HNSC | dac15d7e-3930-4fcb-b752-4a4f00449ddd |
| TCGA-BA-5152-01A-02D-1870-08 | HNSC | 18da68fd-3bfb-45a3-ba28-4c90555b4e68 |
| TCGA-BA-5153-01A-01D-1434-08 | HNSC | 363ccc6f-dab0-413e-bc42-d738ee25abcd |
| TCGA-BA-5555-01A-01D-1512-08 | HNSC | 65dc1531-713b-41ba-a567-caa12340c0cf |
| TCGA-BA-5556-01A-01D-1512-08 | HNSC | d31fda32-363b-44e4-8f2c-834a66f46b87 |
| TCGA-BA-5557-01A-01D-1512-08 | HNSC | 7caa2a2f-3b77-46f0-9886-37f6e4278d83 |
| TCGA-BA-5558-01A-01D-1512-08 | HNSC | 97a47fa4-c857-4483-9572-07012c10e9d5 |
| TCGA-BA-5559-01A-01D-1512-08 | HNSC | c0845927-fc9a-41b2-9431-619952878e18 |
| TCGA-BA-6868-01B-12D-1912-08 | HNSC | 51647474-f538-4e96-babd-e742f1fb793f |
| TCGA-BA-6869-01A-11D-1870-08 | HNSC | b78a2501-f312-41a2-ab19-7c18d8dfbac6 |
| TCGA-BA-6870-01A-11D-1870-08 | HNSC | 2fdd3f42-cb2f-4faf-8a47-b8bfee058265 |
| TCGA-BA-6871-01A-11D-1870-08 | HNSC | a8a04117-0ebc-4c27-83d6-441be47e5fd3 |
| TCGA-BA-6872-01A-11D-1870-08 | HNSC | 182b2a39-4881-402a-a907-b51aa114584a |
| TCGA-BA-6873-01A-11D-1870-08 | HNSC | f65b842c-257e-4ac7-a155-23d3ac12d41c |
| TCGA-BA-7269-01A-11D-2012-08 | HNSC | 2e8ffdfc-48f5-41e0-9192-d761f3b518ef |
| TCGA-BB-4217-01A-11D-2078-08 | HNSC | 5916ef19-7838-4621-a869-de8c2b34931c |
| TCGA-BB-4223-01A-01D-1434-08 | HNSC | c4799ee4-3014-4b2f-ba7e-9771ab5dc3f1 |
| TCGA-BB-4224-01A-01D-1434-08 | HNSC | cfa7d658-031d-4cd4-9ca3-ceaa201f702d |
| TCGA-BB-4225-01A-01D-1434-08 | HNSC | 85fb5611-0dee-4a73-8aa1-1629ad929173 |
| TCGA-BB-4227-01A-01D-1870-08 | HNSC | c1b315bb-773b-4fd0-88ec-d11044996adc |
| TCGA-BB-4228-01A-01D-1434-08 | HNSC | 6fd93146-1026-4362-982b-d1fc70e3c65d |
| TCGA-BB-7861-01A-11D-2229-08 | HNSC | 77cb5c69-f15e-45de-a060-0e8b52648209 |
| TCGA-BB-7862-01A-21D-2229-08 | HNSC | 84c57a23-1428-488e-9275-9f2bc3673476 |
| TCGA-BB-7863-01A-11D-2229-08 | HNSC | 0bf356d5-1259-4042-9860-2f793f5fe32c |
| TCGA-BB-7864-01A-11D-2229-08 | HNSC | 1d6324a3-8bb4-45d1-89b3-134ffca01aec |
| TCGA-BB-7866-01A-11D-2229-08 | HNSC | 8d6ae619-b33e-453c-aa6d-dda14cd5a337 |
| TCGA-BB-7870-01A-11D-2229-08 | HNSC | d584f4ec-09b0-40fe-bba2-256b6cf6974e |
| TCGA-BB-7871-01A-11D-2229-08 | HNSC | 8e13f8a5-5d80-4e34-bffa-54ae808114e7 |
| TCGA-BB-7872-01A-11D-2229-08 | HNSC | c05cb0b5-b288-48fb-bdc0-ee9acd6643a8 |
| TCGA-CN-4723-01A-01D-1434-08 | HNSC | d5d71c48-1a2d-4d7d-8f2c-e3a68352776b |
| TCGA-CN-4725-01A-01D-1434-08 | HNSC | 57ffef9d-193b-48f6-8d5b-3c2eca854d93 |
| TCGA-CN-4726-01A-01D-1434-08 | HNSC | 2201e681-a727-4fd2-adec-cbcb543b2232 |
| TCGA-CN-4727-01A-01D-1434-08 | HNSC | b24fc60a-fe83-4743-a6d3-d90b807412e1 |
| TCGA-CN-4728-01A-01D-1434-08 | HNSC | e450fec8-66dd-4798-8197-4206b8ba7c4d |
| TCGA-CN-4729-01A-01D-1434-08 | HNSC | 7240e742-9315-4fb8-b6f7-28bfe69410a8 |
| TCGA-CN-4730-01A-01D-1434-08 | HNSC | 543bbfe3-4a11-49af-b445-303f0912bfc3 |
| TCGA-CN-4731-01A-01D-1434-08 | HNSC | 31ffd2d8-ee97-4002-9737-08c044878ace |
| TCGA-CN-4733-01A-02D-1870-08 | HNSC | 12880a34-83d1-4075-b62a-9fc61d18ca09 |
| TCGA-CN-4734-01A-01D-1434-08 | HNSC | fd54bbfa-62a2-4d8b-88fb-b74b91e1b958 |
| TCGA-CN-4735-01A-01D-1434-08 | HNSC | 369ebdf4-ee27-414d-978d-3698711fae98 |
| TCGA-CN-4736-01A-01D-1434-08 | HNSC | 788337f5-722c-45d6-8ca4-8037c489cb64 |
| TCGA-CN-4737-01A-01D-1434-08 | HNSC | 4c6857bb-f20f-4ac9-9c2c-cb83c5387a74 |
| TCGA-CN-4738-01A-02D-1512-08 | HNSC | 1d3b16fd-f98b-45ef-a423-861975f098b6 |
| TCGA-CN-4739-01A-02D-1512-08 | HNSC | 7d6cc6ef-6bb0-44ab-bac1-c8f7198d1d8a |
| TCGA-CN-4740-01A-01D-1434-08 | HNSC | 40308868-8d79-484b-85a4-257142763d72 |
| TCGA-CN-4741-01A-01D-1434-08 | HNSC | 3486c689-d7ae-4ce8-8df5-ac8271b4661d |
| TCGA-CN-4742-01A-02D-1512-08 | HNSC | 1fa89bda-b719-445a-85d2-76ce8c484b15 |
| TCGA-CN-5355-01A-01D-1434-08 | HNSC | 0d93e8bc-69d5-47aa-b4bb-bf7b0ade92d6 |
| TCGA-CN-5356-01A-01D-1434-08 | HNSC | aad13fa4-b2e7-4c89-9936-57cf7a5e16a4 |
| TCGA-CN-5358-01A-01D-1512-08 | HNSC | 498c0b1f-678f-4f70-b0d1-aad89bfa2a23 |
| TCGA-CN-5359-01A-01D-1434-08 | HNSC | dcf1e53d-22dc-4b11-9b3f-e421bc28b835 |
| TCGA-CN-5360-01A-01D-1434-08 | HNSC | 174f1ea8-abcf-44ee-b17b-9687b3ab6dae |
| TCGA-CN-5361-01A-01D-1434-08 | HNSC | 5eea0205-e539-48de-b94c-4bb68c74ec96 |
| TCGA-CN-5363-01A-01D-1434-08 | HNSC | 203f8426-6ec5-427a-9ccf-ec2b4683504d |
| TCGA-CN-5364-01A-01D-1434-08 | HNSC | 22078e53-2c9e-4ae4-a166-34488f259ee8 |
| TCGA-CN-5365-01A-01D-1434-08 | HNSC | a419a54c-58b4-4682-aaca-ed85697dd2a0 |
| TCGA-CN-5366-01A-01D-1434-08 | HNSC | 161342fd-4cfa-4fc8-9708-7bb815b137c6 |
| TCGA-CN-5367-01A-01D-1434-08 | HNSC | 57adb398-48c5-4a14-a43e-f79a19befbda |
| TCGA-CN-5369-01A-01D-1434-08 | HNSC | 4c8e6937-9fd7-41cc-ac74-d8b75235d4b3 |
| TCGA-CN-5370-01A-01D-2012-08 | HNSC | f4ca6755-68ca-4702-b08b-65005d31e9be |
| TCGA-CN-5373-01A-01D-1434-08 | HNSC | 00988676-1e9b-4e00-b4aa-a8f86c21b206 |
| TCGA-CN-5374-01A-01D-1434-08 | HNSC | 28d5a97b-3f3d-4595-9034-8491999fcf40 |
| TCGA-CN-6010-01A-11D-1683-08 | HNSC | 2d9693f3-0917-42be-97b8-4dc15cc4d3f6 |
| TCGA-CN-6011-01A-11D-1683-08 | HNSC | 0e0aa5da-2cb2-47b8-b000-83a07d68ed29 |
| TCGA-CN-6012-01A-11D-1683-08 | HNSC | c5d99faa-ef68-4f08-af97-d722bcc383f5 |
| TCGA-CN-6013-01A-11D-1683-08 | HNSC | 992de9b5-c394-48e7-b4e3-4c4aeacb4a23 |
| TCGA-CN-6016-01A-11D-1683-08 | HNSC | fcb6e29c-864d-483f-a848-8a61202d9516 |
| TCGA-CN-6017-01A-11D-1683-08 | HNSC | 7cd89cbe-6bd9-41a2-a042-345fa0a09866 |
| TCGA-CN-6018-01A-11D-1683-08 | HNSC | 33815edd-bb4f-4f05-bc82-94eafe423652 |
| TCGA-CN-6019-01A-11D-1683-08 | HNSC | 00769a89-ffc5-46f5-a42e-25b3eae886c2 |
| TCGA-CN-6020-01A-11D-1683-08 | HNSC | 1f33c4c7-4f08-44a2-91f5-7ed2d7da68f0 |
| TCGA-CN-6021-01A-11D-1683-08 | HNSC | e62a2c4d-18e3-4ec8-8d93-40e055e65be4 |
| TCGA-CN-6022-01A-21D-1683-08 | HNSC | 90cd2296-7133-4cbe-99cb-84b084eb88cd |
| TCGA-CN-6023-01A-11D-1683-08 | HNSC | d03b8f96-c932-4abf-b508-f4e1b50739ee |
| TCGA-CN-6024-01A-11D-1683-08 | HNSC | 0604584e-0654-4b00-94fc-45e76588000c |
| TCGA-CN-6988-01A-11D-1912-08 | HNSC | 230b06a8-5f6e-41db-bb59-19e4e6c9afaf |
| TCGA-CN-6989-01A-11D-1912-08 | HNSC | 61cd2198-d85e-4eae-b9c6-e36be372595b |
| TCGA-CN-6992-01A-11D-1912-08 | HNSC | 7a70356c-74a3-40c3-bd32-3049da642831 |
| TCGA-CN-6994-01A-11D-1912-08 | HNSC | 157b67ad-f092-4ea3-b557-0406839e6905 |
| TCGA-CN-6995-01A-31D-2012-08 | HNSC | c0b6813d-4b3e-479e-81a7-1e5c2de89b0d |
| TCGA-CN-6996-01A-11D-1912-08 | HNSC | c063bec5-c716-4ea2-843a-e9f0bec3b540 |
| TCGA-CN-6997-01A-11D-2012-08 | HNSC | 11b531cc-d9d9-496a-8448-e654ba71c414 |
| TCGA-CN-6998-01A-23D-2012-08 | HNSC | 9c364f7e-5b90-44ef-9f80-250e428989ef |
| TCGA-CQ-5323-01A-01D-1683-08 | HNSC | 892067ef-c465-46ea-8f91-10636dd0081b |
| TCGA-CQ-5324-01A-01D-1683-08 | HNSC | 67b184fe-c4f4-49f3-938e-5370eb6246b9 |
| TCGA-CQ-5325-01A-01D-1683-08 | HNSC | 22b6abf5-aad8-46ab-9b87-e3c12309cb59 |
| TCGA-CQ-5326-01A-01D-1870-08 | HNSC | 199249f9-808d-4565-bb6b-82724f61edaa |
| TCGA-CQ-5327-01A-01D-1683-08 | HNSC | da19d7bc-9748-4cd4-bd54-4792894838f0 |
| TCGA-CQ-5329-01A-01D-1683-08 | HNSC | 5aa9b6fc-4169-4346-98fb-4c711d08d701 |
| TCGA-CQ-5330-01A-01D-1683-08 | HNSC | 4ce7e702-9b62-459e-b2b4-a26cabba3a93 |
| TCGA-CQ-5331-01A-02D-1870-08 | HNSC | d2c2d3db-dbc0-44f1-b625-17f3f819c122 |
| TCGA-CQ-5332-01A-01D-1683-08 | HNSC | 4fdf4f0d-0a55-4b5e-8545-65f1aad37c10 |
| TCGA-CQ-5334-01A-01D-1683-08 | HNSC | 39978192-2119-4910-a2f6-53834a2b1bf2 |
| TCGA-CQ-6218-01A-11D-1912-08 | HNSC | d3717097-7cdb-446f-a020-78c770362656 |
| TCGA-CQ-6219-01A-11D-1912-08 | HNSC | c6263b94-0ffe-40e7-9184-deb427c67802 |
| TCGA-CQ-6220-01A-11D-1912-08 | HNSC | 65e67eda-16a4-4dfd-94a9-546c76d94a02 |
| TCGA-CQ-6221-01A-11D-2078-08 | HNSC | d6166f0d-c0b5-44a3-814d-0c94c5bc41b0 |
| TCGA-CQ-6222-01A-11D-1912-08 | HNSC | de2c492f-5cd8-4330-a5de-36f693ec31af |
| TCGA-CQ-6223-01A-11D-1912-08 | HNSC | be7cb5b4-1d09-479c-8bf2-a9e7abde575f |
| TCGA-CQ-6224-01A-11D-1912-08 | HNSC | c03d51a0-8731-430d-a792-280e01629e8f |
| TCGA-CQ-6225-01A-11D-1912-08 | HNSC | cd311590-3c69-4ff2-8fbd-cb5b0f21975e |
| TCGA-CQ-6227-01A-11D-1912-08 | HNSC | ca62509e-d477-41ca-9bc2-3f20c2dd4e49 |
| TCGA-CQ-6228-01A-11D-1912-08 | HNSC | 655e502b-1a6e-4eab-a948-4120d6c31c29 |
| TCGA-CQ-6229-01A-11D-1912-08 | HNSC | 07e76152-9e83-42a5-9111-c39a2310a2e4 |
| TCGA-CQ-7065-01A-11D-2078-08 | HNSC | 64c422bb-a531-4636-8e68-bdaf212c1f6dc |
| TCGA-CQ-7067-01A-11D-2229-08 | HNSC | 01f46aa2-e15b-4544-add5-c783868b6c26 |
| TCGA-CQ-7068-01A-11D-2078-08 | HNSC | 97a96e61-f2dc-4af4-807a-3925c1ffbf43 |
| TCGA-CR-5243-01A-01D-1512-08 | HNSC | 297e8b35-5b8b-4d5b-b812-86165f949a20 |
| TCGA-CR-5247-01A-01D-2012-08 | HNSC | 3b5b07b4-29ef-4a55-b6ab-93352613f631 |
| TCGA-CR-5248-01A-01D-2012-08 | HNSC | e5af63d7-e8b2-4a76-8b39-6ee652ad8e5f |
| TCGA-CR-5249-01A-01D-1512-08 | HNSC | 42bf9ca3-47d8-45ff-bccf-bda80af58d22 |
| TCGA-CR-5250-01A-01D-1512-08 | HNSC | 49e54f5a-9b3a-47ff-b6cc-a1eaf54fd136 |
| TCGA-CR-6467-01A-11D-1870-08 | HNSC | 2a7f5a16-9330-45a1-9024-1cff1cdb5714 |
| TCGA-CR-6470-01A-11D-1870-08 | HNSC | 30bc4d1e-f0cb-44c5-a32c-b4b690cd6cc5 |
| TCGA-CR-6471-01A-11D-1870-08 | HNSC | c087e87f-867c-45dd-8645-5ab774e4827c |
| TCGA-CR-6472-01A-11D-1870-08 | HNSC | 52f12c71-2473-4411-aad6-318a3496e82c |
| TCGA-CR-6473-01A-11D-1870-08 | HNSC | 9f3396a0-a38d-4069-b65a-c4c4dd6187ad |
| TCGA-CR-6474-01A-11D-1870-08 | HNSC | 6b4369e1-cf03-4a40-9a66-fc67bfb946b3 |
| TCGA-CR-6477-01A-11D-1870-08 | HNSC | e02f3646-a500-4781-ad44-2f62661a883d |
| TCGA-CR-6478-01A-11D-1870-08 | HNSC | c21f40c6-4260-4def-8cca-1c11895b35b0 |
| TCGA-CR-6480-01A-11D-1870-08 | HNSC | 7ee5501e-5463-4481-b798-3d23bfb4f113 |
| TCGA-CR-6481-01A-11D-1870-08 | HNSC | 5e7d2531-81c1-48bb-9c0a-1867d1f83f92 |
| TCGA-CR-6482-01A-11D-1870-08 | HNSC | 684bcd80-30fb-49e5-b72a-09502a9d1468 |
| TCGA-CR-6484-01A-11D-1870-08 | HNSC | e72df726-1575-4789-afac-3b15a7643401 |
| TCGA-CR-6487-01A-11D-1870-08 | HNSC | d4df06d7-97e1-4f22-83a7-993fdcd3a4da |
| TCGA-CR-6488-01A-12D-2078-08 | HNSC | 8bfa9606-b24d-4803-b551-2e86fb02ae5e |
| TCGA-CR-6491-01A-11D-1870-08 | HNSC | a32853ad-b6a3-4147-ae5a-f48fad71581e |
| TCGA-CR-6492-01A-12D-2078-08 | HNSC | d4550d39-4f32-48ab-b049-2fe623332d07 |
| TCGA-CR-6493-01A-11D-1870-08 | HNSC | f061abfa-4554-4328-9e8f-b84dd2aa4b45 |
| TCGA-CR-7364-01A-11D-2012-08 | HNSC | f5047f1e-5088-4d30-927d-e64147fe661d |
| TCGA-CR-7365-01A-11D-2012-08 | HNSC | ec114413-a950-4e74-abc8-98857af8b9ad |
| TCGA-CR-7367-01A-11D-2012-08 | HNSC | b82e34db-7b0e-4bbd-bc42-ba063ac42409 |
| TCGA-CR-7368-01A-11D-2129-08 | HNSC | 4b194ab3-d213-4a7a-be46-909b4f0c7291 |
| TCGA-CR-7369-01A-11D-2129-08 | HNSC | f16a5c08-c9f8-442e-ba13-45681cacda40 |
| TCGA-CR-7370-01A-11D-2129-08 | HNSC | 9f8ec337-85f7-4b01-a2b6-5db9a9e62f30 |
| TCGA-CR-7371-01A-11D-2012-08 | HNSC | 68201be8-a1a9-4c78-ad99-3c767ca8366b |
| TCGA-CR-7372-01A-11D-2012-08 | HNSC | 9032c525-9bed-47f9-b9f2-ecce4593ea37 |
| TCGA-CR-7373-01A-11D-2012-08 | HNSC | 9b1f5f6d-503c-4933-944a-b4fd1cc3fa93 |
| TCGA-CR-7374-01A-11D-2012-08 | HNSC | 2cf33b63-464e-49a0-88f0-6a6d5b0393c4 |
| TCGA-CR-7376-01A-11D-2129-08 | HNSC | a6b11f68-79da-4542-818d-f404116c0bf8 |
| TCGA-CR-7377-01A-11D-2012-08 | HNSC | 93e4eb9a-7643-411b-be90-94b801f23566 |
| TCGA-CR-7379-01A-11D-2012-08 | HNSC | 8cc45c01-a363-4151-9ea0-32c404b79da4 |
| TCGA-CR-7380-01A-11D-2012-08 | HNSC | ac968fdd-970b-41fc-99f7-5670c741bc06 |
| TCGA-CR-7382-01A-11D-2129-08 | HNSC | fdde6828-b9f4-4648-a86b-157c5d46abb2 |
| TCGA-CR-7383-01A-11D-2129-08 | HNSC | 203629ed-2791-4e22-a9da-be647b0cdef5 |
| TCGA-CR-7385-01A-11D-2012-08 | HNSC | 2c00b622-c4a4-4862-b14a-a97b7261f46f |
| TCGA-CR-7386-01A-11D-2012-08 | HNSC | dac99486-00bc-41ad-92b4-8bed1a28b122 |
| TCGA-CR-7388-01A-11D-2012-08 | HNSC | 3eddb2ad-6c75-4ae7-9d27-8ec0e7b4aa55 |
| TCGA-CR-7389-01A-11D-2012-08 | HNSC | 37149937-8131-4dbf-916b-d599d203eba7 |
| TCGA-CR-7390-01A-11D-2012-08 | HNSC | 714399af-e425-43bb-a82a-b62ca6fd735d |
| TCGA-CR-7391-01A-11D-2012-08 | HNSC | 7236609c-34dd-425a-b882-2dff36983f7b |
| TCGA-CR-7392-01A-11D-2012-08 | HNSC | 0616d3e5-9641-4329-a65a-19f4c6918e1c |
| TCGA-CR-7393-01A-11D-2012-08 | HNSC | f59ef1d2-2fc0-44a0-9d2f-c4efd9e79f5d |
| TCGA-CR-7394-01A-11D-2012-08 | HNSC | 1fe9a612-4c9a-432d-b175-e1d8bdbc7c56 |
| TCGA-CR-7395-01A-11D-2012-08 | HNSC | bd0b1b16-ee20-48e5-be11-70eac9c15630 |
| TCGA-CR-7397-01A-11D-2012-08 | HNSC | b93863c2-4657-4ca2-8fce-094fe5df163a |
| TCGA-CR-7398-01A-11D-2012-08 | HNSC | 12c391dc-3138-4e73-bdc7-b06512dd0fa7 |
| TCGA-CR-7399-01A-11D-2012-08 | HNSC | 0a76ba15-f6e5-484f-8a52-9be8351ebdb7 |
| TCGA-CR-7401-01A-11D-2012-08 | HNSC | f8d6968c-2648-4dcf-a0da-77e46878581c |
| TCGA-CR-7402-01A-11D-2012-08 | HNSC | 015b1cc4-6fa5-43c1-9444-4a1af7663f7e |
| TCGA-CR-7404-01A-11D-2129-08 | HNSC | 1c1a8920-9163-4d56-a982-61c4e792cee7 |
| TCGA-CV-5430-01A-02D-1683-08 | HNSC | 4dfcbe35-9e78-4629-8a00-96fee7062d1e |
| TCGA-CV-5431-01A-01D-1512-08 | HNSC | f1a234f0-8890-4cf3-891f-c7a7423b1e75 |
| TCGA-CV-5432-01A-02D-1683-08 | HNSC | 91e9ac70-5524-4b13-9d53-7cec52b38ea5 |
| TCGA-CV-5434-01A-01D-1683-08 | HNSC | 69ef7b45-cd0e-4d59-a0ee-35a8c830120c |
| TCGA-CV-5435-01A-01D-1683-08 | HNSC | ec0a719b-3c3a-4797-9ec5-90d3474da727 |
| TCGA-CV-5436-01A-01D-1512-08 | HNSC | 34dc613e-e4b4-4897-ac4b-13ff46e46d7e |
| TCGA-CV-5439-01A-01D-1683-08 | HNSC | 42a06486-b084-4497-8fe0-a8cff194e020 |
| TCGA-CV-5440-01A-01D-1512-08 | HNSC | 5f5ba5a9-8089-4fe7-92e3-6c31c5fb32d4 |
| TCGA-CV-5441-01A-01D-1512-08 | HNSC | f57f2873-a4ae-4fc0-9d4c-e1f4ef47482e |
| TCGA-CV-5442-01A-01D-1512-08 | HNSC | 4d42594f-c1f4-45ed-8bd2-7701f914d33c |
| TCGA-CV-5443-01A-01D-1512-08 | HNSC | 9d279797-4464-4ef5-8858-640978ccc258 |
| TCGA-CV-5444-01A-02D-1512-08 | HNSC | cf975479-131b-4b37-927e-cacb1f13e62d |
| TCGA-CV-5966-01A-11D-1683-08 | HNSC | 24ad5336-f5ee-49c0-a176-48411285fbe8 |
| TCGA-CV-5970-01A-11D-1683-08 | HNSC | a52dc15f-d06d-46ed-a73e-aa004a2a736a |
| TCGA-CV-5971-01A-11D-1683-08 | HNSC | 881a530b-fdd2-4674-b95d-fded0dfce4ff |
| TCGA-CV-5973-01A-11D-1683-08 | HNSC | b848fbad-1eb3-4bc2-9006-2d0ca559cee8 |
| TCGA-CV-5976-01A-11D-1683-08 | HNSC | 7b643ce3-43bc-4a14-942a-0d6fcffa0312 |
| TCGA-CV-5977-01A-11D-1683-08 | HNSC | 81f3c96a-54bb-4629-a64e-7c8dae66e11a |
| TCGA-CV-5978-01A-11D-1683-08 | HNSC | 791d4f3f-90e0-4fa5-9671-9b5f04ed3eca |
| TCGA-CV-5979-01A-11D-1683-08 | HNSC | c2c31b58-c5b3-4fc3-be99-b978d2961f86 |
| TCGA-CV-6003-01A-11D-1683-08 | HNSC | 9a040a5e-3d2b-433a-9786-7c26b433c0c2 |
| TCGA-CV-6433-01A-11D-1683-08 | HNSC | 16b220fa-a554-43c9-85b0-315331e5ba6e |
| TCGA-CV-6436-01A-11D-1683-08 | HNSC | a5214457-3a86-4b29-b116-3baaa0aa5099 |
| TCGA-CV-6441-01A-11D-1683-08 | HNSC | 22b32736-3b91-4542-affa-46fa90819e69 |
| TCGA-CV-6933-01A-11D-1912-08 | HNSC | 8ef4b02e-4d34-4d58-aa2d-65a7f73982d5 |
| TCGA-CV-6934-01A-11D-1912-08 | HNSC | f5abf385-0372-4faa-9558-8bf02381b68b |
| TCGA-CV-6935-01A-11D-1912-08 | HNSC | fdc0ebce-5ba2-4c18-b594-50b33ef6d116 |
| TCGA-CV-6936-01A-11D-1912-08 | HNSC | 2d4bdd75-d967-40b2-b55d-99e59cc7e125 |
| TCGA-CV-6937-01A-11D-2012-08 | HNSC | 1c78a20e-150f-4c12-8abe-b941f90e730f |
| TCGA-CV-6938-01A-11D-1912-08 | HNSC | b1dcb76e-b98f-4989-90a2-885e50d8174c |
| TCGA-CV-6939-01A-11D-1912-08 | HNSC | e2e84cc1-2944-489e-be1b-0018a4e723e4 |
| TCGA-CV-6940-01A-11D-1912-08 | HNSC | 39f2e005-79f9-4c63-a6d6-0b378481a3ba |
| TCGA-CV-6941-01A-11D-1912-08 | HNSC | 87071681-0058-4081-91f3-f689a150fc94 |
| TCGA-CV-6942-01A-21D-2012-08 | HNSC | c5409f12-e438-4979-b40e-120899c1fa15 |
| TCGA-CV-6943-01A-11D-1912-08 | HNSC | 4fa37ade-3451-406d-b0bb-e135e1591b70 |
| TCGA-CV-6945-01A-11D-1912-08 | HNSC | fcfc9b74-5b8a-45b7-97ca-4e477e941e7c |
| TCGA-CV-6948-01A-11D-1912-08 | HNSC | 03eb2650-4b9f-46d2-b09f-378d8e919ae2 |
| TCGA-CV-6950-01A-11D-1912-08 | HNSC | 4a341860-44fb-493e-bd46-aeb6610842de |
| TCGA-CV-6951-01A-11D-1912-08 | HNSC | 9e1bf26c-6a68-44d2-aaa8-9af2f67828aa |
| TCGA-CV-6952-01A-11D-1912-08 | HNSC | 2d859062-3655-471e-b3dd-e6ff0671c076 |
| TCGA-CV-6953-01A-11D-1912-08 | HNSC | fb79f2be-3dec-4b5a-b5f3-e29e0fb05a98 |
| TCGA-CV-6954-01A-11D-1912-08 | HNSC | 08f56645-763e-4864-a145-c0136dacd4f5 |
| TCGA-CV-6955-01A-11D-2012-08 | HNSC | f2c7fbe1-af36-4c42-b5ae-b9bf1e88fe36 |
| TCGA-CV-6956-01A-21D-2012-08 | HNSC | 9ccee056-124e-40d5-a07d-c208765d8640 |
| TCGA-CV-6959-01A-11D-1912-08 | HNSC | ff4cc4f1-9897-4d04-a3f6-c28a9b928b7a |
| TCGA-CV-6960-01A-41D-2012-08 | HNSC | 750da72e-cabd-4b97-8160-8c4e39272b8b |
| TCGA-CV-6962-01A-11D-1912-08 | HNSC | 0b2767d9-10b4-4ec4-9437-5a5186e284ca |
| TCGA-CV-7089-01A-11D-2012-08 | HNSC | 125ccb76-bf8d-4ce7-a04c-4424d6da0322 |
| TCGA-CV-7090-01A-11D-2012-08 | HNSC | 5c636c2d-f426-43a9-984d-b4455e4388e5 |
| TCGA-CV-7091-01A-11D-2012-08 | HNSC | 563c5a89-6dad-467e-b2ea-e07677574a08 |
| TCGA-CV-7095-01A-21D-2012-08 | HNSC | e4aba107-a048-46e5-b0aa-901f076b6f61 |
| TCGA-CV-7097-01A-11D-2012-08 | HNSC | 23336d44-bb79-4361-b661-ce26eae06692 |
| TCGA-CV-7099-01A-41D-2012-08 | HNSC | 12a04e68-c814-4a18-a469-d7edc76e362d |
| TCGA-CV-7100-01A-11D-2012-08 | HNSC | f21a5e1f-84b8-4e6f-8230-03d31cc7c431 |
| TCGA-CV-7101-01A-11D-2012-08 | HNSC | 511c3fa8-476b-4ee8-8e93-1ab46bc40dbe |
| TCGA-CV-7102-01A-11D-2012-08 | HNSC | eda5514f-3aa1-447c-ad07-55ec307c26e3 |
| TCGA-CV-7103-01A-21D-2012-08 | HNSC | e04f3556-ae16-410d-bc03-1057ae308329 |
| TCGA-CV-7104-01A-11D-2012-08 | HNSC | 4f429401-f71e-4908-9663-2e66bacbebdd |
| TCGA-CV-7177-01A-11D-2012-08 | HNSC | c984165c-88ea-4840-a980-be818db16820 |
| TCGA-CV-7178-01A-21D-2012-08 | HNSC | 3f30774f-2b8c-4057-abd1-a9dd1e49ec78 |
| TCGA-CV-7180-01A-11D-2012-08 | HNSC | 4233a363-ba28-495c-8590-644199c33d64 |
| TCGA-CV-7183-01A-11D-2012-08 | HNSC | 172e7b30-829e-40b2-976e-4971cd1724a9 |
| TCGA-CV-7235-01A-11D-2012-08 | HNSC | 1758147b-cb09-430b-a8cb-6a144744a79f |
| TCGA-CV-7236-01A-11D-2012-08 | HNSC | dc220a9d-1f16-4fe3-8196-d837a909f038 |
| TCGA-CV-7238-01A-11D-2012-08 | HNSC | e9619e49-7185-4158-9e8b-45d446960b60 |
| TCGA-CV-7242-01A-11D-2012-08 | HNSC | 9e07a1bc-f7c7-4cb4-b3b1-92162a79de0e |
| TCGA-CV-7243-01A-11D-2012-08 | HNSC | bc6a2b7c-8a6c-4084-8551-8d1db9072ec2 |
| TCGA-CV-7245-01A-11D-2012-08 | HNSC | 56291b3c-595c-4388-a264-9037a48401d8 |
| TCGA-CV-7247-01A-11D-2012-08 | HNSC | b0ce56d2-8e2b-42b4-ac59-d37ba5a7a2c3 |
| TCGA-CV-7248-01A-11D-2012-08 | HNSC | 8ffc7f9d-16da-4cff-b845-f2ff8df87569 |
| TCGA-CV-7250-01A-11D-2012-08 | HNSC | 14516d2b-47dc-4768-977b-bc3c1fe93722 |
| TCGA-CV-7252-01A-11D-2012-08 | HNSC | 9692c6b2-ce97-4c92-a0dd-f27d01a94e6e |
| TCGA-CV-7253-01A-11D-2012-08 | HNSC | d501a7e5-70e7-4f80-851a-efe8859d603a |
| TCGA-CV-7254-01A-11D-2012-08 | HNSC | fd22e861-571e-44da-82b6-b128e07d1963 |
| TCGA-CV-7255-01A-11D-2012-08 | HNSC | 4dedba61-e137-4ae4-8312-94231e3b1d16 |
| TCGA-CV-7261-01A-11D-2012-08 | HNSC | 9fa7bc79-d05b-41da-8bcc-8d5ad4451b0c |
| TCGA-CV-7263-01A-11D-2012-08 | HNSC | 19a07472-c8b9-4a34-b2cb-11ace35e7903 |
| TCGA-CV-7406-01A-11D-2078-08 | HNSC | 8c9effa8-acb6-4db0-874a-8f0df386924c |
| TCGA-CV-7407-01A-11D-2078-08 | HNSC | 94631dc8-6dcb-49ed-bb68-e1a57a65f1cb |
| TCGA-CV-7409-01A-31D-2229-08 | HNSC | 47fa56f1-0802-403a-a644-913f1a0fdeca |
| TCGA-CV-7410-01A-21D-2078-08 | HNSC | b89c4f94-b07c-485b-95ba-ffe815616d78 |
| TCGA-CV-7411-01A-11D-2078-08 | HNSC | 790e387e-9e87-48d0-bc9d-2bc92f20abc5 |
| TCGA-CV-7413-01A-11D-2078-08 | HNSC | be482a19-0de0-4e60-a831-9ebe8545a6f3 |
| TCGA-CV-7414-01A-11D-2078-08 | HNSC | 7137f980-5301-4b18-9664-d887eaced75e |
| TCGA-CV-7415-01A-11D-2078-08 | HNSC | bb1e4188-130c-4206-8671-d7ce3eb8ee74 |
| TCGA-CV-7418-01A-11D-2078-08 | HNSC | 25a70d04-f533-4e60-b9fc-e74d600db296 |
| TCGA-CV-7421-01A-11D-2078-08 | HNSC | ee675976-b447-48c8-bc67-6878a0d35e07 |
| TCGA-CV-7422-01A-21D-2078-08 | HNSC | 5eb3f291-082c-48a8-b653-09264342adee |
| TCGA-CV-7423-01A-11D-2078-08 | HNSC | a99653e0-2751-4423-93f7-abcf258c9868 |
| TCGA-CV-7424-01A-11D-2078-08 | HNSC | 76d5fc22-fd06-43f6-94a8-943a09db5fd6 |
| TCGA-CV-7425-01A-11D-2078-08 | HNSC | f8cc6696-91d0-4eba-a765-ef7d044238ce |
| TCGA-CV-7427-01A-11D-2078-08 | HNSC | 3fdb4698-4a38-4a81-a403-d1ce5568c225 |
| TCGA-CV-7429-01A-11D-2129-08 | HNSC | 14b42e59-e519-4efc-8105-6f6b83d33353 |
| TCGA-CV-7430-01A-11D-2129-08 | HNSC | 29a4027f-4d4f-4133-b40a-3bfab6d2ac9e |
| TCGA-CV-7432-01A-11D-2129-08 | HNSC | 60da7e3f-4d9c-4cb3-856d-6cc02e381028 |
| TCGA-CV-7433-01A-11D-2129-08 | HNSC | 15380da5-6a0b-4649-b21b-ce1ed7d61b67 |
| TCGA-CV-7434-01A-11D-2129-08 | HNSC | d64e4e80-e6c6-42c8-8bc6-0fafb6475c51 |
| TCGA-CV-7435-01A-11D-2129-08 | HNSC | 16b7fd85-3664-4c4a-9a43-48b107dbcf7f |
| TCGA-CV-7437-01A-21D-2129-08 | HNSC | 53413980-80cc-4c73-8bb6-31a01d6df86e |
| TCGA-CV-7438-01A-21D-2129-08 | HNSC | 6fd3ecf3-c87c-46c3-81f0-11e2f8936d61 |
| TCGA-CV-7440-01A-11D-2129-08 | HNSC | 901c2ed5-8348-4dd9-a84c-6c0b18d6525e |
| TCGA-CX-7082-01A-11D-2012-08 | HNSC | 4c6c96b8-958e-4235-9673-8bf4ce0e6b38 |
| TCGA-CX-7085-01A-21D-2012-08 | HNSC | 4f6ee10b-246d-49cd-8b60-01dcb175e634 |
| TCGA-CX-7086-01A-11D-2078-08 | HNSC | dfcb7c6e-b0f4-4557-9669-4c580d1093a0 |
| TCGA-CX-7219-01A-11D-2012-08 | HNSC | 83f92af6-60ab-402e-8990-e1060ca3cc4c |
| TCGA-D6-6515-01A-21D-1870-08 | HNSC | 15c4d640-884c-4d55-897e-2f68314423fe |
| TCGA-D6-6516-01A-11D-1870-08 | HNSC | 5ab94b24-1a1f-4df7-a5c6-b1dce8ee9be5 |
| TCGA-D6-6517-01A-11D-1870-08 | HNSC | c553e4a2-cbea-43d6-8937-a48836856b5a |
| TCGA-D6-6823-01A-11D-1912-08 | HNSC | e1f4d8ef-f24a-417b-bf22-c03cdb6b5275 |
| TCGA-D6-6824-01A-11D-1912-08 | HNSC | b658aa3f-0812-4812-8254-816d9a4d7c04 |
| TCGA-D6-6825-01A-21D-1912-08 | HNSC | 01f44db3-84dc-4f96-888d-b0370bf582a5 |
| TCGA-D6-6826-01A-11D-1912-08 | HNSC | 368030ac-f855-452a-a3d3-3698ab9a00dd |
| TCGA-D6-6827-01A-11D-1912-08 | HNSC | 059be8f9-9536-40c0-a751-5fe529a2f01f |
| TCGA-DQ-5624-01A-01D-1870-08 | HNSC | 01282192-5bb6-44d6-bbc7-33a42eba416b |
| TCGA-DQ-5625-01A-01D-1870-08 | HNSC | 4e042e1d-8604-484a-b229-94b85745a478 |
| TCGA-DQ-5629-01A-01D-1870-08 | HNSC | e748f828-0b80-47f3-aa92-fb3b2be0dcc2 |
| TCGA-DQ-5630-01A-01D-1870-08 | HNSC | 5aa7ff44-d4ff-4163-81db-9f09bec8d5b0 |
| TCGA-DQ-5631-01A-01D-1870-08 | HNSC | e389975a-e588-48d4-9ed3-548e8ed9de1c |
| TCGA-DQ-7588-01A-11D-2078-08 | HNSC | 6aad9b01-6a99-4f21-955f-7938af25a188 |
| TCGA-DQ-7589-01A-11D-2229-08 | HNSC | de34e28e-942b-442b-b745-7f2a0e56f3ff |
| TCGA-DQ-7590-01A-11D-2229-08 | HNSC | 5cbcfa67-f062-4a03-84ad-dabfbcf14514 |
| TCGA-DQ-7591-01A-11D-2078-08 | HNSC | 4068a2fc-452d-4b2c-88d8-72d30097527b |
| TCGA-DQ-7592-01A-11D-2078-08 | HNSC | d8e20b3b-2666-4b53-aa85-a5056028df98 |
| TCGA-DQ-7594-01A-11D-2229-08 | HNSC | 92e689c0-08ab-472b-aedc-6344fedcbbc0 |
| TCGA-DQ-7595-01A-11D-2229-08 | HNSC | 7d504cd7-09f0-4691-a1b2-55fc7d206439 |
| TCGA-F7-7848-01A-11D-2129-08 | HNSC | ba8a3e47-ee55-4c88-b29f-6d161ffae1d0 |
| TCGA-H7-7774-01A-21D-2078-08 | HNSC | 0eb5b79a-e3be-4b19-aef6-74247986aaf6 |
| TCGA-HD-7229-01A-11D-2012-08 | HNSC | 26b27991-540f-47f4-95f3-a59a493da593 |
| TCGA-HD-7753-01A-11D-2078-08 | HNSC | 7dc33525-6f57-4b12-9b72-c9c845296ae3 |
| TCGA-HD-7754-01A-11D-2078-08 | HNSC | 233ecdc4-0b42-4533-8908-64ac7d3ac33b |
| TCGA-HD-7831-01A-11D-2129-08 | HNSC | ae914215-3b1a-4edb-9f5a-ce4a17154178 |
| TCGA-HD-7832-01A-11D-2129-08 | HNSC | 374f3e37-87e5-4450-a89f-0bde3981a31e |
| TCGA-HD-7917-01A-11D-2229-08 | HNSC | 451948c9-3d16-4771-b006-28b98580db2c |
| TCGA-HL-7533-01A-11D-2229-08 | HNSC | fbf8f4a8-be9e-4713-884d-c80ef662d622 |
| TCGA-IQ-7630-01A-11D-2078-08 | HNSC | 80442509-c2f0-4047-956e-a3633dfd472b |
| TCGA-IQ-7631-01A-11D-2078-08 | HNSC | b2266f1c-1642-4849-9278-41e827691aa7 |
| TCGA-IQ-7632-01A-11D-2078-08 | HNSC | 9fcfc377-a153-401c-95b4-8a4569866096 |
| TCGA-A3-3311-01A-01D-0966-08 | RCC | 9c095b70-9a64-48b0-8a1c-45dd00a70019 |
| TCGA-A3-3316-01A-01D-0966-08 | RCC | e1241cff-4071-482e-be5b-adb9c46a480a |
| TCGA-A3-3317-01A-01D-0966-08 | RCC | cd12847f-695b-4b97-9a56-a4a1ddc58ec4 |
| TCGA-A3-3319-01A-01D-0966-08 | RCC | a771a7ad-8dfa-46ee-849d-4478798c46a6 |
| TCGA-A3-3320-01A-01D-0966-08 | RCC | 5c4cc718-d7b5-453c-89d8-186ab0869e68 |
| TCGA-A3-3322-01A | RCC | 6f329d07-3308-4c84-9113-2bf000e9be3b |
| TCGA-A3-3323-01A-01D-0966-08 | RCC | 21c50574-7496-4be5-b723-1fdb980fb208 |
| TCGA-A3-3326-01A-01D-0966-08 | RCC | 60ed222b-cd0c-4bc5-acd0-39f207be3289 |
| TCGA-A3-3346-01A-01D-0966-08 | RCC | c8a52c11-2278-4f15-80bb-c7115c2cd737 |
| TCGA-A3-3347-01A-02D-1386-10 | RCC | 2f4a6bd7-16ff-4689-b41d-c5fabb87823b |
| TCGA-A3-3349-01A-01D-1251-10 | RCC | c2b257f6-9cb5-4598-89c7-f0b55e24dbb3 |
| TCGA-A3-3357-01A-02D-1421-08 | RCC | db6f5ad9-ae6e-4689-b146-f733f8352c54 |
| TCGA-A3-3358-01A-01D-1534-10 | RCC | fd42afa7-6f0f-48e8-a947-bb9c9f4770ef |
| TCGA-A3-3362-01A-02D-1386-10 | RCC | 03c9042a-0206-4f12-b444-62f435140e8d |
| TCGA-A3-3363-01A-01D-0966-08 | RCC | 34dac639-c2e5-447d-99c5-c6a3e15538fe |
| TCGA-A3-3365-01A | RCC | 8bc46a09-7328-42e0-ad97-e557ec81048e |
| TCGA-A3-3367-01A-02D-1421-08 | RCC | 83a091b9-35cc-4f3b-9d5f-d699b79ac421 |
| TCGA-A3-3370-01A-02D-1421-08 | RCC | 21ce7121-87b4-4686-9bf6-aff71d8b2223 |
| TCGA-A3-3372-01A-01D-0966-08 | RCC | f9f50073-a1d3-4c52-be78-529bd05cbce4 |
| TCGA-A3-3373-01A-02D-1421-08 | RCC | 6cbaac72-ca6e-4c4b-a016-1836959344c8 |
| TCGA-A3-3376-01A-02D-1421-08 | RCC | 31031387-393f-4bf9-ba87-cfe7330afc13 |
| TCGA-A3-3378-01A-01D-0966-08 | RCC | f04f3a00-e743-4fed-a0b0-e6a81bdd6ddd |
| TCGA-A3-3380-01A-01D-0966-08 | RCC | 269d4e2a-a425-4fde-bb51-5880f7f8b2b9 |
| TCGA-A3-3382-01A-01D-0966-08 | RCC | f10e1718-6fb8-4c08-bc28-439f26355cd2 |
| TCGA-A3-3383-01A-01D-0966-08 | RCC | 2ea06f57-c7fa-4881-b9c0-dd3f9c1c4ca0 |
| TCGA-A3-3385-01A | RCC | f780aef6-1c9c-4167-9f55-48885d6e5874 |
| TCGA-A3-3387-01A-01D-1534-10 | RCC | e9e149ff-79e0-48f9-9262-1fbbad865e77 |
| TCGA-AK-3429-01A-02D-1386-10 | RCC | fa51dce9-2101-4af7-9280-4bad56b6848e |
| TCGA-AK-3430-01A | RCC | b16a82ca-2eaf-4b7a-b469-2be4a023fc2a |
| TCGA-AK-3436-01A-02D-1386-10 | RCC | 714cd118-7f2b-47a5-83f6-41b20674ad03 |
| TCGA-AK-3444-01A-01D-0966-08 | RCC | ea794170-156d-4251-b899-abfd60b213b0 |
| TCGA-AK-3451-01A | RCC | 242777f6-a875-4072-9696-8d7f7d718906 |
| TCGA-AK-3455-01A-01D-0966-08 | RCC | 3fbeeda4-a6c4-45a4-a963-dc6ca3f7e0ba |
| TCGA-AK-3456-01A-02D-1386-10 | RCC | d36fe1be-96a5-4001-a95e-d499a6087146 |
| TCGA-AK-3458-01A-01D-1501-10 | RCC | 0198f3c3-78f2-4c19-90d5-c77b74044ca2 |
| TCGA-AS-3778-01A-01D-0966-08 | RCC | 7b56e923-2bc5-4368-8e28-42649d3bf169 |
| TCGA-B0-4700-01A-02D-1534-10 | RCC | 32cb433f-359c-44c3-b2df-d2a64df90175 |
| TCGA-B0-4706-01A-01D-1501-10 | RCC | 040fdd9b-db76-4357-9aed-77a8cbde058d |
| TCGA-B0-4710-01A | RCC | 6fc8cb4b-1dc0-46b8-ae80-7dbd022c9431 |
| TCGA-B0-4712-01A-01D-1501-10 | RCC | 032b33f8-ff79-47de-8cb2-d744eab8bd1a |
| TCGA-B0-4810-01A-01D-1501-10 | RCC | e014eeeb-c48e-42bb-a683-93299087a3cf |
| TCGA-B0-4811-01A-01D-1501-10 | RCC | a46182dc-2481-4911-9f6b-9532666f9f8c |
| TCGA-B0-4815-01A-01D-1501-10 | RCC | fe091054-41d3-44fa-86a2-fad3ae58423f |
| TCGA-B0-4816-01A | RCC | d05c3419-4164-4a69-8b11-ce1f5c29b5d4 |
| TCGA-B0-4818-01A-01D-1501-10 | RCC | 213bf382-c2ca-45d4-95ae-329e6653620f |
| TCGA-B0-4823-01A-02D-1421-08 | RCC | 9f790e7e-3475-4242-82fc-cbdd461ce5ef |
| TCGA-B0-4827-01A-02D-1421-08 | RCC | 02f83f9a-4e4d-44f3-8d67-b4fc2d35102b |
| TCGA-B0-4842-01A-02D-1421-08 | RCC | ae765ade-6a06-439c-a1cd-67222a70f44e |
| TCGA-B0-4852-01A-01D-1501-10 | RCC | 28dbeb57-c919-4f91-aa3c-7b8f3809011e |
| TCGA-B0-4945-01A-01D-1421-08 | RCC | 9fae377f-6c63-4f47-a769-a1396fb15f56 |
| TCGA-B0-5075-01A | RCC | 200819c3-826e-49a1-8824-6d4752e6eb6f |
| TCGA-B0-5077-01A-01D-1462-08 | RCC | 587f2bd8-952a-4f31-98e7-7654c80b8a99 |
| TCGA-B0-5080-01A-01D-1501-10 | RCC | 9adf0a63-1d5c-403a-9e78-cb9d62a249a4 |
| TCGA-B0-5081-01A-01D-1462-08 | RCC | 71a9d096-0e27-4585-b54a-48214d83cd6c |
| TCGA-B0-5085-01A-01D-1462-08 | RCC | a36e36ee-48f3-4674-a9f3-a121a09535c5 |
| TCGA-B0-5088-01A-01D-1462-08 | RCC | e56245d6-c681-44e0-9eb2-504bee3e1b32 |
| TCGA-B0-5092-01A-01D-1421-08 | RCC | 76b9d9e3-6010-4894-8435-debe95a376b5 |
| TCGA-B0-5094-01A-01D-1421-08 | RCC | 8b910c03-86a9-488d-80b4-1f8c214c2941 |
| TCGA-B0-5095-01A-01D-1421-08 | RCC | 93c714f8-acea-4550-92fe-aad4aad65ac9 |
| TCGA-B0-5096-01A-01D-1421-08 | RCC | 261de0a2-6006-4b3b-aac0-37d9b33840aa |
| TCGA-B0-5097-01A-01D-1421-08 | RCC | 3af2978e-b892-4817-be05-39f020c06b5e |
| TCGA-B0-5099-01A-01D-1421-08 | RCC | c3150136-ae55-49d0-9212-86728464167d |
| TCGA-B0-5100-01A-01D-1421-08 | RCC | b20bd619-59c9-4e2a-8e64-7bb44eaa75ce |
| TCGA-B0-5102-01A-01D-1421-08 | RCC | abea5e3e-705a-4d2c-b207-1ab43767a19b |
| TCGA-B0-5104-01A | RCC | ac2cfbde-9d62-49db-9a07-e8166003f10f |
| TCGA-B0-5106-01A-01D-1421-08 | RCC | c0e28603-7204-416d-ba3d-5377a38f677d |
| TCGA-B0-5107-01A | RCC | 4c6f4edb-9a29-48e6-8521-9c5fd2572e2d |
| TCGA-B0-5108-01A-01D-1421-08 | RCC | d1d37af8-d2c3-4825-8e47-1a2e52e3acbb |
| TCGA-B0-5109-01A-02D-1421-08 | RCC | 58d6e408-ed00-4e1f-bffa-e73250cfe4a0 |
| TCGA-B0-5110-01A | RCC | 38041aeb-60fe-4784-a5d8-fd04b5c0c5f8 |
| TCGA-B0-5113-01A-01D-1421-08 | RCC | 64b234e0-74f6-453f-b5cb-280e01fba09b |
| TCGA-B0-5115-01A-01D-1421-08 | RCC | f122b61c-d537-4456-84e8-54e541eec531 |
| TCGA-B0-5116-01A | RCC | 97421d06-b199-4246-b2da-80a9ba313335 |
| TCGA-B0-5119-01A-02D-1421-08 | RCC | 414d47c7-41bb-4c83-8cdf-703fa0a46f01 |
| TCGA-B0-5120-01A-01D-1421-08 | RCC | 6ce58fbc-6742-4ade-84b0-cd025266e030 |
| TCGA-B0-5121-01A-02D-1421-08 | RCC | a2751cb2-8545-490c-92d9-edb9775d32b8 |
| TCGA-B0-5399-01A | RCC | a1dddbed-c780-412a-b563-914f71e5c75d |
| TCGA-B0-5400-01A-01D-1501-10 | RCC | e7128330-77b1-48be-b9f0-be986aa63ea8 |
| TCGA-B0-5402-01A-01D-1501-10 | RCC | ca62bea0-a008-481e-8a91-d0f3a9598255 |
| TCGA-B0-5691-01A-11D-1534-10 | RCC | ac2e1d29-e239-4dab-9d81-77c8d45970eb |
| TCGA-B0-5692-01A-11D-1534-10 | RCC | 1af40135-8357-40b7-b711-478633a70f97 |
| TCGA-B0-5693-01A-11D-1534-10 | RCC | be92ee16-6288-46c0-aaa7-7a27020cd7ca |
| TCGA-B0-5694-01A-11D-1534-10 | RCC | 6edbaa05-b935-4f82-b070-8fc80ea6b609 |
| TCGA-B0-5695-01A | RCC | 86e4862c-7405-40b5-b73f-be0c6c52ea6d |
| TCGA-B0-5696-01A-11D-1534-10 | RCC | 48b270af-07f2-4cb5-ace2-e2676ffaccd9 |
| TCGA-B0-5697-01A-11D-1534-10 | RCC | 9ca4e638-5a95-4eeb-bfc4-257e8ea8fa66 |
| TCGA-B0-5698-01A-11D-1669-08 | RCC | 2ddf2fa6-7871-49fb-be2c-8fce6f8e41ed |
| TCGA-B0-5699-01A | RCC | 086554a9-2172-43a7-9f52-aab7d0888429 |
| TCGA-B0-5701-01A-11D-1534-10 | RCC | 0e1c563a-ee60-478b-9286-ed90e7561892 |
| TCGA-B0-5702-01A-11D-1534-10 | RCC | 780b3f3e-1c49-40de-9131-65c4df9ebba6 |
| TCGA-B0-5703-01A-11D-1534-10 | RCC | 963400a2-d939-41a5-8c42-9fc3a04b8362 |
| TCGA-B0-5705-01A-11D-1534-10 | RCC | d3095df5-5466-4b98-9f6d-f8ae8916ccca |
| TCGA-B0-5706-01A-11D-1534-10 | RCC | b60cf910-2d2e-483a-a9de-ce1e5f8d3825 |
| TCGA-B0-5707-01A-11D-1534-10 | RCC | eb2f9f38-bce2-4746-a3c8-40abc3379b32 |
| TCGA-B0-5709-01A-11D-1534-10 | RCC | bfeaecbe-7148-4642-b69a-b908a248f328 |
| TCGA-B0-5710-01A-11D-1669-08 | RCC | 12f1e370-c269-4b95-a89b-a1f3ae42e876 |
| TCGA-B0-5711-01A-11D-1669-08 | RCC | cf09ae91-5523-494c-8f30-c26f6ba37624 |
| TCGA-B0-5713-01A-11D-1669-08 | RCC | 2f35dbf4-3223-4550-951b-1409a30ece68 |
| TCGA-B0-5812-01A-11D-1669-08 | RCC | 6327ce2c-8a24-45b9-9577-7b7d7b603e68 |
| TCGA-B2-3924-01A | RCC | 21527594-ed75-4654-9caf-83d31f248e67 |
| TCGA-B2-4098-01A | RCC | 6463ae73-a885-4d69-9345-7110ddac0c7e |
| TCGA-B2-4099-01A | RCC | e242adb8-db67-475e-a0e4-52a622666b12 |
| TCGA-B2-4101-01A | RCC | a9947b6c-dbc7-4ba5-af61-7647e11e2973 |
| TCGA-B4-5377-01A-01D-1501-10 | RCC | a615b02d-fd18-47ef-bd66-6dba56de6981 |
| TCGA-B8-4143-01A-01D-1806-10 | RCC | bb186c78-1052-48ec-97f4-c94bddf0df72 |
| TCGA-B8-4146-01B-11D-1669-08 | RCC | 380bdba7-8a12-4136-877a-f54346d2d8a5 |
| TCGA-B8-4148-01A-02D-1386-10 | RCC | fe752e2b-e694-4fa9-99d6-46d5bff9e8cf |
| TCGA-B8-4151-01A-01D-1806-10 | RCC | 3f847558-8bc7-49b0-899d-2a7b8f0e3d1a |
| TCGA-B8-4153-01B-11D-1669-08 | RCC | a66078d8-a6b2-4dc4-bfa3-def5a2e4504f |
| TCGA-B8-4154-01A-01D-1251-10 | RCC | e48f5c14-4b64-4d4b-8273-bebc74182181 |
| TCGA-B8-4620-01A | RCC | e4ec1484-4f77-4520-9ff5-bc4dc8a0fb15 |
| TCGA-B8-4621-01A | RCC | 242a72ad-5968-4bbf-936d-75b398a61b96 |
| TCGA-B8-4622-01A | RCC | 1c86e0f6-a019-47a5-8325-bbb82f76488c |
| TCGA-B8-5158-01A-01D-1421-08 | RCC | 9d730534-98e7-464e-945c-5964cec5362a |
| TCGA-B8-5159-01A-01D-1421-08 | RCC | ed8a9be1-31c6-40e2-9af2-8abd80d00995 |
| TCGA-B8-5163-01A-01D-1421-08 | RCC | 903132ef-877f-4207-ba28-2e9dd765c824 |
| TCGA-B8-5164-01A | RCC | 471ce542-e85b-4bdb-b365-4562a93ef1e5 |
| TCGA-B8-5165-01A-01D-1421-08 | RCC | d1579785-5c42-4bda-9825-15ead235f7f4 |
| TCGA-B8-5545-01A-01D-1669-08 | RCC | 514d2342-64ba-4c9f-9866-63bdbc26fda3 |
| TCGA-B8-5550-01A | RCC | dafed455-98a2-419a-bebc-f90b731e2813 |
| TCGA-B8-5552-01B-11D-1669-08 | RCC | 13b52e49-20df-4e39-9dc9-cf8f7c157bd7 |
| TCGA-B8-5553-01A-01D-1534-10 | RCC | 7c19e63c-770b-4289-aa47-9b2cf261b4ca |
| TCGA-BP-4161-01A | RCC | 154de511-2bba-4959-970b-6a8429f29793 |
| TCGA-BP-4162-01A | RCC | ca4eac28-22c9-48d8-8139-7cda2cfe4ae2 |
| TCGA-BP-4163-01A | RCC | e44de28c-bce0-471d-bd4c-bea710f7c3cc |
| TCGA-BP-4164-01A | RCC | a8fab76e-ae69-43d6-972b-5837aec668fd |
| TCGA-BP-4167-01A-02D-1386-10 | RCC | 79b810e1-4de4-496d-9f70-ab62246e781b |
| TCGA-BP-4770-01A-01D-1501-10 | RCC | aecbc5db-f75a-42d0-a84d-aa0369b08eec |
| TCGA-BP-4782-01A | RCC | a6c21bf2-dd9b-4243-863e-9d53b056666f |
| TCGA-BP-4801-01A-02D-1421-08 | RCC | d3e62cb1-5ced-42cb-a360-479ee01877aa |
| TCGA-BP-4960-01A-01D-1462-08 | RCC | 36d21be3-2f46-47af-84aa-2305f2513aa1 |
| TCGA-BP-4961-01A | RCC | f207131d-8db7-464b-a3e5-44218da1cafc |
| TCGA-BP-4962-01A-01D-1462-08 | RCC | 3454a6fe-2547-4531-a0be-cb27c1879e72 |
| TCGA-BP-4963-01A-01D-1462-08 | RCC | 154bfa5d-0d9a-40c6-a2a5-bde1054702c3 |
| TCGA-BP-4964-01A-01D-1462-08 | RCC | 5b838251-67f5-4e22-a291-8a9e206d56db |
| TCGA-BP-4967-01A-01D-1462-08 | RCC | 75866d14-47d5-4560-a5a0-32ba3e15ac63 |
| TCGA-BP-4968-01A-01D-1462-08 | RCC | d777d5ec-4632-446e-aeac-8ae3e5273fe2 |
| TCGA-BP-4970-01A-01D-1462-08 | RCC | 205e81c6-235a-450f-b1f8-80c518eb3478 |
| TCGA-BP-4971-01A-01D-1462-08 | RCC | c07945e8-8133-4237-9d1f-18c023bc9d2c |
| TCGA-BP-4972-01A-01D-1462-08 | RCC | b2da5d39-33f6-4807-9d1d-92b7cef2a8df |
| TCGA-BP-4973-01A-01D-1462-08 | RCC | 5db95dcc-97e3-42a5-87dd-75a09b9c164a |
| TCGA-BP-4974-01A | RCC | a75c92b2-c67b-42b5-a8c2-7eea1b567ed0 |
| TCGA-BP-4975-01A-01D-1462-08 | RCC | 109d2752-17f8-4b00-a61f-dfd8e2e3ca81 |
| TCGA-BP-4976-01A-01D-1462-08 | RCC | 95bd81ec-3c06-4c4d-9915-5cc3dd7a7155 |
| TCGA-BP-4977-01A-01D-1462-08 | RCC | 7c3bf7c1-07d9-4540-9a5e-614fd60b63ec |
| TCGA-BP-4981-01A-01D-1462-08 | RCC | 64a1f085-50cc-4129-a617-e0f691a58039 |
| TCGA-BP-4982-01A-01D-1462-08 | RCC | 84591a73-bed0-4ad5-9acd-8f31acf27af0 |
| TCGA-BP-4983-01A-01D-1462-08 | RCC | beaafdf9-d5c0-4bc4-b08b-833c3c91c9ae |
| TCGA-BP-4985-01A-01D-1462-08 | RCC | e56acfea-aec6-4102-8fe0-25df396c10ae |
| TCGA-BP-4986-01A-01D-1462-08 | RCC | 4465171a-d048-4078-b1ae-021b2c635ff4 |
| TCGA-BP-4987-01A-01D-1462-08 | RCC | 7924f8ff-8e78-4910-9dc5-db14d5ee7011 |
| TCGA-BP-4988-01A-01D-1462-08 | RCC | 792c9867-ceea-4520-bbb7-5dabe290664f |
| TCGA-BP-4989-01A-01D-1462-08 | RCC | 7096085b-cd5b-4cd1-8957-a6adcf7e818a |
| TCGA-BP-4991-01A-01D-1462-08 | RCC | d54c714e-b1c4-4669-986d-5e13d2fc3cc3 |
| TCGA-BP-4992-01A | RCC | 212717dd-25f1-4c76-a648-b8a7d65caecf |
| TCGA-BP-4993-01A-02D-1421-08 | RCC | 34315bea-6ef2-42ec-b17e-c73eed40647f |
| TCGA-BP-4995-01A-01D-1462-08 | RCC | 93b9afac-e12e-49d2-96ac-274da6581d76 |
| TCGA-BP-4998-01A-01D-1462-08 | RCC | e646f930-967b-43a3-bd70-184e5c38efe5 |
| TCGA-BP-4999-01A-01D-1462-08 | RCC | 86ffb814-7c65-426b-b7b5-7250322c4d01 |
| TCGA-BP-5000-01A-01D-1462-08 | RCC | b9816eaa-3c60-4fbf-abd6-6d869ca9cca7 |
| TCGA-BP-5001-01A | RCC | e863bd35-0382-4979-b599-033a06a1f50b |
| TCGA-BP-5004-01A-01D-1462-08 | RCC | e3d82fe4-b491-4172-86da-429cf16508de |
| TCGA-BP-5006-01A-01D-1462-08 | RCC | 11fb962b-b4b8-46f4-bde4-3f87309e94f3 |
| TCGA-BP-5007-01A | RCC | a44eb1d6-3b5c-42e8-b17a-d71ffc0503d5 |
| TCGA-BP-5008-01A | RCC | 41c094e9-6c23-4993-8d90-338b66efefc1 |
| TCGA-BP-5009-01A-01D-1462-08 | RCC | 3baa3cdc-c63e-4556-baf1-c3b03175b0fa |
| TCGA-BP-5010-01A-02D-1421-08 | RCC | 553cbe18-6dd3-4b34-b7fe-96a6dd2e6943 |
| TCGA-BP-5168-01A-01D-1421-08 | RCC | 9930560d-22e6-43aa-a6f0-02515f7af8f0 |
| TCGA-BP-5169-01A-01D-1429-08 | RCC | 3527b21e-972b-4c31-b5de-8c394ce0e500 |
| TCGA-BP-5170-01A-01D-1429-08 | RCC | 68761b2c-66b9-4adf-9b60-955f79ed0f11 |
| TCGA-BP-5173-01A-01D-1429-08 | RCC | 3ce0a5fc-09ae-412a-8a5b-56d9a44433aa |
| TCGA-BP-5174-01A-01D-1429-08 | RCC | 53b5cf8d-f3cf-4e7e-91ec-b0c907d1c13f |
| TCGA-BP-5175-01A-01D-1429-08 | RCC | 30e58a1e-e7db-43ce-a7e8-a1fd21f4438e |
| TCGA-BP-5176-01A-01D-1429-08 | RCC | 607eb48b-1647-4e35-ac60-f6c50341e304 |
| TCGA-BP-5177-01A-01D-1429-08 | RCC | ad4cc7e3-c4d1-4cc0-9c93-33b47dadaaae |
| TCGA-BP-5178-01A-01D-1429-08 | RCC | 60888dc5-1408-4bfb-bf27-f3e22f5488e4 |
| TCGA-BP-5180-01A-01D-1429-08 | RCC | a776bde5-7503-459c-8419-dc0d744a651e |
| TCGA-BP-5182-01A-01D-1429-08 | RCC | 00523547-da1c-4bb1-a627-c0946849b376 |
| TCGA-BP-5183-01A-01D-1429-08 | RCC | cd4c37c3-95f2-4612-b6a8-9d6d1dfb5fd4 |
| TCGA-BP-5184-01A-01D-1429-08 | RCC | ddebed14-f47f-46e6-ac39-c74ed3363211 |
| TCGA-BP-5185-01A-01D-1429-08 | RCC | 42dc6d82-f52a-4b13-b3bc-c63002b47e98 |
| TCGA-BP-5186-01A-01D-1429-08 | RCC | 02b98f85-07df-4fb2-b27e-efd368c84ec8 |
| TCGA-BP-5187-01A | RCC | 3257e690-9306-434f-b6ac-17da58ab1243 |
| TCGA-BP-5189-01A-02D-1429-08 | RCC | ca98342a-65ec-468a-9cc1-44c7d31a67d6 |
| TCGA-BP-5190-01A-01D-1429-08 | RCC | 5491645b-552c-47a9-b081-e8e508d1df3d |
| TCGA-BP-5191-01A-01D-1429-08 | RCC | 64dd8a08-483e-4dce-90b0-64a751fdbebd |
| TCGA-BP-5192-01A-01D-1429-08 | RCC | 4db23b76-46dd-4ed9-a168-fee43b2fc7d7 |
| TCGA-BP-5194-01A-02D-1429-08 | RCC | 5b52c97e-fdd2-4ae2-b036-297feeb1c7e2 |
| TCGA-BP-5195-01A-02D-1429-08 | RCC | c2ab2f01-3744-434a-b5b6-0f22599c9a17 |
| TCGA-BP-5196-01A-01D-1429-08 | RCC | 201bf07d-0be9-442f-ad66-15ea8c7e812d |
| TCGA-BP-5198-01A-01D-1429-08 | RCC | ac66d658-97d4-416b-8028-0077a1c8a01d |
| TCGA-BP-5199-01A-01D-1429-08 | RCC | 135f3b77-1474-40d8-87a1-15939136e8cd |
| TCGA-BP-5200-01A | RCC | e2557bba-b331-40c2-8389-c52324630bca |
| TCGA-BP-5201-01A-01D-1429-08 | RCC | 243c77a9-1591-45ac-b048-a5687a77c764 |
| TCGA-BP-5202-01A-02D-1429-08 | RCC | accc7214-d441-4a72-a2eb-9f2811c38a3e |
| TCGA-CJ-4634-01A-02D-1386-10 | RCC | 59f18fac-c6f8-4cbf-9259-8c22d6ba0c58 |
| TCGA-CJ-4636-01A | RCC | 5889076d-0a5f-4c3a-8254-a941df3186f7 |
| TCGA-CJ-4637-01A-02D-1386-10 | RCC | b8480571-ee08-4fa1-b509-1331a8fbc075 |
| TCGA-CJ-4638-01A-02D-1386-10 | RCC | cbc187b0-fafe-4b1f-9af0-6714942414ab |
| TCGA-CJ-4639-01A-02D-1386-10 | RCC | 9df6d1b1-5a09-4082-8ec0-61b12b3c8801 |
| TCGA-CJ-4640-01A-02D-1386-10 | RCC | e406036a-eecb-474e-8c76-0fa8b64225be |
| TCGA-CJ-4641-01A-02D-1386-10 | RCC | c00265ac-c6cc-4349-ac30-e2e44582015a |
| TCGA-CJ-4643-01A-02D-1386-10 | RCC | 5e00e420-94fd-4115-9cd9-cef24f6df0eb |
| TCGA-CJ-4644-01A-02D-1386-10 | RCC | 2f2888fb-ae20-4347-87dc-f0eeeeb9b0d5 |
| TCGA-CJ-4882-01A-02D-1429-08 | RCC | b1b7b8e8-cc87-4a52-900a-1f3ef7d449d7 |
| TCGA-CJ-4897-01A-03D-1429-08 | RCC | c1331eec-e2df-4924-918b-7e5134e933c2 |
| TCGA-CJ-4899-01A-01D-1462-08 | RCC | 943ca428-39f6-4ad2-8ca5-220628a6b5bb |
| TCGA-CJ-4901-01A-01D-1429-08 | RCC | a8a8f3ff-0514-4bca-be75-16ad58eb9e72 |
| TCGA-CJ-4902-01A-01D-1429-08 | RCC | 3ef9ea62-85c4-4261-af23-ecb86f192cdf |
| TCGA-CJ-4903-01A-01D-1429-08 | RCC | 3b685193-f1fa-4c1b-949b-bcdb2d1b934c |
| TCGA-CJ-4904-01A-02D-1429-08 | RCC | 9bedcded-0c33-4199-bdce-18681595c2d8 |
| TCGA-CJ-4905-01A-02D-1429-08 | RCC | 22eb9dc5-8d5e-4158-8edc-12ff62a612be |
| TCGA-CJ-4907-01A-01D-1429-08 | RCC | 7c69fcb9-4b94-478a-bcb3-6ebd162d9482 |
| TCGA-CJ-4908-01A-01D-1429-08 | RCC | dbc5420c-5c60-4d1e-8554-9d2f6e55c502 |
| TCGA-CJ-4912-01A-01D-1429-08 | RCC | 894ade93-8feb-4f93-a31a-d9e16eb81743 |
| TCGA-CJ-4913-01A-01D-1429-08 | RCC | 0635f266-c4be-45ea-8347-455ef7ad5648 |
| TCGA-CJ-4916-01A-01D-1429-08 | RCC | 81b0e02c-069c-4c4b-b56f-79c2ebec9927 |
| TCGA-CJ-4918-01A-01D-1429-08 | RCC | 2c5d4600-0271-4c03-ab44-239ac19d8b4d |
| TCGA-CJ-4920-01A-01D-1429-08 | RCC | 12bf3338-f541-45a9-9fb7-e84931ba5ed8 |
| TCGA-CJ-4923-01A-01D-1429-08 | RCC | 19171a1a-6483-4bf3-b0b4-8cd441303c55 |
| TCGA-CJ-5671-01A-11D-1534-10 | RCC | 5b1084bb-3fb2-4f3f-9ca7-7108b0f77994 |
| TCGA-CJ-5672-01A-11D-1534-10 | RCC | 61497c42-78f2-43d4-b2ab-2b1e655271a8 |
| TCGA-CJ-5675-01A | RCC | 26f77108-c3b0-4833-9a1a-df457d7415a9 |
| TCGA-CJ-5676-01A-11D-1534-10 | RCC | 2e8aa293-650b-4661-b130-8b70f0949b86 |
| TCGA-CJ-5677-01A-11D-1534-10 | RCC | 70fe0b18-52d1-40f7-b2a3-c808b3009610 |
| TCGA-CJ-5678-01A-11D-1534-10 | RCC | d49759a2-d2a9-48ba-9447-e42c9d3d64c7 |
| TCGA-CJ-5679-01A | RCC | 17313700-6052-4901-8850-981fead99d6c |
| TCGA-CJ-5680-01A-11D-1534-10 | RCC | 2c718814-9d25-49a6-a430-2019071ec0ab |
| TCGA-CJ-5681-01A-11D-1534-10 | RCC | 9ae0744a-9bc1-4cd7-b7cf-c6569ed9e4aa |
| TCGA-CJ-5682-01A-11D-1534-10 | RCC | deceb0ba-600f-491a-a207-2e0205ff89d2 |
| TCGA-CJ-5683-01A-11D-1534-10 | RCC | b85e29c5-0206-4d65-aa46-179a55c0ceae |
| TCGA-CJ-5684-01A-11D-1534-10 | RCC | 24ee4b71-c2e0-44c3-aaeb-3c488cd26ce7 |
| TCGA-CJ-5686-01A-11D-1669-08 | RCC | 695e2a72-6b97-4fa1-9f57-d7c6e10438ee |
| TCGA-CJ-6027-01A-11D-1669-08 | RCC | b0483455-4cde-408f-b831-17223c03241a |
| TCGA-CJ-6028-01A-11D-1669-08 | RCC | d165717a-cc3d-4533-8194-0029c186f1bb |
| TCGA-CJ-6030-01A-11D-1669-08 | RCC | c904299c-09a8-4a4c-9378-2fee0ac4cd33 |
| TCGA-CJ-6031-01A-11D-1669-08 | RCC | a47debc7-700e-4c64-a9b3-1113609a1ddf |
| TCGA-CJ-6032-01A-11D-1669-08 | RCC | 8c9823f0-69af-474d-adb7-5ec8ef4e5af7 |
| TCGA-CJ-6033-01A-11D-1669-08 | RCC | c7ce9042-f63c-4a93-a82d-f21977bd9bcb |
| TCGA-CW-5580-01A-01D-1669-08 | RCC | 6e4ed3ae-aa80-453a-95be-0af96a7bc4e3 |
| TCGA-CW-5581-01A | RCC | 22be4bab-231e-4784-aaa9-45ae158a5153 |
| TCGA-CW-5583-01A-02D-1534-10 | RCC | 2cb6b578-8543-4a12-8331-1721ddc47303 |
| TCGA-CW-5585-01A-01D-1534-10 | RCC | bd6d9aa8-d0ef-4810-a43c-eacdd846c44e |
| TCGA-CW-5591-01A-01D-1534-10 | RCC | 02ac80cd-caa3-4dbc-9b57-4a324cec0ad4 |
| TCGA-CW-6087-01A-11D-1669-08 | RCC | 65c23a97-1763-47d5-8648-df24cf0226f3 |
| TCGA-CW-6090-01A-11D-1669-08 | RCC | 3b2e654a-4c13-4dab-9e18-1445a43af3e6 |
| TCGA-CW-6093-01A-11D-1669-08 | RCC | 9b1beb37-1ed7-43c0-a532-56df7941111f |
| TCGA-CZ-4853-01A-01D-1429-08 | RCC | bdef62d1-a036-43b4-811b-bf4beab7eca8 |
| TCGA-CZ-4856-01A-02D-1429-08 | RCC | 85e26450-4cb1-4a91-ad86-a6d44890ee97 |
| TCGA-CZ-4859-01A-02D-1429-08 | RCC | 82c0b6e4-cb0f-4870-81c9-b45a93d6f5d3 |
| TCGA-CZ-4863-01A-01D-1501-10 | RCC | 4286d73b-1fb9-41a3-baba-46f23100586a |
| TCGA-CZ-4865-01A-02D-1501-10 | RCC | f8eac30d-1155-44cc-a2ad-95427fecf4bf |
| TCGA-CZ-4866-01A-01D-1501-10 | RCC | a3a06421-7838-4ac2-b5d5-45d2ea651368 |
| TCGA-CZ-5451-01A-01D-1501-10 | RCC | b1923d68-1d1e-4b59-b643-09e2c5969efd |
| TCGA-CZ-5452-01A-01D-1501-10 | RCC | 96bd68cb-5d8e-4de1-88ca-5f30fbdde036 |
| TCGA-CZ-5453-01A-01D-1501-10 | RCC | 605079f6-2d6e-4c38-a214-b4c8875dd166 |
| TCGA-CZ-5454-01A-01D-1501-10 | RCC | d9fd1928-7b7d-4147-aeff-1618393ba26c |
| TCGA-CZ-5455-01A | RCC | d6a730ef-3f0d-47c1-977e-5c80647356d4 |
| TCGA-CZ-5456-01A-01D-1501-10 | RCC | 45d5c746-60e3-4531-8db0-fd648811d45f |
| TCGA-CZ-5457-01A | RCC | 8d54b22b-ee4b-45e0-922e-24e3c20c4c1a |
| TCGA-CZ-5458-01A-01D-1501-10 | RCC | 1737382a-a1c9-45e1-b009-a29be1d93749 |
| TCGA-CZ-5459-01A-01D-1501-10 | RCC | 5711cdaa-7368-4a4f-8639-5df60a2fedac |
| TCGA-CZ-5460-01A-01D-1501-10 | RCC | a6de1551-2a1a-4a43-ba7f-caa436f5f6dd |
| TCGA-CZ-5461-01A-01D-1501-10 | RCC | 79feee74-7b14-48d9-9be7-8d7671c79c83 |
| TCGA-CZ-5462-01A-01D-1501-10 | RCC | 74eed0c6-b3cc-4666-8ef0-194e1bbe1048 |
| TCGA-CZ-5463-01A-01D-1501-10 | RCC | 3732539b-eb77-485b-81a1-83be956a9a87 |
| TCGA-CZ-5465-01A-01D-1806-10 | RCC | 062b7e63-bb4e-4eaa-9aa4-f2af44c2ab37 |
| TCGA-CZ-5466-01A | RCC | 694ca445-7bac-4216-acf5-e227650ae973 |
| TCGA-CZ-5467-01A-01D-1501-10 | RCC | 99c640a3-660f-4723-bf82-36fcb3134356 |
| TCGA-CZ-5468-01A-01D-1501-10 | RCC | 50c6b5a2-cd0e-4adf-b85f-0f9c1847477f |
| TCGA-CZ-5469-01A-01D-1501-10 | RCC | 3df654a0-48b0-45ff-bfe1-b5f78f63b30d |
| TCGA-CZ-5470-01A-01D-1501-10 | RCC | c9a7ca9e-c36e-46c1-926f-4a57a0584cb0 |
| TCGA-CZ-5982-01A-11D-1669-08 | RCC | 2c3c0f78-1c0a-48df-856e-0afbc2b5bceb |
| TCGA-CZ-5984-01A-11D-1669-08 | RCC | 89e8e486-0c93-4056-88ed-83fd0d5a7f2c |
| TCGA-CZ-5985-01A-11D-1669-08 | RCC | ad5eae3d-2f73-49d2-be47-5891e7772bc6 |
| TCGA-CZ-5986-01A-11D-1669-08 | RCC | 0abded91-5a5f-4923-bcf0-7fdda64ae232 |
| TCGA-CZ-5987-01A-11D-1669-08 | RCC | 84a1a8d2-54c6-4771-9092-27c5f7fc4e5c |
| TCGA-CZ-5988-01A-11D-1669-08 | RCC | 668172b3-1e6f-4362-8432-3651925b86a6 |
| TCGA-CZ-5989-01A-11D-1669-08 | RCC | 852e1614-35c0-4ba7-a29c-e8e2a91aa1b7 |
| TCGA-DV-5565-01A-01D-1534-10 | RCC | ee24d408-6043-4ca0-8bde-f29e798cc479 |
| TCGA-DV-5566-01A-01D-1534-10 | RCC | 39a321cd-dbdf-474b-aead-6e69795470e0 |
| TCGA-DV-5568-01A-01D-1534-10 | RCC | ecb100d4-24da-40d9-aee1-2901cf3a655a |
| TCGA-EU-5904-01A-11D-1669-08 | RCC | b13e89f1-683b-4261-94a1-e371d797237f |
| TCGA-EU-5905-01A-11D-1669-08 | RCC | 091c18b6-bfc2-4353-9eba-ebc46c2c18c5 |
| TCGA-EU-5906-01A-11D-1669-08 | RCC | 050dc3b7-e560-44f4-a05c-8c792d8467a8 |
| TCGA-EU-5907-01A-11D-1669-08 | RCC | 5fded36e-05ba-4cce-8303-738f5b04ad16 |
| TCGA-AB-2807-03D-01W-0755-09 | AML | 3d15bdda-bbb7-4e3d-bdd6-7546d2905e95 |
| TCGA-AB-2809-03D-01W-0755-09 | AML | d86f567d-84f8-4a95-af1d-5a26ada92830 |
| TCGA-AB-2814-03D-01W-0755-09 | AML | 604f0c72-efc7-4868-bc54-79d8f3f3507b |
| TCGA-AB-2822-03D-01W-0755-09 | AML | 68b67026-2f30-4839-8579-7a07341b8976 |
| TCGA-AB-2825-03D-01W-0755-09 | AML | e6e4b579-9ddf-4fb1-bb65-db8321294852 |
| TCGA-AB-2840-03D-01W-0755-09 | AML | cb122429-5b01-4fad-b498-b0342230b567 |
| TCGA-AB-2845-03D-01W-0755-09 | AML | 98d27719-6f38-433a-ba0a-a14cb32958d8 |
| TCGA-AB-2853-03D-01W-0755-09 | AML | 9e238bbc-61ba-4966-b30e-ba7ab1a5b11b |
| TCGA-AB-2858-03D-01W-0755-09 | AML | b9dcb0aa-0098-49a9-a0c8-790a06dadea8 |
| TCGA-AB-2863-03D-01W-0755-09 | AML | d4ba0ac2-9d98-430b-bb0d-e1bada2d5486 |
| TCGA-AB-2864-03D-01W-0755-09 | AML | 07f07406-597d-40b7-b218-ef40aad6f0bc |
| TCGA-AB-2872-03A-01W-0732-08 | AML | 495c3e6d-76f1-499d-894b-761d50b70566 |
| TCGA-AB-2909-03A-01W-0755-09 | AML | 39ad6508-a476-4a33-ae8d-6e25fa36369e |
| TCGA-AB-2912-03A-01W-0732-08 | AML | da01cad7-961b-46e2-8a80-9c846694ad5b |
| TCGA-AB-2918-03A-01W-0745-08 | AML | d0833641-77a1-41fd-b635-d216b00d007b |
| TCGA-AB-2921-03A-01W-0755-09 | AML | 779697fe-899a-4bfb-a1d3-44a847487b6b |
| TCGA-AB-2926-03A-01W-0732-08 | AML | 890ea799-3156-40c3-839c-0c60179006d7 |
| TCGA-AB-2927-03A-01W-0755-09 | AML | 46bbb19d-2bc9-4f0a-ac4e-cad7327ca142 |
| TCGA-AB-2934-03A-01W-0755-09 | AML | 7791e140-fe03-44d0-8250-47826ea993df |
| TCGA-AB-2946-03A-01W-0755-09 | AML | f24b41b4-79bf-4736-96c8-83921811bb95 |
| TCGA-AB-2948-03A-01W-0755-09 | AML | 7b0fb197-8465-430b-9da7-322f2d218729 |
| TCGA-05-4244-01A-01D-1105-08 | LUAD | 738c514d-ff0f-4220-9326-236119891df5 |
| TCGA-05-4249-01A-01D-1105-08 | LUAD | 8be717b5-5b65-4631-a175-1f4c063d447e |
| TCGA-05-4250-01A-01D-1105-08 | LUAD | 41c4fe84-8beb-4a3a-920c-e74c7edd2182 |
| TCGA-05-4382-01A-01D-1265-08 | LUAD | 005b918d-e4a9-4971-9588-656a35c33dec |
| TCGA-05-4384-01A-01D-1753-08 | LUAD | 4c71b66b-813f-472b-b866-b34b5b9199e7 |
| TCGA-05-4389-01A-01D-1265-08 | LUAD | c6f382d4-a522-4333-88b5-be7f55fe80f5 |
| TCGA-05-4390-01A-02D-1753-08 | LUAD | d0854b5b-69be-4b84-aa37-ecdd0bc14de9 |
| TCGA-05-4395-01A-01D-1265-08 | LUAD | dc45b4de-4c03-4fe4-89e0-d1cf378084b6 |
| TCGA-05-4396-01A-21D-1855-08 | LUAD | 0176cf1d-0760-4769-a493-277f4bb7585e |
| TCGA-05-4397-01A-01D-1265-08 | LUAD | 4b7be121-49af-4a44-95dd-0a487d47228f |
| TCGA-05-4398-01A-01D-1265-08 | LUAD | 9e4b2be6-e149-4c22-93e1-512c3c6bbea8 |
| TCGA-05-4402-01A-01D-1265-08 | LUAD | 75475a84-582d-4949-a428-1e28ad526d8c |
| TCGA-05-4403-01A-01D-1265-08 | LUAD | 7e25ac0e-94e4-42f6-ae6f-89d0d21ce09f |
| TCGA-05-4405-01A-21D-1855-08 | LUAD | 3ef10eb8-d713-4fda-9e03-bc594b356d77 |
| TCGA-05-4410-01A-21D-1855-08 | LUAD | f85d0d42-436b-4251-a7fd-7d0f5fddd397 |
| TCGA-05-4415-01A-22D-1855-08 | LUAD | 128f52c7-49dc-4a9f-a5bc-1c14684edc9c |
| TCGA-05-4417-01A-22D-1855-08 | LUAD | 57e3657d-7a3c-4d80-a2c2-2de0293f5f05 |
| TCGA-05-4418-01A-01D-1265-08 | LUAD | b07397ae-592b-4eb4-98b3-7c7e81ecb5e0 |
| TCGA-05-4420-01A-01D-1265-08 | LUAD | 0536b000-eaf3-4cb2-b46b-8dd9f23c8199 |
| TCGA-05-4422-01A-01D-1265-08 | LUAD | a5370f18-e8a9-43d8-9eb8-be678ccd4669 |
| TCGA-05-4424-01A-22D-1855-08 | LUAD | fc500ff5-24c8-4965-94da-b4afafafe2dd |
| TCGA-05-4425-01A-01D-1753-08 | LUAD | 4a367804-9934-4241-90da-0ba0245564bd |
| TCGA-05-4426-01A-01D-1265-08 | LUAD | 117c6aff-8899-48f4-9328-746207d38eff |
| TCGA-05-4427-01A-21D-1855-08 | LUAD | 736e0134-8b1a-4ff1-9106-ca09c9812ef6 |
| TCGA-05-4430-01A-02D-1265-08 | LUAD | 23398531-3f4c-45e6-980b-755165c04974 |
| TCGA-05-4432-01A-01D-1265-08 | LUAD | 377ab4af-0958-4b8b-ac0c-4cd49c1e4c2e |
| TCGA-05-4433-01A-22D-1855-08 | LUAD | fab4f1ca-1605-4c30-8b3e-badb44eb6580 |
| TCGA-05-4434-01A-01D-1265-08 | LUAD | f529778c-5968-4d87-80c0-bd14ba2311d0 |
| TCGA-05-5420-01A-01D-1625-08 | LUAD | 8371b6a4-ffea-4fe5-b997-76ece85064a7 |
| TCGA-05-5423-01A-01D-1625-08 | LUAD | 209d853d-6c50-4223-a572-a90d58aee51e |
| TCGA-05-5425-01A-02D-1625-08 | LUAD | 70a3e96b-dd26-419c-9a68-97dea0465d6e |
| TCGA-05-5428-01A-01D-1625-08 | LUAD | 7744a93b-0565-4d83-afad-caa02358f258 |
| TCGA-05-5429-01A-01D-1625-08 | LUAD | 37d0cf1b-1743-4852-8073-372b16b5c17d |
| TCGA-05-5715-01A-01D-1625-08 | LUAD | 62fda17b-1de0-4b7e-bd28-a6793bc36d37 |
| TCGA-17-Z000-01A-01W-0746-08 | LUAD | ba9d9630-fc6c-4ffb-8464-c1a2ddec6579 |
| TCGA-17-Z001-01A-01W-0746-08 | LUAD | d5e77555-9412-4e64-a6aa-65c996e3d521 |
| TCGA-17-Z003-01A-01W-0746-08 | LUAD | 443d768f-b871-4149-9ef0-2d49bc0d05a1 |
| TCGA-17-Z004-01A-01W-0746-08 | LUAD | c1a70a4b-2879-48e8-87e1-b02c57d58705 |
| TCGA-17-Z005-01A-01W-0746-08 | LUAD | 96b0bb86-6092-47ef-8088-fd0a4f261439 |
| TCGA-17-Z007-01A-01W-0746-08 | LUAD | cac5bcd1-f044-4275-89cd-1110d0025537 |
| TCGA-17-Z008-01A-01W-0746-08 | LUAD | 6b6ddf99-f050-4dfa-85a2-d5a3e3ad56b0 |
| TCGA-17-Z009-01A-01W-0746-08 | LUAD | 8c5a3460-c1fa-4b7b-9b31-11f9c7b03255 |
| TCGA-17-Z010-01A-01W-0746-08 | LUAD | c9fb7916-74d0-4266-b5b8-705018e0e76b |
| TCGA-17-Z011-01A-01W-0746-08 | LUAD | d7495a00-b312-4502-9e1b-9e5f3dbf4b5d |
| TCGA-17-Z012-01A-01W-0746-08 | LUAD | 861e9d45-df9a-41a6-9ddf-bc72f85aed80 |
| TCGA-17-Z013-01A-01W-0746-08 | LUAD | ee0cbaf2-a0bb-4e58-9e52-5986b5f4f25e |
| TCGA-17-Z014-01A-01W-0746-08 | LUAD | ca24ac3d-4686-4f0c-a47d-0eff92a623b1 |
| TCGA-17-Z015-01A-01W-0746-08 | LUAD | 770c22ba-b759-433e-8478-b6cf0d685447 |
| TCGA-17-Z016-01A-01W-0746-08 | LUAD | 39bbd67b-52fd-46e5-98cf-b5632400216d |
| TCGA-17-Z017-01A-01W-0746-08 | LUAD | 37049bf1-55cb-44d3-b673-1e270ea835f7 |
| TCGA-17-Z018-01A-01W-0746-08 | LUAD | dd1a61eb-8362-41a9-952d-b7e6887457ad |
| TCGA-17-Z020-01A-01W-0746-08 | LUAD | 7ea20aa3-68cf-4389-9ace-99d6149d16c1 |
| TCGA-17-Z021-01A-01W-0746-08 | LUAD | 9394b536-cd08-414b-86a3-c6491f967709 |
| TCGA-17-Z022-01A-01W-0746-08 | LUAD | 7f07e5b3-bf70-4690-84ba-a9eace798a24 |
| TCGA-17-Z023-01A-01W-0746-08 | LUAD | bd72330a-463f-471b-9eba-2f188524e74c |
| TCGA-17-Z025-01A-01W-0746-08 | LUAD | 99eab29e-32d3-49d5-aa30-56de8be556e7 |
| TCGA-17-Z026-01A-01W-0746-08 | LUAD | bb048ffc-de00-4706-85bb-d052c0fb6496 |
| TCGA-17-Z027-01A-01W-0746-08 | LUAD | 880452fe-00ed-4732-bbcf-14b55c235e61 |
| TCGA-17-Z028-01A-01W-0746-08 | LUAD | 1f55fb6e-342a-41e0-9a8e-7c5156c95eaa |
| TCGA-17-Z030-01A-01W-0746-08 | LUAD | e35e27e8-6cc5-495b-9ae8-89f65d94ebed |
| TCGA-17-Z031-01A-01W-0746-08 | LUAD | 6516244a-dfd8-4568-a2d2-7556cbea52b1 |
| TCGA-17-Z032-01A-01W-0746-08 | LUAD | 92bc438b-02c1-4b81-a90a-4a1302786a81 |
| TCGA-17-Z033-01A-01W-0746-08 | LUAD | 639aea7c-5a38-4641-bf0d-90a9ce8e2980 |
| TCGA-17-Z035-01A-01W-0746-08 | LUAD | a4bcbb2e-594f-4a89-8b72-8c922a64cdef |
| TCGA-17-Z036-01A-01W-0746-08 | LUAD | 374b881a-dbe2-4b4b-bfc0-8431f1aec06c |
| TCGA-17-Z037-01A-01W-0746-08 | LUAD | bffe237d-31b0-4950-a7ab-4ac7047aa3c0 |
| TCGA-17-Z038-01A-01W-0746-08 | LUAD | 8785c362-1c4d-41da-a29e-5cff21dc2a2e |
| TCGA-17-Z040-01A-01W-0746-08 | LUAD | 62d2ca54-b8e0-4907-b75e-cb9786069b52 |
| TCGA-17-Z041-01A-01W-0746-08 | LUAD | c0ead7c7-169e-4932-a987-5461611c95e6 |
| TCGA-17-Z042-01A-01W-0746-08 | LUAD | 3c303c9d-6cda-490d-a64d-21bc40b064f3 |
| TCGA-17-Z043-01A-01W-0746-08 | LUAD | 577f7267-c568-4002-a153-26c09d1eca97 |
| TCGA-17-Z044-01A-01W-0746-08 | LUAD | cb1aaeb8-0c6f-4266-968c-38a3823d85f6 |
| TCGA-17-Z045-01A-01W-0746-08 | LUAD | ec7e65c5-7158-427f-8034-8616077da50b |
| TCGA-17-Z046-01A-01W-0746-08 | LUAD | 7aac0e3f-39fe-4c9a-9482-50f02f1b919d |
| TCGA-17-Z047-01A-01W-0747-08 | LUAD | 2c04cfa8-6e99-46fa-82ad-36fb96e5ffef |
| TCGA-17-Z048-01A-01W-0746-08 | LUAD | 8495e150-796b-4e15-9fa6-1fba558d7b10 |
| TCGA-17-Z049-01A-01W-0746-08 | LUAD | ac31bcc6-6ccc-43b7-96f2-3ab47050be76 |
| TCGA-17-Z050-01A-01W-0747-08 | LUAD | d086dd38-a9e0-466c-b1e5-9a4a879abd55 |
| TCGA-17-Z051-01A-01W-0747-08 | LUAD | 5584878f-0608-45d9-8e28-29c277bf655f |
| TCGA-17-Z052-01A-01W-0747-08 | LUAD | afdf7c82-2a17-4c73-980c-74ec822dc803 |
| TCGA-17-Z053-01A-01W-0747-08 | LUAD | 2d422986-6e91-4299-b6cf-4076f3706c83 |
| TCGA-17-Z054-01A-01W-0747-08 | LUAD | 409dd077-dab9-4f79-9c33-2c3b75b63125 |
| TCGA-17-Z055-01A-01W-0747-08 | LUAD | de78326d-3afc-4f29-af7f-1750da544826 |
| TCGA-17-Z056-01A-01W-0747-08 | LUAD | e6cb3d63-5a55-4eba-84d2-a25917c7b18e |
| TCGA-17-Z057-01A-01W-0747-08 | LUAD | 4236905d-1549-4cbc-b3c6-e62db9ea598b |
| TCGA-17-Z058-01A-01W-0747-08 | LUAD | 6b0b1fca-efce-49d6-9f7b-a2c34bb343e9 |
| TCGA-17-Z059-01A-01W-0747-08 | LUAD | 88ec6fb4-1b81-422e-8204-ef9e8dbf260c |
| TCGA-17-Z060-01A-01W-0747-08 | LUAD | f834dfa4-8d9c-4e0b-861f-a3cc31245237 |
| TCGA-17-Z061-01A-01W-0747-08 | LUAD | 1eb07e6e-6cf8-45e4-9b5c-1a9a5d38d117 |
| TCGA-17-Z062-01A-01W-0747-08 | LUAD | f3280e5f-7d6e-4a18-a5a5-e84b805c9e66 |
| TCGA-35-3615-01A | LUAD | 7407d705-6ec6-4143-93d2-eedcf5a22399 |
| TCGA-35-3621-01A-01D-0969-08 | LUAD | 4a0cc41a-562c-4aea-a7c3-b1186d46cda8 |
| TCGA-35-4122-01A-01D-1105-08 | LUAD | 408e1cb4-64a8-4801-bf58-3b8183ede851 |
| TCGA-35-4123-01A-01D-1105-08 | LUAD | 7ceccaee-df27-4f7f-bfcd-e1c59b365711 |
| TCGA-35-5375-01A-01D-1625-08 | LUAD | 63e76bef-3ef1-445f-b591-649d774729cd |
| TCGA-38-4625-01A-01D-1553-08 | LUAD | 6f317d31-c9a4-4345-b5b1-b75776536402 |
| TCGA-38-4626-01A-01D-1553-08 | LUAD | 85b56ce7-b420-433e-a77d-43ef628d685c |
| TCGA-38-4627-01A-01D-1553-08 | LUAD | abef97da-d7db-495f-b594-fa66577becd6 |
| TCGA-38-4628-01A-01D-1265-08 | LUAD | 67bc44b7-92cf-4e8f-a7f6-c53bf34a17c6 |
| TCGA-38-4629-01A-02D-1265-08 | LUAD | 4797f969-5f4d-4681-9fc5-68f25ba8f4d8 |
| TCGA-38-4630-01A-01D-1265-08 | LUAD | 2b139bb4-5a29-4684-901d-8d966ff79ac2 |
| TCGA-38-4631-01A-01D-1753-08 | LUAD | b3ffc36d-b0b8-4ada-a00a-b48890c0162c |
| TCGA-38-4632-01A-01D-1753-08 | LUAD | 83519ed1-29e2-4f1b-922c-5779f64178bc |
| TCGA-38-6178-01A-11D-1753-08 | LUAD | 7fa467f1-d928-4d81-bd0b-68d67a5c18cf |
| TCGA-44-2655-01A | LUAD | 9fcabda1-ea79-4188-8b3f-7d0fd060a819 |
| TCGA-44-2656-01A | LUAD | 5593f581-3d45-4a4a-a525-bfae1f4753a0 |
| TCGA-44-2657-01A-01D-1105-08 | LUAD | e3aa9b45-13b9-4b61-a30f-ae3f88466040 |
| TCGA-44-2661-01A-01D-1105-08 | LUAD | 3c3a2e7c-9aa0-495e-95c7-87f661b9ed92 |
| TCGA-44-2662-01A | LUAD | d2198941-e96f-40bd-9fbe-82886217d5db |
| TCGA-44-2665-01A | LUAD | a0863fa6-515c-44fa-825f-f9e243f945f1 |
| TCGA-44-2666-01A | LUAD | 27a64f32-69c5-4c49-86b4-c8fc923cae08 |
| TCGA-44-2668-01A | LUAD | dd9a6c68-b8b4-4168-9ff9-72a45f20c44f |
| TCGA-44-3396-01A-01D-1265-08 | LUAD | d68b216c-b304-4b30-9af7-eb3a9a1a55ae |
| TCGA-44-3398-01A-01D-1105-08 | LUAD | 82284bb3-2dfa-4016-a908-3b5994e00d31 |
| TCGA-44-3918-01A-01D-1105-08 | LUAD | 7f456c3f-58e3-43f1-9f76-4422451528a5 |
| TCGA-44-3919-01A | LUAD | 9de8d353-3442-41d8-8bfe-a08c4975eaca |
| TCGA-44-4112-01A | LUAD | 6c206676-e511-4281-91f5-bfe91b3279a4 |
| TCGA-44-5643-01A-01D-1625-08 | LUAD | 44286013-ae97-4890-86d3-1163285ac0cd |
| TCGA-44-5645-01A-01D-1625-08 | LUAD | dac33765-0c88-4a51-8389-c042ccb78c83 |
| TCGA-44-6144-01A-11D-1753-08 | LUAD | f19575fd-eb9d-429f-96ce-c0e8f4bbc593 |
| TCGA-44-6145-01A-11D-1753-08 | LUAD | 220dc947-4afc-4485-bcc7-cea046100b4b |
| TCGA-44-6146-01A-11D-1753-08 | LUAD | d5e90162-d7d2-4a7c-89f0-51c2b32c9ef0 |
| TCGA-44-6147-01A-11D-1753-08 | LUAD | 7b6daa70-492e-4283-b3d2-b26f4e26a8d4 |
| TCGA-44-6148-01A-11D-1753-08 | LUAD | 9c7b3ac8-1352-49cd-8a8c-df6b19f6fd64 |
| TCGA-44-6774-01A-21D-1855-08 | LUAD | f9cc1d71-bece-4693-b953-3e73d1b6c11c |
| TCGA-44-6775-01A-11D-1855-08 | LUAD | 7a70a44f-84f3-440a-b898-dc3a0eff748e |
| TCGA-44-6776-01A-11D-1855-08 | LUAD | 7d3c5101-fae2-4320-a8a2-a93753375368 |
| TCGA-44-6777-01A-11D-1855-08 | LUAD | 32a0f0f3-3879-4b96-b9bb-eeab87827f6e |
| TCGA-44-6778-01A-11D-1855-08 | LUAD | 903182ad-3145-4fa3-869e-62774aedf86c |
| TCGA-44-6779-01A-11D-1855-08 | LUAD | d6990a90-6a99-490b-a476-5298f0c4e4f2 |
| TCGA-49-4486-01A-01D-1265-08 | LUAD | 3ac132c3-4889-4dc3-8b3d-0ef98065a858 |
| TCGA-49-4487-01A-21D-1855-08 | LUAD | 9bd8e303-a81e-4ff8-882b-d46a2f7c55d2 |
| TCGA-49-4488-01A-01D-1753-08 | LUAD | 3635bb9c-a332-4445-ad81-83cec426dd02 |
| TCGA-49-4490-01A-21D-1855-08 | LUAD | 940455cf-aa91-432a-bc39-9dfba206e32b |
| TCGA-49-4494-01A-01D-1265-08 | LUAD | 136bc973-1908-4767-9b22-d43d522b7c71 |
| TCGA-49-4501-01A-01D-1265-08 | LUAD | 0c53bb1b-5e6f-44a8-97a0-f89d43e0e789 |
| TCGA-49-4505-01A-01D-1265-08 | LUAD | e773a2fe-1d80-492d-bba8-105036a14a92 |
| TCGA-49-4506-01A-01D-1265-08 | LUAD | d707f8ad-5ea5-493a-a745-9b5dba64f213 |
| TCGA-49-4507-01A-01D-1265-08 | LUAD | 562a09a1-b491-45c8-a87d-3c2471353c0d |
| TCGA-49-4510-01A-01D-1265-08 | LUAD | b2c12bff-addd-45a2-ada4-c30ac935809c |
| TCGA-49-4512-01A-21D-1855-08 | LUAD | fa6a60f5-8949-4e01-9435-d3117601627f |
| TCGA-49-4514-01A-21D-1855-08 | LUAD | 7751af67-1415-475e-8ec5-66d76f515014 |
| TCGA-49-6742-01A-11D-1855-08 | LUAD | 49dec0c2-8e75-4f44-a253-82b2ea605890 |
| TCGA-49-6743-01A-11D-1855-08 | LUAD | 545c9d29-a8e0-4d2d-8552-d27b46f96070 |
| TCGA-49-6744-01A-11D-1855-08 | LUAD | bf6ba698-7154-4d7f-b076-24ac2f768696 |
| TCGA-49-6745-01A-11D-1855-08 | LUAD | bfb97048-977b-4722-be8f-3dd37370ba30 |
| TCGA-49-6767-01A-11D-1855-08 | LUAD | 9f82f494-042a-4f00-954c-4761fa25b298 |
| TCGA-50-5044-01A-21D-1855-08 | LUAD | ec034986-4bf7-4554-b635-ca6d9c30da28 |
| TCGA-50-5045-01A-01D-1625-08 | LUAD | b0d734ad-1222-4bc0-b02b-1d2262b8ac35 |
| TCGA-50-5049-01A-01D-1625-08 | LUAD | 96358297-0735-4eab-a01c-a6be5d86a3de |
| TCGA-50-5051-01A-21D-1855-08 | LUAD | bb50bc27-fb18-4eee-8785-b8e8b69bcbe6 |
| TCGA-50-5055-01A-01D-1625-08 | LUAD | 12fe153e-a8f7-49ec-9e0c-f680e2311cf6 |
| TCGA-50-5066-01A-01D-1625-08 | LUAD | f5a97315-1906-4774-980e-0879c6ad368e |
| TCGA-50-5068-01A-01D-1625-08 | LUAD | c1efdc48-6ea5-45f0-9fa3-94c42ecf3ab4 |
| TCGA-50-5072-01A-21D-1855-08 | LUAD | 3c6dcba5-1312-40ca-b589-07f7d88b3477 |
| TCGA-50-5930-01A-11D-1753-08 | LUAD | bd3e88b3-b37c-4641-85fa-d8125ba324ca |
| TCGA-50-5931-01A-11D-1753-08 | LUAD | 290847c6-c9d4-4a16-a70f-0488e3718f35 |
| TCGA-50-5932-01A-11D-1753-08 | LUAD | 6726c157-f688-491d-8b56-35628645df89 |
| TCGA-50-5933-01A-11D-1753-08 | LUAD | cc3a9cfe-8a14-4fb4-a60f-3ec795c5d7a1 |
| TCGA-50-5935-01A-11D-1753-08 | LUAD | 9570cd02-3339-4805-855a-74ebe429df96 |
| TCGA-50-5936-01A-11D-1625-08 | LUAD | 82d380d5-4c07-4cf0-a6e9-7ca9e3fc9a08 |
| TCGA-50-5939-01A-11D-1625-08 | LUAD | aa9108d7-5036-4059-ad82-dc64161d5bc3 |
| TCGA-50-5941-01A-11D-1753-08 | LUAD | 86ef12c0-d5fc-4852-9960-593366e717b4 |
| TCGA-50-5942-01A-21D-1753-08 | LUAD | 95475c1b-086d-4e09-a871-47d8f76c1a07 |
| TCGA-50-5944-01A-11D-1753-08 | LUAD | a314ee0c-694b-4ac8-b572-ff1fbbda4765 |
| TCGA-50-5946-01A-11D-1753-08 | LUAD | 142d43e8-10e1-4945-a37c-f2824d53b122 |
| TCGA-50-6590-01A-12D-1855-08 | LUAD | 85de182b-f4ae-41e6-b3fb-f60f46c072e4 |
| TCGA-50-6591-01A-11D-1753-08 | LUAD | bf7462a2-394f-4838-bcb6-4d0126fa48b1 |
| TCGA-50-6592-01A-11D-1753-08 | LUAD | d0303d05-a937-4a7d-9934-ffa93cc1c5de |
| TCGA-50-6593-01A-11D-1753-08 | LUAD | 10e03053-f6e3-42b7-8638-ce58c6e7dfaa |
| TCGA-50-6594-01A-11D-1753-08 | LUAD | e1365c7d-e93e-4478-a8e9-ae2d7ca30bc6 |
| TCGA-50-6595-01A-12D-1855-08 | LUAD | 9913e506-fc98-467d-8601-89595d0475e8 |
| TCGA-50-6597-01A-11D-1855-08 | LUAD | cd0aeed5-93a1-4287-8a88-fe6b7b5e3983 |
| TCGA-55-1592-01A | LUAD | e190a9e4-10ae-4060-a071-4b8b73479023 |
| TCGA-55-1594-01A | LUAD | 2885d4b3-34a6-421d-b20c-eedad721d10a |
| TCGA-55-1595-01A-01D-0969-08 | LUAD | f1be8e08-5201-49bb-abf7-cedc0eff06d6 |
| TCGA-55-1596-01A | LUAD | 9a7a1b22-9df6-438f-ad00-54755c7dbc7c |
| TCGA-55-5899-01A-11D-1625-08 | LUAD | ddaf36f7-7503-4ab4-b7f5-9777c0c1518c |
| TCGA-55-6543-01A-11D-1753-08 | LUAD | ac7ab3b3-eb76-4da9-bfb3-82b90c8d79d6 |
| TCGA-55-6642-01A-11D-1855-08 | LUAD | 3c756f7c-d1f0-4ab1-9c9f-41d2282af3bf |
| TCGA-55-6712-01A-11D-1855-08 | LUAD | bc6eaf2b-9ccc-4ac7-9b19-204b0ff420a3 |
| TCGA-64-1676-01A | LUAD | 4bdf77d2-33cc-46e0-af34-1e66a90a213a |
| TCGA-64-1677-01A-01W-0928-08 | LUAD | 559017d8-4b22-4313-abdd-d3526c889d7f |
| TCGA-64-1678-01A-01W-0928-08 | LUAD | 42e3b592-b57f-4b18-8f62-e7b0a9c0f1db |
| TCGA-64-1680-01A | LUAD | 0bdbe623-cf95-465a-917d-87dfb6a8618e |
| TCGA-64-5774-01A-01D-1625-08 | LUAD | df5957d5-20d3-483e-990b-d6369fb990b8 |
| TCGA-64-5775-01A-01D-1625-08 | LUAD | c209d392-7d3a-481c-8cc7-398a6b90290a |
| TCGA-64-5778-01A-01D-1625-08 | LUAD | 3c540f87-5981-4b7a-b1ab-30c2056c785e |
| TCGA-64-5779-01A-01D-1625-08 | LUAD | 5734711b-52cd-46e6-9c2a-92c0612fee33 |
| TCGA-64-5781-01A-01D-1625-08 | LUAD | fb9cfb49-99cf-4f49-8f3d-e25e762eb3ce |
| TCGA-64-5815-01A-01D-1625-08 | LUAD | e800c8d4-786a-4a9d-ace2-2b779336e557 |
| TCGA-67-3770-01A | LUAD | 74bcf2d5-fd42-423e-bd96-b2de1b0cf778 |
| TCGA-67-3771-01A | LUAD | b0410cd6-693d-41d6-9dad-d1b1c30bf5cb |
| TCGA-67-3772-01A-01W-0928-08 | LUAD | 09226bc4-0202-4405-b3c9-208e8ffb7408 |
| TCGA-67-3773-01A | LUAD | e4cb66f4-e847-40bf-af14-20a3867a1c35 |
| TCGA-67-3774-01A | LUAD | b3585415-9ab9-4614-8b15-8edb66efd1dc |
| TCGA-67-4679-01B-01D-1753-08 | LUAD | 341bf21e-abd5-498e-8c49-111782af842c |
| TCGA-67-6215-01A-11D-1753-08 | LUAD | 68c2a355-862c-4657-b296-5776ed8447b0 |
| TCGA-67-6216-01A-11D-1753-08 | LUAD | 6dc6da8c-2ecf-412f-b2c4-74529adb7c0f |
| TCGA-67-6217-01A-11D-1753-08 | LUAD | cb98d825-668f-4b16-a05e-501e1c94f3fe |
| TCGA-71-6725-01A-11D-1855-08 | LUAD | 3a146eb4-7b9b-4834-b3d0-eac80f9173ec |
| TCGA-73-4658-01A-01D-1753-08 | LUAD | b11151cf-6976-4812-a77e-1a12f9d1245c |
| TCGA-73-4659-01A-01D-1265-08 | LUAD | 13989aec-b1a3-47c2-bc8e-ccf55f8e0c11 |
| TCGA-73-4662-01A-01D-1265-08 | LUAD | 48262c89-ecac-44c6-9a06-7170b7b41058 |
| TCGA-73-4666-01A-01D-1265-08 | LUAD | f49fc77e-03cd-423c-b3e1-18bb19568650 |
| TCGA-73-4668-01A-01D-1265-08 | LUAD | 0fdcb5e9-ada2-4755-ae02-491037ee9c10 |
| TCGA-73-4670-01A-01D-1265-08 | LUAD | 2aea0652-17ae-4dfa-9358-206d4f24f02f |
| TCGA-73-4675-01A-01D-1265-08 | LUAD | 59dad620-51f8-4c12-8b09-e635fbde126e |
| TCGA-73-4676-01A-01D-1753-08 | LUAD | ff368c6d-fedb-49cc-b519-7726816aff8d |
| TCGA-73-4677-01A-01D-1265-08 | LUAD | a9c03165-d534-425e-8370-d1f557b82fa2 |
| TCGA-75-5122-01A-01D-1753-08 | LUAD | e359b24f-7312-432f-b054-68dedc027df2 |
| TCGA-75-5125-01A-01D-1753-08 | LUAD | 4cee9575-3040-4ff1-bf7e-ca8873860c59 |
| TCGA-75-5126-01A-01D-1753-08 | LUAD | 1c1ad138-a59e-4f5d-8382-54c585c9298c |
| TCGA-75-5146-01A-01D-1625-08 | LUAD | 965a2bb7-6cd4-4309-beba-51ae74b8a980 |
| TCGA-75-5147-01A-01D-1625-08 | LUAD | 52910a60-bb15-4ba5-9d09-50d8ee6a445b |
| TCGA-75-6203-01A-11D-1753-08 | LUAD | d9cd7f95-07d3-4b87-be83-87340b08d249 |
| TCGA-75-6205-01A-11D-1753-08 | LUAD | 79c0e183-95aa-4c37-9b15-8567aa87c93a |
| TCGA-75-6206-01A-11D-1753-08 | LUAD | 7a5ca29b-85d3-46b1-a710-6dcd3ce821c8 |
| TCGA-75-6207-01A-11D-1753-08 | LUAD | 5a49e3fd-a47d-4b7d-9485-4238a88f4516 |
| TCGA-75-6211-01A-11D-1753-08 | LUAD | d8c9abbe-b112-4019-a6a3-f582df1379ed |
| TCGA-75-6212-01A-11D-1753-08 | LUAD | 0f2af4c9-05a8-4c97-ac2d-af9241b4ea64 |
| TCGA-80-5611-01A-01D-1625-08 | LUAD | c9bab512-c5c3-4ad3-a9bf-f5258e405966 |
| TCGA-86-6562-01A-11D-1753-08 | LUAD | e48dd11b-89ae-4278-8de0-7956423c8609 |
| TCGA-91-6828-01A-11D-1855-08 | LUAD | 99f819f2-4340-4303-8ff0-fdb03ef0151a |
| TCGA-91-6829-01A-21D-1855-08 | LUAD | 443f5b2d-832e-45cf-bca5-3f064ea3bc50 |
| TCGA-91-6831-01A-11D-1855-08 | LUAD | 1624af6f-05a6-474c-ba49-9754938979c6 |
| TCGA-91-6835-01A-11D-1855-08 | LUAD | 8120c5eb-2917-4053-a5e5-aad53ff45da9 |
| TCGA-91-6836-01A-21D-1855-08 | LUAD | 87045814-366d-4e42-97f2-ad341c620c47 |
| TCGA-18-3406-01A-01D-0983-08 | LUSC | d3320989-71fd-425b-933e-6e8528a016ed |
| TCGA-18-3407-01A-01D-0983-08 | LUSC | c5b09119-0237-4804-a4f9-b67d676b8674 |
| TCGA-18-3408-01A-01D-0983-08 | LUSC | cab7a425-e081-4bae-b666-6cdf8ba4dd70 |
| TCGA-18-3409-01A-01D-0983-08 | LUSC | aa733cb0-37a9-4fef-8d40-d57596ce9e51 |
| TCGA-18-3410-01A-01D-0983-08 | LUSC | 7e6382c3-368a-43a5-9812-c58f54ceba3f |
| TCGA-18-3411-01A-01D-0983-08 | LUSC | 6a9cc303-c7fd-4f40-8933-1636dea99252 |
| TCGA-18-3412-01A-01D-0983-08 | LUSC | 84aca315-8380-4625-887f-a8b3c704c0a9 |
| TCGA-18-3414-01A-01D-0983-08 | LUSC | 239deee9-2791-4163-b777-fdf8c49c9e33 |
| TCGA-18-3415-01A-01D-0983-08 | LUSC | ad0365d1-10b1-41e6-b838-9c5794b9ad42 |
| TCGA-18-3416-01A-01D-0983-08 | LUSC | e03577e7-37be-460b-96e8-5f6e0b49b3aa |
| TCGA-18-3417-01A-01D-1441-08 | LUSC | 024d8a82-06c5-4b82-9a27-c52bc4fd450a |
| TCGA-18-3419-01A-01D-0983-08 | LUSC | c75ed357-d845-4443-8c9e-a2afa8ed30df |
| TCGA-18-3421-01A-01D-0983-08 | LUSC | 9f0e482e-e72d-4c57-b4f7-4580edabd390 |
| TCGA-18-4083-01A-01D-1352-08 | LUSC | 0b87a82d-096c-4dd7-80c4-b4054fc1eba2 |
| TCGA-18-4086-01A-01D-1352-08 | LUSC | 9bbdf36b-6804-416f-977d-fce772972bcc |
| TCGA-18-4721-01A-01D-1441-08 | LUSC | d2ab2555-7288-47a4-a80c-bf62d65b67b8 |
| TCGA-18-5592-01A-01D-1632-08 | LUSC | 1a6da454-8faf-4725-a702-55d29da461a5 |
| TCGA-18-5595-01A-01D-1632-08 | LUSC | 973b8ed8-2295-4fb0-b857-f4433dfc785a |
| TCGA-21-1070-01A-01D-1521-08 | LUSC | 9e300205-b16d-4f40-bf1b-f47410678f6d |
| TCGA-21-1071-01A-01D-1521-08 | LUSC | e01302f9-c5d6-4745-9c5d-d8bb8d278a77 |
| TCGA-21-1076-01A-02D-1521-08 | LUSC | 504d4cb0-d2dd-420d-82e6-9ec14434a0fc |
| TCGA-21-1077-01A-01D-1521-08 | LUSC | a71d74cb-5b10-4787-a654-7049cbb49a92 |
| TCGA-21-1078-01A-01D-1521-08 | LUSC | 8cf9b32d-3d6f-4898-8c7a-89511b754021 |
| TCGA-21-1081-01A-01D-1521-08 | LUSC | 811f7a11-635c-4606-91fd-3729b97ffd8e |
| TCGA-21-5782-01A-01D-1632-08 | LUSC | 4c2ad4a0-5d57-4e27-9f35-058b2f205f50 |
| TCGA-21-5784-01A-01D-1632-08 | LUSC | f79285af-c364-4ec3-97d3-70a7d9b5800b |
| TCGA-21-5786-01A-01D-1632-08 | LUSC | d7404e0f-d171-419b-97d3-807570aba129 |
| TCGA-21-5787-01A-01D-1632-08 | LUSC | 7cb79e4b-c1f1-434d-b13b-6c2eb7760ee8 |
| TCGA-22-0944-01A-01D-1521-08 | LUSC | 818a6f09-a7fd-4cce-8373-adb4bcb5bc8c |
| TCGA-22-1002-01A-01D-1521-08 | LUSC | 7c7604fe-8321-46cb-ac34-0e7994b8853b |
| TCGA-22-1011-01A-01D-1521-08 | LUSC | c9924f9f-fd86-434c-a83d-393d65272e64 |
| TCGA-22-1012-01A-01D-1521-08 | LUSC | 3b75368a-d57f-4787-a0ef-3f478c7d22bc |
| TCGA-22-1016-01A-01D-1521-08 | LUSC | 935b113e-f5ed-4a07-8e1d-1603daba7f40 |
| TCGA-22-4591-01A-01D-1267-08 | LUSC | bcfb93d4-8653-477b-b5d2-c2832a0e3d92 |
| TCGA-22-4593-01A-21D-1817-08 | LUSC | b4a48075-92fd-43ab-95f3-476bcea88d7b |
| TCGA-22-4595-01A-01D-1267-08 | LUSC | 7fcf5123-2d1b-4666-9d39-a1aaf63cf954 |
| TCGA-22-4599-01A-01D-1441-08 | LUSC | 08732b51-8ec8-4888-b0c8-a0cb83181cb9 |
| TCGA-22-4601-01A-01D-1441-08 | LUSC | 6c05b3f5-65e9-4e7d-9f99-a694006f2ed0 |
| TCGA-22-4604-01A-01D-1267-08 | LUSC | db2614fb-109c-4ce1-af4c-f648a0d417fb |
| TCGA-22-4607-01A-01D-1267-08 | LUSC | d8c6bb83-ebdd-4547-9077-3eba5c8bb9f0 |
| TCGA-22-4613-01A-01D-1441-08 | LUSC | 5d1d538a-57d3-42ec-9fa3-0fad10b0f52f |
| TCGA-22-5471-01A-01D-1632-08 | LUSC | 665e98bf-6163-4d18-9665-ba93df9ecf6d |
| TCGA-22-5472-01A-01D-1632-08 | LUSC | be780766-483f-42f5-b0d0-11d23a940156 |
| TCGA-22-5473-01A-01D-1632-08 | LUSC | c107ca1d-5e35-470a-8c39-80dc7624e306 |
| TCGA-22-5474-01A-01D-1632-08 | LUSC | 1eda33fc-80e5-4c5f-8c61-43976ca0106f |
| TCGA-22-5477-01A-01D-1632-08 | LUSC | e7ebc6fb-0926-4c8a-a67b-0c6b9c1ffaba |
| TCGA-22-5478-01A-01D-1632-08 | LUSC | 0ac704eb-d722-4c27-bfb4-fea6ca7af240 |
| TCGA-22-5480-01A-01D-1632-08 | LUSC | 24e426fb-219a-4a4d-a45c-c9b0896d0e88 |
| TCGA-22-5482-01A-01D-1632-08 | LUSC | b57c316e-1cae-4286-bdbb-8b65c020b3fa |
| TCGA-22-5485-01A-01D-1632-08 | LUSC | 448af8b4-e071-48b0-a65b-b4ad17afdc0c |
| TCGA-22-5489-01A-01D-1632-08 | LUSC | c4eb6681-7ec3-4688-b06a-c47a0043f3fb |
| TCGA-22-5491-01A-01D-1632-08 | LUSC | ed4b5a8c-1dae-41a3-8a2a-f54fa51be4b8 |
| TCGA-22-5492-01A-01D-1632-08 | LUSC | abc94013-71f5-4ac6-88a4-01b4ef9f9d2f |
| TCGA-33-4532-01A-01D-1267-08 | LUSC | c8baeba2-2a73-41d7-9226-b89a8f42e18f |
| TCGA-33-4533-01A-01D-1267-08 | LUSC | 52b8c7c1-2cfe-410d-a738-1dec43109e24 |
| TCGA-33-4538-01A-01D-1267-08 | LUSC | e04814f8-a51f-4b6b-a4e9-bd8d2291817c |
| TCGA-33-4547-01A-01D-1267-08 | LUSC | 7e622fc2-06c5-4686-a885-e407725c2f08 |
| TCGA-33-4566-01A-01D-1441-08 | LUSC | ddd84ea3-dd5e-4f95-97c3-84c107c19cad |
| TCGA-33-4582-01A-01D-1441-08 | LUSC | 4cb06585-62f9-4aae-969a-2085b4d514c3 |
| TCGA-33-4583-01A-01D-1441-08 | LUSC | fb901997-6e46-436f-ad34-74aadc344245 |
| TCGA-33-4586-01A-01D-1441-08 | LUSC | e6bf4288-9fdd-4c56-b6d2-fa2f5ee542b6 |
| TCGA-33-6737-01A-11D-1817-08 | LUSC | 3b21ce38-16c6-4c68-9104-fa11f1b619b1 |
| TCGA-34-2596-01A-01D-1522-08 | LUSC | 66e35f68-f4db-46ee-876e-e770ea616ef3 |
| TCGA-34-2600-01A-01D-1522-08 | LUSC | 167e0f4e-e7d3-4942-885a-cf06419bbe6d |
| TCGA-34-2608-01A-02D-1522-08 | LUSC | 3c90209b-b6f6-40b2-a374-6cd37d6d3895 |
| TCGA-34-5231-01A-21D-1817-08 | LUSC | c9862ed2-4ba6-434d-a205-b1bda292d218 |
| TCGA-34-5232-01A-21D-1817-08 | LUSC | f32fff2f-0bbf-475f-b088-3f1699203c31 |
| TCGA-34-5234-01A-01D-1632-08 | LUSC | 7b19ae84-2cab-47e7-87df-46c497da17e0 |
| TCGA-34-5236-01A-21D-1817-08 | LUSC | 46cb2de7-bbe1-4444-b17e-4c5677a05249 |
| TCGA-34-5239-01A-21D-1817-08 | LUSC | 6e596912-2146-4c4f-97b6-70b610f5d4b4 |
| TCGA-34-5240-01A-01D-1441-08 | LUSC | 4c3840df-9824-40db-879e-6d24adc8c155 |
| TCGA-34-5241-01A-01D-1441-08 | LUSC | 0bcdbc37-cde8-47df-9184-621b2b47da5b |
| TCGA-34-5927-01A-11D-1817-08 | LUSC | d717b13a-e487-4cad-9aae-4b0d649236c4 |
| TCGA-34-5928-01A-11D-1817-08 | LUSC | 9e2d032e-f982-44fc-b6e0-3be82f029689 |
| TCGA-34-5929-01A-11D-1817-08 | LUSC | a25de54e-c13d-4973-864a-e307fbe7324a |
| TCGA-37-3783-01A-01D-1267-08 | LUSC | 711e9b21-bd8c-4058-a0ce-5ff4dc23b527 |
| TCGA-37-3789-01A-01D-0983-08 | LUSC | d732196f-ef85-43ea-aac7-7c9060bf19c5 |
| TCGA-37-4133-01A-01D-1352-08 | LUSC | a678cc49-9009-4027-826f-e17f4533538d |
| TCGA-37-4135-01A-01D-1352-08 | LUSC | 754dda66-fceb-4f63-bc99-c98aaa86b0c2 |
| TCGA-37-4141-01A-02D-1352-08 | LUSC | 3d4f4555-d71a-4c7d-8667-c42dcc20c076 |
| TCGA-37-5819-01A-01D-1632-08 | LUSC | edf2a2c0-3829-4da2-8960-598fbd5c4c07 |
| TCGA-39-5016-01A-01D-1441-08 | LUSC | d63a0a46-7676-40f5-8e03-b8317d243c73 |
| TCGA-39-5019-01A-01D-1817-08 | LUSC | 6aecd71e-84f1-4b4d-bff6-ede33026f58b |
| TCGA-39-5021-01A-01D-1441-08 | LUSC | 4d8b4c6f-e6eb-4799-b64d-119afc691e3d |
| TCGA-39-5022-01A-21D-1817-08 | LUSC | f60928ab-0cb1-4483-8d61-48a5333defbf |
| TCGA-39-5024-01A-21D-1817-08 | LUSC | 388478e9-8c1f-43f8-88c4-811bf3cc2500 |
| TCGA-39-5027-01A-21D-1817-08 | LUSC | 32c14926-b510-4714-90b2-b0bd68569cd4 |
| TCGA-39-5028-01A-01D-1441-08 | LUSC | 015b9329-ecf2-4410-b7b6-f9313b5d2adb |
| TCGA-39-5029-01A-01D-1441-08 | LUSC | aa02c83c-7ef0-400d-bd8d-729dacda6352 |
| TCGA-39-5030-01A-01D-1441-08 | LUSC | 9e7b63f2-6080-4bb0-b45d-a0d40dffcbe0 |
| TCGA-39-5031-01A-01D-1441-08 | LUSC | 3eab4096-8e8e-459d-a2bb-6ef03f414315 |
| TCGA-39-5035-01A-01D-1441-08 | LUSC | 035fe73e-56b4-4afe-b70e-dd3c34027f2d |
| TCGA-39-5036-01A-01D-1441-08 | LUSC | a1aa5fba-f179-4777-8d49-345a366d12fa |
| TCGA-39-5037-01A-01D-1441-08 | LUSC | 825bd82c-f8f8-4776-a7f5-713b3a574955 |
| TCGA-39-5039-01A-01D-1441-08 | LUSC | 0c14e914-abd4-4406-be82-a810b10a1320 |
| TCGA-43-2578-01A-01D-1522-08 | LUSC | 7ce90b30-d372-4edb-9807-b71cb5eb4cb7 |
| TCGA-43-3394-01A-01D-0983-08 | LUSC | bb72e789-f8ad-4ab5-805b-a9ac21cef0e3 |
| TCGA-43-3920-01A-01D-0983-08 | LUSC | a97333f4-d289-493f-8dff-88e52719fa86 |
| TCGA-43-5668-01A-01D-1632-08 | LUSC | f01dfe80-aee9-44f6-b32d-3591fbc3c0f5 |
| TCGA-43-6143-01A-11D-1817-08 | LUSC | 3874253f-7168-4cd6-b1d6-f426fa207313 |
| TCGA-43-6647-01A-11D-1817-08 | LUSC | 90b97948-26f7-4431-be89-af8c432baae0 |
| TCGA-43-6770-01A-11D-1817-08 | LUSC | 404ca8c2-f1bb-4749-8abd-87f491a8111c |
| TCGA-43-6771-01A-11D-1817-08 | LUSC | 20735861-1f84-4141-a467-f598108e1e41 |
| TCGA-46-3765-01A-01D-0983-08 | LUSC | 6c4bb09f-46c8-4a42-bf4f-8bad5316603d |
| TCGA-46-3766-01A-01D-0983-08 | LUSC | 0a691892-2209-4f3c-ab16-c2560e4928b4 |
| TCGA-46-3767-01A-01D-0983-08 | LUSC | db4ea3ec-e926-4e75-a97b-a527c101b3b9 |
| TCGA-46-3768-01A-01D-0983-08 | LUSC | 30666313-cc29-4fce-8308-b04fb932083c |
| TCGA-46-3769-01A-01D-0983-08 | LUSC | 108a1360-a545-4573-a775-49b3420814e2 |
| TCGA-46-6025-01A-11D-1817-08 | LUSC | 767a9ae0-2aa4-467b-b9c3-fb3bf701b642 |
| TCGA-46-6026-01A-11D-1817-08 | LUSC | 42a4a60c-257e-4bf6-a9ba-6f162dbca94a |
| TCGA-51-4079-01A-01D-1458-08 | LUSC | 0a43aade-225c-4a29-b1d8-6b930eb8a1db |
| TCGA-51-4080-01A-01D-1458-08 | LUSC | 2498ada2-b8d3-4220-8283-45af67a8119a |
| TCGA-51-4081-01A-01D-1458-08 | LUSC | 1492c429-1041-4d86-9358-c9b9babd1401 |
| TCGA-56-1622-01A-01D-1521-08 | LUSC | 0bbc7ede-5022-4084-925c-d65baaf7abc2 |
| TCGA-56-5897-01A-11D-1632-08 | LUSC | 056acb55-f3ba-4ce0-9735-3cfe6516df55 |
| TCGA-56-5898-01A-11D-1632-08 | LUSC | aaf47efe-4a0a-40d1-b70f-9c9168cbdae0 |
| TCGA-56-6545-01A-11D-1817-08 | LUSC | 16756a08-8308-4ad3-9e21-2cea0cd7028e |
| TCGA-56-6546-01A-11D-1817-08 | LUSC | 87e71949-5bd9-458c-95f7-4b19882c2b4f |
| TCGA-60-2698-01A-01D-1522-08 | LUSC | 2045c788-9ea8-4ea5-a5e3-65fc16a62adb |
| TCGA-60-2707-01A-01D-1522-08 | LUSC | 5d1fa470-2789-4576-9743-0362af682c1d |
| TCGA-60-2708-01A-01D-1522-08 | LUSC | a371189b-5808-4408-824e-8dacec925cc5 |
| TCGA-60-2709-01A-21D-1817-08 | LUSC | 4f321c92-ae27-4253-bd8b-4505ba8c7dc4 |
| TCGA-60-2710-01A-01D-1522-08 | LUSC | faecb1fe-b4ef-434d-818c-81ad2167dd25 |
| TCGA-60-2711-01A-01D-1522-08 | LUSC | 2ed85cc9-31bc-4cea-9e54-13b7c0e645fa |
| TCGA-60-2712-01A-01D-1522-08 | LUSC | 6662dd1b-3e4f-4b7a-b603-cfa7fd92fc30 |
| TCGA-60-2713-01A-01D-1522-08 | LUSC | 79eb7bba-f0d8-462c-add7-20a2fb7843e1 |
| TCGA-60-2715-01A-01D-1522-08 | LUSC | 8e05a30d-2177-45e0-90fd-8c5961268c39 |
| TCGA-60-2719-01A-01D-1522-08 | LUSC | ee6cc68e-8d2a-41ee-82c6-0fecdf7e6259 |
| TCGA-60-2720-01A-01D-1522-08 | LUSC | 3b435ddf-a496-40a2-82e8-6b10391aae5d |
| TCGA-60-2721-01A-01D-1522-08 | LUSC | 8defff62-9395-47cb-bb19-4b8487d9ea8e |
| TCGA-60-2722-01A-01D-1522-08 | LUSC | eb955f72-83bf-4635-a7ed-89e4d66e08f4 |
| TCGA-60-2723-01A-01D-1522-08 | LUSC | 8a6aa45a-ef6d-4005-b7c9-e15240dc6dd4 |
| TCGA-60-2724-01A-01D-1522-08 | LUSC | 387c6519-6529-4074-a5ab-00f8052a5732 |
| TCGA-60-2725-01A-01D-1267-08 | LUSC | f3ed705b-e5aa-4756-9794-e4b85303693a |
| TCGA-60-2726-01A-01D-1522-08 | LUSC | a96eddfc-3afb-4bf8-a440-c91778113fbd |
| TCGA-63-5128-01A-01D-1441-08 | LUSC | d3b9b51e-eeea-4355-829d-ee35bdd2cf5b |
| TCGA-63-5131-01A-01D-1441-08 | LUSC | b290a86e-22da-4f10-a421-2616bb47bc1b |
| TCGA-63-6202-01A-11D-1817-08 | LUSC | a3c568a6-0c43-47a7-a35a-3225fedeeb44 |
| TCGA-66-2727-01A-01D-0983-08 | LUSC | c2b2c909-1461-42ce-8fd9-736147dcacd8 |
| TCGA-66-2734-01A-01D-0983-08 | LUSC | 9f7a24a2-10e2-4039-ad27-13d7ec28ff36 |
| TCGA-66-2742-01A-01D-0983-08 | LUSC | 07047a99-45bd-4df6-ad6f-934a48e8e213 |
| TCGA-66-2744-01A-01D-0983-08 | LUSC | 43be1a37-b18e-4e96-89e6-ed6ee1d8e65a |
| TCGA-66-2754-01A-01D-0983-08 | LUSC | c34a64c8-3746-44f8-a7ee-77f502b6256c |
| TCGA-66-2755-01A-01D-1522-08 | LUSC | 177d64a9-65dc-4aa1-8774-bd8208e40f04 |
| TCGA-66-2756-01A-01D-1522-08 | LUSC | 472c95e6-eccb-4988-be16-fdace73b2ed8 |
| TCGA-66-2757-01A-01D-1522-08 | LUSC | 1886dba0-4662-4342-84ac-96af0beb2393 |
| TCGA-66-2758-01A-02D-1522-08 | LUSC | 71c4e854-a704-4787-a37a-fa6642ca5dac |
| TCGA-66-2759-01A-01D-1522-08 | LUSC | fecd0a2b-d176-438a-be95-306f453fde40 |
| TCGA-66-2763-01A-01D-1522-08 | LUSC | d6493c56-5322-4961-a693-8e8a62b0f7f1 |
| TCGA-66-2765-01A-01D-1522-08 | LUSC | 85d7e094-ca96-4090-83aa-2f318ae6e954 |
| TCGA-66-2766-01A-01D-1522-08 | LUSC | 452b75d0-1818-46aa-8804-9cfc0bd66449 |
| TCGA-66-2767-01A-01D-1522-08 | LUSC | ca748128-272c-4fad-9a1f-01328b93b3f4 |
| TCGA-66-2768-01A-01D-1522-08 | LUSC | 5d458cef-965d-4d27-b754-31df67ed6eaa |
| TCGA-66-2770-01A-01D-1522-08 | LUSC | e417903d-ab76-44f0-aae9-3a91fa9a8d3c |
| TCGA-66-2771-01A-01D-0983-08 | LUSC | 58c73372-223f-400a-a2df-073a78c58b62 |
| TCGA-66-2773-01A-01D-1267-08 | LUSC | fb0b515b-afc4-40c3-abe6-e90c442f0249 |
| TCGA-66-2777-01A-01D-1267-08 | LUSC | 2ea52fb8-d7c9-48ce-9aef-50df7c42e5d5 |
| TCGA-66-2778-01A-02D-1522-08 | LUSC | 5215060d-5ffd-49f3-a7a7-73167e7af74a |
| TCGA-66-2780-01A-01D-1522-08 | LUSC | d088bd17-a1a0-4bd9-bfe1-d57b5725c53b |
| TCGA-66-2781-01A-01D-1522-08 | LUSC | bfb33630-c8a8-4ec4-9eee-8bef349339ea |
| TCGA-66-2782-01A-01D-1522-08 | LUSC | 640ff507-203c-45aa-8bc1-030ee8639b5d |
| TCGA-66-2783-01A-01D-1267-08 | LUSC | f574d3b7-4ae4-49bc-9e05-f965fbc86119 |
| TCGA-66-2785-01A-01D-1522-08 | LUSC | 57debe39-f57d-400a-a860-3de357d6bec1 |
| TCGA-66-2786-01A-01D-1522-08 | LUSC | 999a6582-33cf-47ca-b268-9b2da102e99b |
| TCGA-66-2787-01A-01D-0983-08 | LUSC | c59e5971-e243-4b00-b5f0-f4bca18530d6 |
| TCGA-66-2788-01A-01D-0983-08 | LUSC | 2466d424-98bb-4380-9967-36abaa0e69d7 |
| TCGA-66-2789-01A-01D-0983-08 | LUSC | fab8faeb-35b3-42f0-b0af-4dfb1325a21a |
| TCGA-66-2791-01A-01D-0983-08 | LUSC | dd468431-2fa4-45ab-be1f-90671891c5c4 |
| TCGA-66-2792-01A-01D-0983-08 | LUSC | b704a17a-9ee9-4555-b2bb-250ac1ec5bed |
| TCGA-66-2793-01A-01D-1267-08 | LUSC | 7dc5f8ba-0080-43d3-8426-bd527a970761 |
| TCGA-66-2794-01A-01D-1267-08 | LUSC | 2c58fa70-8fef-4a49-8cde-bfdc92e77919 |
| TCGA-66-2795-01A-02D-0983-08 | LUSC | 73825564-8731-4137-972a-330490aceadc |
| TCGA-66-2800-01A-01D-1267-08 | LUSC | 803ec3a5-4347-41c3-a7b6-7eb00427a48c |
| TCGA-70-6722-01A-11D-1817-08 | LUSC | e81f1bb5-2d06-44b3-998a-e7a0b818467c |
| TCGA-70-6723-01A-11D-1817-08 | LUSC | 7483ea9f-8587-41e7-9ae5-d9223b76f33e |
| TCGA-85-6175-01A-11D-1817-08 | LUSC | 2ba53bf0-a4e1-4b46-b258-610522aac7ee |
| TCGA-85-6560-01A-11D-1817-08 | LUSC | a5a156b8-2c8a-4ed0-8bae-b60cdc95698f |
| TCGA-85-6561-01A-11D-1817-08 | LUSC | f5aa0f1c-da19-4c04-b695-01ed5b20e79e |
| TCGA-04-1332-01A-01W-0488-09 | OV | b52e5d90-dc57-438c-9c38-e043308c24ac |
| TCGA-04-1336-01A-01W-0488-09 | OV | 586101df-93c9-4d0b-ba0e-58df7a2f9598 |
| TCGA-04-1343-01A-01W-0488-09 | OV | fbbc3d80-aff2-463e-8eb3-c4361ad7cb98 |
| TCGA-04-1346-01A-01W-0488-09 | OV | 9f494df7-f64f-4935-ae42-eeb0b94624dc |
| TCGA-04-1347-01A-01W-0488-09 | OV | 21b50b8c-781a-4e15-a4ad-715f416f0fa2 |
| TCGA-04-1348-01A-01W-0494-09 | OV | 1f4dee42-8f3d-4307-b6e5-3381d77d201c |
| TCGA-04-1349-01A-01W-0494-09 | OV | e456f707-f0a0-4624-98bc-e9dfe779182b |
| TCGA-04-1361-01A-01W-0494-09 | OV | 0fc567bd-2201-4f3d-820e-2c0dbe58da6f |
| TCGA-04-1362-01A-01W-0494-09 | OV | 830e207f-458e-4628-b7bc-287c2f2e12e5 |
| TCGA-04-1542-01A-01W-0553-09 | OV | 317a63af-e862-43df-8ef5-7c555b2cb678 |
| TCGA-09-0366-01A-01W-0372-09 | OV | 62269d21-50dc-42b0-b1e4-75ed8010080a |
| TCGA-09-0369-01A-01W-0372-09 | OV | 633f5c4d-c224-404c-9f68-24daafd1fc84 |
| TCGA-10-0930-01A-02W-0421-09 | OV | ec98ed86-1d2f-4e54-b2d4-5976469bf0b8 |
| TCGA-10-0933-01A-01W-0421-09 | OV | 3ec4215f-b57d-4ae7-b247-55ea1f7e97d3 |
| TCGA-10-0935-01A-03W-0421-09 | OV | af0edbf4-9d90-4373-a9ce-0875ebbe1d04 |
| TCGA-13-0723-01A-02W-0372-09 | OV | 6f9e5a76-5d2a-4bb0-babf-3f365a177236 |
| TCGA-13-0724-01A-01W-0372-09 | OV | 2b6aa1c8-5150-4d8f-af59-d5a826321308 |
| TCGA-13-0726-01A-01W-0372-09 | OV | 201415c2-5b5a-4bb8-8005-bf2c78d4d88e |
| TCGA-13-0755-01A-01W-0372-09 | OV | 9bd227fa-e52a-4805-bd04-ad63df0930af |
| TCGA-13-0760-01A-01W-0372-09 | OV | 5181630f-246a-4cb4-88c2-1534b5fb8e37 |
| TCGA-13-0765-01A-01W-0372-09 | OV | 5bcfe3ea-d95e-47ff-9718-6b123d3acaef |
| TCGA-13-0791-01A-01W-0372-09 | OV | 70f63e2f-9bc6-4ed9-8d91-f1889287d7b7 |
| TCGA-13-0795-01A-01W-0372-09 | OV | b266a007-694a-4580-ad67-48b0f709bc43 |
| TCGA-13-0800-01A-01W-0372-09 | OV | 757862e3-0392-4e05-a242-25e3d2094ee8 |
| TCGA-13-0804-01A-01W-0372-09 | OV | 7f39610d-45b8-45ae-806e-16b7acebafa6 |
| TCGA-13-0807-01B-02W-0421-09 | OV | f80466d9-6cc8-461b-acc2-addee22bd42a |
| TCGA-13-0884-01B-01W-0494-09 | OV | c5f0aa38-556b-401c-b4da-ac82cdc2e637 |
| TCGA-13-0885-01A-02W-0421-09 | OV | a530d9a9-b21e-47be-b4d8-1707b71f360a |
| TCGA-13-0887-01A-01W-0421-09 | OV | e05146f2-688d-416b-a992-e2c7a2b7b244 |
| TCGA-13-0890-01A-01W-0421-09 | OV | 15b867fb-7a7b-4158-9abd-91870ba77eb7 |
| TCGA-13-0893-01B-01W-0494-09 | OV | a335ab49-84b7-4d3b-a03d-9c3931904ca5 |
| TCGA-13-0894-01B-01W-0494-09 | OV | eb57990e-702f-4fac-9ef5-7447ecb45cec |
| TCGA-13-0897-01A-01W-0421-09 | OV | f48ed68f-a833-4b78-971a-3c746c563d24 |
| TCGA-13-0903-01A-01W-0421-09 | OV | 854167b5-03ab-4867-af34-9c92e385822e |
| TCGA-13-0910-01A-01W-0421-09 | OV | 26cebe0b-b7a7-431e-bc12-7fda22af72f3 |
| TCGA-13-0912-01A-01W-0421-09 | OV | 517f4d7f-c962-414f-8824-f2a7ae19cb6d |
| TCGA-13-0920-01A-01W-0421-09 | OV | 2e28969b-c9a9-41ec-80bf-f583197b7f92 |
| TCGA-13-0924-01A-01W-0421-09 | OV | 510dda3c-6a1f-4781-972f-c9c270608c72 |
| TCGA-13-1403-01A-01W-0494-09 | OV | acbc77ba-7cc0-4af2-9ab6-0c835ce33998 |
| TCGA-13-1404-01A-01W-0494-09 | OV | 692e4b24-daf0-4771-b4a6-b0599f122ad8 |
| TCGA-13-1405-01A-01W-0494-09 | OV | c0d1de72-4cce-4d74-93f0-29c462dc1426 |
| TCGA-13-1411-01A-01W-0494-09 | OV | e254d7f4-1edf-4054-9ca6-9fe058a05484 |
| TCGA-13-1412-01A-01W-0494-09 | OV | f7edafe2-3eab-4bac-9d25-ed5c223b4aee |
| TCGA-13-1481-01A-01W-0549-09 | OV | f9eab025-5518-4240-b1a8-19f8ff8354f0 |
| TCGA-13-1482-01A-01W-0549-09 | OV | a68927d4-e827-49c9-9c3a-23ce0543261b |
| TCGA-13-1483-01A-01W-0549-09 | OV | 52280c07-44f5-4e9c-8601-7455b5b0de7a |
| TCGA-13-1488-01A-01W-0549-09 | OV | 886a8c10-63cf-4cb2-83d2-5a99bbda193d |
| TCGA-13-1489-01A-01W-0549-09 | OV | 395c1d93-7216-4c9d-bfad-26ff95fb8afe |
| TCGA-13-1491-01A-01W-0549-09 | OV | fb7d1c2b-3e87-4d05-a58b-92d0e1016986 |
| TCGA-13-1497-01A-01W-0549-09 | OV | 04e814c6-ea28-4ade-bc8f-a618552943da |
| TCGA-13-1498-01A-01W-0549-09 | OV | b00d9680-4099-43fe-87de-b3cc8b9e70c8 |
| TCGA-13-1499-01A-01W-0549-09 | OV | b4ce07b1-677e-4a9c-8f8e-2b7762487692 |
| TCGA-13-1506-01A-01W-0549-09 | OV | 7534b542-88f8-445c-ae4a-9f44fb6798a8 |
| TCGA-13-1507-01A-01W-0549-09 | OV | 5423db1a-5b59-4a5b-a676-00a54570b04a |
| TCGA-13-1509-01A-01W-0549-09 | OV | 4d3fab96-bc22-48d0-a3ef-1844ad894d0f |
| TCGA-23-1021-01B-01W-0488-09 | OV | 4f14d366-4750-471f-98a1-a01934365ee1 |
| TCGA-23-1022-01A-02W-0488-09 | OV | 160a0e7d-315e-4de3-a7d4-928412fd909c |
| TCGA-23-1117-01A-02W-0488-09 | OV | 3a4b0c6a-1f43-437c-b715-fc50c1c0303d |
| TCGA-23-1118-01A-01W-0488-09 | OV | 00c41845-6b48-40fa-82e9-1b436e7d91c3 |
| TCGA-23-1123-01A-01W-0488-09 | OV | 22cfe2c8-5e1f-4b64-854d-2a7a02bf10fe |
| TCGA-23-1124-01A-01W-0488-09 | OV | 8a4061a0-77f2-4bb4-a3da-9b3d9f0314b9 |
| TCGA-24-0966-01A-01W-0977-09 | OV | dc069342-661a-4012-9bda-0c67469e117d |
| TCGA-24-0980-01A-01W-0421-09 | OV | 87d32a92-a8d2-4656-a100-798328338486 |
| TCGA-24-0982-01A-01W-0488-09 | OV | 7667c0e6-e44a-448f-b118-6e2171a99b6c |
| TCGA-24-1103-01A-01W-0488-09 | OV | 47b7427c-a91a-4872-bc08-50c07ba60512 |
| TCGA-24-1104-01A-01W-0488-09 | OV | 9cdb7821-fe43-46cd-94f3-b9d68b9ce21f |
| TCGA-24-1413-01A-01W-0494-09 | OV | 1b2d2cde-4553-472e-82f1-8224745ac1eb |
| TCGA-24-1416-01A-01W-0549-09 | OV | 21f5e805-c0b4-487b-9ccd-02963e2369ff |
| TCGA-24-1417-01A-01W-0549-09 | OV | f6f43d04-a9e3-48c8-a276-3bebcaf416d7 |
| TCGA-24-1418-01A-01W-0549-09 | OV | 6093bcb5-4889-4cb9-9b01-e4e4278e72aa |
| TCGA-24-1424-01A-01W-0549-09 | OV | 2849f3e8-85d8-4d42-953b-3190b0ca98fc |
| TCGA-24-1425-01A-02W-0553-09 | OV | f8d4c37d-5b4d-4f5a-8022-7da2b32cc1b0 |
| TCGA-24-1426-01A-01W-0549-09 | OV | 063f8696-2c9d-4af4-a863-df10c42a5ea8 |
| TCGA-24-1427-01A-01W-0549-09 | OV | 6511d3d4-722c-4702-a644-29bb98e5e5c3 |
| TCGA-24-1428-01A-01W-0549-09 | OV | 52866517-eddf-4d63-a121-a296d6b2d264 |
| TCGA-24-1435-01A-01W-0549-09 | OV | 28d236f6-dddc-48c2-be30-b1568a4d6055 |
| TCGA-24-1436-01A-01W-0549-09 | OV | adeff0f5-d2a3-41c5-a509-298f702266bb |
| TCGA-24-1463-01A-01W-0549-09 | OV | c01ca9e7-ee9b-4698-8e4d-920ad7bfbe5f |
| TCGA-24-1464-01A-01W-0549-09 | OV | 01ec3cbb-c68a-4874-b396-f5e34876e04a |
| TCGA-24-1469-01A-01W-0553-09 | OV | 990c4b9d-608d-4b85-959c-5cc12f4e10fc |
| TCGA-24-1470-01A-01W-0553-09 | OV | 1d2bf111-910b-4ce9-8638-ab992b414e65 |
| TCGA-24-1549-01A-01W-0553-09 | OV | b2e252bd-895f-4b28-9367-dd527331010f |
| TCGA-24-1562-01A-01W-0553-09 | OV | 5e49bcea-9c1d-4cfd-a64c-4b84859bdda5 |
| TCGA-24-1563-01A-01W-0553-09 | OV | b6c46b53-f94d-4936-9005-518c8f1c1449 |
| TCGA-24-1616-01A-01W-0553-09 | OV | c464b2f6-9cfe-463a-b5e3-9a76cd4480c5 |
| TCGA-25-1315-01A-01W-0494-09 | OV | 52f45b5e-af86-454c-be63-a56c6c21b730 |
| TCGA-25-1316-01A-01W-0494-09 | OV | d75a0b16-04e4-4ba3-a695-132c5ace698b |
| TCGA-25-1322-01A-01W-0494-09 | OV | 626f1798-fb15-4b01-8d8f-db19777d72e9 |
| TCGA-AF-3913-01A-02W-1073-09 | READ | 4ebe7cf9-ce4f-485d-9332-ea9b536e38e2 |
| TCGA-AG-3887-01A-01W-1073-09 | READ | 6d2de0f5-e812-4d3f-903b-7febdcfcd2f7 |
| TCGA-AG-3890-01A-01W-1073-09 | READ | 042e984f-c106-4b23-9908-5abaf407e694 |
| TCGA-AG-3892-01A-01W-1073-09 | READ | 26acdae6-b01a-4dbd-b0b8-f6d97fe01808 |
| TCGA-AG-3893-01A-01W-1073-09 | READ | 0faa6d28-c01c-4847-9552-912733485610 |
| TCGA-AG-3894-01A-01W-1073-09 | READ | e508d0c8-cdaf-463f-bb03-47af1bc41866 |
| TCGA-AG-3896-01A-01W-1073-09 | READ | 22c7d09a-e69b-44be-8d8e-0a0cc9adf57c |
| TCGA-AG-3898-01A-01W-1073-09 | READ | cc3516ba-2941-4efa-80fc-7b5041194d52 |
| TCGA-AG-3901-01A-01W-1073-09 | READ | 84859471-1136-4f42-ab75-b27a4ef27199 |
| TCGA-AG-3902-01A-01W-1073-09 | READ | b679f02d-f48d-49eb-b245-65f341e4c181 |
| TCGA-AG-3909-01A-01W-1073-09 | READ | f5ece3cf-39eb-4277-8975-986e548bc1ea |
| TCGA-AG-3999-01A-01W-1073-09 | READ | 0445426d-b9c0-4ce5-b1cc-cb236d4381cf |
| TCGA-AG-4001-01A-02W-1073-09 | READ | 55075176-07a4-4183-9f8f-9f472b15a6b4 |
| TCGA-AG-4005-01A-01W-1073-09 | READ | be1d3bda-de1a-4768-a2e4-22c07326ddc3 |
| TCGA-AG-4007-01A-01W-1073-09 | READ | 6fcfdc8f-22c0-4c3a-9e46-58c0a68e818e |
| TCGA-AG-4008-01A-01W-1073-09 | READ | 83cd3c15-8eab-4d46-b9a2-36ee719f6774 |
| TCGA-AG-4015-01A-01W-1073-09 | READ | cf6f8e0f-04bf-4a0d-933e-8034ba6c1607 |
| TCGA-AG-A008-01A-01W-A005-10 | READ | 2221cfc4-b324-4329-ad37-3dd9a5adf36e |
| TCGA-AG-A00C-01A-01W-A005-10 | READ | 1a4f95be-32d3-4202-a0e7-507181b3fb86 |
| TCGA-AG-A00H-01A-01W-A00E-09 | READ | fdc4c8ac-fee2-4801-ae94-94c5d8058a9f |
| TCGA-AG-A00Y-01A-02W-A005-10 | READ | b50ae1df-ee6f-4a5e-ba4b-c962d740ab22 |
| TCGA-AG-A011-01A | READ | b5dd8f49-26fc-48d9-a964-d8ebdcca9e19 |
| TCGA-AG-A014-01A | READ | fbfa61fe-4fb7-4b2a-9bf0-33140fd41873 |
| TCGA-AG-A015-01A-01W-A005-10 | READ | abb751f0-c4df-4556-ac9b-ad1e1971cccf |
| TCGA-AG-A016-01A-01W-A005-10 | READ | f20ae301-b10b-41fa-9169-04bc6c3d103a |
| TCGA-AG-A01L-01A | READ | b034c90b-d0bd-466a-88ba-b61efd36c6e4 |
| TCGA-AG-A025-01A-01W-A00E-09 | READ | 7b5a3c33-cd13-4e4d-a1f8-3405dab5998f |
| TCGA-AG-A02G-01A-01W-A00E-09 | READ | 954527dc-8a7d-474d-b580-82199e86cb5a |
| TCGA-AG-A02X-01A-01W-A00E-09 | READ | 9ffb8919-a98c-40bd-bdad-146b1ccc14ef |
| TCGA-AG-A032-01A-01W-A00E-09 | READ | 7522eb6b-797a-4964-8aca-6d70590b5f9f |

Pipeline for prediction of peptides derived from gene mutations with binding to personal HLA alleles: MHC-binding affinity was predicted across all possible 9-mer and 10-mer peptides generated from each somatic mutation and the corresponding wildtype peptide using NetMHCpan (version 2.4). These tiled peptides were analyzed for their binding affinities (IC50 nM) to each class I alleles in the patients' HLA profile. An IC50 value of less than 150 nM was considered a predicted strong to intermediate binder, an IC50 of 150-500 nM was considered a predicted weak binder, while an IC50>500 nM was considered a non-binder. Experimental confirmation of predicted peptides binding to HLA molecules (IC50<500 nM) was performed using a competitive MHC class I allele-binding assay and has been described in detail elsewhere (Cai et al. 28 and Sidney et al. 2001).

Sources of antigen: Peptides were synthesized to >95% purity (confirmed by high performance liquid chromatography) from New England Peptide (Gardner, Mass.); or RS Synthesis, (Louisville, Ky.). Peptides were reconstituted in DMSO (10 mg/ml) and stored at −80° C. until use. Minigenes comprised of a sequence of 300 bp encompassing mut or wt FNDC3B were PCR-cloned from Pt 2's tumor into the expression vector pcDNA3.1 using the following primers: 5′primer: GACGTCGGATCCCACCATGGGTCCCGGAATTAAGAAAACAGAG; 3′ primer: CCCGGGGCGGCCGCCTAATGGTGATGGTGATGGTGACATTCTAATTCTTCTCCACTG TAAA. Minigenes were expressed in antigen-presenting target cells by introducing 20 μg of the plasmid into 2 million K562 cells (ATCC) stably transfected with HLA-A2 by Amaxa nucleofection (Solution V, Program T16, Lonza Inc; Walkersville, Md.). Cells were incubated in RPMI media (Cellgro; Manassas, Va.), supplemented with 10% fetal bovine serum (Cellgro), 1% HEPES buffer (Cellgro), and 1% L-glutamine (Cellgro). The cells were harvested 2 days following nucleofection for immune assays.

Analysis of gene expression in CLL cases: previously reported microarray data (NCI Gene Expression Omnibus accession GSE37168) was reanalyzed. Affymetrix CEL files were processed using the affy package in R. The Robust Multichip Analysis (RMA) algorithm was used for background correction which models the observed intensities as a mixture of exponentially distributed signal and normally distributed noise. This was followed by quantile normalization across arrays to facilitate comparison of gene expression under different conditions. The individual probe-level was finally summarized using the median polish approach to get robust probeset-level values. Gene-level values were obtained by selecting the probe with the maximal average expression for each gene. Batch effects in the data were removed by using the Combat program.

Generation and detection of antigen specific T cells from patient PBMCs: Autologous dendritic cells (DCs) were generated from immunomagnetically-isolated CD14⁺ cells (Miltenyi, Auburn Calif.) that were cultured in RPMI (Cellgro) supplemented with 3% fetal bovine serum, 1% penicillin-streptomycin (Cellgro), 1% L-glutamine and 1% HEPES buffer in the presence of 120 ng/ml GM-CSF and 70 ng/ml IL-4 (R&D Systems, Minneapolis, Minn.). On days three and five, additional GM-CSF and IL-4 were added. On day six, cells were exposed to 30 μg/ml Poly I:C (Sigma Aldrich, St Louis, Mo.) to undergo maturation (for 48 hours), in addition to adding IL-4 and GM-CSF. CD19⁺ B cells were isolated from patient PBMCs by immunomagnetic selection (CD19⁺ microbeads; Miltenyi, Auburn, Calif.), and seeded at 1×10⁶ cells/well in a 24-well plate. B cells were cultured in B cell media (Iscoves modified Dulbecco medium (IMDM; Life Technologies, Woburn, Mass.), supplemented with 10% human AB serum (GemCell, Sacramento, Calif.), 5 μg/mL insulin (Sigma Chemical, St Louis, Mo.), 15 μg/mL gentamicin, IL-4 (2 ng/ml, R&D Systems, Minneapolis, Minn.) and CD40L-Tri (1 μg/ml), CD40L-Tri was replenished every 3-4 days. For some experiments, CD40L-Tri activated and expanded. CD19⁺ B cells were:used as APCs.

Generation of antigen-specific T cells from patient PBMCs: To generate peptide-reactive T cells from CLL patients, immunomagnetically selected CD8⁺ T cells (5×10⁶/well) from pre- and post-transplant PBMCs (CD8+ Microbeads, Miltenyi, Auburn, Calif.) were cultured with autologous peptide pool-pulsed DCs (at 40:1 ratio) or CD40L-Tri-activated irradiated B cells (at 4:1 ratio) respectively, in complete medium supplemented with 10% FBS and 5-10 ng/mL IL-7, IL-12 and IL-15. APCs were pulsed for 3 hours with peptide pools (10 μM/peptide/pool). CD8⁺ T cells were re-stimulated weekly (for 1-3 weeks, starting on day 7) with APCs.

Detection of antigen-specific T cells: T cell specificity against peptide pools was tested by IFN-γ ELISPOT assay, 10 days following 2^{nd} and 4^{th} stimulations. IFN-γ release was detected using test and control peptide-pulsed CD40L-activated B cells (50,000 cells/well) co-incubated with 50,000 CD8⁺ T cells/well (Millipore, Billerica, Mass.) for 24 hours on ELISPOT plate. IFN-γ was detected using capture and detection antibodies, as directed (Mabtech AB, Mariemont, Ohio), and imaged (ImmunoSpot Series Analyzer; Cellular Technology, Cleveland, Ohio). To test T cell reactivity dependence on MHC class I, ELISPOT plates were first coated with APCs co-incubated with class I blocking antibody (W6/32) for 2 hours at 37° C., prior to introduction of T cells into the wells. MHC class I tetramer was used to test specificity of T cells where indicated (Emory University, Atlanta Ga.). For tetramer staining, 5×10⁵ cells were incubated for 60 minutes at 4° C. with 1 μg/mL PE-labeled tetramer, and then incubated with the addition of anti-CD3-FITC and anti-CD8-APC antibodies (BD Biosciences, San Diego Calif.) for another 30 minutes at 4° C. A minimum of 100,000 events were acquired per sample. Secretion of GM-CSF and IL-2 from cultured CD8⁺ T cells was detected by analysis of culture supernatants using a Luminex multiplex bead-based technology, per the manufacturer's recommendations (EMD Millipore, Billerica, Mass.). In brief, fluorescent-labeled microspheres were coated with specific cytokine capture antibodies. After incubation with the culture supernatant sample, captured cytokines were detected by a biotinylated detection antibody followed by a streptavidin-PE conjugate and median fluorescence intensity (MFI) was measured (Luminex 200 Bead Array instrument; Luminex Corporation, Austin Tex.). Based on a standard curve, cytokine levels were calculated in the Bead View Software program (Upstate, EMD Millipore, Billerica, Mass.). For detection and quantitation of TCR Vβ clonotypes, mut-FNDC3B specific T cells were enriched from Pt 2's T cell lines using the IFN-γ secretion assay (Miltenyi, Auburn, Calif.) according to the manufacturer's instructions and as previously described.

Statistical considerations: Two-way ANOVA models were constructed for T cells reactivity against mut vs wt peptide in the form of IFN-gamma, GM-CSF, and IL-2 release and included concentration and mutational status as fixed effects along with an interaction term as appropriate. P-values for these models were adjusted for multiple comparisons post-hoc using the Tukey method. For normalized comparisons of IFN-gamma, a t-test was performed to test the hypothesis that the normalized ratio equaled one. For other comparisons of continuous measures between groups, a Welch t-test was used. All P-values reported are two-sided and considered significant at the 0.05 level with appropriate adjustment for multiple comparisons. Analysis was performed in SAS v9.2.

Detection and quantitation of TCR Vβ clonotypes: To detect mut-FNDC3B specific TCR Vβ, a two-step nested PCR from peptide-specific IFN-γ enriched T cell populations was performed. In short, the dominant Vβ subfamily was identified among the 24 known Vβ subfamilies. First, 5 pools of Vβ forward primers (pool 1: Vβ 1-5.1; pool 2: Vβ 5.2-9; pool 3: Vβ 10-13.2; pool 4: Vβ 14-19; and pool 5: Vβ 20, 22-25) were generated. RNA extracted from the T cell clones (QIAamp RNA Blood Mini-kit; Qiagen, Valencia, Calif.), was reverse transcribed into cDNA (Superscript, GIBCO BRL, Gaithersburg, Md.) using random hexamers, and PCR-amplified in five separate 20 μl volume reactions. Second, T cell clone-derived cDNA was re-amplified, with each of the 5 individual primers contained within a positive pool together with a FAM-conjugated Cβ reverse (internal) primer. Subsequently, 4 μl of this PCR product was amplified with 1 μl of the clone CDR3 region-specific primer and probe, and 10 μl of Taqman Fast Universal PCR Master Mix (Applied Biosystems, Foster City, Calif.) in a total volume of 20 μl. The PCR amplification conditions were: 95° C. for 20 minutes×1 cycle, and 40 cycles of 95° C. for 3 seconds followed by 60° C. for 30 seconds (7500 Fast Real-time PCR cycler; Applied Biosystems, Foster City, Calif.). Test transcripts were quantified relative to S18 ribosomal RNA transcripts by calculating 2\hat{ }(S18 rRNA CT-target CT) as described previously.

Detection of molecular tumor burden: The clonotypic IgH sequence of Pt 2 was identified using a panel of VH-specific PCR primers, as previously described. Based on this sequence, a quantitative Taqman PCR assay was designed such that a sequence-specific probe was located in the region of junctional diversity (Applied Biosystems; Foster City, Calif.). This Taqman assay was applied to cDNA from tumor. All PCR reactions consisted of: 50° C. for 1 minute×1 cycle, 95° C. for 10 minutes×1 cycle, and 40 cycles of 95° C. for 15 seconds followed by 60° C. for 1 minute. All reactions were performed using a 7500 Fast Real-time PCR cycler (Applied Biosystems, Foster City, Calif.). Test transcripts were quantified relative to GAPDH.

### REFERENCES

Zhang G L, Ansari H R, Bradley P, et al. Machine learning competition in immunology—Prediction of HLA class I binding peptides. J Immunol Methods. Nov. 30, 2011; 374(1-2):1-4.
Kawai T, Akira S. TLR signaling. Seminars in immunology. February 2007; 19(1):24-32.
Adams S. Toll-like receptor agonists in cancer therapy. Immunotherapy. November 2009; 1(6):949-964.
Cheever M A. Twelve immunotherapy drugs that could cure cancers. Immunological reviews. April 2008; 222:357-368.
Bogunovic D, Manches O, Godefroy E, et al. TLR4 engagement during TLR3-induced proinflammatory signaling in dendritic cells promotes IL-10-mediated suppression of antitumor immunity. Cancer Res. Aug. 15, 2011; 71(16):5467-5476.
Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4):e1000373.
Boscardin S B, Hafalla J C, Masilamani R F, et al. Antigen targeting to dendritic cells elicits long-lived T cell help for antibody responses. The Journal of experimental medicine. Mar. 20, 2006; 203(3):599-606.
Soares H, Waechter H, Glaichenhaus N, et al. A subset of dendritic cells induces CD4+ T cells to produce IFN-gamma by an IL-12-independent but CD70-dependent mechanism in vivo. The Journal of experimental medicine. May 14, 2007; 204(5):1095-1106.
Trumpfheller C, Caskey M, Nchinda G, et al. The microbial mimic poly IC induces durable and protective CD4+ T cell immunity together with a dendritic cell targeted vaccine. Proc Natl Acad Sci USA. Feb. 19, 2008; 105(7):2574-2579.
Trumpfheller C, Finke J S, Lopez C B, et al. Intensified and protective CD4+ T cell immunity in mice with anti-dendritic cell HIV gag fusion antibody vaccine. The Journal of experimental medicine. Mar. 20, 2006; 203(3):607-617.
Salem M L, Kadima A N, Cole D J, Gillanders W E. Defining the antigen-specific T-cell response to vaccination and poly(I:C)/TLR3 signaling: evidence of enhanced primary and memory CD8 T-cell responses and antitumor immunity. J Immunother. May-June 2005; 28(3):220-228.
Tough D F, Borrow P, Sprent J. Induction of bystander T cell proliferation by viruses and type I interferon in vivo. Science. Jun. 28, 1996; 272(5270):1947-1950.
Zhu X, Nishimura F, Sasaki K, et al. Toll like receptor-3 ligand poly-ICLC promotes the efficacy of peripheral vaccinations with tumor antigen-derived peptide epitopes in murine CNS tumor models. Journal of translational medicine. 2007; 5:10.
Flynn B J, Kastenmuller K, Wille-Reece U, et al. Immunization with HIV Gag targeted to dendritic cells followed by recombinant New York vaccinia virus induces robust T-cell immunity in nonhuman primates. Proc Natl Acad Sci USA. Apr. 26, 2011; 108(17):7131-7136.
Gaucher D, Therrien R, Kettaf N, et al. Yellow fever vaccine induces integrated multilineage and polyfunctional immune responses. The Journal of experimental medicine. Dec. 22, 2008; 205(13):3119-3131.
Caskey M, Lefebvre F, Filali-Mouhim A, et al. Synthetic double-stranded RNA induces innate immune responses similar to a live viral vaccine in humans. The Journal of experimental medicine. Nov. 21, 2011; 208(12):2357-2366.
Sabbatini P, Tsuji T, Ferran L, et al. Phase I trial of overlapping long peptides from a tumor self-antigen and poly-ICLC shows rapid induction of integrated immune response in ovarian cancer patients. Clinical cancer research : an official journal of the American Association for Cancer Research. Dec. 1, 2012; 18(23):6497-6508.
Robinson R A, DeVita V T, Levy H B, Baron S, Hubbard S P, Levine A S. A phase I-II trial of multiple-dose polyriboinosic-polyribocytidylic acid in patients with leukemia or solid tumors. Journal of the National Cancer Institute. September 1976; 57(3):599-602.
Siegel R, Naishadham D, Jemal A. Cancer statistics, 2013. CA: a cancer journal for clinicians. January 2013; 63(1):11-30.
Balch C M, Gershenwald J E, Soong S J, et al. Final version of 2009 AJCC melanoma staging and classification. Journal of clinical oncology: official journal of the American Society of Clinical Oncology. Dec. 20, 2009; 27(36):6199-6206.
Eggermont A M, Suciu S, Testori A, et al. Ulceration and stage are predictive of interferon efficacy in melanoma: results of the phase III adjuvant trials EORTC 18952 and EORTC 18991. Eur J Cancer. January 2012; 48(2):218-225.
Sosman J A, Moon J, Tuthill R J, et al. A phase 2 trial of complete resection for stage IV melanoma: results of Southwest Oncology Group Clinical Trial S9430. Cancer. Oct. 15, 2011; 117(20):4740-4706.
Flaherty K T, Hodi F S, Fisher D E. From genes to drugs: targeted strategies for melanoma. Nat Rev Cancer. May 2012; 12(5):349-361.
Mocellin S, Pasquali S, Rossi C R, Nitti D. Interferon alpha adjuvant therapy in patients with high-risk melanoma: a systematic review and meta-analysis. Journal of the National Cancer Institute. Apr. 7, 2010; 102(7):493-501.
Pirard D, Heenen M, Melot C, Vereecken P. Interferon alpha as adjuvant postsurgical treatment of melanoma: a meta-analysis. Dermatology. 2004; 208(1):43-48.
Wheatley K, Ives N, Hancock B, Gore M, Eggermont A, Suciu S. Does adjuvant interferon-alpha for high-risk melanoma provide a worthwhile benefit? A meta-analysis of the randomised trials. Cancer treatment reviews. August 2003; 29(4):241-252.
Buckwalter M R, Srivastava P K. “It is the antigen(s), stupid” and other lessons from over a decade of vaccitherapy of human cancer. Seminars in immunology. October 2008; 20(5):296-300.
Baurain J F, Colau D, van Baren N, et al. High frequency of autologous anti-melanoma CTL directed against an antigen generated by a point mutation in a new helicase gene. J Immunol. Jun. 1, 2000; 164(11):6057-6066.
Chiari R, Foury F, De Plaen E, Baurain J F, Thonnard J, Coulie P G. Two antigens recognized by autologous cytolytic T lymphocytes on a melanoma result from a single point mutation in an essential housekeeping gene. Cancer Res. Nov. 15, 1999; 59(22):5785-5792.
Huang J, El-Gamil M, Dudley M E, Li Y F, Rosenberg S A, Robbins P F. T cells associated with tumor regression recognize frameshifted products of the CDKN2A tumor suppressor gene locus and a mutated HLA class I gene product. J Immunol. May 15, 2004; 172(10):6057-6064.
Karanikas V, Colau D, Baurain J F, et al. High frequency of cytolytic T lymphocytes directed against a tumor-specific mutated antigen detectable with HLA tetramers in the blood of a lung carcinoma patient with long survival. Cancer Res. May 1, 2001; 61(9):3718-3724.
Lennerz V, Fatho M, Gentilini C, et al. The response of autologous T cells to a human melanoma is dominated by mutated neoantigens. Proc Natl Acad Sci USA. Nov. 1, 2005; 102(44): 16013-16018.
Zorn E, Hercend T. A natural cytotoxic T cell response in a spontaneously regressing human melanoma targets a neoantigen resulting from a somatic point mutation. Eur J Immunol. February 1999; 29(2):592-601.
Kannan S, Neelapu S S. Vaccination strategies in follicular lymphoma. Current hematologic malignancy reports. October 2009; 4(4):189-195.
Kenter G G, Welters M J, Valentijn A R, et al. Phase I immunotherapeutic trial with long peptides spanning the E6 and E7 sequences of high-risk human papillomavirus 16 in end-stage cervical cancer patients shows low toxicity and robust immunogenicity. Clinical cancer research : an official journal of the American Association for Cancer Research. Jan. 1, 2008; 14(1): 169-177.
Kenter G G, Welters M J, Valentijn A R, et al. Vaccination against HPV-16 oncoproteins for vulvar intraepithelial neoplasia. The New England journal of medicine. Nov. 5, 2009; 361(19):1838-1847.
Welters M J, Kenter G G, Piersma S J, et al. Induction of tumor-specific CD4+ and CD8+ T-cell immunity in cervical cancer patients by a human papillomavirus type 16 E6 and E7 long peptides vaccine. Clinical cancer research: an official journal of the American Association for Cancer Research. Jan. 1, 2008; 14(1):178-187.
Lundegaard C, Lund O, Nielsen M. Prediction of epitopes using neural network based methods. J Immunol Methods. Nov. 30, 2011; 374(1-2):26-34.
Schreiber R D, Old L J, Smyth M J. Cancer immunoediting: integrating immunity's roles in cancer suppression and promotion. Science. Mar. 25, 2011; 331(6024):1565-1570.
Berger, M. et al. Melanoma genome sequencing reveals frequent PREX2 mutations. Nature 485, 502-6 (2012).
Carter, S L. et al. Absolute quantification of somatic DNA alterations in human cancer. Nat Biotechnol 30, 413-21 (2012).
Chapman, M. A. et al. Initial genome sequencing and analysis of multiple myeloma. Nature 471, 467-72 (2011).
Cibulskis, K. et al. ContEst: estimating cross-contamination of human samples in next-generation sequencing data. Bioinformatics 27, 2601-2 (2011).
Cibulskis, K. et al. Sensitive detection of somatic point mutations in impure and heterogeneous cancer samples. Nature Biotech (2013) February 10. doi: 10.1038/nbt.2514. [Epub ahead of print].
DeLuca, D S. et al. RNA-SeQC: RNA-seq metrics for quality control and process optimization. Bioinformatics 28, 1530-2 (2012).
DePristo, M. et al. A framework for variation discovery and genotyping using next-generation DNA sequencing data. Nature Genetics 43, 491-498 (2011).
Landau, D A. et al. Evolution and impact of subclonal mutations in chronic lymphocytic leukemia. Cell 152, 714-26 (2013).
Langmead, B. et al. Ultrafast and memory-efficient alignment of short DNA sequences to the human genome. Genome Biology 10, R25 (2009).
Li, B. et al. RSEM: accurate transcript quantification from RNA-Seq data with or without a reference genome. BMC Bioinformatics 12, 323 (2011).
Li, H. et al. The Sequence Alignment/Map format and SAMtools. Bioinformatics 25, 2078-9 (2009).
Li H. and Durbin R. Fast and accurate short read alignment with Burrows-Wheeler Transform. Bioinformatics 25, 1754-60 (2009).
McKenna, A. et al. The Genome Analysis Toolkit: a MapReduce framework for analyzing next-generation DNA sequencing data. Genome Res 20, 1297-303 (2010).
Robinson, J T. et al. Integrative genomics viewer. Nature Biotech 29, 24-26 (2011).
Stransky, N. et al. The mutational landscape of head and neck squamous cell carcinoma. Science 333, 1157-60 (2011).
Garraway, L. A. and Lander, E. S, Lessons from the cancer genome. Cell. 153, 17-37 (2013).
Lundegaard, C. et al. Prediction of epitopes using neural network based methods. J Immunol Methods. 374, 26-34 (2011).
Sette, A. et al., The relationship between class I binding affinity and immunogenicity of potential cytotoxic T cell epitopes. J Immunol. 153, 5586-5592 (1994).
Lu, Y. C. et al., Mutated regions of nucleophosmin 1PPP1R3B Is Recognized by T Cells Used To Treat a Melanoma Patient Who Experienced a Durable Complete Tumor Regression. J Immunol. 190, 6034-6042 (2013).
Sykulev, Y. et al., Evidence that a single peptide-MHC complex on a target cell can elicit a cytolytic T cell response. Immunity. 4, 565-571 (1996).
Carter, S. L. et al., Absolute quantification of somatic DNA alterations in human cancer. Nat. Biotechnology 30: 413-21 (2012).
Sidney, J. et al., HLA class I supertypes: a revised and updated classification. BMC Immunol. 9, 1 (2008).
Comprehensive genomic characterization of squamous cell lung cancers. Nature. 489, 519-525 (2012).
Ding, L. et al., Somatic mutations affect key pathways in lung adenocarcinoma. Nature. 455, 1069-1075 (2008).
Stransky, N. et al., The mutational landscape of head and neck squamous cell carcinoma. Science 333, 1157-1160 (2011).
Comprehensive molecular characterization of human colon and rectal cancer. Nature. 487, 330-337 (2012).
Comprehensive genomic characterization defines human glioblastoma genes and core pathways. Nature. 455, 1061-1068 (2008).
Integrated genomic analyses of ovarian carcinoma. Nature. 474, 609-615 (Jun. 30, 2011).
Comprehensive molecular characterization of clear cell renal cell carcinoma. Nature. 499, 43-49 (2013).
Genomic and epigenomic landscapes of adult de novo acute myeloid leukemia. N Engl J Med. 368, 2059-2074 (2013).
Comprehensive molecular portraits of human breast tumours. Nature. 490, 61-70 (2012).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

### INCORPORATION BY REFERENCE

All patents and publications mentioned in this specification are herein incorporated by reference to the same extent as if each independent patent and publication was specifically and individually indicated to be incorporated by reference.

1-39. (canceled)

40. A method of making a subject-specific neoplasia vaccine for a subject diagnosed as having a neoplasia, comprising:
(a) identifying a plurality of sequences comprising missense and/or neoORF mutations in the neoplasia, wherein identifying comprises sequencing a genome, transcriptome, or proteome of the neoplasia;
(b) ranking at least 20 neo-antigenic epitopes encoded by the plurality of sequences comprising missense and/or neoORF mutations sequences comprising the neo-antigenic mutations in an order of decreasing priority, the order comprising:
(i) a neoORF epitope that binds to an HLA of the subject with a Kd of ≤500 nM encoded by a sequence comprising a neoORF mutation,
(ii) an epitope that binds to an HLA of the subject with a Kd of ≤150 nM encoded by a sequence comprising a missense mutation, wherein the native cognate epitope has a Kd of ≥1000 nM,
(iii) an epitope that binds to an HLA of the subject with a Kd of ≤150 nM encoded by a sequence comprising a missense mutation, wherein the native cognate epitope has a Kd of ≤150 nM,
(iv) a neoORF epitope that binds to an HLA of the subject with a Kd of >500 nM encoded by a sequence comprising a neoORF mutation,
(v) an epitope that binds to an HLA of the subject with a Kd of from greater than 150 nM to 500 nM encoded by a sequence comprising a missense mutation, wherein the native cognate epitope has a Kd of from greater than 150 nM to 500 nM; and
(c) producing a subject-specific neoplasia vaccine that comprises (A) one or more polypeptides comprising at least two of five top ranked neo-antigenic epitopes encoded by the plurality of sequences comprising missense and/or neoORF mutations based on the ranking, or (B) a polynucleotide encoding one or more polypeptides comprising the at least two of five top ranked neo-antigenic epitopes of (A).

41. The method according to claim 40, wherein the subject-specific neoplasia vaccine comprises one or more polypeptides comprising at least 10 neo-antigenic epitopes encoded by the plurality of sequences comprising missense or neoORF mutations or a polynucleotide encoding the one or more polypeptides comprising at least 10 neo-antigenic epitopes encoded by the plurality of sequences comprising missense or neoORF mutations.

42. The method according to claim 40, wherein the subject-specific neoplasia vaccine comprises an RNA polynucleotide encoding the one or more polypeptides comprising the at least two of five top ranked neo-antigenic epitopes of (A).

43. The method according to claim 40, wherein each of the one or more polypeptides comprises a neo-antigenic epitope and is from 5 to 50 amino acids in length.

44. The method according to claim 40, wherein each of the one or more polypeptides comprises a neo-antigenic epitope and is from 15 to 35 amino acids in length.

45. The method according to claim 40, wherein producing the subject-specific neoplasia vaccine comprises producing two or more sub-pools, each containing a neo-antigenic epitope such that a number of individual neo-antigenic epitopes in the sub-pool targeting any single patient HLA is one or at most about five.

46. The method according to claim 45, wherein each sub-pool comprises at least two neo-antigenic epitopes selected to optimize the interactions of individual epitopes with the HLA alleles each epitope is predicted to interact with such that antigenic competition is reduced between the neo-antigenic epitopes in the same pool that bind to the same HLA allele.

47. The method according to claim 40, wherein the subject-specific neoplasia vaccine further comprises an adjuvant.

48. The method according to claim 47, wherein the adjuvant is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM, vector system, PLGA microparticles, resiquimod, SRL172, Virosomes and other Virus-like particles, YF-17D, VEGF trap, R848, beta-glucan, Pam3Cys, Aquila's QS21 stimulon, vadimezan, and AsA404 (DMXAA).

49. A method of treating a subject diagnosed as having a neoplasia with a subject-specific neoplasia vaccine, comprising:
(a) identifying a plurality of sequences comprising missense and/or neoORF mutations in the neoplasia, wherein identifying comprises sequencing a genome, transcriptome, or proteome of the neoplasia;
(b) ranking at least 20 neo-antigenic epitopes encoded by the plurality of sequences comprising missense and/or neoORF mutations sequences comprising the neo-antigenic mutations in an order of decreasing priority, the order comprising:
(i) a neoORF epitope that binds to an HLA of the subject with a Kd of ≤500 nM encoded by a sequence comprising a neoORF mutation,
(ii) an epitope that binds to an HLA of the subject with a Kd of ≤150 nM encoded by a sequence comprising a missense mutation, wherein the native cognate epitope has a Kd of ≥1000 nM,
(iii) an epitope that binds to an HLA of the subject with a Kd of ≤150 nM encoded by a sequence comprising a missense mutation, wherein the native cognate epitope has a Kd of ≤150 nM,
(iv) a neoORF epitope that binds to an HLA of the subject with a Kd of ≥500 nM encoded by a sequence comprising a neoORF mutation,
(v) an epitope that binds to an HLA of the subject with a Kd of from greater than 150 nM to 500 nM encoded by a sequence comprising a missense mutation, wherein the native cognate epitope has a Kd of from greater than 150 nM to 500 nM; and
(c) administering a subject-specific neoplasia vaccine that comprises (A) at least two of five top ranked neo-antigenic epitopes encoded by the plurality of sequences comprising missense and/or neoORF mutations based on the ranking, or (B) a polynucleotide encoding one or more polypeptides comprising the at least two of five top ranked neo-antigenic epitopes of (A).

50. The method according to claim 49, wherein the subject-specific neoplasia vaccine comprises one or more polypeptides comprising at least 10 neo-antigenic epitopes encoded by the sequences comprising missense or neoORF mutations, or a polynucleotide encoding the one or more polypeptides comprising at least 10 neo-antigenic epitopes encoded by the sequences comprising missense or neoORF mutations.

51. The method according to claim 49, wherein the subject-specific neoplasia vaccine comprises an RNA polynucleotide encoding the one or more polypeptides comprising the at least two of five top ranked neo-antigenic epitopes of (A).

52. The method according to claim 49, wherein each of the one or more polypeptides comprises a neo-antigenic epitope and is from 5 to 50 amino acids in length.

53. The method according to claim 49, wherein each of the one or more polypeptides comprises a neo-antigenic epitope and is from 15 to 35 amino acids in length.

54. The method according to claim 49, wherein administering comprises injecting two or more sub-pools into a different location of the patient, wherein each of the two or more sub-pools contains a different neo-antigenic epitope.

55. The method according to claim 54, wherein each of the sub-pools injected into a different location comprises neo-antigenic polypeptides such that a number of individual neo-antigenic epitopes in the sub-pool targeting any single patient HLA is one or at most about five.

56. The method according to claim 54, wherein each sub-pool comprises at least two neo-antigenic polypeptides selected to optimize the interactions of individual epitopes with the HLA alleles each epitope is predicted to interact with.

57. The method according to claim 56, wherein optimizing comprises reducing antigenic competition between the neo-antigenic polypeptides in the same pool that bind to the same HLA allele.

58. The method according to claim 49, wherein administering comprises administering a dendritic cell (DC) vaccine, wherein the DC vaccine comprises DCs loaded with the one or more polypeptides comprising at least two of the top five neo-antigenic epitopes encoded by the plurality of sequences comprising missense or neoORF mutations based on the ranking.
